(19) United States
(12) Patent Application Publication
(10) Pub. No.: US 2026/0092318 A1
(43) Pub. Date: Apr. 2, 2026

(54) METHODS, COMPOSITIONS, AND SYSTEMS FOR TARGET DETECTION IN SITU

(71) Applicant: Stellaromics, Inc., Boston, MA (US)

(72) Inventors: Ye FU, Belmont, MA (US); Mark PRATT, Bozeman, MT (US); Riley SHAMLOUFARD, Boston, MA (US)

(21) Appl. No.: 19/384,976

(22) Filed: Nov. 10, 2025

Related U.S. Application Data
(63) Continuation of application No. PCT/US2025/040318, filed on Aug. 1, 2025.
(60) Provisional application No. 63/678,972, filed on Aug. 2, 2024.

(51) Int. Cl. C12Q 1/6841; C12Q 1/6825; C12Q 1/6834; C12Q 1/6844

(52) U.S. Cl. CPC C12Q 1/6841; C12Q 1/6825; C12Q 1/6834; C12Q 1/6844

(57) ABSTRACT

Provided herein are methods, compositions, and systems that may be useful for the detection of one or more analytes within a biological sample. The methods may comprise providing a cell. The cell may comprise an analyte. The method may comprise contacting the cell with a binding moiety. The binding moiety may recognize and/or bind to the analyte. The method may comprise contacting the binding moiety or derivative thereof with a detection probe to form a complex. The complex may be detected using an imaging system. A signal of the complex may be detected. The signal may have a full width at half maximum.

### CROSS-REFERENCE

This application is a continuation of International Patent Application No. PCT/US2025/040318, filed Aug. 1, 2025, which claims priority to U.S. Provisional Patent Application No. 63/678,972, filed Aug. 2, 2024, which is entirely incorporated herein by reference.

### BACKGROUND

The detection of biological targets may be important for various applications, such as disease (e.g., cancer) diagnostics and the discovery of therapeutics for a disease (e.g., cancer). Such biological targets may be messenger ribonucleic acid (mRNA), deoxyribonucleic acid (DNA), or protein.

There are approaches for detecting biological targets in situ (e.g., within a biological sample, such as a cell) or ex situ (e.g., outside of the biological sample).

### SUMMARY

Aspects disclosed herein provide methods for detecting a target in situ, the method comprising: (a) providing a cell, wherein the cell comprises an analyte; (b) contacting the cell with a binding moiety, wherein the binding moiety recognizes and binds to the analyte; (c) contacting the binding moiety or derivative thereof with a detection probe to form a complex; and (d) using an imaging system to detect a signal associated with the complex and to thereby detect the analyte, wherein the signal has a full width at half maximum below 400 nanometers (nm).

In some embodiments, the diameter of the complex is less than the diffraction limit of the imaging system. In some embodiments, the diameter of the complex is more than the diffraction limit of the imaging system. In some embodiments, the analyte is within the cell. In some embodiments, the cell is within a sample. In some embodiments, the sample is a tissue sample. In some embodiments, the tissue sample is a fresh-frozen tissue sample. In some embodiments, the tissue sample is a formalin-fixed paraffin embedded tissue sample. In some embodiments, the sample is 5-250 μm thick. In some embodiments, the sample is 10-200 μm thick. In some embodiments, the sample is 25-150 μm thick. In some embodiments, the analyte comprises a nucleic acid. In some embodiments, the nucleic acid is a ribonucleic acid. In some embodiments, the ribonucleic acid is a messenger ribonucleic acid. In some embodiments, the ribonucleic acid is a ribosomal ribonucleic acid. In some embodiments, the nucleic acid is a deoxyribonucleic acid. In some embodiments, the analyte comprises a polypeptide. In some embodiments, the polypeptide comprises a protein. In some embodiments, the binding moiety comprises a nucleic acid. In some embodiments, the comprises a ribonucleic acid. In some embodiments, the nucleic acid comprises a deoxyribonucleic acid. In some embodiments, the binding moiety comprises a polypeptide. In some embodiments, the polypeptide comprises a protein. In some embodiments, the polypeptide comprises an antibody or antibody fragment. In some embodiments, the polypeptide comprises a nanobody. In some embodiments, the moiety comprises an antibody or antibody fragment conjugated to a nucleic acid. In some embodiments, the binding moiety comprises a barcode. In some embodiments, the barcode comprises a nucleic acid.

In some embodiments, after (b), the method further comprises contacting the cell with a probe. In some embodiments, the probe comprises a nucleic acid and binds to the binding moiety. In some embodiments, the method further comprises ligating the probe to form a circular nucleic acid. In some embodiments, the after (b) the method further comprises performing a ligation reaction, wherein the ligation reaction comprises ligating a nucleic acid associated with the binding moiety to form a circular nucleic acid. In some embodiments, after (b) the method further comprises performing an amplification reaction. In some embodiments, the amplification reaction comprises a rolling circle amplification reaction using the circular nucleic acid to form an amplicon. In some embodiments, the amplicon comprises a first reactive chemical moiety. In some embodiments, the first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking two copies of the first reactive chemical moiety. In some embodiments, the cross-linking comprises use of a linker. In some embodiments, the linker comprises a polyethylene glycol. In some embodiments, the linker comprises a methylene group. In some embodiments, a diameter of the amplicon is reduced after cross-linking. In some embodiments, the amplicon comprises a second reactive chemical moiety. In some embodiments, the second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking the first reactive chemical moiety and the second reactive chemical moiety. In some embodiments, the cross-linking comprises use of a linker. In some embodiments, the linker comprises a polyethylene glycol. In some embodiments, the linker comprises a methylene group. In some embodiments, the detection probe binds to the amplicon. In some embodiments, the derivative thereof comprises a reverse complement of a nucleic acid associated with the binding moiety. In some embodiments, the imaging system comprises a microscope. In some embodiments, (d) comprises imaging the cell. In some embodiments, the full width at half maximum is less than 300 nm.

Aspects disclosed herein provide methods for detecting transcripts in situ, the method comprising: (a) providing a fresh-frozen sample, wherein the fresh-frozen sample comprises a plurality of cells, and wherein the plurality of cells comprises a plurality of transcripts; (b) contacting the fresh-frozen sample with a plurality of binding moieties, wherein each binding moiety of the plurality of binding moieties recognizes and binds to a transcript of the plurality of transcripts; (c) subsequent to (b), contacting the plurality of binding moieties or derivatives thereof with a plurality of detection probes to form a plurality of complexes; and (d) detecting the plurality of complexes to thereby identify the plurality of transcripts, wherein identifying the plurality of transcripts comprises identifying more than 200 transcripts on average per cell.

In some embodiments, the fresh-frozen sample is a fresh-frozen tissue sample. In some embodiments, the fresh-frozen tissue sample is 5-250 μm thick. In some embodiments, the fresh-frozen tissue sample is 10-200 μm thick. In some embodiments, the fresh-frozen tissue sample is 25-150 μm thick. In some embodiments, a binding moiety of the plurality of binding moieties comprises a nucleic acid. In some embodiments, the nucleic acid comprises a ribonucleic acid. In some embodiments, the nucleic acid comprises a deoxyribonucleic acid. In some embodiments, a binding moiety of the plurality of binding moieties comprises a polypeptide. In some embodiments, the polypeptide comprises a protein. In some embodiments, the polypeptide comprises an antibody or antibody fragment. In some embodiments, the polypeptide comprises a nanobody. In some embodiments, a binding moiety of the plurality of binding moieties comprises an antibody or antibody fragment conjugated to a nucleic acid. In some embodiments, a binding moiety of the plurality of binding moieties comprises a barcode.

In some embodiments, the barcode comprises a nucleic acid. In some embodiments, after (b), the method further comprises contacting the fresh-frozen sample with a probe. In some embodiments, the probe comprises a nucleic acid and binds to the binding moiety. In some embodiments, the method further comprises ligating the probe to form a circular nucleic acid. In some embodiments, after (b) the method further comprises performing a ligation reaction, wherein the ligation reaction comprises ligating a nucleic acid associated with the binding moiety to form a circular nucleic acid. In some embodiments, after (b) the method further comprises performing an amplification reaction. In some embodiments, the amplification reaction comprises a rolling circle amplification reaction using the circular nucleic acid to form an amplicon. In some embodiments, the amplicon comprises a first reactive chemical moiety. In some embodiments, the first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking two copies of the first reactive chemical moiety. In some embodiments, the cross-linking comprises use of a linker. In some embodiments, the linker comprises a polyethylene glycol. In some embodiments, the linker comprises a methylene group. In some embodiments, a diameter of the amplicon is reduced after cross-linking. In some embodiments, the amplicon comprises a second reactive chemical moiety. In some embodiments, the second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking the first reactive chemical moiety and the second reactive chemical moiety. In some embodiments, the cross-linking comprises use of a linker. In some embodiments, the linker comprises a polyethylene glycol. In some embodiments, the linker comprises a methylene group. In some embodiments, the detection probe binds to the amplicon. In some embodiments, the derivative thereof comprises a reverse complement of a nucleic acid associated with the binding moiety. In some embodiments, the imaging system comprises a microscope. In some embodiments, (d) comprises imaging the cell.

Aspects disclosed herein provide methods for detecting transcripts in situ, the method comprising: (a) providing a formalin-fixed paraffin embedded sample, wherein the formalin-fixed paraffin embedded sample comprises a plurality of cells, and wherein the plurality of cells comprises a plurality of transcripts; (b) contacting the formalin-fixed paraffin embedded sample with a plurality of binding moieties, wherein each binding moiety of the plurality of binding moieties recognizes and binds to a transcript of the plurality of transcripts; (c) subsequent to (b), contacting the plurality of binding moieties or derivatives thereof with a plurality of detection probes to form a plurality of complexes; and (d) detecting the plurality of complexes to thereby identify the plurality of transcripts, wherein identifying the plurality of transcripts comprises identifying more than 120 transcripts on average per cell.

In some embodiments, the formalin-fixed paraffin embedded sample is a fresh-frozen tissue sample. In some embodiments, the formalin-fixed paraffin embedded tissue sample is 5-250 μm thick. In some embodiments, the formalin-fixed paraffin embedded tissue sample is 10-200 μm thick. In some embodiments, the formalin-fixed paraffin embedded tissue sample is 25-150 μm thick. In some embodiments, the fresh-frozen tissue sample is embedded in a hydrogel. In some embodiments, a binding moiety of the plurality of binding moieties comprises a nucleic acid. In some embodiments, the nucleic acid comprises a ribonucleic acid. In some embodiments, the nucleic acid comprises a deoxyribonucleic acid. In some embodiments, a binding moiety of the plurality of binding moieties comprises a polypeptide. In some embodiments, the polypeptide comprises a protein. In some embodiments, the polypeptide comprises an antibody or antibody fragment. In some embodiments, the polypeptide comprises a nanobody. In some embodiments, a binding moiety of the plurality of binding moieties comprises an antibody or antibody fragment conjugated to a nucleic acid. In some embodiments, a binding moiety of the plurality of binding moieties comprises a barcode.

In some embodiments, the barcode comprises a nucleic acid. In some embodiments, after (b), the method further comprises contacting formalin-fixed paraffin embedded sample with a probe. In some embodiments, the probe comprises a nucleic acid and binds to the binding moiety. In some embodiments, the method further comprises ligating the probe to form a circular nucleic acid. In some embodiments, after (b) the method further comprises performing a ligation reaction, wherein the ligation reaction comprises ligating a nucleic acid associated with the binding moiety to form a circular nucleic acid. In some embodiments, the after (b) the method further comprises performing an amplification reaction. In some embodiments, the amplification reaction comprises a rolling circle amplification reaction using the circular nucleic acid to form an amplicon. In some embodiments, the amplicon comprises a first reactive chemical moiety. In some embodiments, the first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking two copies of the first reactive chemical moiety. In some embodiments, the cross-linking comprises use of a linker. In some embodiments, the linker comprises a polyethylene glycol. In some embodiments, the linker comprises a methylene group. In some embodiments, a diameter of the amplicon is reduced after cross-linking. In some embodiments, the amplicon comprises a second reactive chemical moiety. In some embodiments, the second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking the first reactive chemical moiety and the second reactive chemical moiety. In some embodiments, the cross-linking comprises use of a linker. In some embodiments, the linker comprises a polyethylene glycol. In some embodiments, the linker comprises a methylene group. In some embodiments, the detection probe binds to the amplicon. In some embodiments, the derivative thereof comprises a reverse complement of a nucleic acid associated with the binding moiety. In some embodiments, the imaging system comprises a microscope. In some embodiments, wherein (d) comprises imaging the cell.

Aspects disclosed herein provide methods for detecting transcripts in situ, the method comprising: (a) providing a sample, wherein the sample comprises a plurality of cells, and wherein the plurality of cells comprise a plurality of transcripts; (b) contacting the sample with a plurality of binding moieties, wherein each binding moiety of the plurality of binding moieties recognizes and binds to a transcript of the plurality of transcripts; (c) subsequent to (b), contacting the plurality of binding moieties or derivatives thereof with a plurality of detection probes to form a plurality of complexes; and (d) detecting the plurality of complexes and to thereby identify the plurality of transcripts, wherein more than 10 transcripts may be detected in at least 98% of cells of the plurality of cells.

In some embodiments, the sample is a tissue sample. In some embodiments, the tissue sample is 5-250 μm thick. In some embodiments, the tissue sample is 10-200 μm thick. In some embodiments, the tissue sample is 25-150 μm thick. In some embodiments, the tissue sample is embedded in a hydrogel. In some embodiments, a binding moiety of the plurality of binding moieties comprises a nucleic acid. In some embodiments, the nucleic acid comprises a ribonucleic acid. In some embodiments, the nucleic acid comprises a deoxyribonucleic acid. In some embodiments, a binding moiety of the plurality of binding moieties comprises a polypeptide. In some embodiments, the polypeptide comprises a protein. In some embodiments, the polypeptide comprises an antibody or antibody fragment. In some embodiments, a binding moiety of the plurality of binding moieties comprises an antibody or antibody fragment conjugated to a nucleic acid. In some embodiments, a binding moiety of the plurality of binding moieties comprises a barcode.

In some embodiments, the barcode comprises a nucleic acid. In some embodiments, after (b), the method further comprises contacting formalin-fixed paraffin embedded sample with a probe. In some embodiments, the probe comprises a nucleic acid and binds to the binding moiety. In some embodiments, the method further comprises ligating the probe to form a circular nucleic acid. In some embodiments, after (b) the method further comprises performing a ligation reaction, wherein the ligation reaction comprises ligating a nucleic acid associated with the binding moiety to form a circular nucleic acid. In some embodiments, after (b) the method further comprises performing an amplification reaction. In some embodiments, the amplification reaction comprises a rolling circle amplification reaction using the circular nucleic acid to form an amplicon. In some embodiments, the amplicon comprises a first reactive chemical moiety. In some embodiments, the first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking two copies of the first reactive chemical moiety. In some embodiments, the cross-linking comprises use of a linker. In some embodiments, the linker comprises a polyethylene glycol. In some embodiments, the linker comprises a methylene group. In some embodiments, a diameter of the amplicon is reduced after cross-linking. In some embodiments, the amplicon comprises a second reactive chemical moiety. In some embodiments, the second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method further comprises cross-linking the first reactive chemical moiety and the second reactive chemical moiety. In some embodiments, the cross-linking comprises use of a linker. In some embodiments, the linker comprises a polyethylene glycol. In some embodiments, the linker comprises a methylene group. In some embodiments, the detection probe binds to the amplicon. In some embodiments, the derivative thereof comprises a reverse complement of a nucleic acid associated with the binding moiety. In some embodiments, the imaging system comprises a microscope. In some embodiments, (d) comprises imaging the cell.

Aspects disclosed herein provide methods for detecting an analyte in situ, the method comprising: (a) providing a cell, wherein the cell comprises the analyte; (b) contacting the cell with a probe, wherein the probe recognizes and binds to the analyte; (c) amplifying the probe to generate an amplicon, wherein the amplicon comprises a first reactive chemical moiety and a second reactive chemical moiety; (d) incubating the amplicon under conditions sufficient to form a conjugate between the first reactive chemical moiety and the second reactive chemical moiety; and (e) detecting the amplicon to thereby detect the analyte.

In some embodiments, the analyte is within the cell. In some embodiments, the analyte is on a surface of the cell. In some embodiments, the cell is within a sample. In some embodiments, the sample is a tissue sample. In some embodiments, the tissue sample is a fresh-frozen tissue sample. In some embodiments, the tissue sample is a formalin-fixed paraffin embedded tissue sample. In some embodiments, the tissue sample is 5-250 μm thick. In some embodiments, the tissue sample is 10-200 μm thick. In some embodiments, the tissue sample is 25-150 μm thick. In some embodiments, the analyte comprises a nucleic acid. In some embodiments, the nucleic acid is a ribonucleic acid. In some embodiments, the ribonucleic acid is a messenger ribonucleic acid. In some embodiments, the ribonucleic acid is a ribosomal ribonucleic acid. In some embodiments, the nucleic acid is a deoxyribonucleic acid. In some embodiments, the analyte comprises a polypeptide. In some embodiments, the polypeptide comprises a protein. In some embodiments, the probe comprises a nucleic acid. In some embodiments, the nucleic acid comprises a ribonucleic acid. In some embodiments, the nucleic acid comprises a deoxyribonucleic acid. In some embodiments, the probe comprises a polypeptide. In some embodiments, the polypeptide comprises a protein. In some embodiments, the polypeptide comprises an antibody or antibody fragment. In some embodiments, the polypeptide comprises a nanobody. In some embodiments, the probe comprises an antibody or antibody fragment conjugated to a nucleic acid. In some embodiments, the probe comprises a barcode. In some embodiments, the barcode comprises a nucleic acid. In some embodiments, the amplifying comprises performing rolling circle amplification. In some embodiments, the first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the conjugate comprises a linker. In some embodiments, the linker comprises a polyethylene glycol. In some embodiments, the linker comprises a methylene group.

Aspects disclosed herein provide methods comprising identifying at least 100 individual targets within a cell of a plurality of cells in situ at an accuracy of at least 80% in a time period of at most 2 days, wherein the plurality of cells comprises at least one million cells.

Aspects disclosed herein provide methods comprising: (a) generating a plurality of volumetric images of a plurality of cells; and (b) processing the plurality of volumetric images to identify at least 500 targets in a cell of the plurality of cells at an accuracy of at least 80%.

Aspects disclosed herein provide methods comprising: (a) generating a plurality of volumetric images of a plurality of cells; and (b) processing the plurality of volumetric images to identify at least 100 targets in a cell of the plurality of cells at an accuracy of at least 80%, wherein the plurality of cells comprises at least one million cells.

Aspects disclosed herein provide methods comprising of detecting analytes in a sample, the methods comprising: (a) providing the sample, wherein the sample comprises a first analyte and a second analyte; (b) contacting the first analyte with a first binding agent, wherein the first binding agent comprises a barcode; (c) contacting the sample with a compaction agent, wherein the compaction agent binds to the barcode or reverse complement thereof; and (d) detecting a reverse complement of the barcode with greater than 90% accuracy, wherein the reverse complement of the barcode is produced only when the first analyte is proximal to the second analyte.

Aspects disclosed herein provide methods for detecting an analyte in situ, the method comprising: (a) providing a cell, wherein the cell comprises an analyte; (b) contacting the cell with a binding moiety, wherein the binding moiety binds to the analyte to form a first binding complex; (c) contacting the first binding complex or derivative thereof with a detection probe to form a second binding complex comprising the first binding complex and the detection probe; and (d) using an imaging system to detect a signal associated with the second binding complex and to thereby detect the analyte, wherein the signal has diameter less than 200 nanometers (nm).

Aspects disclosed herein provide methods for detecting an analyte in situ, the method comprising: (a) providing a cell, wherein the cell comprises an analyte; (b) contacting the cell with a probe, wherein the probe binds to an analyte or the analyte; (c) amplifying the probe to generate an amplicon; (d) contacting the amplicon with a compaction reagent, wherein the compaction reagent is configured to form one or more covalent interactions with the amplicon; and (e) using an imaging system to detect a signal, wherein the signal is associated with the amplicon, wherein the signal comprises a roundness value, and wherein the roundness value is 1.1 or less.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a computer system that is programmed or otherwise configured to implement methods provided herein.

FIG. 2 shows a schematic of an amplicon comprising azide groups and reacting with an alkyne containing linker to form a compacted amplicon, according to some embodiments of the present disclosure.

FIG. 3 shows a schematic of an amplicon comprising azide and alkyne groups and the azide and alkyne groups reacting with each other via proximity-mediated click reactions to form a compacted amplicon, according to some embodiments of the present disclosure.

FIG. 4 shows a schematic of an amplicon comprising azide and alkyne groups with a condensing oligonucleotide and the azide and alkyne groups reacting with each other via proximity-mediated click reactions to form a compacted amplicon, according to some embodiments of the present disclosure.

FIG. 5 shows a schematic of an amplicon comprising alkyne groups and reacting with an azide containing linker to form a compacted amplicon, according to some embodiments of the present disclosure.

FIG. 6 shows a schematic of an amplicon comprising azide and alkyne groups or a hydrophobic DBCO group with a condensing oligonucleotide, according to some embodiments of the present disclosure. The azide and alkyne groups react with each other via proximity-mediated click reactions or DBCO and undergo hydrophobic stacking to form a compacted amplicon.

FIG. 7 shows amplicon size and shape refinement of amplicons comprising alkyne groups that react with an azide-containing crosslinker to form a compacted amplicon in 20 μm mouse brain tissue slices probed with 250 gene panel probes, according to some embodiments of the present disclosure. The amplicons were embedded in hydrogel without covalent crosslinking to the hydrogel.

FIG. 8 shows amplicon size and shape refinement of amplicons comprising DBCO-alkyne groups and azide groups that form a compacted amplicon in 20 μm mouse brain tissue slices probed with 250 gene panel probes, according to some embodiments of the present disclosure. The amplicons were embedded in hydrogel without covalent crosslinking to the hydrogel. 5 rounds of sequencing were performed showing a majority of the amplicons were retained in hydrogel without chemical crosslinking to the hydrogel.

FIG. 9 shows significantly increased signal intensities of amplicons comprising hydrophobic DBCO groups or DBCO and Azide groups that form a compacted amplicon, according to some embodiments of the present disclosure.

FIGS. 10A-10D show schematic drawings of example microscopy systems for use as described herein. FIG. 10A shows a schematic drawing of an example upright microscopy system for use as described herein. FIG. 10B shows a schematic drawing of an example inverted microscopy system with a confocal filter for use as described herein. FIG. 10C shows a schematic drawing of an example inverted microscopy system for use as described herein. FIG. 10D shows a schematic drawing of an example inverted microscopy system where a sample is held or provided in a flow cell for use as described herein.

FIG. 11 shows an example schematic drawing of a path of movement of the objective lens for imaging of a sample, according to some embodiments of the present disclosure.

FIG. 12 shows an example schematic drawing of a z-stack image comprising multiple object planes in one field of view, according to some embodiments of the present disclosure.

FIG. 13 shows an example schematic drawing of z-stack images comprising multiple adjacent fields of view, according to some embodiments of the present disclosure.

FIG. 14 shows a flowchart of a method of determining a property of a plurality of cells, according to some embodiments of the present disclosure.

FIG. 15 shows a flowchart of a method of imaging a plurality of cells, according to some embodiments of the present disclosure.

FIG. 16 shows a flowchart of a method, according to some embodiments of the present disclosure.

FIG. 17 shows a flowchart of a method of taking a volume video of a sample, according to some embodiments of the present disclosure.

FIG. 18 shows a schematic of the sample stage and objective lens, according to some embodiments of the present disclosure.

FIG. 19 shows a schematic of confocal microscopy system, according to some embodiments of the present disclosure.

FIGS. 20A-20C show schematics of binding moieties contacting a sample, generating an amplicon, and the amplicon being compacted.

FIGS. 21A-21I show schematics of binding moieties or binding moieties and probes.

FIG. 22 shows a schematic for generating a compacted amplicon.

FIG. 23 shows the average FWHM of amplicons across different fluorescent channels, with each data point representing the average FWHM of all amplicons in one field of view.

FIG. 24 shows the average FWHM of amplicons organized by fluorescent intensity bins for a single fluorescence channel.

FIG. 25 shows the average FWHM of amplicons organized by fluorescent intensity bins for a single fluorescence channel.

FIG. 26 shows the average FWHM of amplicons organized by fluorescent intensity bins for a single fluorescence channel.

FIG. 27 shows the average FWHM of amplicons organized by fluorescent intensity bins for a single fluorescence channel.

FIG. 28 shows the number of amplicons identified in a field of view across different fluorescent channels.

FIG. 29 shows the number of identified (decoded) transcripts in samples with different experimental conditions.

FIG. 30 shows traces of absorbance measurements over time for different experimental conditions.

FIG. 31 shows a schematic of amplicon hydrogel embedding.

FIG. 32 shows a schematic of amplicon generation.

FIG. 33 shows a schematic of amplicon generation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

As used herein, the term “confocal” can refer to an optical imaging system having an intermediate focal plane comprising one or more spatial filters (pin holes) used to control inbound illumination and outbound scattered or fluorescent light. The spatial filters may be used to reject out of object plane light.

As used herein, the term “duty cycle” can refer to the fraction of time, often over a cycle, spent doing useful work. In this context, duty cycle may refer to the fraction of a data acquisition cycle spent integrating, e.g., collecting (integrating) photons. In the larger context of the instrument, it may refer to the fraction of run time spent acquiring data or more specifically collecting photons.

As used herein, the term “frame” can refer to an image in a video or a z-stack.

As used herein, the term “frame rate” can refer to the number of frames acquired per unit time, typically reported in Hz. In high duty cycle imaging, the integration time may be roughly the reciprocal of the frame rate.

As used herein, the term “image” can refer to a representation of the sample. In some cases, the image does not have to be a visual representation. The image can be a digital representation, e.g., comprising signals and coordinates of signals. The signals can be a number of photon-electrons captured at a given wavelength over a given period of time. The coordinates can be coordinates with respect to the sample given a mathematical transform accounting for the imaging system, e.g., magnifications and reflections.

The image may refer to 2-dimensional data collected on a sensor. Typically, an optical system may focus an object on the sensor where the image is collected in the form of photoelectrons. If the optical system is static, the focused image may comprise a representation of a thin slice of the object (object plane) within the depth-of-focus of the system. If the sample and optical system are in relative z-motion, then the image may comprise a representation of a slice of the object whose depth includes the depth-of-focus, and the relative z-distance traveled during the integration time (the time during which photoelectrons are captured). The resulting 2-dimensional data may comprise pixels integrated over the same time interval (global shutter), or pixels integrated over staggered and overlapping intervals (rolling shutter). Image (the verb) can refer to a process of acquiring an image (the noun).

As used herein, the term “integration” in the context of imaging can refer to a collection or accumulation of photons. For a single frame, this may comprise the time a physical shutter is open or the time that an object is illuminated. Electronic shutters (global or rolling) may also define the time interval, although in the latter case the interval is time shifted throughout the image.

As used herein, the term “object plane” or “object surface” can refer to the surface or slice within the object (sample e.g.) corresponding to a two-dimensional image. If the image is acquired with global shutter, the object plane or slice may be orthogonal to the optical axis in the sample. If the image is acquired with rolling shutter, the object plane may correspond to one (or more) planes slightly skew to the optical axis in the sample. In some cases, the image may be divided into blocks of rows, each of which may read out in parallel, thereby creating multiple planes. These multiple planes may comprise a sawtooth arrangement. Rolling shutter systems may subdivide the sensor into zones and may be read out in parallel. Each zone may comprise a plane in this context. Generally, an image may correspond to a depth about an object surface, where the depth comprises a depth of focus in addition to any motion of the of the system during integration. Acceleration during acquisition may create non-planar object surfaces.

As used herein, the term “rolling shutter” can refer to a method of image capture in which a still picture (in a still camera) or each frame of a video (in a video camera) is captured not by taking a snapshot of the entire scene at a single instant in time but rather by scanning across the scene rapidly, vertically, horizontally, or rotationally. In other words, not all parts of the image of the scene may be recorded over the same time interval. However, during playback, the entire image of the scene may be displayed at once, as if it represents a single instant in time. This may produce predictable distortions of fast-moving objects or rapid flashes of light. This is in contrast with “global shutter”, in which the entire frame may be integrated over the same interval.

As used herein, the term “skew angle” can refer to the deviation of a rolling shutter object plane from perpendicular to the optical axis. For continuous integration, this may amount to one z voxel over the image row width, typically thousands of pixels.

As used herein, the term “tissue” can refer to one or more portions of an organism comprising cells, optionally including a natural or artificial extracellular matrix. The tissue can be chemically or physically modified from its native state, including by clearing opaque matter and stabilizing with hydrogel. In some cases, tissue may refer to a group of epithelial cells, muscle cells, or nerve cells. Tissue may include tissue collected from any subject. For example, tissue may be collected from a biopsy or an autopsy. In some instances, a tissue may be visualized or imaged immediately or may be stored prior to analysis.

As used herein, the term “voxel” can refer to a 3-dimensional sample of a volume. Typically, this refers to a pixel in a 3-dimensional image stack. The size of a voxel may be set in the x-y plane by the size of the sensor pixel and the transverse magnification of the optical system. The z-size of a voxel may be set by one or more of the depths of focus, the z-separation of images in an image stack, or the z-distance traveled during image integration.

Provided herein are methods, systems, compositions, or kits that may be useful for the detection of one or more targets within a biological sample. The detection of the one or more targets within a biological sample may comprise compacting amplified material used for detecting signals from one or more targets of the biological sample. Compared to bulk measurements, the methods described herein may provide information regarding both the identity of the one or more targets and its spatial context. Bulk measurements may comprise measurements acquired in a sample comprising a mixture of components. The sample comprising a mixture of components may not comprise individual cells (e.g., the sample comprising a mixture of components may comprise components of individual cells mixed together). Additionally, compared to other in situ detection technologies, the methods describe herein may enable high-throughput detection of numerous targets with high accuracy.

### Methods

An aspect of the present provides a method for identifying a plurality of analytes (e.g., at least 100 individual analytes). The plurality of analytes may be within a cell of a plurality of cells. The identifying of the plurality of analytes (e.g., at least 100 individual analytes) may be performed in situ (e.g., within a sample). The identifying of the plurality of analytes (e.g., at least 100 individual analytes) may be performed with an accuracy of at least 70%. The identifying of the plurality of analytes (e.g., at least 100 individual analytes) may be performed within 4 days. The plurality of cells may comprise at least one million cells.

Another aspect of the disclosure provides a method for detecting analytes. The method may comprise detecting the analytes in situ. The method may comprise identifying greater than or equal to 1, 2, 3, 4, 5, 6, 7, 8, 9, 10, 20, 30, 40, 50, 60, 70, 80, 90, 100, 200, 300, 400, 500, 600, 700, 800, 900, or 1000 individual targets (e.g., different targets) within a cell of a plurality of cells (e.g., in situ). Identifying the individual targets with the cell may be achieved at an accuracy of greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. Identifying the individual targets with the cell may be achieved in a time period of less than or equal to 3 days or 2 days or 1 day, wherein the plurality of cells comprises greater or equal to 1, 2, 3, 4, 5, 6, 7, 8, 9, 10, 20, 30, 40, or 50 million cells.

In some aspects, the methods described herein may comprise generating one or more amplicons associated with the individual targets. The one or more amplicons may be compacted using any one of the methods described herein. Compacting the one or more amplicons may enable a higher-throughput detection of the one or more amplicons, a faster acquisition of images associated with the one or more amplicons, or a combination thereof. For example, compacting one or more amplicons may generate brighter fluorescence signals of the one or more amplicons. Brighter fluorescence signals of the one or more amplicons may enable faster imaging (e.g., shorter exposure times). In some cases, compacting the one or more amplicons may enable detecting more analytes in a unit area. For example, a unit area may comprise a cell of the plurality of cells. The cell may comprise analytes of the plurality of individual analytes. Compacting the one or more amplicons of the sample may enable detecting more analytes of the plurality of individual analytes as compared to the number of analytes that may be detected without compacting the one or more amplicons.

In some aspects, the methods described herein may comprise imaging the cell using an imaging system. The imaging system may comprise an objective lens configured to transmit photons from one or more object planes within the cell to one or more sensors. The methods described herein may comprise moving the objective lens relative to the cell while simultaneously using the imaging module to acquire a series of images corresponding to a plurality of object planes within the cell. Imaging the cell using the imaging system as described herein may provide certain advantages. For example, imaging the cell using the imaging system as described herein may contribute to the accuracy of identifying the individual analytes within a period of time (e.g., 100 individual analytes with an accuracy of 80%).

In some cases, the plurality of individual analytes (e.g., at least 100 individual analytes) may be identified using any one of the methods described herein. For example, a tissue sample may be obtained. The tissue sample may comprise the plurality of individual analytes (e.g., at least 100 individual analytes). The sample may be contacted with binding moieties, as described herein. The binding moieties may recognize and/or bind to the plurality of individual analytes (e.g., at least 100 individual analytes). The binding moieties may be used to generate amplicons. For example, the binding moieties may bind to probes. The probes may be ligated to form circular nucleic acids. The circular nucleic acids may be amplified using rolling circle amplification (RCA). The binding moieties may be used as primers for the RCA. The RCA may generate amplicons. The amplicons may be detected using an imaging system to identify the plurality of individual analytes (e.g., at least 100 individual analytes). The imaging system may comprise any one of the imaging systems described herein.

In some cases, the plurality of individual analytes (e.g., at least 100 individual analytes) may comprise at least 100 analytes of different types. In some cases, different types may refer to different types of biological entities (e.g. polypeptides may be one type of biological entity and nucleic acids may be another type of biological entity). For example, the plurality of individual analytes (e.g., at least 100 individual analytes) may comprise at least 100 different messenger ribonucleic acids that each comprise a unique sequence. In some cases, multiple copies of the same analyte may be detected. In some cases, multiple copies of the same analyte may be considered the same individual analyte. In some cases, the plurality of individual analytes (e.g., at least 100 individual analytes) may comprise more than 100 individual analytes. In some cases, the plurality of individual analytes (e.g. at least 100 individual analytes) may comprise at least about 100 individual analytes, at least about 125 individual analytes, at least about 150 individual analytes, at least about 200 individual analytes, at least about 250 individual analytes, at least about 300 individual analytes, at least about 400 individual analytes, at least about 500 individual analytes, at least about 600 individual analytes, at least about 700 individual analytes, at least about 800 individual analytes, at least about 900 individual analytes, at least about 1000 individual analytes, at least about 5000 individual analytes, at least about 10000 individual analytes, or more. In some cases, the plurality of individual analytes (e.g. at most 100 individual analytes) may comprise at most about 100 individual analytes, at most about 125 individual analytes, at most about 150 individual analytes, at most about 200 individual analytes, at most about 250 individual analytes, at most about 300 individual analytes, at most about 400 individual analytes, at most about 500 individual analytes, at most about 600 individual analytes, at most about 700 individual analytes, at most about 800 individual analytes, at most about 900 individual analytes, at most about 1000 individual analytes, at most about 5000 individual analytes, at most about 10000 individual analytes, or more. In some cases, the plurality of individual analytes (e.g., at most 100 individual analytes) may comprise about 100-10000 individual analytes, about 125-5000 individual analytes, about 150-1000 individual analytes, about 200-900 individual analytes, about 250-800 individual analytes, about 300-700 individual analytes, or about 400-600 individual analytes.

Identifying the plurality of individual analytes (e.g., at least about 100 individual analytes) may be performed within about 2 days using any one of the methods described herein. The time duration of any one of the methods described herein may comprise the time duration of obtaining a sample, contacting the sample with one or more moieties (e.g., one or more binding moieties and/or one or more probes), performing a ligation reaction, performing an amplification reaction, performing a cross-linking reaction, preparing a hydrogel of the sample, detecting signals associated with one or more amplicons using an imaging system, performing multiple imaging cycles, processing image data, comparing image data to a codebook, or any combination thereof. In some cases, the time duration of any one of the methods described herein may comprise the time associated with performing imaging, image processing, or a combination thereof. In some cases, the time duration of any one of the methods described herein may comprise the time associated with obtaining a sample, processing a sample (e.g., generating amplicons on the sample), imaging the sample, processing image data, or a combination thereof. Identifying the plurality of individual analytes (e.g. at least 100 individual analytes) may be performed within less than about 2 days, less than about 48 hours, less than about 44 hours, less than about 40 hours, less than about 35 hours, less than about 30 hours, less than about 24 hours, less than about 20 hours, less than about 15 hours, less than about 12 hours, less than about 10 hours, less than about 8 hours, less than about 6 hours, less than about 4 hours, less than about 2 hours, less than about 1 hour, or less. Identifying the plurality of individual analytes (e.g. at least 100 individual analytes) may be performed within more than about 2 days, more than about 48 hours, more than about 44 hours, more than about 40 hours, more than about 35 hours, more than about 30 hours, more than about 24 hours, more than about 20 hours, more than about 15 hours, more than about 12 hours, more than about 10 hours, more than about 8 hours, more than about 6 hours, more than about 4 hours, more than about 2 hours, more than about 1 hour, or more. Identifying the plurality of individual analytes (e.g., at least 100 individual analytes) may be performed within about 1-48 hours, about 2-40 hours, about 4-35 hours, about 8-30 hours, about 12-24 hours, or about 15-20 hours.

Another aspect of the disclosure provides a method for processing a plurality of volumetric images. The method may comprise generating a plurality of volumetric images. The volumetric images may be of a plurality of cells. The method may comprise processing the plurality of volumetric images to identify a plurality of analytes (e.g., at least 500 analytes) in a cell of the plurality of cells. Identifying the plurality of analytes (e.g., at least 500 analytes) in a cell of the plurality of cells may be performed with an accuracy of at least 80%.

Another aspect of the disclosure provides a method for detecting analytes. The analytes may be detected in situ. The method may comprise generating a plurality of volumetric images of a plurality of cells. The plurality of volumetric images may be processed to identify greater or equal to 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 analytes (e.g., different analytes) in a cell of the plurality of cells at an accuracy of greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments, the plurality of volumetric images may be processed to identify greater or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, greater than or equal to 100, greater than or equal to 200, greater than or equal to 300, greater than or equal to 400, greater than or equal to 500, greater than or equal to 600, greater than or equal to 700, greater than or equal to 800, greater than or equal to 900, greater than or equal to 1000, greater than or equal to 1100, greater than or equal to 1200, greater than or equal to 1300, greater than or equal to 1400, greater than or equal to 1500, greater than or equal to 1600, greater than or equal to 1700, greater than or equal to 1800, greater than or equal to 1900, or greater than or equal to 2000 targets (e.g., different targets) in a cell of the plurality of cells. In some embodiments, the accuracy of greater than or equal to 60%, greater than or equal to 61%, greater than or equal to 62%, greater than or equal to 63%, greater than or equal to 64%, greater than or equal to 65%, greater than or equal to 66%, greater than or equal to 67%, greater than or equal to 68%, greater than or equal to 69%, greater than or equal to 70%, greater than or equal to 71%, greater than or equal to 72%, greater than or equal to 73%, greater than or equal to 74%, greater than or equal to 75%, greater than or equal to 76%, greater than or equal to 77%, greater than or equal to 78%, greater than or equal to 79%, greater than or equal to 80%, greater than or equal to 81%, greater than or equal to 82%, greater than or equal to 83%, greater than or equal to 84%, greater than or equal to 85%, greater than or equal to 86%, greater than or equal to 87%, greater than or equal to 88%, greater than or equal to 89%, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100%.

The volumetric images may be images of three-dimensional (3D) information from a volume of space. The volumetric images may include high spatial resolution in all directions. In some embodiments, the volumetric images comprise obtaining 2D images in sequence. In some embodiments, the volumetric images are obtained from scanned-focus techniques. In some embodiments, the volumetric images are obtained from multi-focus techniques. In some embodiments, the volumetric images are obtained from extended-focus techniques.

The volumetric images may comprise data related to in situ detection of targets within the plurality of cells. The data related to in situ detection of targets within the plurality of cells may be generated by contacting the plurality of cells with one or more binding moieties, as described herein. The plurality of cells may be contacted with one or more probes. The one or more probes may bind to the one or more binding moieties. The one or more probes may comprise a nucleic acid. The one or more probes may be ligated to form one or more circular nucleic acids. A rolling circle amplification reaction may be performed using the one or more circular nucleic acids to generate one or more amplicons. The one or more amplicons may be compacted using one or more compaction agents. The one or more amplicons may be contacted with one or more detection probes. The plurality of cells may be imaged to detect data associated with the one or more detection probes of the one or more amplicons.

In some aspects, the methods described herein may comprise generating one or more amplicons associated with the analytes (e.g., at least 500 analytes). The one or more amplicons may be compacted using any one of the methods described herein. Compacting the one or more amplicons may enable a higher-throughput detection of the one or more amplicons, a faster acquisition of images associated with the one or more amplicons, or a combination thereof. In some cases, compacting the one or more amplicons may enable detecting more analytes of the plurality of analytes (e.g., at least 500 analytes) in a unit area. For example, a unit area may comprise a cell of the plurality of cells. The cell may comprise analytes of the plurality of individual analytes. Compacting the one or more amplicons of the sample may enable detecting more analytes of the plurality of individual analytes as compared to the number of analytes that may be detected without compacting the one or more amplicons.

In some aspects, the methods described herein may comprise imaging the plurality of cells using an imaging system. The imaging system may comprise an objective lens configured to transmit photons from one or more object planes within the plurality of cells to one or more sensors. The methods described herein may comprise moving the objective lens relative to the cell while simultaneously using the imaging module to acquire a series of images corresponding to a plurality of object planes within the plurality of cells. Imaging the cell using the imaging system as described herein may provide certain advantages. For example, imaging the plurality of cells using the imaging system as described herein may contribute to the accuracy of identifying the analytes within a period of time (e.g., at least 500 analytes with an accuracy of 80%).

In some cases, the plurality of individual analytes (e.g., at least 500 individual analytes) may be identified using any one of the methods described herein. For example, a tissue sample may be obtained. The tissue sample may comprise the plurality of analytes (e.g., at least 500 analytes). The sample may be contacted with binding moieties, as described herein. The binding moieties may recognize and/or bind to the plurality of analytes (e.g., at least 500 analytes). The binding moieties may be used to generate amplicons. For example, the binding moieties may bind to probes. The probes may be ligated to form circular nucleic acids. The circular nucleic acids may be amplified using rolling circle amplification (RCA). The binding moieties may be used as primers for the RCA. The RCA may generate amplicons. The amplicons may be detected using an imaging system to identify the plurality of analytes (e.g., at least 500 analytes). The imaging system may comprise any one of the imaging systems described herein.

The plurality of analytes (e.g., at least 500 analytes) may comprise one or more of the analytes described herein. In some cases, the plurality of analytes (e.g., at least 500 analytes) may comprise multiple copies of the same analyte (e.g., multiple copies of the messenger RNA for actin beta). In some cases, the plurality of analytes (e.g., at least 500 analytes) may comprise different analytes (e.g., mRNA comprising sequences for different transcripts). The plurality of analytes may comprise at least 500 analytes may comprise at least about 500 analytes, at least about 600 analytes, at least about 700 analytes, at least about 800 analytes, at least about 900 analytes, at least about 1000 analytes, at least about 5000 analytes, at least about 10000 analytes, at least about 50000 analytes, at least about 100000 analytes, at least about 500000 analytes, at least about 1000000 analytes, or more. The plurality of analytes may comprise at most 500 analytes may comprise at most about 500 analytes, at most about 600 analytes, at most about 700 analytes, at most about 800 analytes, at most about 900 analytes, at most about 1000 analytes, at most about 5000 analytes, at most about 10000 analytes, at most about 50000 analytes, at most about 100000 analytes, at most about 500000 analytes, at most about 1000000 analytes, or less. The plurality of analytes may comprise about 500-1000000 analytes, about 600-500000 analytes, about 700-100000 analytes, about 800-50000 analytes, about 900-10000 analytes, or about 1000-5000 analytes.

Another aspect of the disclosure provides a method for detecting an analyte in situ. The method may comprise providing a cell. The cell may comprise an analyte. The cell may be contacted with a binding moiety. The binding moiety may bind to the analyte to form a first binding complex. The first binding complex or derivative thereof may be contacted with a detection probe to form a second binding complex comprising the first binding complex and the detection probe. An imaging system may be used to detect a signal associated with the second binding complex and to thereby detect the analyte. The signal may have diameter less than or equal to 1000, 900, 800, 700, 600, 500, 400, 300, 200, 100, 90, 80, 70, 60, 50, 40, 30, 20, 10, 9, 8, 7, 6, 5, 4, 3, 2, or 1 nm.

In some embodiments, the diameter may be less than or equal to 1000 nm, less than or equal to 900 nm, less than or equal to 800 nm, less than or equal to 700 nm, less than or equal to 600 nm, less than or equal to 500 nm, less than or equal to 400 nm, less than or equal to 300 nm, less than or equal to 200 nm, less than or equal to 100 nm, less than or equal to 90 nm, less than or equal to 80 nm, less than or equal to 70 nm, less than or equal to 60 nm, less than or equal to 50 nm, less than or equal to 40 nm, less than or equal to 30 nm, less than or equal to 20 nm, less than or equal to 10 nm, less than or equal to 9 nm, less than or equal to 8 nm, less than or equal to 7 nm, less than or equal to 6 nm, less than or equal to 5 nm, less than or equal to 4 nm, less than or equal to 3 nm, less than or equal to 2 nm, less than or equal to 1 nm, from 0.1 nm to 1000 nm, from 1 nm to 900 nm, from 2 nm to 800 nm, from 3 nm to 700 nm, from 4 nm to 600 nm, from 5 nm to 500 nm, from 6 nm to 400 nm, from 7 nm to 300 nm, from 8 nm to 200 nm, from 9 nm to 100 nm, from 10 nm to 90 nm, from 20 nm to 80 nm, from 30 nm to 70 nm, from 40 nm to 60 nm.

The method for detecting an analyte in situ may comprise contacting a cell with a binding moiety. The binding moiety may comprise a nucleic acid, a polypeptide, or a combination thereof. In some cases, the binding moiety may comprise an antibody or antibody fragment. The antibody or antibody fragment may comprise a nucleic acid. The nucleic acid of the antibody or antibody fragment may be conjugated to it. The binding moiety may bind to an analyte within the cell to corm a binding complex. For example, the binding moiety may comprise a nucleic acid and binding to the analyte may comprise hybridizing the nucleic acid of the binding moiety to the analyte. The analyte may comprise a nucleic acid, a polypeptide, a signaling molecule, a lipid, or a combination thereof. In some cases, the binding moiety may be amplified to generate one or more amplicons. For example, the binding moiety may comprise a nucleic acid that hybridizes to the analyte. The one end of the nucleic acid of the binding moiety may be ligated to another end of the nucleic acid of the binding moiety to form a circular nucleic acid. The circular nucleic acid may be amplified using rolling circle amplification to generate one or more amplicons. In some other cases, the binding moiety may be contacted by a probe. The probe may be amplified to form one or more amplicons. The one or more amplicons may comprise a derivative of the binding moiety and or binding complex. The one or more amplicons may be contacted with a detection probe. The detection probe may bind to the one or more amplicons to create a binding complex (e.g., a second binding complex). The detection probe may comprise a nucleic acid, a polypeptide, or a combination thereof. In some cases, the detection probe may comprise a label. A signal associated with the label of the binding complex (e.g., a second binding complex) may be detected using an imaging system. The imaging system may comprise a microscope, a scanner, or a combination thereof. In some cases, the signal associate with the binding complex comprising the detection probe may be used to identify and thereby detect an analyte. For example, the signal of the label, which binds to the binding moiety, may correspond to the analyte. In some cases, one or more signals associate with one or more detection probes may be detected and the signal of the one or more detection probes may correspond to the analyte.

Another aspect of the disclosure provides a method for detecting an analyte in situ. The method may comprise providing a cell. The cell may comprise an analyte. The cell may be contacted with a probe. A probe of the plurality of probes may bind to the analyte. The probe may be amplified to generate an amplicon. The amplicon may be contacted with a compaction agent (e.g., compaction reagent). The compaction agent may be configured to form one or more covalent interactions with the amplicon. An imaging system may be used to detect a signal. The signal may be associated with the amplicon. The signal of the amplicon may comprise a roundness value. The roundness value may be less than or equal to 10, 9, 8, 7, 6, 5, 4, 3, 2, 1.5, 1.1, 1, 0.9, 0.8, 0.7, 0.6, 0.5, 0.4, 0.3, 0.2, or 0.1.

The methods described herein related to detecting an analyte in situ may comprise contacting a cell with a probe. The cell may comprise an analyte. The analyte of the cell may comprise a nucleic acid, a polypeptide, a small molecule, or a combination thereof. The probe may comprise a nucleic acid, a polypeptide, or a combination thereof. In some cases, the probe may comprise an antibody or antibody fragment. The antibody or antibody fragment of the probe may comprise a nucleic acid. The probe may bind to the analyte. For example, the probe may comprise a nucleic acid and the nucleic acid of the probe may hybridize to the analyte. The probe may be ligated using a ligase. For example, the probe may comprise a nucleic acid and bind to the analyte. Upon binding to the analyte, the nucleic acid of the probe may be ligated such that one end of the nucleic acid of the probe is ligated to another end of the nucleic acid of the probe. Ligating one end of the nucleic acid of the probe to another end of the nucleic acid of the probe may be performed by a ligase. Ligating one end of the nucleic acid of the probe to another end of the nucleic acid of the probe may generate a circular nucleic acid. The circular nucleic acid may be amplified to generate an amplicon. Amplification of the circular nucleic acid may be performed using rolling circle amplification. The amplicon may comprise a barcode. In some cases, amplification of the circular nucleic acid may comprise incorporating of non-natural nucleotides. The non-natural nucleotides may comprise an azide, an alkyne, an amine, a thiol, a hydroxyl, or a combination thereof. The amplicon may be contacted with one or more compaction agents. In some cases, the one or more compaction agent may comprise an azide, an alkyne, an amine, a thiol, an NHS-ester, a maleimide, or a combination thereof. The one or more compaction agents may comprise a linker. The one or more linkers may react with the non-natural nucleotides incorporated during amplification. The one or more compaction agents may comprise a nucleic acid that binds to the amplicon. The barcode may be contacted with a detection probe and the detection probe may bind to the barcode. For example, a detection probe may comprise a nucleic acid. The nucleic acid of the detection probe may hybridize to the barcode. The detection probe may comprise a label, as described herein. The detection probe bound to the barcode (e.g., hybridized to the barcode). The amplicon may be imaged using an imaging system (e.g., a microscope).

Another aspect of the disclosure provides a method for processing a plurality of volumetric images. The method may comprise generating a plurality of volumetric images. The volumetric images may be of a plurality of cells. The method may comprise processing the plurality of volumetric images to identify at least 100 analytes in a cell of the plurality of cells. Identifying the plurality of analytes (e.g., at least 100 analytes) in a cell of the plurality of cells may be performed with an accuracy of at least 80%. The plurality of cells may comprise at least one-million cells.

Another aspect of the disclosure provides a method for detecting analytes. The analytes may be detected in situ. The method may comprise generating a plurality of volumetric images of a plurality of cells. The plurality of volumetric images may be processed to identify greater or equal to 1, 2, 3, 4, 5, 6, 7, 8, 9, 10, 20, 30, 40, 50, 60, 70, 80, 90, 100, 200, 300, 400, 500, 600, 700, 800, 900, or 1000 analytes (e.g., different analytes) in a cell of the plurality of cells at an accuracy of greater than or equal to 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. The plurality of cells may comprise greater or equal to 1, 2, 3, 4, 5, 6, 7, 8, 9, 10, 20, 30, 40, or 50 million cells.

In some embodiments, the method may comprise identifying greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, greater than or equal to 100, greater than or equal to 200, greater than or equal to 300, greater than or equal to 400, greater than or equal to 500, greater than or equal to 600, greater than or equal to 700, greater than or equal to 800, greater than or equal to 900, or greater than or equal to 1000 individual targets (e.g., different targets) within a cell of a plurality of cells in situ. In some cases, the methods described herein may involve identifying individual targets that are the same. For example, a transcript encoding a gene may be a target of the method and one or more copies of the transcript may be identified. In some cases, the methods described herein may involve identifying individual targets that are different. For example, two transcripts, each encoding a different gene, may be a target of the method and one or more copies of each transcript may be identified. In some embodiments, the accuracy of identifying one or more target may be greater than or equal to 60%, greater than or equal to 61%, greater than or equal to 62%, greater than or equal to 63%, greater than or equal to 64%, greater than or equal to 65%, greater than or equal to 66%, greater than or equal to 67%, greater than or equal to 68%, greater than or equal to 69%, greater than or equal to 70%, greater than or equal to 71%, greater than or equal to 72%, greater than or equal to 73%, greater than or equal to 74%, greater than or equal to 75%, greater than or equal to 76%, greater than or equal to 77%, greater than or equal to 78%, greater than or equal to 79%, greater than or equal to 80%, greater than or equal to 81%, greater than or equal to 82%, greater than or equal to 83%, greater than or equal to 84%, greater than or equal to 85%, greater than or equal to 86%, greater than or equal to 87%, greater than or equal to 88%, greater than or equal to 89%, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% in a time period. In some embodiments, the time period may be less than or equal to 2 days, less than or equal to 1 day, less than or equal to 12 hours, less than or equal to 10 hours, or shorter. The accuracy of identifying one or more targets may refer to the accuracy of detecting the correct target and not the incorrect target. The accuracy of identifying one or more targets may refer to the specificity of the method. The accuracy of identifying one or more targets may refer to identifying a percentage of transcripts of a given target that are present in a cell. In some embodiments, the plurality of cells may comprise greater or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, or greater than or equal to 50 million cells.

In some embodiments, the plurality of volumetric images may be processed to identify greater or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, greater than or equal to 100, greater than or equal to 200, greater than or equal to 300, greater than or equal to 400, greater than or equal to 500, greater than or equal to 600, greater than or equal to 700, greater than or equal to 800, greater than or equal to 900, or greater than or equal to 1000 targets (e.g., different targets) in a cell of the plurality of cells. In some embodiments, the accuracy is greater than or equal to 60%, greater than or equal to 61%, greater than or equal to 62%, greater than or equal to 63%, greater than or equal to 64%, greater than or equal to 65%, greater than or equal to 66%, greater than or equal to 67%, greater than or equal to 68%, greater than or equal to 69%, greater than or equal to 70%, greater than or equal to 71%, greater than or equal to 72%, greater than or equal to 73%, greater than or equal to 74%, greater than or equal to 75%, greater than or equal to 76%, greater than or equal to 77%, greater than or equal to 78%, greater than or equal to 79%, greater than or equal to 80%, greater than or equal to 81%, greater than or equal to 82%, greater than or equal to 83%, greater than or equal to 84%, greater than or equal to 85%, greater than or equal to 86%, greater than or equal to 87%, greater than or equal to 88%, greater than or equal to 89%, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100%.

In some aspects, the methods described herein may comprise generating one or more amplicons associated with the analytes (e.g., the at least 100 analytes). The one or more amplicons may be compacted using any one of the methods described herein. Compacting the one or more amplicons may enable a higher-throughput detection of the one or more amplicons, a faster acquisition of images associated with the one or more amplicons, or a combination thereof. In some cases, compacting the one or more amplicons may enable detecting more analytes of the plurality of analytes (e.g., at least 500 analytes) in a unit area. For example, a unit area may comprise a cell of the plurality of cells. The cell may comprise analytes of the plurality of individual analytes. Compacting the one or more amplicons of the sample may enable detecting more analytes of the plurality of individual analytes as compared to the number of analytes that may be detected without compacting the one or more amplicons.

In some aspects, the methods described herein may comprise imaging the plurality of cells using an imaging system. The imaging system may comprise an objective lens configured to transmit photons from one or more object planes within the plurality of cells to one or more sensors. The methods described herein may comprise moving the objective lens relative to the cell while simultaneously using the imaging module to acquire a series of images corresponding to a plurality of object planes within the plurality of cells. Imaging the cell using the imaging system as described herein may provide certain advantages. For example, imaging the plurality of cells using the imaging system as described herein may contribute to the accuracy of identifying the analytes within a period of time (e.g., at least 100 analytes with an accuracy of 80%).

In some cases, the plurality of individual analytes (e.g., at least 100 individual analytes) may be identified using any one of the methods described herein. For example, a tissue sample may be obtained. The tissue sample may comprise the at least 100 analytes. The sample may be contacted with binding moieties, as described herein. The binding moieties may recognize and/or bind to the plurality of analytes (e.g., at least 100 analytes). The binding moieties may be used to generate amplicons. For example, the binding moieties may bind to probes. The probes may be ligated to form circular nucleic acids. The circular nucleic acids may be amplified using rolling circle amplification (RCA). The binding moieties may be used as primers for the RCA. The RCA may generate amplicons. The amplicons may be detected using an imaging system to identify the plurality of analytes (e.g., at least 100 analytes). The imaging system may comprise any one of the imaging systems described herein.

The plurality of analytes (e.g., at least 100 analytes) may comprise one or more of the analytes described herein. In some cases, the plurality of analytes (e.g., at least 100 analytes) may comprise multiple copies of the same analyte (e.g., multiple copies of the messenger RNA for actin beta). In some cases, the plurality of analytes (e.g., at least 100 analytes) may comprise different analytes (e.g., mRNA comprising sequences for different transcripts). The plurality of analytes (e.g. at least 100 analytes) may comprise at least about 100 analytes, at least about 125 analytes, at least about 150 analytes, at least about 200 analytes, at least about 250 analytes, at least about 300 analytes, at least about 400 analytes, at least about 500 analytes, at least about 600 analytes, at least about 700 analytes, at least about 800 analytes, at least about 900 analytes, at least about 1000 analytes, at least about 5000 analytes, at least about 10000 analytes, at least about 50000 analytes, at least about 100000 analytes, at least about 500000 analytes, at least about 1000000 analytes, at least about 1×10⁷ analytes, at least about 1×10⁸ analytes, at least about 1×10⁹ analytes, at least about 1×10¹⁰ analytes, at least about 1×10¹¹ analytes, at least about 1×10¹² analytes, or more analytes. The plurality of analytes (e.g. at most 100 analytes) may comprise at most about 100 analytes, at most about 125 analytes, at most about 150 analytes, at most about 200 analytes, at most about 250 analytes, at most about 300 analytes, at most about 400 analytes, at most about 500 analytes, at most about 600 analytes, at most about 700 analytes, at most about 800 analytes, at most about 900 analytes, at most about 1000 analytes, at most about 5000 analytes, at most about 10000 analytes, at most about 50000 analytes, at most about 100000 analytes, at most about 500000 analytes, at most about 1000000 analytes, at most about 1×10⁷ analytes, at most about 1×10⁸ analytes, at most about 1×10⁹ analytes, at most about 1×10¹⁰ analytes, at most about 1×10¹¹ analytes, at most about 1×10¹² analytes, or fewer analytes. The plurality of analytes (e.g., at least 100 analytes) may comprise about 100-1000000 analytes, about 125-500000 analytes, about 150-100000 analytes, about 200-50000 analytes, about 250-10000 analytes, about 300-5000 analytes, about 400-1000 analytes, about 500-900 analytes, or about 600-800 analytes.

The methods described herein may comprise identifying and/or detecting one or more analytes within cells of a plurality of cells. The plurality of cells may be part of a tissue sample (e.g., a tissue slice). In some cases, the plurality of cells may be a cell spread. The cell spread may be placed onto a substrate or surface (e.g., a glass slide or well plate). The plurality of cells may comprise cells from cell culture. For example, cells may be grown in a flask and transferred to a substrate and/or surface for analysis in any one of the methods described herein. In some cases, the plurality of cells may be extracted from any one of the samples described herein. The plurality of cells may comprise one or more cell types. For example, the plurality of cells may comprise lymphocytes, neurons, skin cells, blood cells, fat cells, cancer cells, stem cells, or a combination thereof. The plurality of cells may comprise human cells, mouse cells, non-human primate cells, or a combination thereof. The plurality of cells may comprise at least about 100 cells, at least about 125 cells, at least about 150 cells, at least about 200 cells, at least about 250 cells, at least about 300 cells, at least about 400 cells, at least about 500 cells, at least about 600 cells, at least about 700 cells, at least about 800 cells, at least about 900 cells, at least about 1000 cells, at least about 5000 cells, at least about 10000 cells, at least about 50000 cells, at least about 100000 cells, at least about 500000 cells, at least about 1000000 cells, or more. The plurality of cells may comprise at most about 100 cells, at most about 125 cells, at most about 150 cells, at most about 200 cells, at most about 250 cells, at most about 300 cells, at most about 400 cells, at most about 500 cells, at most about 600 cells, at most about 700 cells, at most about 800 cells, at most about 900 cells, at most about 1000 cells, at most about 5000 cells, at most about 10000 cells, at most about 50000 cells, at most about 100000 cells, at most about 500000 cells, at most about 1000000 cells, or fewer cells. The plurality of cells may comprise about 100-1000000 cells, about 125-500000 cells, about 150-100000 cells, about 200-50000 cells, about 250-10000 cells, about 300-5000 cells, about 400-1000 cells, about 500-900 cells, or about 600-800.

The methods described herein may identify analytes within a sample (e.g., within a plurality of cells) with an accuracy. The accuracy may may refer to the accuracy of detecting the correct analyte and not the incorrect analyte. For example, the accuracy may be determined using a control analyte to determine a frequency of detecting the incorrect analyte. The accuracy may refer to a percent frequency of detecting the correct analyte relative to the incorrect analyte. The frequency of detecting the incorrect analyte may be used to determine the accuracy of the methods described herein. The accuracy of identifying one or more analytes may refer to the specificity of the method. For example, the accuracy of identifying one or more analytes may refer to the accuracy of identifying one or more analytes relative to a background measurement. The accuracy of identifying one or more analytes may refer to a percentage of analytes of a given analytes that are present in a cell or plurality of cells. For example, an analyte may be detected using any one of the methods described herein. A measurement of the total amount and/or copies of that analyte may be performed using a different method (e.g., a mass spectrometry, sequencing, and/or imaging method). The measurement of the total amount and/or copies of that analyte may be compared to the analyte detected using any one of the methods described herein to determine an accuracy of any one of the methods described herein. The accuracy of any one of the methods described herein may be at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. The accuracy of any one of the methods described herein may be at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, or at most about 99%, or less. The accuracy of any one of the methods described herein may be about 60-100%, about 70-90%, about 75-85%, or about 78-85%.

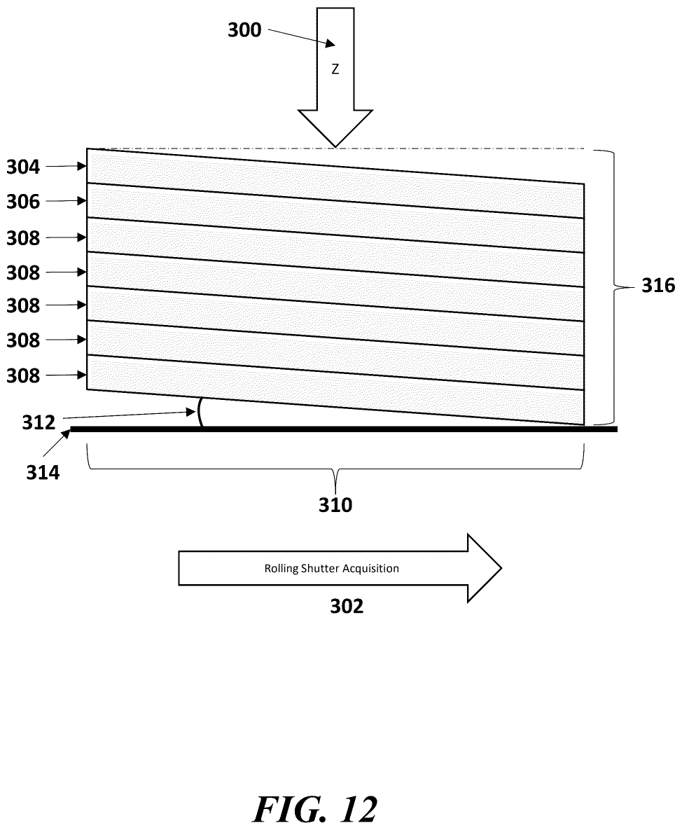

The methods described herein may involve processing volumetric images (e.g., a plurality of volumetric images). Volumetric images may comprise three-dimensional information about a sample. For example, volumetric images may comprise an image stack. Continuously moving the objective while simultaneously using the imaging module, imager, imaging device, imaging system, or any combination thereof to acquire a series of images can produce a volumetric image of the sample at a plurality of object planes of the sample. FIG. 12 shows a schematic of a volumetric (z-stack) image comprising multiple object planes in one field of view. The volumetric image can be a video. Here, the objective may be moved relative to the sample in the z-direction 300. Simultaneously, the sensor may be operating in rolling shutter mode 302, reading the pixels from left to right. FIG. 12 shows a cross-sectional diagram, e.g., showing the width of several objective planes 304, 306, 308 for a single field of view 310. Each object plane may be angled 312 in relation to the stage 314.

In other words, the object plane may not be perfectly orthogonal to the optical axis (e.g., the tilt of the object plane in relation to the optical axis may be a small angle). This angle can be any suitable angle, such as less than about 1 milliradian. Additional angles are described below. As described herein, the method can further comprise applying a mathematical transformation to the series of images to correct for the angle relative to the optical axis. In the case of rolling shutter, the mathematical transformation can include correcting for the skew angle and re-sampling in cartesian axes.

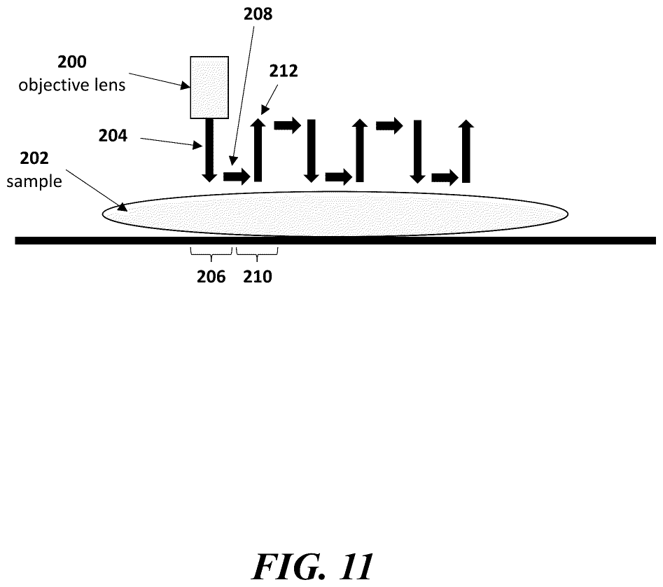

Continuing with FIG. 12, once the rolling shutter acquisition has proceeded across all pixels, the full field of view may have been imaged for the first object plane 304. The sensors can integrate one or more pixels of the sensor during a period of time (e.g., except when the one or more pixels of the sensor are being read), such that the second object plane 306 can begin being read once the first object plane 304 is completely imaged. This can be continued for subsequent object planes 308, up to a chosen depth of imaging 316. This z-stack image may be acquired while the objective moves in the z-direction relative to the sample (e.g., 204 or 212 in FIG. 11).

Following imaging of a field of view, the objective can be moved to a position configured to image a second field of view. The second field of view may be adjacent to the first field of view (e.g., movement 208 in FIG. 11).

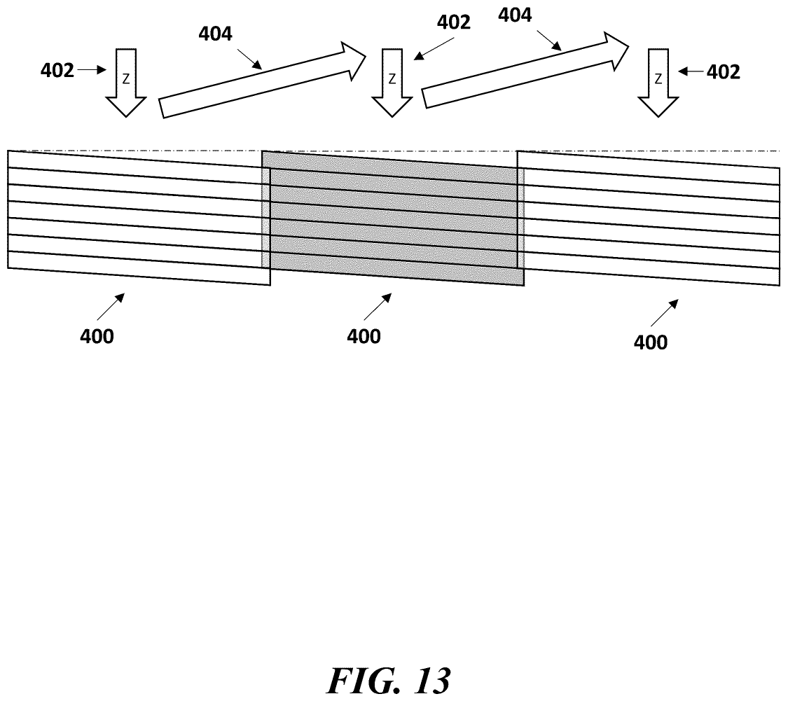

FIG. 13 shows an example schematic drawing of z-stack images (volume videos) comprising three adjacent fields of view 400, according to some embodiments of the present disclosure. These volume videos may be taken using a rolling shutter sensor, resulting in angled object planes. In this example, the imaging may be performed when the objective is moving toward the sample 402. The objective may then return 404 to the original separation distance between imaging fields. The plurality of fields of view (imaged at a depth, to create volumes) may be acquired by a single sensor in a plurality of passes or by a plurality of sensors in a single pass.

Another aspect of the disclosure provides a method for detecting an analyte. The method may comprise providing a cell. The cell may comprise an analyte. The method may comprise contacting the cell with a binding moiety. The binding moiety may recognize the analyte. The binding moiety may bind to the analyte. The binding moiety may recognize and bind to the analyte. The method may comprise contacting the binding moiety or derivative thereof with a detection probe to form a complex. The method may comprise using an imaging system to detect a signal associated with the complex. Using the imaging system to detect a signal associated with the complex may comprise detecting the analyte. The signal associated with the complex may have a full width at half maximum below 400 nanometers (nm).

Another aspect of the disclosure provides a method for detecting an analyte. The analyte may be detected in situ. The method may comprise providing a cell. The cell may comprise an analyte. The cell may be contacted with a binding moiety. The binding moiety may recognize and bind to the analyte. The binding moiety or derivative thereof may be contacted with a detection probe to form a complex. An imaging system may be used to detect a signal associated with the complex and to thereby detect the analyte. The signal may have a full width at half maximum less than or equal to 1000, 900, 800, 700, 600, 500, 400, 300, 200, 100, 90, 80, 70, 60, 50, 40, 30, 20, 10, 9, 8, 7, 6, 5, 4, 3, 2, or 1 nanometers (nm). The signal may have a full width at half maximum greater than or equal to 1000, 900, 800, 700, 600, 500, 400, 300, 200, 100, 90, 80, 70, 60, 50, 40, 30, 20, 10, 9, 8, 7, 6, 5, 4, 3, 2, or 1 nm.

The full width at half maximum (FWHM) of the signal associated with a detection probe (e.g., a detection probe complexed with a probe or binding moiety or derivative thereof) may be a measure of sharpness of an image with the signal. It some cases, it may be useful to acquire images with signals having a relatively low FWHM in order to detect more signals in a unit area. For example, an image may comprise one or more signals corresponding to one or more analytes within a sample. The one or more signals may be densely packed within the image. Signals with relatively low FWHM may enable detection of additional signals within an image of a sample, thereby detecting more analytes. The FWHM may be a measure of the sharpness or an image. The FWHM may be a measure of the resolution of an image. The FWHM may be a measure of the width of an intensity distribution at the point where an intensity of a signal is at its highest. In some embodiments, the FWHM may be expressed as a frequency. The FWHM may be expressed as a wavelength.

In some cases, a signal may associated with a complex, amplicon, detection probe, analyte, or any combination thereof may have a FWHM of less than or equal to about 1000 nanometers (nm), less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 100 nm, less than or equal to about 90 nm, less than or equal to about 80 nm, less than or equal to about 70 nm, less than or equal to about 60 nm, less than or equal to about 50 nm, less than or equal to about 40 nm, less than or equal to about 30 nm, less than or equal to about 20 nm, less than or equal to about 10 nm, less than or equal to about 9 nm, less than or equal to about 8 nm, less than or equal to about 7 nm, less than or equal to about 6 nm, less than or equal to about 5 nm, less than or equal to about 4 nm, less than or equal to about 3 nm, less than or equal to about 2 nm, less than or equal to about or 1 nm, or less. In some cases, a signal may associated with a complex, amplicon, detection probe, analyte, or any combination thereof may have a FWHM of more than or equal to about 1000 nm, more than or equal to about 900 nm, more than or equal to about 800 nm, more than or equal to about 700 nm, more than or equal to about 600 nm, more than or equal to about 500 nm, more than or equal to about 400 nm, more than or equal to about 300 nm, more than or equal to about 200 nm, more than or equal to about 100 nm, more than or equal to about 90 nm, more than or equal to about 80 nm, more than or equal to about 70 nm, more than or equal to about 60 nm, more than or equal to about 50 nm, more than or equal to about 40 nm, more than or equal to about 30 nm, more than or equal to about 20 nm, more than or equal to about 10 nm, more than or equal to about 9 nm, more than or equal to about 8 nm, more than or equal to about 7 nm, more than or equal to about 6 nm, more than or equal to about 5 nm, more than or equal to about 4 nm, more than or equal to about 3 nm, more than or equal to about 2 nm, more than or equal to about or 1 nm, or more. In some cases, a signal may associated with a complex, amplicon, detection probe, analyte, or any combination thereof may have a FWHM from about 1 nm to about 1000 nm, from about 10 nm to about 900 nm, from about 20 nm to about 800 nm, from about 30 nm to about 700 nm, from about 40 nm to about 600 nm, from about 50 nm to about 500 nm, from about 60 nm to about 500 nm, from about 70 nm to about 400 nm, from about 80 nm to about 300 nm, from about 90 nm to about 200 nm, or from about 100 nm to about 150 nm.

Another aspect of the disclosure provides a method for detecting an analyte. The method may comprise providing a cell. The cell may comprise the analyte. The method may comprise contacting the cell with a probe. The probe may recognize the analyte. The probe may bind the analyte. The probe may recognize and bind to the analyte. The method may comprise amplifying the probe to generate an amplicon. The amplicon may comprise one or more chemical reactive moieties. For example, the amplicon may comprise a first chemical reactive moiety and a second chemical reactive moiety. The method may comprise incubating the amplicon under conditions sufficient to form a conjugate between chemical reactive moieties. For example, the method may comprise incubating the amplicon under conditions sufficient to form a conjugate between the first chemical reactive moiety of the amplicon and the second chemical reactive moiety of the amplicon. The method may comprise detecting the amplicon to thereby detect the analyte.

Another aspect of the disclosure provides a method for detecting an analyte. The analyte may be detected in situ. The method may comprise providing a cell. The cell may comprise the analyte. The cell may be contacted with a probe. The probe may recognize and bind to the analyte. The probe may be amplified to generate an amplicon. The amplicon may comprise a first reactive chemical moiety and a second reactive chemical moiety. The amplicon may be incubated under conditions sufficient to form a conjugate between the first reactive chemical moiety and the second reactive chemical moiety. The amplicon may be detected to thereby detect the analyte.

The methods described herein may comprise detecting one or more amplicons. The one or more amplicons may be compacted using any one of the methods described herein. For example, the one or more amplicons may be contacted with a compaction agent, thereby decreasing the diameter of the one or more amplicons. Detecting the one or more amplicons may comprise imaging the one or more amplicons using any one of the imaging systems described herein. In some cases, detecting the one or more amplicons may comprise imaging one or more fluorescence signals associated with the one or more amplicons. For example, the methods may comprise contacting the one or more amplicons with one or more detection probes. The one or more detection probes may comprise a fluorescence dye. The fluorescence dye may emit a fluorescence signal when imaged with any one of the imaging systems described herein. The imaging system may be used to generate a signal associated with the one or more amplicons from fluorescence signal emitted from the one or more detection probes. The signal associated with the one or more amplicons captured by any one of the imaging systems may be captured as an image, e.g., a volumetric image. The image of the signal associated with the one or more amplicons may be used to identify one or more analytes associated with one or more amplicons.

One or more amplicons of the methods described herein may comprise one or more chemical reactive moieties (e.g., a first chemical reactive moiety and a second chemical reactive moiety.) The one or more reactive moieties may comprise any one of the chemical reactive moieties as described herein. For example, an amplicon of the one or more amplicons may comprise an alkyne and an azide. An amplicon of the one or more amplicons may be incubated under conditions sufficient to form a conjugate between two chemical reactive moieties of the amplicon (e.g., a first chemical reactive moiety and a second chemical reactive moiety.) The conditions sufficient to form a conjugate between two chemical reactive moieties of the amplicon may comprise incubating the amplicon for a period time, incubating the amplicon at one or more temperatures, incubating the amplicon at a pH, incubating the amplicon in the presence of one or more buffers, incubating the amplicon with one or more solvents, or a combination thereof.

The conditions sufficient to form a conjugate between two chemical reactive moieties of the amplicon may comprise incubating the amplicon for at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days, at least about 4 days, or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days, at most about 4 days, or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours.

The conditions sufficient to form a conjugate between two chemical reactive moieties of the amplicon may comprise incubating the amplicon at one or more temperatures. The one or more temperatures may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperatures may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The conditions sufficient to form a conjugate between two chemical reactive moieties of the amplicon may comprise incubating the amplicon with one or more buffers. The one or more buffers may comprise MES (4-Morpholineethanesulfonic acid), Bis-Tris (Bis(2-hydroxyethyl)amino-tris(hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The one or more buffers may comprise a concentration of one or more of the buffers listed herein of at least about 1 mM buffer, at least about 5 mM buffer, at least about 10 mM buffer, at least about 25 mM buffer, at least about 50 mM buffer, at least about 100 mM buffer, at least about 200 mM buffer, at least about 500 mM buffer, at least about 750 mM buffer, at least about 1 M buffer, or more. The one or more buffers may comprise a concentration of one or more of the buffers listed herein of at most about 1 mM buffer, at most about 5 mM buffer, at most about 10 mM buffer, at most about 25 mM buffer, at most about 50 mM buffer, at most about 100 mM buffer, at most about 200 mM buffer, at most about 500 mM buffer, at most about 750 mM buffer, at most about 1 M buffer, or more. The one or more buffers may comprise a concentration of about 1 mM to about 1 M buffer, of about 5 mM to about 750 mM buffer, of about 25 mM to about 500 mM buffer, or of about 100 mM to about 250 mM buffer.

The conditions sufficient to form a conjugate between two chemical reactive moieties of the amplicon may comprise incubating the amplicon with one or more salts. The one or more salts may comprise NaCl, CaCl₂, MgCl₂, or a combination thereof. The conditions sufficient to form a conjugate between two chemical reactive moieties of the amplicon may comprise incubating the amplicon with one or more detergents. The one or more detergents may comprise SDS, Triton X-100, CHAPS, NP-40, Tween-20, Digitonin, or a combination thereof. The conditions sufficient to form a conjugate between two chemical reactive moieties of the amplicon may comprise incubating the amplicon with one or more solvents. The one or more solvents may comprise methanol, ethanol, ethyl acetate, DMSO, acetonitrile, water, or a combination thereof.

The conditions sufficient to form a conjugate between two chemical reactive moieties of the amplicon may comprise incubating the amplicon at a pH. The pH may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, or higher. The pH may be at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12, or less

The chemical reactive moieties may form a conjugate during incubation under conditions sufficient to form a conjugate. The conjugate may comprise one or more covalent bonds, one or more non-covalent bonds, or a combination thereof. In some cases, the conjugate may comprise a linker. The linker may comprise a polyethylene glycol, a methylene, or a combination thereof. The conjugate may bind one part of the amplicon to another part of an amplicon. For example, a first chemical reactive moiety of the amplicon may react with a second chemical reactive moiety of the amplicon to form a conjugate. The conjugate may comprise a newly formed covalent bond between the first chemical reactive moiety and the second chemical reactive moiety. Formation of the covalent bond between the first chemical reactive moiety and the second chemical reactive moiety may compact the amplicon (e.g., increasing the density of the amplicon, decreasing the size of the amplicon, or a combination thereof.)

Another aspect of the disclosure provides a method for detecting an analyte. The method may comprise providing a cell. The cell may comprise the analyte. The method may comprise contacting the cell with a binding moiety. The binding moiety may bind to the analyte. The method may comprise generating an amplicon with the aid of the binding moiety. The amplicon may comprise a barcode. The method may comprise contacting the amplicon with a detection probe under conditions sufficient to form a complex between the detection probe and the amplicon. The method may comprise using the imaging system to detect a signal associated with the complex, thereby detecting the analyte. The signal may have a diameter of less than 200 nanometers (nm).

The methods described herein may comprise contacting one or more amplicons with one or more detection probes under conditions sufficient to form a complex between an amplicon and a detection probe. For example, a detection probe comprising a nucleic acid may hybridize to an amplicon under conditions sufficient to form a complex between the detection probe and the amplicon. The conditions sufficient to form a complex between a detection probe and an amplicon may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days, at least about 4 days, or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days, at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperatures may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The conditions sufficient to form a complex between a detection probe and an amplicon may comprise one or more buffers. The one or more buffers may comprise MES (4-Morpholineethanesulfonic acid), Bis-Tris (Bis(2-hydroxyethyl)amino-tris (hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The conditions sufficient to form a complex between a detection probe and an amplicon may comprise one or more salts. The one or more salts may comprise NaCl, CaCl2, MgCl2, or a combination thereof. The conditions sufficient to form a complex between a detection probe and an amplicon may comprise one or more detergents. The one or more detergents may comprise SDS, Triton X-100, CHAPS, NP-40, Tween-20, Digitonin, or a combination thereof. The conditions sufficient to form a complex between a detection probe and an amplicon may comprise one or more solvents. The one or more solvents may comprise methanol, ethanol, ethyl acetate, DMSO, acetonitrile, water, or a combination thereof. The conditions sufficient to form a complex between a detection probe and an amplicon may comprise a pH. The pH may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, or higher. The pH may be at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12, or less. Contacting one or more amplicons with one or more detection probes under conditions sufficient to form a complex between an amplicon of the one or more amplicons and a detection probe of the one or more detection probes may result in a complex. The complex may be stable for a period of time (e.g., the complex may be stable for at least one hour). The one or more amplicons may be compacted using any one of the methods described herein. For example, the one or more detection probes may contact the one or more amplicons after compacting the one or more amplicons (e.g., by contacting the one or more amplicons with a compacting agent and/or incubating the one or more amplicons under conditions sufficient to promote the formation of a conjugate between a first chemical reactive moiety of an amplicon of the one or more amplicons and a second chemical reactive moiety of an amplicon of the one or more amplicons.)

The methods described herein may comprise detecting a signal associated with one or more amplicons, one or more complexes formed between an amplicon and a detection probe, or a combination thereof. In some cases, the signal may be a fluorescence signal. The signal may be imaged using any one of the imaging systems described herein. The signal may comprise a diameter. For example, the signal may be defined by the boundaries on the image where a fluorescence signal comprises a value above a threshold. For example, an image may be acquired of the sample using any one of the imaging systems described herein. The image may comprise pixels. The pixels may comprise a fluorescence intensity. In some cases, the fluorescence intensity of each pixel may have a unique fluorescence intensity. A signal may be determined by identifying a boundary around pixels, where the pixels may have a fluorescence intensity above a threshold. The boundary may define the shape of the signal. In some cases, the signal may comprise a diameter. The diameter may be a measure across the boundary of the signal. For example, in some cases, the signal may be circular in shape and have a diameter. In some cases, the signal may have a diameter of less than or equal to 1000 nm, less than or equal to 900 nm, less than or equal to 800 nm, less than or equal to 700 nm, less than or equal to 600 nm, less than or equal to 500 nm, less than or equal to 400 nm, less than or equal to 300 nm, less than or equal to 200 nm, less than or equal to 100 nm, less than or equal to 90 nm, less than or equal to 80 nm, less than or equal to 70 nm, less than or equal to 60 nm, less than or equal to 50 nm, less than or equal to 40 nm, less than or equal to 30 nm, less than or equal to 20 nm, less than or equal to 10 nm, less than or equal to 9 nm, less than or equal to 8 nm, less than or equal to 7 nm, less than or equal to 6 nm, less than or equal to 5 nm, less than or equal to 4 nm, less than or equal to 3 nm, less than or equal to 2 nm, less than or equal to 1 nm, or less. In some cases, the signal may have a diameter of more than or equal to 1000 nm, more than or equal to 900 nm, more than or equal to 800 nm, more than or equal to 700 nm, more than or equal to 600 nm, more than or equal to 500 nm, more than or equal to 400 nm, more than or equal to 300 nm, more than or equal to 200 nm, more than or equal to 100 nm, more than or equal to 90 nm, more than or equal to 80 nm, more than or equal to 70 nm, more than or equal to 60 nm, more than or equal to 50 nm, more than or equal to 40 nm, more than or equal to 30 nm, more than or equal to 20 nm, more than or equal to 10 nm, more than or equal to 9 nm, more than or equal to 8 nm, more than or equal to 7 nm, more than or equal to 6 nm, more than or equal to 5 nm, more than or equal to 4 nm, more than or equal to 3 nm, more than or equal to 2 nm, more than or equal to 1 nm, or more. In some cases, the signal may have a diameter from about 0.1 nm to about 1000 nm, from about 1 nm to about 900 nm, from about 2 nm to about 800 nm, from about 3 nm to about 700 nm, from about 4 nm to about 600 nm, from about 5 nm to about 500 nm, from about 6 nm to about 400 nm, from about 7 nm to about 300 nm, from about 8 nm to about 200 nm, from about 9 nm to about 100 nm, from about 10 nm to about 90 nm, from about 20 nm to about 80 nm, from about 30 nm to about 70 nm, or from about 40 nm to about 60 nm. The diameter of the signal may greater than the diffraction limit of the imaging system used to image the sample, less than the diffraction limit of the imaging system used to image the sample, or the same as the diffraction limit of the imaging system used to image the sample.

The diffraction of the imaging system used to image the sample may refer to the smallest distance between two objects that the two objects can be distinguished as separate entities. In some embodiments, the diffraction limit of the imaging system may be less than about 100 micrometers (μm), less than about 80 μm, less than about 60 μm, less than about 40 μm, less than about 20 μm, less than about 10 μm, less than about 5 μm, less than about 2 μm, less than about 1 μm, less than about 0.1 μm, less than about 0.01 μm, or less. In some embodiments, the diffraction limit of the imaging system may be more than about 100 micrometers (μm), more than about 80 μm, more than about 60 μm, more than about 40 μm, more than about 20 μm, more than about 10 μm, more than about 5 μm, more than about 2 μm, more than about 1 μm, more than about 0.1 μm, or more than about 0.01 μm. In some embodiments, the diffraction limit of the imaging system may be more than about 100 μm, more than about 80 μm, more than about 60 μm, more than about 40 μm, more than about 20 μm, more than about 10 μm, more than about 5 μm, more than about 2 μm, more than about 1 μm, more than about 0.1 μm, or more.

Another aspect of the disclosure provides a method for detecting an amplicon. The method may comprise providing a sample. The sample may comprise one or more analytes (e.g., a first analyte and a second analyte). The sample may comprise a first analyte and a second analyte. The method may comprise contacting the first analyte with a binding moiety. The binding moiety may recognize the first analyte. The biding moiety may bind to the first analyte. The binding moiety may bind to and recognize the first analyte. The method may comprise generating an amplicon with the aid of the binding moiety. The method may comprise detecting the amplicon with greater than 90% accuracy. The amplicon may be produced when the first analyte is proximal to the second analyte. In some cases, the amplicon may be produced only when the first analyte is proximal to the second analyte.

Another aspect of the disclosure provides a method for detecting analytes in a sample. The method may comprise providing the sample. The sample may comprise a first analyte and a second analyte. The first analyte may be contacted with a first binding agent. The first binding agent may comprise a barcode. The sample may be contacted with a compaction agent. The compaction agent may bind to the barcode or reverse complement thereof. A reverse complement of the barcode may be detected with greater than or equal to 80%, 81%, 82%, 83%, 84%, 85%, 86%, 87%, 88%, 89%, 90%, 91%, 92%, 93%, 94%, 95%, 96%, 97%, 98%, 99%, or 100% accuracy. The reverse complement of the barcode may be produced when the first analyte is proximal to the second analyte.

In some embodiments, the distance between the first analyte and the second analyte is less than or equal to 2000 nm, less than or equal to 1900 nm, less than or equal to 1800 nm, less than or equal to 1700 nm, less than or equal to 1600 nm, less than or equal to 1500 nm, less than or equal to 1400 nm, less than or equal to 1300 nm, less than or equal to 1200 nm, less than or equal to 1100 nm, less than or equal to 1000 nm, less than or equal to 900 nm, less than or equal to 800 nm, less than or equal to 700 nm, less than or equal to 600 nm, less than or equal to 500 nm, less than or equal to 400 nm, less than or equal to 300 nm, less than or equal to 200 nm, less than or equal to 100 nm, less than or equal to 50 nm, less than or equal to 10 nm, less than or equal to 1 nm, less than or equal to 0.1 nm, from 0.1 nm to 2000 nm, from 1 nm to 1900 nm, from 10 nm to 1800 nm, from 50 nm to 1700 nm, from 100 nm to 1600 nm, from 200 nm to 1500 nm, from 300 nm to 1400 nm, from 400 nm to 1300 nm, from 500 nm to 1200 nm, from 600 nm to 1100 nm, from 700 nm to 1000 nm, from 800 nm to 900 nm, from 0.1 nm to 500 nm, from 200 nm to 800 nm, from 500 nm to 1000 nm, from 800 nm to 1400 nm, from 1000 nm to 1600 nm, from 1200 nm to 1800 nm, or from 1500 nm to 2000 nm.

The sample described herein may be contacted with another binding agent (e.g., a second binding agent). The other binding agent may bind to the second analyte. The other binding agent may comprise a nucleic acid. The nucleic acid of the other binding agent may be linked to a polypeptide, for example an antibody. The other binding agent and the first binding agent may interact to form a complex. The complex of the other binding agent and the first binding agent may comprise a nucleic acid. The nucleic acid of the complex of the other binding agent and the first binding agent may be ligated using a ligase upon binding of the nucleic acid to the other binding agent and the first binding agent to form a circular nucleic acid. The circular nucleic acid formed from a ligation reaction as described herein may comprise one or more barcodes or one or more reverse complements of one or more barcodes. A rolling circle amplification reaction may be performed to generate one or more amplicons. The one or more amplicons may be contacted with a compaction agent. The compaction agent may bind to the one or more barcodes or one or more reverse complements of one or more barcodes. The one or more amplicons may be contacted with one or more detection probes. The one or more detection probes may bind to the one or more amplicons. The sample may be imaged using an imaging system to detect the one or more detection probes.

The methods described herein may comprise generating one or more amplicons. The one or more amplicons may be generated with the aid of one or more binding moieties. In some cases, the one or more binding moieties may comprise a nucleic acid. The nucleic acid of the one or more binding moieties may bind to one or more analytes. The one or more binding moieties may be ligated to form a circular nucleic acid (e.g., a first end of a binding moiety may be ligated to a second end of a binding moiety to generate a circular nucleic acid). The circular nucleic acid may be amplified (e.g., using rolling circle amplification) to generate one or more amplicons. The one or more binding moieties may aid in generating one or more amplicons by binding one or more probes. In some cases, a binding moiety of the one or more binding moieties may comprise a nucleic acid. The nucleic acid may bind to an analyte. In some cases, the nucleic acid may be connected to another component of the binding moiety that binds to the analyte. For example, the binding moiety may comprise an antibody that is connected to the nucleic acid. The antibody may bind to the analyte and the nucleic acid may be localized to the analyte. A probe may be added to the sample. The probe may comprise a nucleic acid. The probe may bind to the nucleic acid of the binding moiety. The probe may be ligated to generate a circular nucleic acid. The circular nucleic acid may be amplified (e.g., using rolling circle amplification) to generate one or more amplicons. In some cases, the binding moiety may comprise a nucleic acid sequence that binds to the analyte and a nucleic acid sequence that does not bind to the analyte. For example, a binding moiety may comprise a sequence that hybridizes to an RNA within a sample. The binding moiety may comprise a sequence that does not hybridize to the RNA within the sample. A probe may be added to the sample. The probe may bind to (e.g., hybridize to) the sequence that does not bind to the RNA of the binding moiety. The probe may be ligated to generate a circular nucleic acid. The circular nucleic acid may be amplified (e.g., using rolling circle amplification) to generate one or more amplicons.

The methods described herein may identify amplicons within a sample (e.g., within a plurality of cells) with an accuracy. The accuracy may may refer to the accuracy of detecting the correct amplicon and not the incorrect amplicon. For example, the accuracy may be determined using a control amplicon to determine a frequency of detecting the incorrect amplicon. The frequency of detecting the incorrect amplicon may be used to determine the accuracy of the methods described herein. The accuracy of identifying one or more amplicons may refer to the specificity of the method. For example, the accuracy of identifying one or more amplicons may refer to the accuracy of identifying one or more amplicons relative to a background measurement. The accuracy of identifying one or more analytes may refer to identifying a percentage of amplicons of a given amplicons that are present in a cell or plurality of cells. For example, an amplicon may be detected using any one of the methods described herein. A measurement of the total amount and/or copies of that amplicon may be performed using a different method (e.g., a mass spectrometry, sequencing, and/or imaging method). The measurement of the total amount and/or copies of that amplicon may be compared to the amplicon detected using any one of the methods described herein to determine an accuracy of any one of the methods described herein. The accuracy of any one of the methods described herein may be at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100%. The accuracy of any one of the methods described herein may be at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 96%, at most about 97%, at most about 98%, or at most about 99%, or less. The accuracy of any one of the methods described herein may be about 60-100%, about 70-90%, about 75-85%, or about 78-85%.

The methods described herein may be used to detect a proximity between analytes. For example, the methods described herein may be used to detect a proximity of a first analyte and a second analyte in a sample. Methods for detecting a proximity between analytes may be useful for determining relationships between analytes in a sample, a biological state of a sample, identifying binding partners within a sample, or a combination thereof. In some cases, determining a proximity between analytes may reveal transcription and/or translation information about the sample. For example, determining a proximity between a transcription factor and a transcript may provide information about the transcriptional state of a sample. The methods described herein may comprise compacting amplicons in addition to detection a proximity of analytes. For example, one or more amplicons may be generated based on the proximity of a first analyte to a second analyte to the sample. The one or more amplicons may be compacted using any one of the methods described herein. Compacting the one or more amplicons may be useful for identifying more amplicons associated with the proximity of analytes, for identifying a stronger signal associated with the analytes, acquiring imaging data of the amplicons using less time (e.g., by using a lower exposure time) using any one or the imaging systems described herein, or a combination thereof.

In some cases, proximity of analytes may be determined by generating one or more amplicons. For example, one or more amplicons may be generated when a first analyte is proximal to (e.g., next to) a second analyte in a sample. The first analyte may be contacted with a first binding moiety. The first binding moiety may bind to the first analyte. The second analyte may be contacted with a second binding moiety. The second binding moiety may bind to the second analyte. The sample comprising the first analyte and second analyte may be contacted with a probe. The probe may bind to the first binding moiety and the second binding moiety. The probe may be ligated upon binding to the first binding moiety and the second binding moiety. The probe may be ligated to generate a circular nucleic acid. The circular nucleic acid may be amplified (e.g., using rolling circle amplification) to generate one or more amplicons. The one or more amplicons may be compacted using any one of the methods described herein. The one or more amplicons may be detected (e.g., imaged) using any one of the imaging systems described herein. Detecting the one or more amplicons may enable detecting the proximity between the first analyte and the second analyte.

Another aspect of the disclosure provides a method for detecting an analyte. The method may comprise providing a cell. The cell may comprise the analyte. The method may comprise contacting the cell with a binding moiety. The binding moiety may bind to the analyte. The method may comprise amplifying at least a portion of the binding moiety to generate an amplicon. The method may comprise contacting the amplicon with a compaction agent. The compaction agent may be configured to form one or more covalent interactions with the amplicon. The method may comprise using an imaging system to detect a signal. The signal may be associated with the amplicon. The signal may comprise a roundness value. The roundness value may be 1.1 or less.

The methods described herein may comprise detecting (e.g., imaging) a sample to generate an image. A signal associated with one or more amplicons of the methods described herein may comprise a signal amplitude, a shape, or a combination thereof. The shape of the signal may be analyzed using image analysis algorithms that measure the radius of the shape of the signal, the diameter of the shape of the signal, the area of the shape of the signal, or a combination thereof. The image may comprise one or more signals. The one or more signals may comprise a roundness value. The roundness value may provide information related to have closely the shape of an object approaches that of a circle (e.g., a mathematically perfect circle). The roundness value may be determined based on the shape of the signal. The roundness value may be determined based on a ratio of the radii of inscribed to circumscribed circles that fit inside the shape of the signal. The roundness value may be used for characterizing 2-D shapes. The roundness value may be calculated using the formula below:

Roundness=Perimeter²/4π×Area

The roundness value of a signal detected (e.g., imaged) using any one of the methods described herein may be less than or equal to 10 μm, less than or equal to 9 μm, less than or equal to 8 μm, less than or equal to 7 μm, less than or equal to 6 μm, less than or equal to 5 μm, less than or equal to 4 μm, less than or equal to 3 μm, less than or equal to 2 μm, less than or equal to 1.5 μm, less than or equal to 1.1 μm, less than or equal to 1 μm, less than or equal to 0.9 μm, less than or equal to 0.8 μm, less than or equal to 0.7 μm, less than or equal to 0.6 μm, less than or equal to 0.5 μm, less than or equal to 0.4 μm, less than or equal to 0.3 μm, less than or equal to 0.2 μm, less than or equal to 0.1 μm, or less. The roundness value of a signal detected (e.g., imaged) using any one of the methods described herein may be greater than or equal to 10 μm, greater than or equal to 9 μm, greater than or equal to 8 μm, greater than or equal to 7 μm, greater than or equal to 6 μm, greater than or equal to 5 μm, greater than or equal to 4 μm, greater than or equal to 3 μm, greater than or equal to 2 μm, greater than or equal to 1.5 μm, greater than or equal to 1.1 μm, greater than or equal to 1 μm, greater than or equal to 0.9 μm, greater than or equal to 0.8 μm, greater than or equal to 0.7 μm, greater than or equal to 0.6 μm, greater than or equal to 0.5 μm, greater than or equal to 0.4 μm, greater than or equal to 0.3 μm, greater than or equal to 0.2 μm, greater than or equal to 0.1 μm, or greater. The roundness value of a signal detected (e.g., imaged) using any one of the methods described herein may from about 0.1 μm to about 10 μm, from about 0.2 μm to about 9 μm, from about 0.3 μm to about 8 μm, from about 0.3 μm to about 7 μm, from about 0.4 μm to about 6 μm, from about 0.5 μm to about 5 μm, from about 0.6 μm to about 4 μm, from about 0.7 μm to about 3 μm, from about 0.8 μm to about 2 μm, or from about 0.9 μm to about 1 μm.

Another aspect of the disclosure provides a method for detecting analytes. The method may comprise providing a fresh-frozen sample. The fresh-frozen sample may comprise a plurality of cells. The plurality of cells may comprise a plurality of analytes. The method may comprise contacting the fresh-frozen sample with a plurality of binding moieties. A binding moiety of the plurality of binding moieties may recognize an analyte of the plurality of analytes. The binding moiety of the plurality binding moieties may bind to the analyte of the plurality of analytes. The binding moiety of the plurality binding moieties may bind to and recognize the analyte of the plurality of analytes. The method may comprise contacting the fresh-frozen sample with a plurality of detection probes to form a plurality of complexes. The plurality of complexes may be formed between binding moieties of the plurality of binding moieties and detection probes of the plurality of detection probes. The plurality of complexes may be formed between derivatives of binding moieties of the plurality of binding moieties and detection probes of the plurality of detection probes. The method may comprise detecting the plurality of complexes. Identifying the plurality of complexes may identify the plurality of analytes. Identifying the plurality of analytes may comprise identifying more than 200 analytes on average per cell.

Another aspect of the disclosure provides a method for detecting analytes. The analytes may be detected in situ. The method may comprise providing a fresh-frozen sample. The fresh-frozen sample may comprise a plurality of cells. The plurality of cells may comprise a plurality of analytes. The fresh-frozen sample may be contacted with a plurality of binding moieties. A binding moiety of the plurality of binding moieties may recognize and bind to a transcript of the plurality of analytes. Next, the plurality of binding moieties or derivatives thereof may be contacted with a plurality of detection probes to form a plurality of complexes. The plurality of complexes may be detected to thereby identify the plurality of analytes. Identifying the plurality of analytes may comprise identifying greater than or equal to 1, 2, 3, 4, 5, 6, 7, 8, 9, 10, 20, 30, 40, 50, 60, 70, 80, 90, 100, 200, 300, 400, 500, 600, 700, 800, 900, or 1000 analytes (e.g., different transcripts) on average per cell.

The methods described herein may comprise providing a fresh-frozen sample. The fresh-frozen sample may comprise a plurality of cells, a tissue sample, a blood sample, a biopsy sample, or a combination thereof. The fresh-frozen sample may not have been treated with a fixative (e.g., a cross-linking agent). The fresh-frozen sample may comprise a tissue slice or specimen. The fresh-frozen sample may be sliced from a tissue sample embedded in optimal cutting temperature (OCT) compound. The fresh-frozen sample may be placed on a surface or a substrate. For example, a tissue slice may be placed on a microscope slide, within a well plate, within a sample well, within a flow cell, or a combination thereof.

In some embodiments, the fresh-frozen sample may be a fresh-frozen tissue sample. The fresh-frozen sample may comprise a tissue slice obtained from a tissue bloc. The fresh-frozen tissue sample may be mounted on a surface used for analysis. In some cases, the surface used for analysis may comprise a coverslip, a slide, a well of a well plate, or a combination thereof. The fresh-frozen sample may have a variety of thicknesses including but not limited to about 5 to about 250 μm thick, from about 5 to about 200 μm thick, from about 5 to about 150 μm thick, from about 5 to about 100 μm thick, from about 5 to about 50 μm thick, from about 5 to about 10 μm thick, from about 10 to about 250 μm thick, from about 10 to about 200 μm thick, from about 10 to about 150 μm thick, from about 10 to about 100 μm thick, from about 10 to about 50 μm thick, from about 25 to about 250 μm thick, from about 25 to about 200 μm thick, from about 25 to about 150 μm thick, from about 25 to about 100 μm thick, or from about 25 to about 50 μm thick. In some cases, the fresh-frozen sample may be at least about 5 μm thick, at least about 10 μm thick, at least about 25 μm thick, at least about 50 μm thick, at least about 100 μm thick, at least about 150 μm thick, at least about 200 μm thick, at least about 250 μm thick, or greater. In some cases, the fresh-frozen sample may be at most about 5 μm thick, at most about 10 μm thick, at most about 25 μm thick, at most about 50 μm thick, at most about 100 μm thick, at most about 150 μm thick, at most about 200 μm thick, at most about 250 μm thick, or less.

In some embodiments, identifying the plurality of analytes may comprise identifying greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, greater than or equal to 100, greater than or equal to 200, greater than or equal to 300, greater than or equal to 400, greater than or equal to 500, greater than or equal to 600, greater than or equal to 700, greater than or equal to 800, greater than or equal to 900, or greater than or equal to 1000 analytes (e.g., different analytes) on average per cell. In some embodiments, the plurality of analytes may comprise messenger RNA (mRNA). In some embodiments, the plurality of analytes may comprise pre-mRNA.

In some embodiments, the method may comprise contacting the fresh-frozen sample with a probe. In some cases, contacting the fresh-frozen sample with a probe may take place after contacting the fresh-frozen sample with a plurality of binding moieties. In some cases, contacting the fresh-frozen sample with a probe may take place before contacting the fresh-frozen sample with a plurality of binding moieties. In some cases, contacting the fresh-frozen sample with a probe may take place at the same time as contacting the fresh-frozen sample with a plurality of binding moieties. The probe may comprise nucleic acid, a polypeptide, or a combination thereof. In some cases, the nucleic acid of the probe may comprise deoxyribonucleic acid, ribonucleic acid, or a combination thereof. The nucleic acid of the probe may bind to a binding moiety of the plurality of binding moieties. For example, a nucleic acid may bind to the binding moiety at two locations of the binding moiety. The two locations of the binding moiety (e.g., two regions of a nucleic acid of the binding moiety) may be proximal to one another. Once bound to the binding moiety, two ends of the nucleic acid may be directly adjacent to one another. In some embodiments, the method may comprise performing a ligation reaction. In some cases, the ligation reaction may take place after contacting the fresh-frozen sample with a plurality of binding moieties. In some cases, the ligation reaction may take place before contacting the fresh-frozen sample with a plurality of binding moieties. In some cases, the ligation reaction may take place at the same time as contacting the fresh-frozen sample with a plurality of binding moieties. The ligation reaction may comprise ligating a nucleic acid (e.g., the probe) associated with a binding moiety of the plurality of binding moieties to form a circular nucleic acid. In some embodiments, the method may comprise performing an amplification reaction (e.g., a rolling circle amplification reaction). In some cases, performing the amplification reaction may take place after contacting the fresh-frozen sample with a plurality of binding moieties. In some cases, performing the amplification reaction may take place before contacting the fresh-frozen sample with a plurality of binding moieties. In some cases, performing the amplification reaction may take place at the same time as contacting the fresh-frozen sample with a plurality of binding moieties. In some embodiments, detecting the plurality of complexes may comprise imaging the cell using a microscope.

The methods described herein may be used to detect and/or identify a plurality of analytes. The plurality of analytes may be detected and/or identified by imaging one or more amplicons generated as described herein. In some cases, the one or more amplicons may be compacted (e.g., reduced in size and/or increased in density) using any one of the methods described herein. The plurality of analytes detected and/or identified may comprise at least about 200 analytes, at least about 250 analytes, at least about 300 analytes, at least about 350 analytes, at least about 400 analytes, at least about 450 analytes, at least about 500 analytes, at least about 600 analytes, at least about 700 analytes, at least about 800 analytes, at least about 900 analytes, at least about 1000 analytes, at least about 1500 analytes, at least about 2000 analytes, at least about 2500 analytes, at least about 5000 analytes, at least about 10000 analytes, at least about 15000 analytes, at least about 20000 analytes, at least about 50000 analytes, at least about 100000 analytes, or more. The plurality of analytes detected and/or identified may comprise at most about 200 analytes, at most about 250 analytes, at most about 300 analytes, at most about 350 analytes, at most about 400 analytes, at most about 450 analytes, at most about 500 analytes, at most about 600 analytes, at most about 700 analytes, at most about 800 analytes, at most about 900 analytes, at most about 1000 analytes, at most about 1500 analytes, at most about 2000 analytes, at most about 2500 analytes, at most about 5000 analytes, at most about 10000 analytes, at most about 15000 analytes, at most about 20000 analytes, at most about 50000 analytes, at most about 100000 analytes, or fewer. The plurality of analytes detected and/or identified may comprise about 200-100000 analytes, about 250-50000 analytes, about 300-20000 analytes, about 350-15000 analytes, about 400-10000 analytes, about 450-5000 analytes, about 500-2500 analytes, about 600-2000 analytes, about 700-1500 analytes, or about 800-1000 analytes.

Another aspect of the disclosure provides a method for detecting analytes. The method may comprise providing a formalin-fixed paraffin embedded sample. The formalin-fixed paraffin embedded sample may comprise a plurality of cells. The plurality of cells may comprise a plurality of analytes. The method may comprise contacting the formalin-fixed paraffin embedded sample with a plurality of binding moieties. A binding moiety of the plurality of binding moieties may recognize an analyte of the plurality of analytes. The binding moiety of the plurality binding moieties may bind to the analyte of the plurality of analytes. The binding moiety of the plurality binding moieties may bind to and recognize the analyte of the plurality of analytes. The method may comprise contacting the formalin-fixed paraffin embedded sample with a plurality of detection probes to form a plurality of complexes. The plurality of complexes may be formed between binding moieties of the plurality of binding moieties and detection probes of the plurality of detection probes. The plurality of complexes may be formed between derivatives of binding moieties of the plurality of binding moieties and detection probes of the plurality of detection probes. The method may comprise detecting the plurality of complexes. Identifying the plurality of complexes may identify the plurality of analytes. Identifying the plurality of analytes may comprise identifying more than 120 analytes on average per cell.

Another aspect of the disclosure provides a method for detecting analytes. The analytes may be detected in situ. The method may comprise providing a formalin-fixed paraffin embedded sample. The formalin-fixed paraffin embedded sample may comprise a plurality of cells. The plurality of cells may comprise a plurality of analytes. The formalin-fixed paraffin embedded sample may be contacted with a plurality of binding moieties. A binding moiety of the plurality of binding moieties may recognize and bind to a transcript of the plurality of analytes. Next, the plurality of binding moieties or derivatives thereof may be contacted with a plurality of detection probes to form a plurality of complexes. The plurality of complexes may be detected to thereby identify the plurality of analytes. Identifying the plurality of analytes may comprise identifying greater than or equal to 1, 2, 3, 4, 5, 6, 7, 8, 9, 10, 20, 30, 40, 50, 60, 70, 80, 90, 100, 120, 150, 200, 300, 400, 500, 600, 700, 800, 900, or 1000 analytes (e.g., different transcripts) on average per cell.

In some embodiments, the analyte may be within the cell. In some embodiments, the analyte may be on a surface of the cell. In some embodiments, the cell may be within a sample. In some embodiments, sample may be a tissue sample. In some embodiments, the tissue sample may be a fresh-frozen tissue sample. In some embodiments, the tissue sample may be a formalin-fixed paraffin embedded tissue sample. In some embodiments, the sample may be from about 5 to about 250 μm thick, from about 5 to about 200 μm thick, from about 5 to about 150 μm thick, from about 5 to about 100 μm thick, from about 5 to about 50 μm thick, from about 5 to about 10 μm thick, from about 10 to about 250 μm thick, from about 10 to about 200 μm thick, from about 10 to about 150 μm thick, from about 10 to about 100 μm thick, from about 10 to about 50 μm thick, from about 25 to about 250 μm thick, from about 25 to about 200 μm thick, from about 25 to about 150 μm thick, from about 25 to about 100 μm thick, or from about 25 to about 50 μm thick. In some cases, the sample may be at least about 5 μm thick, at least about 10 μm thick, at least about 25 μm thick, at least about 50 μm thick, at least about 100 μm thick, at least about 150 μm thick, at least about 200 μm thick, at least about 250 μm thick, or greater. In some cases, the sample may be at most about 5 μm thick, at most about 10 μm thick, at most about 25 μm thick, at most about 50 μm thick, at most about 100 μm thick, at most about 150 μm thick, at most about 200 μm thick, at most about 250 μm thick, or less.

The diameter of the complex may have a variety of sizes. In some embodiments, the diameter of the complex may be less than the diffraction limit of the imaging system. In some embodiments, the diameter of the complex may be more than the diffraction limit of the imaging system. In some embodiments, the diffraction limit of the imaging system may be less than about 100 μm, less than about 80 μm, less than about 60 μm, less than about 40 μm, less than about 20 μm, less than about 10 μm, less than about 5 μm, less than about 2 μm, less than about 1 μm, less than about 0.1 μm, or less than about 0.01 μm. In some embodiments, the diffraction limit of the imaging system may be more than about 100 μm, more than about 80 μm, more than about 60 μm, more than about 40 μm, more than about 20 μm, more than about 10 μm, more than about 5 μm, more than about 2 μm, more than about 1 μm, more than about 0.1 μm, or more.

In some embodiments, the method may comprise contacting the cell with a probe. The cell may be contacted with a probe after contacting the cell with a binding moiety. The cell may be contacted with a probe before contacting the cell with a binding moiety. The cell may be contacted with a probe at the same time as contacting the cell with a binding moiety. In some embodiments, the method may comprise performing a ligation reaction. In some cases, the ligation reaction may be performed after contacting the cell with a binding moiety. In some cases, the ligation reaction may be performed before contacting the cell with a binding moiety. In some cases, the ligation reaction may be performed at the same time as contacting the cell with a binding moiety. The ligation reaction may comprise ligating a nucleic acid associated with the binding moiety to form a circular nucleic acid. For example, the binding moiety may be contacted with a nucleic acid that binds to the binding moiety at two locations. The two locations may be located proximal to one another such that upon binding, one end of the nucleic acid is directly adjacent to another end of the nucleic acid and allows for ligation by an enzyme. In some embodiments, the method may comprise performing an amplification reaction. In some cases, performing an amplification reaction may take place after contacting the cell with a binding moiety. In some cases, performing an amplification reaction may take place before contacting the cell with a binding moiety. In some cases, performing an amplification reaction may take place at the same time as contacting the cell with a binding moiety. The amplification reaction may be a rolling circle amplification (RCA) reaction. The amplification reaction may generate one or more amplicons. In some embodiments, using the imaging system may comprise imaging the cell using a microscope.

The amplicons described herein may be compacted to generate a compacted amplicon. A compacted amplicon may refer to an amplicon that is reduced in size, increased in density of nucleic acid, changed in size or a combination thereof. In some cases, a compacted amplicon may be more circular than an amplicon that is not compacted. The amplicons described herein may be compacted to generate a compacted amplicon in a variety of ways. In some cases, a compacted amplicon may be generated by reacting a reactive chemical moiety with another reactive chemical moiety. The reactive chemical moieties may be part of the amplicon. In some cases, a compacted amplicon may be generated using linkers and/or polymers that bind to the amplicon and thereby decrease the size of the amplicon. For example, a nucleic acid may be used to hybridize to an amplicon at one or more locations and the amplicon may become smaller as a result. In some cases, an amplicon may be compacted to generate a compacted amplicon in one or more ways. For example, an amplicon may be compacted by reacting one or more reactive chemical moieties of the amplicon with each other and adding a nucleic acid that hybridizes to the amplicon at one or more locations. The compacted amplicons may result in one or more advantages including but not limited to a reduced size of the detected signal, an increased signal amplitude of the detected signal, an increased signal to noise ratio of the detected signal relative to background signal, a more circular (e.g. round) shape of the detected signal, or a combination thereof. The one or more advantages of the compacted amplicons may result in detecting more analytes within a sample, detecting analytes with higher accuracy within a sample, or a combination thereof.

The amplicons described herein may comprise one or more barcodes or derivatives thereof (e.g., reverse complements thereof). The one or more barcodes may comprise a nucleic acid. The nucleic acid of the one or more barcodes may comprise a combination of nucleotides including but not limited to A, C, G, T, U, or a combination thereof. The one or more barcodes may provide information related to identity of an analyte bound by a binding moiety. For example, a binding moiety may comprise an antibody that binds to a protein and the antibody may comprise a nucleic acid. The nucleic acid of the antibody may be contacted by a probe. The probe that may contact the nucleic acid of the antibody may comprise a barcode. The probe that may contact the nucleic acid of the antibody may be amplified to generate an amplicon. The amplicon may comprise one or more reverse complements of the barcode. The one or more reverse complements of the barcode may be detected, thereby identifying the analyte.

The methods described herein may comprise providing a fixed sample (e.g., a formalin-fixed paraffin embedded sample). The fixed sample may comprise a plurality of cells, a tissue sample, a blood sample, a biopsy sample, or a combination thereof. The fixed sample may have been treated with a fixative. For example, the fixed sample may have been treated with a chemical fixative, including but not limited to formaldehyde, glutaraldehyde, formalin, or a combination thereof. The fixed sample may be embedded in paraffin. The fixed sample may comprise a tissue block or a tissue slice. The fixed sample may be placed on a surface or a substrate. For example, a fixed tissue slice may be placed on a microscope slide, within a well plate, within a sample well, within a flow cell, or a combination thereof.

The plurality of analytes detected and/or identified may comprise at least about 200 analytes, at least about 250 analytes, at least about 300 analytes, at least about 350 analytes, at least about 400 analytes, at least about 450 analytes, at least about 500 analytes, at least about 600 analytes, at least about 700 analytes, at least about 800 analytes, at least about 900 analytes, at least about 1000 analytes, at least about 1500 analytes, at least about 2000 analytes, at least about 2500 analytes, at least about 5000 analytes, at least about 10000 analytes, at least about 15000 analytes, at least about 20000 analytes, at least about 50000 analytes, at least about 100000 analytes, or more. The plurality of analytes detected and/or identified may comprise at most about 200 analytes, at most about 250 analytes, at most about 300 analytes, at most about 350 analytes, at most about 400 analytes, at most about 450 analytes, at most about 500 analytes, at most about 600 analytes, at most about 700 analytes, at most about 800 analytes, at most about 900 analytes, at most about 1000 analytes, at most about 1500 analytes, at most about 2000 analytes, at most about 2500 analytes, at most about 5000 analytes, at most about 10000 analytes, at most about 15000 analytes, at most about 20000 analytes, at most about 50000 analytes, at most about 100000 analytes, or fewer. The plurality of analytes detected and/or identified may comprise about 200-100000 analytes, about 250-50000 analytes, about 300-20000 analytes, about 350-15000 analytes, about 400-10000 analytes, about 450-5000 analytes, about 500-2500 analytes, about 600-2000 analytes, about 700-1500 analytes, or about 800-1000 analytes. Another aspect of the disclosure provides a method for detecting analytes. The method may comprise providing a sample. The formalin-fixed paraffin embedded sample may comprise a plurality of cells. The plurality of cells may comprise a plurality of analytes. The method may comprise contacting the sample with a plurality of binding moieties. A binding moiety of the plurality of binding moieties may recognize an analyte of the plurality of analytes. The binding moiety of the plurality binding moieties may bind to the analyte of the plurality of analytes. The binding moiety of the plurality binding moieties may bind to and recognize the analyte of the plurality of analytes. The method may comprise contacting the sample with a plurality of detection probes to form a plurality of complexes. The plurality of complexes may be formed between binding moieties of the plurality of binding moieties and detection probes of the plurality of detection probes. The plurality of complexes may be formed between derivatives of binding moieties of the plurality of binding moieties and detection probes of the plurality of detection probes. The method may comprise detecting the plurality of complexes. Identifying the plurality of complexes may identify the plurality of analytes. Identifying the plurality of analytes may comprise identifying more than 10 analytes. The plurality of analytes may be detected in at least 98% of cells of the plurality of cells.

Another aspect of the disclosure provides a method for detecting transcripts in situ. The method may comprise providing a sample. The sample may comprise a plurality of cells. The plurality of cells may comprise a plurality of transcripts. The sample may be contacted with a plurality of binding moieties. A binding moiety of the plurality of binding moieties may recognize and bind to a transcript of the plurality of transcripts. Next, the plurality of binding moieties or derivatives thereof may be contacted with a plurality of detection probes to form a plurality of complexes. The plurality of complexes may be detected to thereby identify the plurality of transcripts. In some cases, more than 1, 2, 3, 4, 5, 6, 7, 8, 9, 10, 20, 30, 40, 50, 60, 70, 80, 90, 100, 150, or 200 transcripts (e.g., different transcripts) may be detected in at least 90%, 91%, 92%, 93%, 94%, 95%, 96%, 97%, 98%, 99%, or 100% of cells of the plurality of cells.

In some embodiments, the formalin-fixed paraffin embedded sample may be a formalin-fixed paraffin embedded tissue sample. The formalin-fixed paraffin embedded sample may comprise a tissue slice obtained from a tissue bloc. The formalin-fixed paraffin embedded tissue sample may be mounted on a surface used for analysis. In some cases, the surface used for analysis may comprise a coverslip, a slide, a well of a well plate, or a combination thereof. The formalin-fixed paraffin embedded sample may have a variety of thicknesses including but not limited to about 5 to about 250 μm thick, from about 5 to about 200 μm thick, from about 5 to about 150 μm thick, from about 5 to about 100 μm thick, from about 5 to about 50 μm thick, from about 5 to about 10 μm thick, from about 10 to about 250 μm thick, from about 10 to about 200 μm thick, from about 10 to about 150 μm thick, from about 10 to about 100 μm thick, from about 10 to about 50 μm thick, from about 25 to about 250 μm thick, from about 25 to about 200 μm thick, from about 25 to about 150 μm thick, from about 25 to about 100 μm thick, or from about 25 to about 50 μm thick. In some cases, the formalin-fixed paraffin embedded sample may be at least about 5 μm thick, at least about 10 μm thick, at least about 25 μm thick, at least about 50 μm thick, at least about 100 μm thick, at least about 150 μm thick, at least about 200 μm thick, at least about 250 μm thick, or greater. In some cases, the formalin-fixed paraffin embedded sample may be at most about 5 μm thick, at most about 10 μm thick, at most about 25 μm thick, at most about 50 μm thick, at most about 100 μm thick, at most about 150 μm thick, at most about 200 μm thick, at most about 250 μm thick, or less.

In some embodiments, identifying the plurality of transcripts may comprise identifying greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, greater than or equal to 10, greater than or equal to 20, greater than or equal to 30, greater than or equal to 40, greater than or equal to 50, greater than or equal to 60, greater than or equal to 70, greater than or equal to 80, greater than or equal to 90, greater than or equal to 100, greater than or equal to 120, greater than or equal to 200, greater than or equal to 300, greater than or equal to 400, greater than or equal to 500, greater than or equal to 600, greater than or equal to 700, greater than or equal to 800, greater than or equal to 900, or greater than or equal to 1000 transcripts (e.g., different transcripts) on average per cell. In some embodiments, the plurality of transcripts may comprise messenger RNA (mRNA). In some embodiments, the plurality of transcripts may comprise pre-mRNA.

In some embodiments, the method may comprise contacting the formalin-fixed paraffin embedded sample with a probe. In some cases, contacting the formalin-fixed paraffin embedded sample with a probe may take place after contacting the formalin-fixed paraffin embedded sample with a plurality of binding moieties. In some cases, contacting the formalin-fixed paraffin embedded sample with a probe may take place before contacting the formalin-fixed paraffin embedded sample with a plurality of binding moieties. In some cases, contacting the formalin-fixed paraffin embedded sample with a probe may take place at the same time as contacting the formalin-fixed paraffin embedded sample with a plurality of binding moieties. The probe may comprise nucleic acid, a polypeptide, or a combination thereof. In some cases, the nucleic acid of the probe may comprise deoxyribonucleic acid, ribonucleic acid, or a combination thereof. The nucleic acid of the probe may bind to the binding moiety of the plurality of binding moieties. For example, a nucleic acid may bind to the binding moiety at two locations of the binding moiety. The two locations of the binding moiety (e.g., two regions of a nucleic acid of the binding moiety) may be proximal to one another. Once bound to the binding moiety, two ends of the nucleic acid may be directly adjacent to one another. In some embodiments, the method may comprise performing a ligation reaction. In some cases, the ligation reaction may be performed after contacting the formalin-fixed paraffin embedded sample with a plurality of binding moieties. In some cases, the ligation reaction may be performed before contacting the formalin-fixed paraffin embedded sample with a plurality of binding moieties. In some cases, the ligation reaction may be performed at the same time as contacting the formalin-fixed paraffin embedded sample with a plurality of binding moieties. The ligation reaction may comprise ligating a nucleic acid (e.g., the probe) associated with a binding moiety of the plurality of binding moieties to form a circular nucleic acid. In some embodiments, the method may comprise performing an amplification reaction (e.g., a rolling circle amplification reaction). In some cases, performing the amplification reaction may take place after contacting the formalin-fixed paraffin embedded sample with a plurality of binding moieties. In some cases, performing the amplification reaction may take place before contacting the formalin-fixed paraffin embedded sample with a plurality of binding moieties. In some cases, performing the amplification reaction may take place at the same time as contacting the formalin-fixed paraffin embedded sample with a plurality of binding moieties. In some embodiments, detecting the plurality of complexes may comprise imaging the cell using a microscope.

In some cases, a plurality of cells may be analyzed using any one of the methods described herein. The plurality of cells may be from cell culture. The plurality of cells may be part of a tissue sample (e.g., a tissue slice). A number of analytes may be identified in cells of the plurality of cells. For example, in some cases at least about 10 analytes, at least about 15 analytes, at least about 20 analytes, at least about 25 analytes, at least about 50 analytes, at least about 100 analytes, at least about 150 analytes, at least about 200 analytes, at least about 250 analytes, at least about 500 analytes, or at least about 1000 analytes may be identified in at least about 80% of the plurality of cells, at least about 81% of the plurality of cells, at least about 82% of the plurality of cells, at least about 83% of the plurality of cells, at least about 84% of the plurality of cells, at least about 85% of the plurality of cells, at least about 86% of the plurality of cells, at least about 87% of the plurality of cells, at least about 88% of the plurality of cells, at least about 89% of the plurality of cells, at least about 90% of the plurality of cells, at least about 91% of the plurality of cells, at least about 92% of the plurality of cells, at least about 93% of the plurality of cells, at least about 94% of the plurality of cells, at least about 95% of the plurality of cells, at least about 96% of the plurality of cells, at least about 97% of the plurality of cells, at least about 98% of the plurality of cells, at least about 99% of the plurality of cells, or 100% of the plurality of cells.

The methods described herein may comprise contacting a sample with a plurality of detection probes to form a plurality of complexes. In some cases, a detection probe may be added to a sample to form a complex. The detection probes of the plurality of detection probes may bind to, recognize, or recognize and bind to one or more amplicons or one or more binding moieties of the methods described herein. For example, in some cases, the detection probes of the plurality of detection probes may bind to binding moieties of the plurality of binding moieties to form binding complexes. In some cases, one or more amplicons may be generated with the aid of a binding moiety of the plurality of binding moieties. The one or more amplicons may be compacted using any one of the methods described herein. The one or more amplicons may comprise a reverse complement (e.g., a derivative) of at least a portion of one or more binding moieties of the plurality of binding moieties.

Another aspect of the disclosure provides a method for detecting analytes. The method may comprise providing a sample. The formalin-fixed paraffin embedded sample may comprise a plurality of cells. The plurality of cells may comprise a plurality of analytes. The method may comprise contacting the sample with a plurality of binding moieties. A binding moiety of the plurality of binding moieties may recognize an analyte of the plurality of analytes. The binding moiety of the plurality binding moieties may bind to the analyte of the plurality of analytes. The binding moiety of the plurality binding moieties may bind to and recognize the analyte of the plurality of analytes. The method may comprise contacting the sample with a plurality of detection probes to form a plurality of complexes. The plurality of complexes may be formed between binding moieties of the plurality of binding moieties and detection probes of the plurality of detection probes. The plurality of complexes may be formed between derivatives of binding moieties of the plurality of binding moieties and detection probes of the plurality of detection probes. The method may comprise detecting the plurality of complexes. Identifying the plurality of complexes may identify the plurality of analytes. Identifying the plurality of analytes may comprise identifying more than 10 analytes may be detected in at least 90% of cells of the plurality of cells.

Another aspect of the disclosure provides a method for detecting transcripts in situ. The method may comprise providing a sample. The sample may comprise a plurality of cells. The plurality of cells may comprise a plurality of transcripts. The sample may be contacted with a plurality of binding moieties. A binding moiety of the plurality of binding moieties may recognize and bind to a transcript of the plurality of transcripts. Next, the plurality of binding moieties or derivatives thereof may be contacted with a plurality of detection probes to form a plurality of complexes. The plurality of complexes may be detected to thereby identify the plurality of transcripts. In some cases, more than 1, 2, 3, 4, 5, 6, 7, 8, 9, 10, 20, 30, 40, 50, 60, 70, 80, 90, 100, 150, or 200 transcripts (e.g., different transcripts) may be detected in at least 90%, 91%, 92%, 93%, 94%, 95%, 96%, 97%, 98%, 99%, or 100% of cells of the plurality of cells.

In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 1 transcript. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 2 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 3 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 4 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 5 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 6 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 7 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 8 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 9 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 10 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 20 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 30 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 40 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 50 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 60 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 70 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 80 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 90 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 100 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 150 transcripts. In some embodiments, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, greater than or equal to 99%, or greater than or equal to 100% of cells of the plurality of cells comprises greater than or equal to 200 transcripts.

In some embodiments, the method may comprise contacting the sample with a probe. In some cases, contacting the sample with the probe may take place after contacting the sample with a plurality of binding moieties. In some cases, contacting the sample with the probe may take place before contacting the sample with a plurality of binding moieties. In some cases, contacting the sample with the probe may take place at the same time as contacting the sample with a plurality of binding moieties. The probe may comprise nucleic acid, a polypeptide, or a combination thereof. In some cases, the nucleic acid of the probe may comprise deoxyribonucleic acid, ribonucleic acid, or a combination thereof. The nucleic acid of the probe may bind to the binding moiety of the plurality of binding moieties. For example, a nucleic acid may bind to the binding moiety at two locations of the binding moiety. The two locations of the binding moiety (e.g., two regions of a nucleic acid of the binding moiety) may be proximal to one another. Once bound to the binding moiety, two ends of the nucleic acid may be directly adjacent to one another. In some embodiments, the method may comprise performing a ligation reaction. The ligation reaction comprises ligating a nucleic acid (e.g., the probe) associated with the binding moiety to form a circular nucleic acid. In some embodiments, the method may comprise performing an amplification reaction (e.g., a rolling circle amplification reaction). In some embodiments, detecting the plurality of complexes may comprise imaging the cell using a microscope.

### Sample

The methods described herein relate to analyzing one or more analytes in a sample. In some aspects, the sample comprising the analyte may comprise a biological specimen extracted from a subject. The subject may be a mammal, a bacteria, a fungus, or a combination thereof. The subject may be a human subject. The subject may be a mouse. In some cases, the sample may comprise cells grown ex vivo, e.g., using cell culture methods. In some embodiments, the sample may comprise a cell, a tissue, a bodily fluid, or a combination thereof.

In some instances, the sample comprises or is derived from a naturally occurring cell (e.g., cells from a primary source) or naturally occurring cell populations. In some cases, the sample comprises a genetically engineered cell or cell lines. In some cases, the sample comprises cells derived from a transgenic animal or animals. In some instances, the sample comprises bacterial, fungal, plant or animal cells. In some instances, the sample comprises mammalian cells. In some cases, the sample comprises cells or tissue derived from blood, bone marrow, liver, pancreas, neural tissue, bone marrow, or skin. In some instances, the sample comprises cultured cells.

The sample comprising the analyte may be a variety of formats and/or may comprise a variety or features and/or characteristics. The sample may comprise one or more cells. In some embodiments, the sample may comprise one or more cells, one or more tissue samples, one or more bodily fluids, or a combination thereof. The cells of the sample may be cultured cells. The cultured cells may be cultured in vivo, ex vivo or in vitro. The sample may comprise a tissue sample. The tissue sample may be fresh, fresh-frozen, fixed, fixed-frozen, formalin-fixed, paraffin embedded, or a combination thereof. In some cases, the tissue sample may be fixed using a cross-linking reagent. In some cases, the tissue sample may be fixed using a preservative. In some cases, the cross-linking reagent may comprise formaldehyde, formalin, glutaraldehyde, or a combination thereof.

In some instances, the sample comprises a single cell type. In some cases, the sample comprises a plurality of cell types. In some cases, the sample comprises neuronal (e.g., excitatory or inhibitory) cells or non-neuronal cells. In some cases, the sample comprises neuronal (e.g., excitatory or inhibitory) cells and non-neuronal cells. In some instances, the sample comprises neuronal cells (e.g., excitatory or inhibitory neurons). In some instances, the sample comprises glial cells. In some cases, the sample comprises oligodendrocytes. In some cases, the sample comprises astrocytes.

In some instances, the sample includes brain tissue (e.g., visual cortex slices). In some instances, the sample comprises smooth muscle cells. In some cases, the sample comprises endothelial cells. In some instances, the sample is a biological tissue comprising epithelial tissue, connective tissue, muscle tissue, or nervous tissue. In some cases, the sample comprises epithelial tissue. In some instances, the sample comprises connective tissue. In some cases, the sample comprises muscle tissue. In some instances, the sample comprises nervous tissue.

The sample may comprise a tissue sample that has been sliced from a tissue block. The tissue sample may be immobilized onto a substrate. The substrate may be a well-plate, a slide, a coverslip, a well, a surface, a flow cell, or a combination thereof. The slide may be a microscope slide. The tissue sample may have a variety of thicknesses. The tissue sample may be at least at least about 1 μm thick, at least about 2 μm thick, at least about 3 μm thick, at least about 4 μm thick, at least about 5 μm thick, at least about 6 μm thick, at least about 7 μm thick, at least about 8 μm thick, at least about 9 μm thick, at least about 10 μm thick, at least about 11 μm thick, at least about 12 μm thick, at least about 13 μm thick, at least about 14 μm thick, at least about 15 μm thick, at least about 16 μm thick, at least about 17 μm thick, at least about 18 μm thick, at least about 19 μm thick, at least about 20 μm thick, at least about 21 μm thick, at least about 22 μm thick, at least about 23 μm thick, at least about 24 μm thick, at least about 25 μm thick, at least about 26 μm thick, at least about 27 μm thick, at least about 28 μm thick, at least about 29 μm thick, at least about 30 μm thick, at least about 31 μm thick, at least about 32 μm thick, at least about 33 μm thick, at least about 34 μm thick, at least about 35 μm thick, at least about 36 μm thick, at least about 37 μm thick, at least about 38 μm thick, at least about 39 μm thick, at least about 40 μm thick, at least about 41 μm thick, at least about 42 μm thick, at least about 43 μm thick, at least about 44 μm thick, at least about 45 μm thick, at least about 46 μm thick, at least about 47 μm thick, at least about 48 μm thick, at least about 49 μm thick, at least about 50 μm thick, at least about 51 μm thick, at least about 52 μm thick, at least about 53 μm thick, at least about 54 μm thick, at least about 55 μm thick, at least about 56 μm thick, at least about 57 μm thick, at least about 58 μm thick, at least about 59 μm thick, at least about 60 μm thick, at least about 61 μm thick, at least about 62 μm thick, at least about 63 μm thick, at least about 64 μm thick, at least about 65 μm thick, at least about 66 μm thick, at least about 67 μm thick, at least about 68 μm thick, at least about 69 μm thick, at least about 70 μm thick, at least about 71 μm thick, at least about 72 μm thick, at least about 73 μm thick, at least about 74 μm thick, at least about 75 μm thick, at least about 76 μm thick, at least about 77 μm thick, at least about 78 μm thick, at least about 79 μm thick, at least about 80 μm thick, at least about 81 μm thick, at least about 82 μm thick, at least about 83 μm thick, at least about 84 μm thick, at least about 85 μm thick, at least about 86 μm thick, at least about 87 μm thick, at least about 88 μm thick, at least about 89 μm thick, at least about 90 μm thick, at least about 91 μm thick, at least about 92 μm thick, at least about 93 μm thick, at least about 94 μm thick, at least about 95 μm thick, at least about 96 μm thick, at least about 97 μm thick, at least about 98 μm thick, at least about 99 μm thick, at least about 100 μm thick, at least about 105 μm thick, at least about 110 μm thick, at least about 115 μm thick, at least about 120 μm thick, at least about 125 μm thick, at least about 130 μm thick, at least about 135 μm thick, at least about 140 μm thick, at least about 145 μm thick, at least about 150 μm thick, at least about 155 μm thick, at least about 160 μm thick, at least about 165 μm thick, at least about 170 μm thick, at least about 175 μm thick, at least about 180 μm thick, at least about 185 μm thick, at least about 190 μm thick, at least about 195 μm thick, at least about 200 μm thick, at least about 210 μm thick, at least about 220 μm thick, at least about 230 μm thick, at least about 240 μm thick, at least about 250 μm thick, at least about 260 μm thick, at least about 270 μm thick, at least about 280 μm thick, at least about 290 μm thick, at least about 300 μm thick, at least about 320 μm thick, at least about 340 μm thick, at least about 360 μm thick, at least about 380 μm thick, at least about 400 μm thick, at least about 420 μm thick, at least about 440 μm thick, at least about 460 μm thick, at least about 480 μm thick, at least about 500 μm thick, or more. The tissue sample may be at most at most about 1 μm thick, at most about 2 μm thick, at most about 3 μm thick, at most about 4 μm thick, at most about 5 μm thick, at most about 6 μm thick, at most about 7 μm thick, at most about 8 μm thick, at most about 9 μm thick, at most about 10 μm thick, at most about 11 μm thick, at most about 12 μm thick, at most about 13 μm thick, at most about 14 μm thick, at most about 15 μm thick, at most about 16 μm thick, at most about 17 μm thick, at most about 18 μm thick, at most about 19 μm thick, at most about 20 μm thick, at most about 21 μm thick, at most about 22 μm thick, at most about 23 μm thick, at most about 24 μm thick, at most about 25 μm thick, at most about 26 μm thick, at most about 27 μm thick, at most about 28 μm thick, at most about 29 μm thick, at most about 30 μm thick, at most about 31 μm thick, at most about 32 μm thick, at most about 33 μm thick, at most about 34 μm thick, at most about 35 μm thick, at most about 36 μm thick, at most about 37 μm thick, at most about 38 μm thick, at most about 39 μm thick, at most about 40 μm thick, at most about 41 μm thick, at most about 42 μm thick, at most about 43 μm thick, at most about 44 μm thick, at most about 45 μm thick, at most about 46 μm thick, at most about 47 μm thick, at most about 48 μm thick, at most about 49 μm thick, at most about 50 μm thick, at most about 51 μm thick, at most about 52 μm thick, at most about 53 μm thick, at most about 54 μm thick, at most about 55 μm thick, at most about 56 μm thick, at most about 57 μm thick, at most about 58 μm thick, at most about 59 μm thick, at most about 60 μm thick, at most about 61 μm thick, at most about 62 μm thick, at most about 63 μm thick, at most about 64 μm thick, at most about 65 μm thick, at most about 66 μm thick, at most about 67 μm thick, at most about 68 μm thick, at most about 69 μm thick, at most about 70 μm thick, at most about 71 μm thick, at most about 72 μm thick, at most about 73 μm thick, at most about 74 μm thick, at most about 75 μm thick, at most about 76 μm thick, at most about 77 μm thick, at most about 78 μm thick, at most about 79 μm thick, at most about 80 μm thick, at most about 81 μm thick, at most about 82 μm thick, at most about 83 μm thick, at most about 84 μm thick, at most about 85 μm thick, at most about 86 μm thick, at most about 87 μm thick, at most about 88 μm thick, at most about 89 μm thick, at most about 90 μm thick, at most about 91 μm thick, at most about 92 μm thick, at most about 93 μm thick, at most about 94 μm thick, at most about 95 μm thick, at most about 96 μm thick, at most about 97 μm thick, at most about 98 μm thick, at most about 99 μm thick, at most about 100 μm thick, at most about 105 μm thick, at most about 110 μm thick, at most about 115 μm thick, at most about 120 μm thick, at most about 125 μm thick, at most about 130 μm thick, at most about 135 μm thick, at most about 140 μm thick, at most about 145 μm thick, at most about 150 μm thick, at most about 155 μm thick, at most about 160 μm thick, at most about 165 μm thick, at most about 170 μm thick, at most about 175 μm thick, at most about 180 μm thick, at most about 185 μm thick, at most about 190 μm thick, at most about 195 μm thick, at most about 200 μm thick, at most about 210 μm thick, at most about 220 μm thick, at most about 230 μm thick, at most about 240 μm thick, at most about 250 μm thick, at most about 260 μm thick, at most about 270 μm thick, at most about 280 μm thick, at most about 290 μm thick, at most about 300 μm thick, at most about 320 μm thick, at most about 340 μm thick, at most about 360 μm thick, at most about 380 μm thick, at most about 400 μm thick, at most about 420 μm thick, at most about 440 μm thick, at most about 460 μm thick, at most about 480 μm thick, at most about 500 μm thick, or less. The tissue sample may be about 1 to about 500 μm thick, about 2 to about 480 μm thick, about 3 to about 460 μm thick, about 4 to about 440 μm thick, about 5 to about 420 μm thick, about 6 to about 400 μm thick, about 7 to about 380 μm thick, about 8 to about 360 μm thick, about 9 to about 340 μm thick, about 10 to about 320 μm thick, about 11 to about 300 μm thick, about 12 to about 290 μm thick, about 13 to about 280 μm thick, about 14 to about 270 μm thick, about 15 to about 260 μm thick, about 16 to about 250 μm thick, about 17 to about 240 μm thick, about 18 to about 230 μm thick, about 19 to about 220 μm thick, about 20 to about 210 μm thick, about 21 to about 200 μm thick, about 22 to about 195 μm thick, about 23 to about 190 μm thick, about 24 to about 185 μm thick, about 25 to about 180 μm thick, about 26 to about 175 μm thick, about 27 to about 170 μm thick, about 28 to about 165 μm thick, about 29 to about 160 μm thick, about 30 to about 155 μm thick, about 31 to about 150 μm thick, about 32 to about 145 μm thick, about 33 to about 140 μm thick, about 34 to about 135 μm thick, about 35 to about 130 μm thick, about 36 to about 125 μm thick, about 37 to about 120 μm thick, about 38 to about 115 μm thick, about 39 to about 110 μm thick, about 40 to about 105 μm thick, about 41 to about 100 μm thick, about 42 to about 99 μm thick, about 43 to about 98 μm thick, about 44 to about 97 μm thick, about 45 to about 96 μm thick, about 46 to about 95 μm thick, about 47 to about 94 μm thick, about 48 to about 93 μm thick, about 49 to about 92 μm thick, about 50 to about 91 μm thick, about 51 to about 90 μm thick, about 52 to about 89 μm thick, about 53 to about 88 μm thick, about 54 to about 87 μm thick, about 55 to about 86 μm thick, about 56 to about 85 μm thick, about 57 to about 84 μm thick, about 58 to about 83 μm thick, about 59 to about 82 μm thick, about 60 to about 81 μm thick, about 61 to about 80 μm thick, about 62 to about 79 μm thick, about 63 to about 78 μm thick, about 64 to about 77 μm thick, about 65 to about 76 μm thick, about 66 to about 75 μm thick, about 67 to about 74 μm thick, about 68 to about 73 μm thick, about 69 to about 72 μm thick, or about 70 to about 71 μm thick. In some cases, the tissue sample is about 5 to about 250 μm thick, about 10 to about 100 μm thick, or about 25 to about 150 μm thick.

In some instances, the sample may comprise a tissue region (e.g., a tissue region based on the Allen Mouse Brain Reference Atlas). In some cases, the tissue region is selected from: anterior cingulate area; agranular insular area; agranular insular area, posterior part; alveus; accessory olfactory bulb, granule layer; anterior olfactory nucleus; anterior olfactory nucleus, medial part; cerebral aqueduct; arcuate hypothalamic nucleus; auditory area; basolateral amygdalar nucleus; basomedial amygdalar nucleus; bed nuclei of the stria terminalis anterior division ventral nucleus; bed nuclei of the stria terminalis posterior division dorsal nucleus; field CA1, pyramidal layer; field CA2, pyramidal layer; field CA3, pyramidal layer; cerebellar cortex, dorsal part, granular layer; cerebellar cortex, molecular layer; cerebellar cortex, Purkinje layer; cerebellar cortex, ventral part, granular layer; corpus callosum; central amygdalar nucleus; central amygdalar nucleus, lateral part; choroid plexus; cingulum bundle; claustrum; cortical amygdalar area, anterior part; cortical amygdalar area, posterior part; caudoputamen; CTX, cerebral cortex; cortical subplate; dorsal fornix; dentate gyrus; 4 dentate gyrus, dorsal part, granule cell layer; dentate gyrus, molecular layer/polymorph layer; dentate gyrus, ventral part, granule cell layer; dorsomedial nucleus of the hypothalamus; dorsal motor nucleus of the vagus nerve; dorsal nucleus raphe; ectorhinal area; entorhinal area, lateral part; entorhinal area, medial part; endopiriform nucleus; Edinger-Westphal nucleus; forebrain; fiber tracts; fasciola cinerea; fimbria; hindbrain; hindbrain lateral part; hippocampal formation; hippocampal formation stratum lacunosum-moleculare/stratum radiatum/stratum oriens; hypothalamus, anterior-lateral enriched; HYam, hypothalamus, anterior medial enriched; hypothalamus, posterior-medial part enriched; IA, intercalated amygdalar nucleus; IC, inferior colliculus; IG, indusium griseum; III, oculomotor nucleus; ILA, infralimbic area; int, internal capsule; IO, inferior olivary complex; IPN, interpeduncular nucleus; L1l, cerebral cortical layer 1, lateral part; L1m, cerebral cortical layer 1, medial part; L2/3, layer 2/3; L4, layer 4; L5, layer 5; L6, layer 6; L6a, layer 6a; L6b, layer 6b; LA, lateral amygdalar nucleus; locus coeruleus; laterodorsal tegmental nucleus; LH, lateral habenula; lateral hypothalamic area; lateral septal nucleus; medial amygdalar nucleus; medial habenula; medial habenula, dorsal part; medial habenula, ventral part; medial mammillary nucleus, anterior part; medial mammillary nucleus, posterior part; meninges; mo, molecular layer; somatomotor areas; primary motor area; main olfactory bulb, granule layer; medial prefrontal cortex; medulla, anterior enriched; medulla, dorsal part; medulla, medial enriched; medulla, posterior enriched; medial vestibular nucleus; nucleus of the solitary tract; olfactory bulb, glomerular layer; olfactory bulb, mitral layer; olfactory bulb, outer plexiform layer; olfactory nerve layer of main olfactory bulb; pons; posterior amygdalar nucleus; pallidum, dorsal region; pallidum, medial region; pallidum, ventral region; periaqueductal gray, dorsal part enriched; periaqueductal gray, posterior ventral part; pontine central gray; perirhinal area; piriform area; prelimbic area; pons, medial part; pedunculopontine nucleus; polymorph layer; postsubiculum; presubiculum; principal sensory nucleus of the trigeminal; paraventricular hypothalamic nucleus; periventricular hypothalamic nucleus, posterior part; nucleus of reuniens; nucleus raphe obscurus; nucleus raphe pallidus; midbrain reticular nucleus, retrorubral area; retrosplenial cortex; reticular nucleus of the thalamus; striatum-like amygdalar nuclei; superior colliculus; suprachiasmatic nucleus; subcommissural organ; subependymal zone; subfornical organ; granule cell layer; stratum lacunosummoleculare; substantia nigra, compact part; substantia nigra, reticular part; stratum oriens; pyramidal layer; spinal nucleus of the trigeminal; stratum radiatum; somatosensory area; primary SS; secondary SS; subthalamus nucleus; striatum; periventricular area of striatum; dorsal striatum, anterior-lateral enriched; dorsal striatum, posterior-medial enriched; striatum ventral region; ventral striatum, anterior-lateral enriched; ventral striatum, islands of Calleja; ventral striatum, olfactory tubercle; ventral striatum, posterior-medial enriched; subiculum, pyramidal layer; subiculum stratum radiatum; temporal association area; thalamus; lateral TH; anterior-medial TH; thalamus medial part; posterior medial TH; tuberomammillary nucleus; triangular nucleus of septum; taenia tecta, dorsal part; taenia tecta, ventral part; motor nucleus of 5 trigeminal; third ventricle; facial motor nucleus; visual area; visceral area; lateral ventricle; ventromedial hypothalamic nucleus; ventral tegmental area; ventricular wall; or zona incerta.

The methods described herein may comprise detecting the analyte within the sample (e.g., a tissue sample). The methods described herein may comprise detecting the analyte on a tissue surface. In some cases, the methods described herein may comprise detecting an analyte outside of a tissue sample (e.g., a cytokine outside of a tissue sample). The sample may be embedded in a hydrogel. The hydrogel may be formed by polymerizing monomers in the presence of the sample. The hydrogel may comprise one or more polymers. The one or more polymers may comprise poly (vinyl alcohol) (PVA), poly (ethylene glycol) (PEG), poly (ethylene oxide) (PEO), poly (2-hydroxyethyl methacrylate) (PHEMA), poly (acrylic acid) (PAA), poly (acrylamide) (PAAm), or a combination thereof. Hydrogel may be formed during any operation of the methods described herein.

The sample may be configured to be imaged using any one of the imaging systems described herein. For example, the sample may be placed on a surface and/or substrate. The sample may be placed on a microscope slide, on a coverslip, in a sample well, in a flow cell, or a combination thereof. The sample may be treated with a fixative (e.g., formaldehyde) after being placed on a substrate configured for imaging. In some cases, the sample may be treated with one or more solvents. The one or more solvents may comprise ethanol, methanol, acetone, acetonitrile, or a combination thereof.

The sample may comprise a plurality of cells. The plurality of cells may comprise at least about 100 cells, at least about 500 cells, at least about 1,000 cells, at least about 5,000 cells, at least about 10,000 cells, at least about 20,000 cells, at least about 30,000 cells, at least about 40,000 cells, at least about 50,000 cells, at least about 60,000 cells, at least about 70,000 cells, at least about 80,000 cells, at least about 90,000 cells, at least about 100,000 cells, at least about 250,000 cells, at least about 500,000 cells, at least about 750,000 cells, at least about 1,000,000 cells, at least about 2,000,000 cells, at least about 5,000,000 cells, at least about 10,000,000 cells, or more cells. The plurality of cells may comprise at most about 100 cells, at most about 500 cells, at most about 1,000 cells, at most about 5,000 cells, at most about 10,000 cells, at most about 20,000 cells, at most about 30,000 cells, at most about 40,000 cells, at most about 50,000 cells, at most about 60,000 cells, at most about 70,000 cells, at most about 80,000 cells, at most about 90,000 cells, at most about 100,000 cells, at most about 250,000 cells, at most about 500,000 cells, at most about 750,000 cells, at most about 1,000,000 cells, at most about 2,000,000 cells, at most about 5,000,000 cells, at most about 10,000,000 cells, or fewer cells. The plurality of cells may comprise about 100-10,000,000 cells, about 500-5,000,000 cells, about 1,000-2,000,000 cells, about 5,000-1,000,000 cells, about 10,000-750,000 cells, about 20,000-500,000 cells, about 30,000-250,000 cells, about 40,000-100,000 cells, about 50,000-90,000 cells, or about 60,000-80,000 cells.

In some cases, the sample has a length in the z-direction of at least about 5 micrometers (μm), at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, at least about 120 μm, at least about 130 μm, at least about 140 μm, at least about 150 μm, at least about 160 μm, at least about 170 μm, at least about 180 μm, at least about 190 μm, at least about 200 μm, at least about 210 μm, at least about 220 μm, at least about 230 μm, at least about 240 μm, at least about 250 μm, at least about 260 μm, at least about 270 μm, at least about 280 μm, at least about 290 μm, at least about 300 μm, at least about 310 μm, at least about 320 μm, at least about 330 μm, at least about 340 μm, at least about 350 μm, at least about 360 μm, at least about 370 μm, at least about 380 μm, at least about 390 μm, at least about 400 μm, at least about 410 μm, at least about 420 μm, at least about 430 μm, at least about 440 μm, at least about 450 μm, at least about 460 μm, at least about 470 μm, at least about 480 μm, at least about 490 μm, at least about 500 μm, at least about 510 μm, at least about 520 μm, at least about 530 μm, at least about 540 μm, at least about 550 μm, at least about 560 μm, at least about 570 μm, at least about 580 μm, at least about 590 μm, at least about 600 μm, at least about 610 μm, at least about 620 μm, at least about 630 μm, at least about 640 μm, at least about 650 μm, at least about 660 μm, at least about 670 μm, at least about 680 μm, at least about 690 μm, at least about 700 μm, at least about 710 μm, at least about 720 μm, at least about 730 μm, at least about 740 μm, at least about 750 μm, at least about 760 μm, at least about 770 μm, at least about 780 μm, at least about 790 μm, at least about 800 μm, at least about 810 μm, at least about 820 μm, at least about 830 μm, at least about 840 μm, at least about 850 μm, at least about 860 μm, at least about 870 μm, at least about 880 μm, at least about 890 μm, at least about 900 μm, at least about 910 μm, at least about 920 μm, at least about 930 μm, at least about 940 μm, at least about 950 μm, at least about 960 μm, at least about 970 μm, at least about 980 μm, at least about 990 μm, at least about 1000 μm, or more.

In some cases, at most about the sample has a length in the z-direction of less than 5 μm, at most about 10 μm, at most about 20 μm, at most about 30 μm, at most about 40 μm, at most about 50 μm, at most about 60 μm, at most about 70 μm, at most about 80 μm, at most about 90 μm, at most about 100 μm, at most about 110 μm, at most about 120 μm, at most about 130 μm, at most about 140 μm, at most about 150 μm, at most about 160 μm, at most about 170 μm, at most about 180 μm, at most about 190 μm, at most about 200 μm, at most about 210 μm, at most about 220 μm, at most about 230 μm, at most about 240 μm, at most about 250 μm, at most about 260 μm, at most about 270 μm, at most about 280 μm, at most about 290 μm, at most about 300 μm, at most about 310 μm, at most about 320 μm, at most about 330 μm, at most about 340 μm, at most about 350 μm, at most about 360 μm, at most about 370 μm, at most about 380 μm, at most about 390 μm, at most about 400 μm, at most about 410 μm, at most about 420 μm, at most about 430 μm, at most about 440 μm, at most about 450 μm, at most about 460 μm, at most about 470 μm, at most about 480 μm, at most about 490 μm, at most about 500 μm, at most about 510 μm, at most about 520 μm, at most about 530 μm, at most about 540 μm, at most about 550 μm, at most about 560 μm, at most about 570 μm, at most about 580 μm, at most about 590 μm, at most about 600 μm, at most about 610 μm, at most about 620 μm, at most about 630 μm, at most about 640 μm, at most about 650 μm, at most about 660 μm, at most about 670 μm, at most about 680 μm, at most about 690 μm, at most about 700 μm, at most about 710 μm, at most about 720 μm, at most about 730 μm, at most about 740 μm, at most about 750 μm, at most about 760 μm, at most about 770 μm, at most about 780 μm, at most about 790 μm, at most about 800 μm, at most about 810 μm, at most about 820 μm, at most about 830 μm, at most about 840 μm, at most about 850 μm, at most about 860 μm, at most about 870 μm, at most about 880 μm, at most about 890 μm, at most about 900 μm, at most about 910 μm, at most about 920 μm, at most about 930 μm, at most about 940 μm, at most about 950 μm, at most about 960 μm, at most about 970 μm, at most about 980 μm, at most about 990 μm, at most about 1000 μm, or less.

In some cases, the sample is treated by a method disclosed in US Patent Publication No.: 20210164039, PCT Publication No.: WO2023278409, or PCT Publication No.: WO2023018756, each of which is incorporated herein by reference in its entirety.

### Analytes

The methods described herein may comprise identifying one or more analytes. A variety of different types of analytes may be identified, including, but not limited to, nucleic acids, polypeptides, lipids, small molecules, cells, or a combination thereof. In some cases, one or more analytes of the same type may be identified (e.g., one or more protein analytes). For example, two different proteins may be identified. In some cases, one or more analytes of different types of analytes may be analyzed (e.g., different analyte forms or compositions). For example, a protein and/or a nucleic acid may be identified.

In some aspects, the one or more analytes that are analyzed detected, and/or identified using any one of the methods described herein may comprise one or more polypeptides. The one or more polypeptides may comprise one or more proteins, one or more peptides, or a combination thereof. In some cases, the one or more polypeptides may comprise one or more proteins and the one or more proteins may comprise one or more enzymes, one or more antibodies, one or more transcription factors, one or more structural proteins, one or more defense proteins, one or more signaling proteins, one or more receptors, one or more soluble proteins, one or more transmembrane proteins, or a combination thereof. In cases where the one or more proteins may comprise one or more signaling proteins, the one or more signaling proteins may comprise one or more cytokines, one or more chemokines, or a combination thereof. The one or more polypeptides may be implicated in a disease state or mechanism of interest. In cases where the one or more analytes that are identified comprise one or more polypeptides, the binding moiety used to recognize and/or bind to the one or more polypeptides may comprise an antibody, a nanobody, an antibody fragment, or any combination thereof.

In some aspects, the one or more analytes that are analyzed detected, and/or identified using any one of the methods described herein may comprise one or more lipids. The one or more lipids may comprise one or more phospholipids, one or more sterols, one or more triglycerides or a combination thereof. In cases where the one or more analytes that are identified comprise one or more lipids, a binding moiety that binds to and/or recognizes the one or more lipids may comprise a moiety that detects, couples to, binds to, or otherwise recognizes the one or more lipids. For example, the binding moiety may comprise an antibody, a nanobody, a lipid binding protein, or a combination thereof. In some cases, the lipid binding protein may comprise Lipid A.

In some aspects, the one or more analytes that are analyzed detected, and/or identified using any one of the methods described herein may comprise one or more small molecules. The one or more small molecules may comprise one or more signaling molecules, hormones, or a combination thereof. In cases where the one or more analytes that are identified comprise one or more small molecules, a binding moiety that binds to and/or recognizes the one or more small molecules may comprise a moiety that detects, couples to, binds to, or otherwise recognizes the one or more small molecules. For example, the binding moiety may comprise an antibody, a nanobody, or a combination thereof.

In some aspects, the one or more analytes that are analyzed detected, and/or identified using any one of the methods described herein may comprise one or more cells. The one or more cells may comprise a cell that expresses a marker unique to or indicative of the cell. For example, the one or more cells that are identified may comprise a cancer cell, and the cancer cell may comprise an RNA and/or protein that is over or under expressed. The RNA and/or protein that is over or under expressed may be used to identify the cell as cancerous.

In some aspects, the one or more analytes that are analyzed, detected, and/or identified using any one of the methods described herein may comprise one or more nucleic acids. The one or more nucleic acids of the one or more analytes may comprise one or more deoxyribonucleic acids (DNA). The one or more nucleic acids of the one or more analytes may comprise one or more ribonucleic acids (RNA). In some embodiments, the one or more nucleic acids may comprise both DNA and RNA. In cases wherein the one or more nucleic acids may comprise one or more RNAs. The one or more RNAs may comprise a variety of types of RNAs, including, but not limited to, messenger RNA (mRNA), ribosomal RNA (rRNA), transfer RNA (tRNA), micro RNA (miRNA), or a combination thereof. The one or more nucleic acids may comprise one or more sequences native to the sample. For example, the one or more nucleic acids of the one or more analytes may comprise one or more endogenous RNAs. In some cases, the one or more nucleic acids of the one or more analytes may comprise one or more sequences exogenous to the sample. For example, the one or more sequences exogenous to the sample may have been delivered to the sample. The one or more nucleic acids of the one or more analytes may comprise one or more sequences associated with one or more therapeutic agents. For example, the one or more sequences associated with one or more therapeutic agents may comprise one or more sequences or portions thereof of one or more gene therapies, one or more CAR T cells, one or more genes or transcripts encoding an antibody, or a combination thereof.

In some embodiments, the one or more nucleic acids of the one or more analytes may comprise one or more modifications. In some cases, the one or more modifications of the one or more nucleic acids of the one or more analytes may be associated with a 5′ end of a nucleic acid of the one or more nucleic acids, a 3′ end of one a nucleic acid of the one or more nucleic acids, one or more internal nucleotides of a nucleic acid of the one or more nucleic acids, or a combination thereof. The one or more modifications may comprise one or more phosphorylation groups, one or more methyl groups, one or more fluorescent modifications, one or more reactive chemical moieties, or a combination thereof. The one or more nucleic acids of the one or more analytes may comprise one or more nucleotides. The one of more nucleotides of the one or more nucleic acids of the one or more analytes may comprise one or more natural nucleotides, one or more non-natural nucleotides, or a combination thereof. The one of more nucleotides of the one or more nucleic acids of the one or more analytes may comprise one or more adenines, one or more guanines, one or more thymines, one or more cytosines, one or more uracils, one or more xanthines, one or more hypoxanthines, one or more 8-azapurines, one or more purines substituted at the 8 position with methyl or bromine, 9-oxo-N6-methyladenine, one or more 2-aminoadenines, one or more 7-deazaxanthines, one or more 7-deazaguanines, one or more 7-deaza-adenines, one or more N4-ethanocytosines, 2,6-diaminopurines, one or more N6-ethano-2,6-diaminopurines, one or more 5-methylcytosines, one or more 5-(C3-C6)-alkynylcytosines, one or more 5-alkynyluracils, one or more 5-fluorouracils, one or more 5-bromouracils, one or more thiouracils, one or more pseudoisocytosines, one or more 2-hydroxy-5-methyl-4-triazolopyridines, one or more isocytosines, one or more isoguanines, one or more inosines, one or more 7,8-dimethylalloxazines, one or more 6-dihydrothymines, one or more 5,6-dihydrouracils, one or more 4-methyl-indoles, ethenoadenines, or a combination thereof.

In some cases, the one or more analytes may comprise a nucleic acid with one or more genetic aberrations. The one or more genetic aberration may comprise one or more insertions, one or more deletions, one or more single nucleotide polymorphisms, one or more single nucleotide variations, one or more copy number variations, or a combination thereof.

The one or more analytes may comprise one or more nucleic acids. The one or more nucleic acids of the one or more analytes may be bound by and/or recognized by one or more binding moieties. The one or more binding moieties may comprise nucleic acid. In some cases, the one or more binding moieties may comprise a nucleic acid sequence that binds to the one or more analytes, a nucleic acid sequence that does not bind to the one or more analytes, or a combination thereof. For example, a binding moiety of the one or more binding moieties may comprise a sequence that hybridizes to a nucleic acid analyte within the sample. The binding moiety may comprise a sequence that does not hybridize to a nucleic acid analyte within the sample. The nucleic acid sequence that does not hybridize to the nucleic acid analyte may hybridize to a probe.

### Binding Moieties

The methods described herein may comprise use of one or more binding moieties. The one or more binding moieties may be useful for detecting and/or identifying one or more analytes as described herein. The one or more binding moieties may be used to bind to one or more analytes in a sample. The binding moieties may comprise one or more barcodes that may be correlated with the one or more analytes. For example, binding the one or more binding moieties to one or more analytes and detecting the one or more barcodes or derivatives thereof may enable detection of the one or more analytes. In some cases, the one or more binding moieties may be amplified to generate one or more amplicons. The one or more amplicons may be detected. Detecting the one or more amplicons may enable identifying the one or more analytes.

The methods described herein may comprise contacting a sample with one or more binding moieties. The one or more binding moieties may recognize one or more analytes in a sample, bind to one or more analytes in a sample, or bind and recognize one or more analytes in a sample. In some cases, the one or more binding moieties may be cross-linked to the sample. The one or more binding moieties may aid in the generation of one or more amplicons. For example, in some cases, at least a portion of the one or more binding moieties may be amplified to generate one or more amplicons. In some cases, the sample may be contacted with one or more probes. The one or more probes may bind to the one or more binding moieties. At least a portion of the one or more probes may be amplified to generate one or more amplicons. The one or more amplicons may be compacted using any one of the methods described herein.

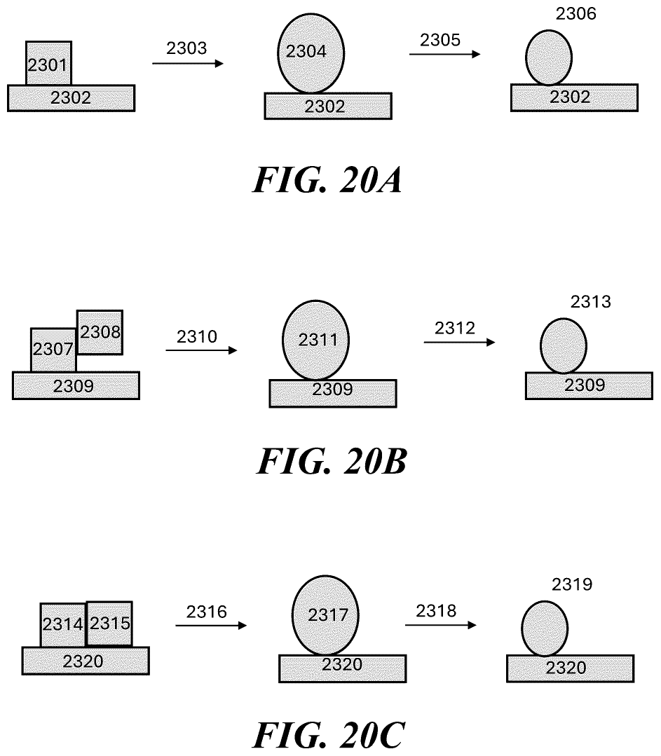

The one or more binding moieties may aid in the generation of one or more amplicons. In some cases, the one or more binding moieties may aid in the generation of one or more compacted amplicons. FIG. 20A shows a schematic for generating an amplicon (e.g., a compacted amplicon). A binding moiety (2301) may bind to an analyte in a sample (2302). At least a portion of the binding moiety may be amplified (2303) to generate an amplicon (2304). The amplicon may be coupled to the analyte in the sample (2302). The amplicon may be compacted (2305) to generate a compacted amplicon (2306). FIG. 20B shows a schematic for generating a compacted amplicon. A binding moiety (2307) may bind to a sample (2309). A probe (2308) may be added to the sample. The probe may couple to the binding moiety (2307). At least a portion of the probe (2308) may be amplified (2310) to generate an amplicon (2311). The amplicon may be compacted (2312) to generate a compacted amplicon (2313). FIG. 20C shows a schematic for generating a compacted amplicon. A binding moiety (2314) may bind to a sample (2320). A probe (2315) may be added to the sample. The probe may couple to the binding moiety (2314). The probe (2315) may couple to the sample (2320). At least a portion of the probe (2315) may be amplified (2316) to generate an amplicon (2317). The amplicon may be compacted (2318) to generate a compacted amplicon (2319).

The one or more binding moieties of the present disclosure may couple to, hybridize to, associate with, or otherwise couple or bind to one or more analytes of the samples described herein. For example, the binding moiety may comprise an antibody that couples to a protein, and/or the antibody may comprise an analyte-binding region that comprises an epitope that recognizes the protein. As another example, the probe may comprise a nucleic acid that comprises a sequence that hybridizes to an mRNA within the sample. The one or more binding moieties may comprise a nucleic acid sequence that hybridizes to one or more analytes. The nucleic acid sequence that hybridizes to one or more analytes of the one or more binding moieties may have a variety of lengths. The nucleic acid sequence that hybridizes to one or more analytes of the one or more binding moieties may comprise about 1 to about 300 nucleotides, about 2 to about 250 nucleotides, about 3 to about 200 nucleotides, about 4 to about 150 nucleotides, about 5 to about 100 nucleotides, about 6 to about 95 nucleotides, about 7 to about 90 nucleotides, about 8 to about 85 nucleotides, about 9 to about 80 nucleotides, about 10 to about 75 nucleotides, about 11 to about 70 nucleotides, about 12 to about 65 nucleotides, about 13 to about 60 nucleotides, about 14 to about 55 nucleotides, about 15 to about 50 nucleotides, about 16 to about 45 nucleotides, about 17 to about 40 nucleotides, about 18 to about 35 nucleotides, about 19 to about 30 nucleotides, or about 20 to about 25 nucleotides. The nucleic acid sequence that hybridizes to one or more analytes of the one or more binding moieties may comprise at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 150 nucleotides, at least about 200 nucleotides, at least about 250 nucleotides, at least about 300 nucleotides, or more nucleotides. The nucleic acid sequence that hybridizes to one or more analytes of the one or more binding moieties may comprise at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100 nucleotides, at most about 150 nucleotides, at most about 200 nucleotides, at most about 250 nucleotides, or at most about 300 nucleotides, or fewer nucleotides

In some cases, the one or more binding moieties may comprise a nucleic acid sequence that couples to a probe. The nucleic acid sequence that couples to a probe may be coupled to a binding moiety of the one or more binding moieties. In some cases, the nucleic acid sequence that couples to a probe may be coupled to the binding moiety through one or more non-covalent interactions, one or more covalent interactions, or a combination thereof. The nucleic acid sequence that couples to a probe may couple to the probe at one or more locations. In some cases, the nucleic acid sequence that couples to a probe may couple to the probe at a single location. In some cases, the nucleic acid sequence that couples to a probe may couple to the probe at two locations. The two locations may be adjacent to each other. In some cases, the two locations may not be adjacent to each other (e.g., may be separated by one or more nucleotides of the probe). In some cases, the two locations may be a distance of at least about 1 nucleotide apart, at least about 2 nucleotides apart, at least about 5 nucleotides apart, at least about 10 nucleotides apart, or more. The nucleic acid sequence that couples to a probe may comprise DNA, RNA, or a combination thereof. In some cases, the nucleic acid sequence that couples to a probe may comprise one or more modifications. The nucleic acid sequence that couples to a probe may comprise a length of about 1 to about 300 nucleotides, about 2 to about 250 nucleotides, about 3 to about 200 nucleotides, about 4 to about 150 nucleotides, about 5 to about 100 nucleotides, about 6 to about 95 nucleotides, about 7 to about 90 nucleotides, about 8 to about 85 nucleotides, about 9 to about 80 nucleotides, about 10 to about 75 nucleotides, about 11 to about 70 nucleotides, about 12 to about 65 nucleotides, about 13 to about 60 nucleotides, about 14 to about 55 nucleotides, about 15 to about 50 nucleotides, about 16 to about 45 nucleotides, about 17 to about 40 nucleotides, about 18 to about 35 nucleotides, about 19 to about 30 nucleotides, or about 20 to about 25 nucleotides. The nucleic acid sequence that couples to a probe may comprise a length of at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 150 nucleotides, at least about 200 nucleotides, at least about 250 nucleotides, at least about 300 nucleotides, or more nucleotides. The nucleic acid sequence that couples to a probe may comprise a length of at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100 nucleotides, at most about 150 nucleotides, at most about 200 nucleotides, at most about 250 nucleotides, at most about 300 nucleotides, or fewer nucleotides.

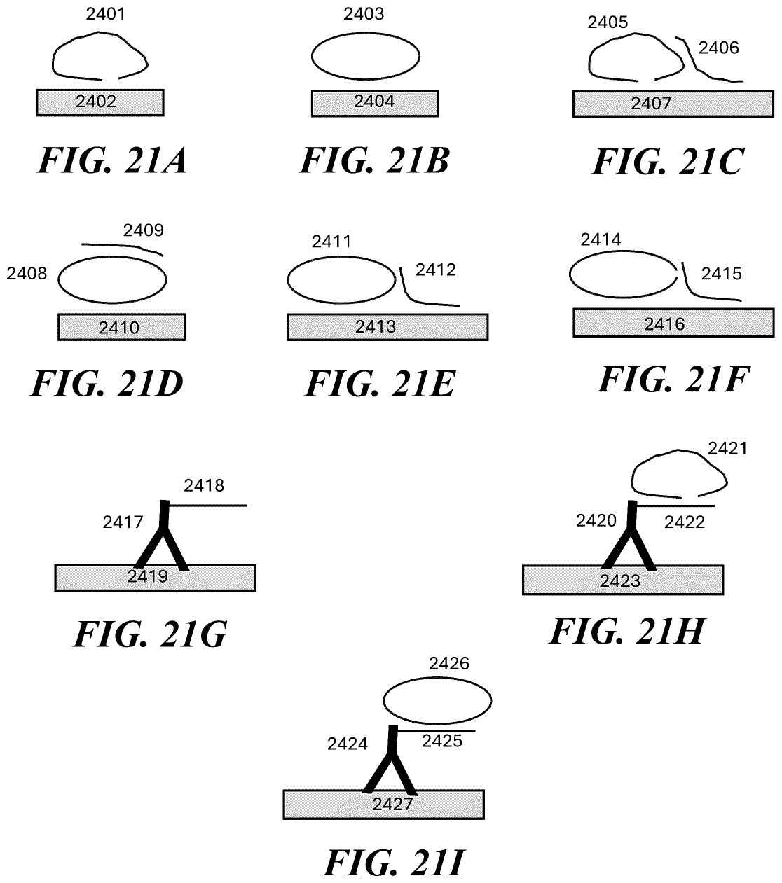

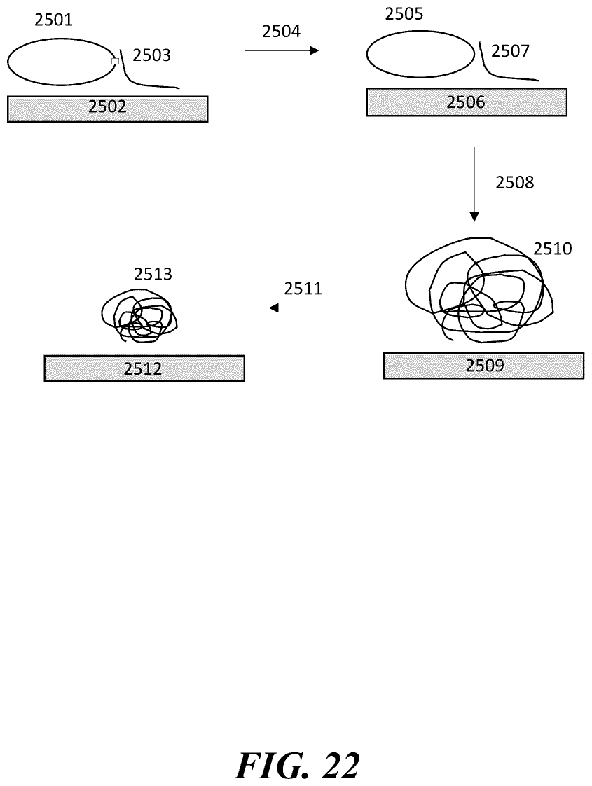

A binding moiety of the one or more binding moieties of the present disclosure may comprise a variety of formats. Schematics of example binding moieties are shown in FIG. 21A-I. For example, FIG. 21A shows a binding moiety comprising nucleic acid (2401). The binding moiety may bind to the sample (2402) at two locations. For example, the binding moiety may comprise a padlock probe nucleic acid sequence where a first end of the binding moiety binds to a first location of an analyte and a second end of the binding moiety binds to a second location of the analyte. The first location of the analyte and the second location of the analyte may be adjacent to each other (e.g., not separated by nucleotides). The binding moiety may be amplified to generate an amplicon. The amplicon may be compacted using any one of the methods described herein. FIG. 21B shows a binding moiety comprising nucleic acid (2403). The nucleic acid of the binding moiety may comprise a circular nucleic acid. The circular nucleic acid may be bound to an analyte in the sample (2404). The circular nucleic acid may be used to generate an amplicon. The amplicon may be compacted using any one of the methods described herein. FIG. 21C shows a schematic of a binding moiety (2405) and a probe (2406). The binding moiety may comprise nucleic acid. The binding moiety may comprise a padlock probe that binds to the sample (2407) at two locations. The probe may bind to the binding moiety (2405). The probe may bind to the sample. The probe may be a primer that amplifies the binding moiety to generate an amplicon. The amplicon may be compacted using any one of the methods described herein. At least a portion of the binding moiety may be amplified, at least a portion of the probe may be amplified, or a combination thereof. FIG. 21D shows a schematic of a binding moiety (2408) comprising nucleic acid. The binding moiety may bind to the sample (2410). A probe (2409) may bind to the binding moiety (2408). The probe may be a primer for amplifying the binding moiety to generate an amplicon. The amplicon may be compacted using any one of the methods described herein. At least a portion of the binding moiety may be amplified, at least a portion of the probe may be amplified, or a combination thereof. FIG. 21E shows a schematic of a binding moiety (2411) comprising nucleic acid. The binding moiety may comprise a circular nucleic acid. The binding moiety may bind to the sample (2413). A probe (2412) may bind to the binding moiety (2411). The probe may bind to the sample (2413). The probe may be a primer for amplifying the binding moiety to generate an amplicon. The amplicon may be compacted using any one of the methods described herein. At least a portion of the binding moiety may be amplified, at least a portion of the probe may be amplified, or a combination thereof. FIG. 21F shows a schematic of a binding moiety (2414) comprising nucleic acid. The binding moiety may comprise a padlock probe. The binding moiety may bind to the sample (2416). A probe (2415) may bind to the binding moiety (2414) at two locations. The two locations may be adjacent to each other. The probe may bind to the sample (2416). The probe may enable ligation of a first end of the binding moiety to a second end of the binding moiety to generate a circular nucleic acid. The probe may be a primer for amplifying the circular nucleic acid to generate an amplicon. The amplicon may be compacted using any one of the methods described herein. At least a portion of the circular nucleic acid may be amplified, at least a portion of the probe may be amplified, or a combination thereof.

In some cases, the binding moiety of the one or more binding moieties of the methods described herein may comprise an antibody, an antibody fragment, or a combination thereof. For example, FIG. 21G shows a schematic of a binding moiety comprising an antibody (2417). The binding moiety may comprise a nucleic acid sequence (2418). The nucleic acid sequence may bind to one or more probes of the methods described herein. The antibody may be bound to the sample (2419). FIG. 21H shows a schematic of a binding moiety comprising an antibody (2420). The antibody may be bound to the sample (2423). For example, the antibody may be bound to an analyte in the sample. The binding moiety may comprise nucleic acid sequence (2422). The nucleic acid sequence of the binding moiety may bind to a probe (2421). The probe may comprise a nucleic acid. The probe may comprise a padlock probe nucleic acid. The padlock probe nucleic acid may be ligated to generate a circular nucleic acid. The circular nucleic acid may be amplified to generate an amplicon. The amplicon may be compacted using any one of the methods described herein. FIG. 21I shows a schematic of a binding moiety comprising an antibody (2424). The antibody may be bound to the sample (2427). For example, the antibody may be bound to an analyte in the sample. The binding moiety may comprise a nucleic acid sequence (2425). The nucleic acid sequence may be coupled to the antibody. For example, the nucleic acid sequence may be conjugated to the antibody through one or more covalent bonds. A probe (2426) may bind to the nucleic acid sequence (2425) of the binding moiety. The probe may comprise a circular nucleic acid. The nucleic acid sequence of the binding moiety may be a primer to amplify the circular nucleic acid to generate an amplicon. The amplicon may be amplified using any one of the methods described herein.

The one or more binding moieties may comprise one or more modifications. In some cases, the one or more binding moieties may comprise one or more padlock probes. The one or more padlock probes may comprise one or more phosphorylation modifications. The one or more phosphorylation modifications may be located at a 3′ end of a binding moiety of the one or more binding moieties. The one or more phosphorylation modifications may be located at a 5′ end of a binding moiety of the one or more binding moieties. In some cases, 5′ end of a binding moiety may be ligated to the 3′ end of a binding moiety.

The methods described herein may comprise contacting the one or more analytes of the sample with one or more binding moieties. The contacting of the one or more analytes with the one or more binding moieties may comprise conditions that promote binding or coupling of the one or more binding moieties to the one or more analytes. For example, the contacting may result in a binding moiety of the one or more binding moieties coupling to or binding to an analyte of the one or more analytes. The conditions that promote binding between the one or more binding moieties and the one or more analytes may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperature may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The reaction mixture to promote coupling of one or more binding moieties to one or more analytes may comprise one or more buffers. The one or more buffers of the reaction mixture may comprise MES (4-Morpholineethanesulfonic acid), Bis-Tris (Bis(2-hydroxyethyl)amino-tris (hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The reaction mixture to promote coupling of one or more binding moieties to one or more analytes may comprise one or more salts. The one or more salts of the reaction mixture may comprise NaCl, CaCl2, MgCl2, or a combination thereof. The reaction mixture to promote coupling of one or more binding moieties to one or more analytes may comprise one or more detergents. The one or more detergents of the reaction mixture may comprise SDS, Triton X-100, CHAPS, NP-40, Tween-20, Digitonin, or a combination thereof. The reaction mixture to promote coupling of one or more binding moieties to one or more analytes may comprise one or more solvents. The one or more solvents of the reaction mixture may comprise methanol, ethanol, ethyl acetate, DMSO, acetonitrile, water, or a combination thereof. The one or more chaotropic agents of the ligation reaction conditions may comprise DMSO, formamide, urea, thiourea, 2-propanol, guanidinium chloride, n-butanol, or a combination thereof. The reaction mixture to promote coupling of one or more binding moieties to one or more analytes may comprise a pH. The pH of the reaction mixture may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, or higher. The pH of the reaction mixture may at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12, or lower.

In some cases, the methods described herein may comprise contacting a sample with a plurality of binding moieties. The plurality of binding moieties may comprise binding moieties as described herein. In some cases, the plurality of binding moieties may be configured to bind to a plurality of analytes. In some cases, more than one binding moiety may bind to an analyte of a sample, described herein. In some cases, a binding moiety may bind to an analyte of a sample as described herein. For example, five binding moieties may bind to an analyte, where each of the five binding moieties binds to a different location of the analyte.

The plurality of binding moieties may comprise a variety of quantities. In some cases, the plurality of binding moieties may comprise at least about 2 binding moieties, at least about 5 binding moieties, at least about 10 binding moieties, at least about 15 binding moieties, at least about 20 binding moieties, at least about 25 binding moieties, at least about 50 binding moieties, at least about 100 binding moieties, at least about 150 binding moieties, at least about 200 binding moieties, at least about 300 binding moieties, at least about 400 binding moieties, at least about 500 binding moieties, at least about 750 binding moieties, at least about 1000 binding moieties, at least about 1500 binding moieties, at least about 2000 binding moieties, at least about 2500 binding moieties, at least about 5000 binding moieties, at least about 7500 binding moieties, at least about 10000 binding moieties, at least about 15000 binding moieties, at least about 20000 binding moieties, at least about 50000 binding moieties, at least about 100000 binding moieties, or more binding moieties. In some cases, the plurality of binding moieties may comprise at most about 2 binding moieties, at most about 5 binding moieties, at most about 10 binding moieties, at most about 15 binding moieties, at most about 20 binding moieties, at most about 25 binding moieties, at most about 50 binding moieties, at most about 100 binding moieties, at most about 150 binding moieties, at most about 200 binding moieties, at most about 300 binding moieties, at most about 400 binding moieties, at most about 500 binding moieties, at most about 750 binding moieties, at most about 1000 binding moieties, at most about 1500 binding moieties, at most about 2000 binding moieties, at most about 2500 binding moieties, at most about 5000 binding moieties, at most about 7500 binding moieties, at most about 10000 binding moieties, at most about 15000 binding moieties, at most about 20000 binding moieties, at most about 50000 binding moieties, at most about 100000 binding moieties, or fewer binding moieties. In some cases, the plurality of binding moieties may comprise about 2-100000 binding moieties, about 5-50000 binding moieties, about 10-20000 binding moieties, about 15-15000 binding moieties, about 20-10000 binding moieties, about 25-7500 binding moieties, about 50-5000 binding moieties, about 100-2500 binding moieties, about 150-2000 binding moieties, about 200-1500 binding moieties, about 300-1000 binding moieties, or about 400-750 binding moieties.

Contacting Sample with Probe(s)

The methods described herein may comprise contacting a sample with one or more probes. A probe of the one or more probes may couple to a binding moiety, the sample, or a combination thereof. Examples of probe configurations are shown in FIGS. 20 and 21 and described above. In some cases, the probe may be a primer that amplifies at least a portion of a binding moiety to generate an amplicon. The amplicon may be compacted using any one of the methods described herein.

The probe may of the one or more probes may comprise a nucleic acid. In some cases, the nucleic acid of the probe may bind to (e.g., hybridize to) a binding moiety. In some cases, the probe may hybridize to a binding moiety comprising a circular nucleic acid or a padlock probe. For example, the probe may comprise a nucleic acid sequence that hybridizes to a padlock probe sequence. The padlock probe sequence may hybridize to the probe at one or more locations. In some cases, a first end of the padlock probe sequence may hybridize to the probe at a first location and a second end of the padlock probe sequence may hybridize to the probe at a second location. The padlock probe sequence of the binding moiety may be ligated as a result of binding to the probe. The probe may be bound to the sample. For example, in some cases, the probe may be bound to the sample and the padlock probe sequence of the binding moiety. In some cases, the probe may bind to (e.g., hybridize to) a binding moiety comprising a nucleic acid sequence coupled to an antibody. For example, the probe may comprise a circular nucleic acid or a padlock probe. The circular nucleic acid or the padlock probe may be amplified to generate one or more amplicons.

The methods described herein may comprise contacting the sample with one or more probes under conditions to promote binding between the one or more probes and the sample, the one or more probes and one or more binding moieties, or a combination thereof. For example, the contacting may result in a binding moiety of the one or more binding moieties coupling to or binding to a probe of the one or more probes and/or a probe of the one or more probes binding to and/or coupling to the sample. The conditions to promote binding between the one or more probes and the sample, the one or more probes and one or more binding moieties, or a combination thereof may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days, or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days, at most about 4 days, or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperature may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperature may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The reaction mixture to promote binding between the one or more probes and the sample, the one or more probes and one or more binding moieties, or a combination thereof may comprise one or more buffers. The one or more buffers of the reaction mixture may comprise MES (4-Morpholineethanesulfonic acid), Bis-Tris (Bis(2-hydroxyethyl)amino-tris (hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The reaction mixture to promote binding between the one or more probes and the sample, the one or more probes and one or more binding moieties, or a combination thereof may comprise one or more salts. The one or more salts of the reaction mixture may comprise NaCl, CaCl2, MgCl2, or a combination thereof. The reaction mixture to promote binding between the one or more probes and the sample, the one or more probes and one or more binding moieties, or a combination thereof may comprise one or more detergents. The one or more detergents of the reaction mixture may comprise SDS, Triton X-100, CHAPS, NP-40, Tween-20, Digitonin, or a combination thereof. The reaction mixture to promote binding between the one or more probes and the sample, the one or more probes and one or more binding moieties, or a combination thereof may comprise one or more solvents. The one or more solvents of the reaction mixture may comprise methanol, ethanol, ethyl acetate, DMSO, acetonitrile, water, or a combination thereof. The one or more chaotropic agents of the ligation reaction conditions may comprise DMSO, formamide, urea, thiourea, 2-propanol, guanidinium chloride, n-butanol, or a combination thereof. The reaction mixture to promote binding between the one or more probes and the sample, the one or more probes and one or more binding moieties, or a combination thereof may comprise a pH. The pH of the reaction mixture may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, or higher. The pH of the reaction mixture may be at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, or at most about 12, or lower.

A probe of the one or more probes may comprise nucleic acid. The nucleic acid of the probe may comprise DNA, RNA, or a combination thereof. The probe may comprise single-stranded nucleic acid, double-stranded nucleic acid, or a combination thereof. In some cases, the nucleic acid of the probe may comprise a primer. For example, the probe may bind to a binding moiety and initiate an amplification reaction (e.g., a rolling circle amplification reaction). In some cases, the probe may bind to the binding moiety and not the sample. In some cases, the probe may bind to the binding moiety and the sample. The probe of the one or more probes may comprise a variety of lengths. The length of the probe of the one or more probes may be about 1 to about 300 nucleotides, about 2 to about 250 nucleotides, about 3 to about 200 nucleotides, about 4 to about 150 nucleotides, about 5 to about 100 nucleotides, about 6 to about 95 nucleotides, about 7 to about 90 nucleotides, about 8 to about 85 nucleotides, about 9 to about 80 nucleotides, about 10 to about 75 nucleotides, about 11 to about 70 nucleotides, about 12 to about 65 nucleotides, about 13 to about 60 nucleotides, about 14 to about 55 nucleotides, about 15 to about 50 nucleotides, about 16 to about 45 nucleotides, about 17 to about 40 nucleotides, about 18 to about 35 nucleotides, about 19 to about 30 nucleotides, or about 20 to about 25 nucleotides. The length of the probe of the one or more probes may be at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 150 nucleotides, at least about 200 nucleotides, at least about 250 nucleotides, at least about 300 nucleotides, or more nucleotides. The length of the probe of the one or more probes may be at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, at most about 20 nucleotides, at most about 25 nucleotides, at most about 30 nucleotides, at most about 35 nucleotides, at most about 40 nucleotides, at most about 45 nucleotides, at most about 50 nucleotides, at most about 55 nucleotides, at most about 60 nucleotides, at most about 65 nucleotides, at most about 70 nucleotides, at most about 75 nucleotides, at most about 80 nucleotides, at most about 85 nucleotides, at most about 90 nucleotides, at most about 95 nucleotides, at most about 100 nucleotides, at most about 150 nucleotides, at most about 200 nucleotides, at most about 250 nucleotides, at most about 300 nucleotides, or fewer nucleotides.

In some embodiments, the binding moiety comprises a nucleic acid. In some embodiments, the nucleic acid comprises a ribonucleic acid. In some embodiments, the nucleic acid comprises a deoxyribonucleic acid. The binding moiety comprising a nucleic acid may bind to the target by hybridization at one or more locations. In some embodiments, the binding moiety may comprise a polypeptide. In some embodiments, the polypeptide may comprise a protein. In some embodiments, the polypeptide may comprise an antibody or antibody fragment. The binding moiety may comprise an antibody or antibody fragment and a nucleic acid. The antibody or antibody fragment and the nucleic acid may be conjugated to each other with one or more covalent bonds. The antibody or antibody fragment and the nucleic acid may be complexed with each other through non-covalent interactions. In some cases, the binding moiety may comprise an antibody or antibody fragment and a nucleic acid that are connected through at least one covalent bond and one or more non-covalent interactions. In some embodiments, the polypeptide may comprise a nanobody. In some embodiments, the binding moiety comprises an antibody or antibody fragment conjugated to a nucleic acid. In some embodiments, the binding moiety comprises one or more barcodes. The barcode may denote a target that is detected by the corresponding binding moiety. For example, the barcode may be conjugated to the binding moiety. The barcode may be amplified, and the binding moiety may bind to the target and the barcode or derivative thereof (e.g. a reverse complement of the barcode) may be detected as a corollary to the target. In some embodiments, the barcode may comprise a nucleic acid. The binding moiety may comprise a nucleic acid and the nucleic acid of the binding moiety may comprise the barcode. For example, the binding moiety may comprise a nucleic acid sequence that hybridizes to a target in a cell (e.g., a transcript) the nucleic acid of the binding moiety may comprise a barcode that comprises a nucleic acid sequence that denotes the target detected by the binding moiety. In some embodiments, the nucleic acid of the barcode may comprise a ribonucleic acid. In some embodiments, the nucleic acid of the barcode may comprise a deoxyribonucleic acid. In some embodiments, the nucleic acid of the barcode may be from 1 to 1000, from 2 to 900, from 5 to 800, from 10 to 700, from 15 to 600, from 20 to 500, from 25 to 400, from 30 to 300, from 40 to 200, from 50 to 100, from 60 to 90, from 70 to 80 nucleotides in length.

In some embodiments, the probe may comprise a nucleic acid. In some embodiments, the probe may bind to the binding moiety. For example, the probe may comprise a nucleic acid that hybridizes to the binding moiety. The probe may comprise a nucleic acid that binds to the binding moiety at one or more locations. For example, the probe may comprise a nucleic acid that hybridizes to the binding moiety at two locations. The two locations where the nucleic acid of the probe hybridizes to the binding moiety may be directly adjacent to each other. The two locations where the nucleic acid of the probe hybridizes to the binding moiety may be separated by one or more nucleotides of the binding moiety. In some embodiments, the nucleic acid of the probe may comprise a ribonucleic acid. In some embodiments, the nucleic acid or the probe may comprise a deoxyribonucleic acid. In some embodiments, the nucleic acid of the probe may be from 1 to 1000, from 2 to 900, from 5 to 800, from 10 to 700, from 15 to 600, from 20 to 500, from 25 to 400, from 30 to 300, from 40 to 200, from 50 to 100, from 60 to 90, from 70 to 80 nucleotides in length. The probe may comprise one or more modifications. The one or more modifications may comprise a phosphorylation modification, a hydroxyl modification, or a combination thereof.

### Ligation

The methods described herein may comprise one or more ligation reactions. For example, the methods described herein may comprise ligating a first end of a padlock probe to a second end of the padlock probe to generate a circular nucleic acid, ligating an end of a first detection probe to an end of a second detection probe, or a combination thereof. In some cases, the padlock probe that is ligated may be a binding moiety. In some cases, the padlock probe that is ligated may be a probe. The ligation reactions as described herein may be facilitated by binding of a nucleic acid sequence to the sequence(s) that may be ligated. For example, a binding moiety comprising a padlock probe may bind to (e.g., hybridize to) an analyte in a sample. The analyte in the sample may comprise nucleic acid. Upon binding of the padlock probe of the binding moiety to the nucleic acid of the analyte, a first end of the padlock probe may be ligated to a second end of the padlock probe using an enzyme (e.g., a ligase) to generate a circular nucleic acid. Another example provides a probe comprising a nucleic acid. The nucleic acid may bind to the sample and a binding moiety. The binding moiety may comprise a padlock probe. The padlock probe of the binding moiety may bind to the probe at two locations. A first end of the padlock probe of the binding moiety may bind to a first location of the probe. A second end of the padlock probe of the binding moiety may bind to a second location of the probe. Upon binding of the first end and the second end of the padlock probe of the binding moiety to the probe, the padlock probe may be ligated to generate a circular nucleic acid. The circular nucleic acid may be amplified to generate one or more amplicons, as described herein. The one or more amplicons may be compacted using any one of the methods described herein.

The one or more ligation reactions of the methods described herein may comprise contacting the sample with one or more ligases. In some cases, the one or more ligases may comprise a mammalian ligase, a bacterial ligase, or a combination thereof. The one or more ligases may comprise a deoxyribonucleic acid (DNA) ligase I, DNA ligase II, DNA ligase III, DNA ligase IV, or a combination thereof. In some cases, the one or more ligases may comprise an RNA ligase. In some cases, the one or more ligases may ligate a 3′ nucleotide of one nucleic acid to a 5′ nucleotide of a different nucleic acid (e.g., a different nucleic acid molecule). In some cases, the one or more ligases may ligate a 3′ end of a nucleic acid (e.g., a padlock probe) to a 5′ end of the nucleic acid. In some cases, the ligation reaction may generate a circular nucleic acid. In some cases, the ligase may ligate a 3′ end of a detection probe to a 5′ end of the same detection probe. The one or more ligases may ligate two nucleotides that are part of a double-stranded nucleic acid. For example, the double-stranded nucleic acid may comprise a nick, and the location of the nick may be ligated by the one or more ligases. In some embodiments, the double-stranded nucleic acid may comprise a DNA/DNA duplex. In some embodiments, the double-stranded nucleic acid may comprise an RNA/DNA duplex. The ligase may comprise one or more of the following: T4 DNA ligase, SplintR ligase, T3 DNA ligase, T7 DNA ligase, E. coli DNA ligase, Taq ligase, RtcB ligase, or a combination thereof. A ligation reaction may be carried out using the one or more ligases. The ligation reaction may involve incubating a sample comprising an analyte comprising one or more detection probes. The sample may be incubated with one or more ligases for a period of time, for example at least 10 minutes, at least 20 minutes, at least 30 minutes, at least 1 hour, at least 2 hours, or more. The sample may be incubated with one or more ligases for at most 10 minutes, at most 20 minutes, at most 30 minutes, at most 1 hour, at most 2 hours, or more. The ligation reaction may comprise incubation with one or more components, including, but not limited to, one or more buffers, one or more salts, one or more detergents, one or more solvents, one or more chaotropic reagents, or a combination thereof. The one or more buffers of the ligation reaction conditions may comprise MES (4-Morpholineethanesulfonic acid), Bis-Tris (Bis(2-hydroxyethyl)amino-tris (hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The one or more salts of the ligation reaction conditions may comprise NaCl, CaCl₂), MgCl₂, or a combination thereof. The one or more detergents of the ligation reaction conditions may comprise SDS, Triton X-100, CHAPS, NP-40, Tween-20, Digitonin, or a combination thereof. The one or more solvents of the ligation reaction may comprise methanol, ethanol, ethyl acetate, DMSO, acetonitrile, water, or a combination thereof. The one or more chaotropic agents of the ligation reaction conditions may comprise DMSO, formamide, urea, thiourea, 2-propanol, guanidinium chloride, n-butanol, or a combination thereof. The ligation reaction may include an incubation at one or more temperatures. The one or more temperatures of the ligation reaction may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperatures may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

In some embodiments, the ligation reaction of the methods described herein may comprise ligating one end of the probe to another end of the probe. For example, the probe may comprise a nucleic acid and 3′ end of the nucleic acid of the probe may be ligated to the 5′ end of the nucleic acid of the probe. The ligation reaction may be performed at a temperature of from about 4° C. to about 8° C., from about 15° C. to about 20° C., from about 40° C. to about 50° C., from about 0° C. to about 80° C., from about 10° C. to about 70° C., from about 20° C. to about 60° C., or from about 30° C. to about 50° C. In some embodiments, the ligation reaction is mediated by a ligase or a T4 DNA ligase enzyme. In some embodiments, the ligation reaction is free of an enzyme. The ligation reaction may generate a circular nucleic acid.

### Amplification/Amplicon

The methods described herein may comprise amplifying one or more binding moieties, one or more probes, or a combination thereof. Amplifying one or more binding moieties, one or more probes, or a combination thereof may provide certain advantages. For example, the one or more binding moieties and/or the one or more probes may comprise one or more barcodes corresponding to an analyte. Detecting the one or more barcodes or derivatives thereof (e.g., reverse complements thereof) may enable identification of the analyte. Amplifying the one or more binding moieties and/or the one or more probes may generate one or more amplicons with more than one copy of the one or more barcodes or derivative thereof. Detecting more than one copy of the one or more barcodes or derivatives thereof may result in a stronger signal (e.g., a brighter fluorescence signal), a faster experimental time, use of a shorter exposure time for detecting, or a combination thereof. In some cases, amplifying the one or more binding moieties and/or the one or more probes may enable detection of an analyte with low expression. For example, a low expressing analyte in a sample may be undetectable without amplification and may be detectable with amplification.

The amplifying may generate one or more amplicons. The one or more amplicons may comprise multiple copies of a sequence associated with the one or more binding moieties, the one or more probes, or a combination thereof. In some cases, an amplicon of the one or more amplicons, may comprise at least a portion of one or binding moieties or a reverse complement thereof, at least a portion of a probe of the one or more probes or a reverse complement thereof, or a combination thereof. The one or more amplicons may comprise one or more barcodes or reverse complement thereof. The one or more barcodes or reverse complement thereof (e.g., a derivative thereof). The one or more amplicons may be correlated with one or more analytes of the sample. For example, an analyte of the one or more analytes of the sample may be contacted with a binding moiety. The binding moiety may comprise a barcode. The sample may be contacted with a probe. The probe may bind to the binding moiety and initiate amplification of the binding moiety to generate an amplicon. The amplicon may comprise multiple copies of a reverse complement of the barcode. The reverse complement of the barcode may be detected using the methods described herein. The amplicon may comprise at least about 1, at least about 2, at least about 5, at least about 10, at least about 15, at least about 20, at least about 25, at least about 50, at least about 100, at least about 150, at least about 200, at least about 300, at least about 400, at least about 500, at least about 750, at least about 1000, at least about 1500, at least about 2000, at least about 2500, at least about 5000, at least about 7500, at least about 10000, or more copies of a barcode or reverse complement of the barcode The amplicon may comprise at most about 1, at most about 2, at most about 5, at most about 10, at most about 15, at most about 20, at most about 25, at most about 50, at most about 100, at most about 150, at most about 200, at most about 300, at most about 400, at most about 500, at most about 750, at most about 1000, at most about 1500, at most about 2000, at most about 2500, at most about 5000, at most about 7500, at most about 10000, or fewer copies of a barcode or reverse complement of the barcode. The amplicon may comprise about 1-10000, about 2-7500, about 5-5000, about 10-2500, about 15-2000, about 20-1500, about 25-1000, about 50-750, about 100-500, about 150-400, or about 200-300 copies of a barcode or reverse complement of the barcode. The amplicon may comprise a nucleic acid barcode. The nucleic acid barcode of the amplicon may comprise about 1 to about 20 nucleotides, about 2 to about 19 nucleotides, about 3 to about 18 nucleotides, about 4 to about 17 nucleotides, about 5 to about 16 nucleotides, about 6 to about 15 nucleotides, about 7 to about 14 nucleotides, about 8 to about 13 nucleotides, about 9 to about 12 nucleotides, or about 10 to about 11 nucleotides. The nucleic acid barcode of the amplicon may comprise at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, or more nucleotides. The nucleic acid barcode of the amplicon may comprise at most about 1 nucleotide, at most about 2 nucleotides, at most about 3 nucleotides, at most about 4 nucleotides, at most about 5 nucleotides, at most about 6 nucleotides, at most about 7 nucleotides, at most about 8 nucleotides, at most about 9 nucleotides, at most about 10 nucleotides, at most about 11 nucleotides, at most about 12 nucleotides, at most about 13 nucleotides, at most about 14 nucleotides, at most about 15 nucleotides, at most about 16 nucleotides, at most about 17 nucleotides, at most about 18 nucleotides, at most about 19 nucleotides, or at most about 20 nucleotides, or fewer nucleotides.

The one or more barcodes or derivatives thereof (e.g., reverse complements thereof) may be detected using one or more detection probes. The one or more barcodes or derivatives thereof (e.g., reverse complements thereof) may comprise nucleic acid. The nucleic acid of the one or more barcodes or derivatives thereof (e.g., reverse complements thereof) may couple with (e.g., hybridize to) nucleic acid of one or more detection probes. For example, an amplicon may be generated, and the amplicon may comprise at least 50 copies of a barcode. A detection probe may bind to the barcode. The detection probe may be imaged using any one of the imaging systems described herein.

The amplifying of the methods described herein may comprise rolling circle amplification. The rolling circle amplification may comprise contacting the sample with a binding moiety. The binding moiety may comprise a padlock probe. The padlock probe may be ligated to generate a circular nucleic acid. The sample may be contacted with a probe. The probe may bind to the binding moiety. In some cases, the probe may enable ligating an end of the padlock probe to another end of the padlock probe to generate the circular nucleic acid. In some cases, the analyte bound by the binding moiety may enable ligating an end of the padlock probe to another end of the padlock probe to generate the circular nucleic acid. The probe may initiate amplification to generate an amplicon. The amplicon may comprise multiple copies of at least a portion of the probe. The amplicon may comprise multiple copies of at least a portion of the binding moiety. The rolling circle amplification may comprise contacting a sample with a binding moiety comprising an antibody. The antibody may bind to an analyte in a sample. The binding moiety may comprise a nucleic acid sequence. A probe may be added to the sample. The probe may bind to the nucleic acid sequence of the binding moiety. The probe may comprise a padlock probe or a circular nucleic acid. The probe comprising the padlock probe may be ligated to generate a circular nucleic acid. The circular nucleic acid may be amplified using the nucleic acid sequence of the binding moiety as a primer to generate an amplicon. The amplicon may comprise multiple copies of at least a portion of the probe. The amplicon may comprise multiple copies of at least a portion of the binding moiety.

The rolling circle amplification reaction may be performed using a polymerase. The polymerase may be a DNA polymerase. The DNA polymerase may comprise Q5 High-Fidelity DNA Polymerase, Q5U Hot Start High-Fidelity DNA Polymerase, Phusion High-Fidelity DNA Polymerase*, Routine PCR, OneTaq DNA Polymerase, Taq DNA Polymerase, LongAmp Taq DNA Polymerase, Hemo KlenTaq, Epimark Hot Start Taq DNA Polymerase, Isothermal Amplification and Strand Displacement, Bst DNA Polymerase, Bst DNA Polymerase, Bst 2.0 DNA Polymerase, Bst 3.0 DNA Polymerase, Bsu DNA Polymerase, Large Fragment, phi29 DNA Polymerase, phi29-XT DNA Polymerase, T7 DNA Polymerase (unmodified), Sulfolobus DNA Polymerase IV, Therminator™ DNA Polymerase, DNA Polymerase I (E. coli), DNA Polymerase I, Large (Klenow) Fragment’, Klenow Fragment (3′→5′ exo−), T4 DNA Polymerase, Legacy Polymerases, Vent DNA Polymerase, Vent (exo−) DNA Polymerase, Deep Vent DNA Polymerase, Deep Vent (exo−) DNA Polymerase, or a combination thereof. The polymerase may comprise phi29 DNA Polymerase, phi29-XT DNA Polymerase, or a combination thereof.

The rolling circle amplification reaction may be performed using a buffer. The buffer may comprise a variety of components including, but not limited to, MgCl₂, NaCl, CaCl₂), ethylenediaminetetraacetic acid (EDTA), 2-[4-(2,4,4-trimethylpentan-2-yl) phenoxy]ethanol (Triton X-100), polysorbate 20 (Tween 20), sodium lauryl sulfate (SDS), 2-Amino-2-hydroxymethyl-propane-1,3-diol (tris), sheared DNA, water, or a combination thereof.

The rolling circle amplification reaction may be performed at a temperature. The temperature may be about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., or higher. The temperature may be about 4 to about 95° C., about 5 to about 94° C., about 6 to about 93° C., about 7 to about 92° C., about 8 to about 91° C., about 9 to about 90° C., about 10 to about 89° C., about 11 to about 88° C., about 12 to about 87° C., about 13 to about 86° C., about 14 to about 85° C., about 15 to about 84° C., about 16 to about 83° C., about 17 to about 82° C., about 18 to about 81° C., about 19 to about 80° C., about 20 to about 79° C., about 21 to about 78° C., about 22 to about 77° C., about 23 to about 76° C., about 24 to about 75° C., about 25 to about 74° C., about 26 to about 73° C., about 27 to about 72° C., about 28 to about 71° C., about 29 to about 70° C., about 30 to about 69° C., about 31 to about 68° C., about 32 to about 67° C., about 33 to about 66° C., about 34 to about 65° C., about 35 to about 64° C., about 36 to about 63° C., about 37 to about 62° C., about 38 to about 61° C., about 39 to about 60° C., about 40 to about 59° C., about 41 to about 58° C., about 42 to about 57° C., about 43 to about 56° C., about 44 to about 55° C., about 45 to about 54° C., about 46 to about 53° C., about 47 to about 52° C., about 48 to about 51° C., or about 49 to about 50° C.

The rolling circle amplification reaction may be performed for a length of time. The length of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days, at least about 4 days, or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days, at most about 4 days, or less. The length of time may be about 5 minutes to about 24 hours, about 10 minutes to about 23 hours, about 15 minutes to about 22 hours, about 20 minutes to about 21 hours, about 25 minutes to about 20 hours, about 30 minutes to about 19 hours, about 40 minutes to about 18 hours, about 45 minutes to about 17 hours, about 50 minutes to about 16 hours, about 55 minutes to about 15 hours, about 60 minutes to about 14 hours, about 1 hour to about 13 hours, about 2 hours to about 12 hours, about 3 hours to about 11 hours, about 4 hours to about 10 hours, about 5 hours to about 9 hours, or about 6 hours to about 8 hours.

The rolling circle amplification reaction of the methods described herein may comprise use of modified nucleotides. The modified nucleotides may comprise chemical reactive moieties. The chemical reactive moieties may be used to compact one or more amplicons generated using the methods described herein. For example, modified nucleotides may be included during the rolling circle amplification. Unmodified nucleotides (e.g., adenines, cytosines, guanines, thymines, and uracils) may be included in combination with modified nucleotides during rolling circle amplification. The one or more amplicons generated using the methods described herein may incorporate modified nucleotides, unmodified nucleotides, or a combination thereof during rolling circle amplification. For example, a modified nucleotide comprising an azide may be added to a mixture of unmodified nucleotides during rolling circle amplification. The azide may be incorporated into one or more amplicons generated from the rolling circle amplification. Chemical reactive moieties that may be incorporated into one or more amplicons include one or more of an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof.

The chemical reactive moieties (e.g., the first and second chemical reactive moieties) may be incorporated into one or more amplicons using the methods described herein. For example, a non-natural nucleotide may be added to the rolling circle amplification reaction conditions and the non-natural nucleotide may be incorporated into the amplicon. The non-natural nucleotide may comprise an azide, an alkyne, a polyethylene glycol, or a combination thereof.

The first reactive chemical moiety and the second reactive chemical moiety may form a conjugate. The conjugate may comprise one or more covalent bonds, one or more non-covalent interactions, or a combination thereof. For example, the first reactive chemical moiety may comprise a nucleophile and the second reactive chemical moiety may comprise an electrophile. The first reactive chemical moiety may be reacted with the second reactive chemical moiety to form a covalent bond between the first reactive chemical moiety and the second reactive chemical moiety. the conjugate comprises a linker. In some embodiments, the linker may comprise a polyethylene glycol, a methylene group, an ethylene group, an ethylene group, an ether, or a combination thereof.

In some cases, a first chemical reactive moiety (e.g., the first chemical reactive moiety) may be incorporated into an amplicon. In some cases, the first reactive chemical moiety may comprise an azide. In some cases, the first reactive chemical moiety may comprise an alkyne (e.g., a cyclooctyne). In some cases, the first reactive chemical moiety may comprise an amine. In some cases, the first reactive chemical moiety may comprise a carboxyl. In some cases, the first reactive chemical moiety may comprise a sulfhydryl. In some cases, the first reactive chemical moiety may comprise a carboxylic acid. In some cases, the first reactive chemical moiety may comprise a maleimide. In some cases, the first reactive chemical moiety may comprise an NHS-ester. In some cases, the first reactive chemical moiety may comprise a carbodiimide. In some cases, the first reactive chemical moiety may comprise an imidoester. In some cases, the first reactive chemical moiety may comprise a haloacetyl. In some cases, the first reactive chemical moiety may comprise a pyridyldisulfide. In some cases, the first reactive chemical moiety may comprise a hydrazide. In some cases, the first reactive chemical moiety may comprise an alkoxyamine. In some cases, the first reactive chemical moiety may comprise a diazirine. In some cases, the first reactive chemical moiety may comprise a phosphine. In some cases, the first reactive chemical moiety may comprise an epoxide. In some cases, the first reactive chemical moiety may comprise an aldehyde. In some embodiments, the first reactive chemical moiety may comprise an azide and an alkyne. In some embodiments, the first reactive chemical moiety may comprise an azide and an amine. In some embodiments, the first reactive chemical moiety may comprise an azide and a carboxyl. In some embodiments, the first reactive chemical moiety may comprise an azide and a sulfhydryl. In some embodiments, the first reactive chemical moiety may comprise an azide and a carboxylic acid. In some embodiments, the first reactive chemical moiety may comprise an azide and a maleimide. In some embodiments, the first reactive chemical moiety may comprise an azide and an NHS-ester. In some embodiments, the first reactive chemical moiety may comprise an azide and a carbodiimide. In some embodiments, the first reactive chemical moiety may comprise an azide and an imidoester. In some embodiments, the first reactive chemical moiety may comprise an azide and a haloacetyl. In some embodiments, the first reactive chemical moiety may comprise an azide and a pyridyldisulfide. In some embodiments, the first reactive chemical moiety may comprise an azide and a hydrazide. In some embodiments, the first reactive chemical moiety may comprise an azide and an alkoxyamine. In some embodiments, the first reactive chemical moiety may comprise an azide and a diazirine. In some embodiments, the first reactive chemical moiety may comprise an azide and a phosphine. In some embodiments, the first reactive chemical moiety may comprise an azide and an epoxide. In some embodiments, the first reactive chemical moiety may comprise an azide and an aldehyde. In some embodiments, the first reactive chemical moiety may comprise an alkyne and an amine. In some embodiments, the first reactive chemical moiety may comprise an alkyne and a carboxyl. In some embodiments, the first reactive chemical moiety may comprise an alkyne and a sulfhydryl. In some embodiments, the first reactive chemical moiety may comprise an alkyne and a carboxylic acid. In some embodiments, the first reactive chemical moiety may comprise an alkyne and a maleimide. In some embodiments, the first reactive chemical moiety may comprise an alkyne and an NHS-ester. In some embodiments, the first reactive chemical moiety may comprise an alkyne and a carbodiimide. In some embodiments, the first reactive chemical moiety may comprise an alkyne and an imidoester. In some embodiments, the first reactive chemical moiety may comprise an alkyne and a haloacetyl. In some embodiments, the first reactive chemical moiety may comprise an alkyne and a pyridyldisulfide. In some embodiments, the first reactive chemical moiety may comprise an alkyne and a hydrazide. In some embodiments, the first reactive chemical moiety may comprise an alkyne and an alkoxyamine. In some embodiments, the first reactive chemical moiety may comprise an alkyne and a diazirine. In some embodiments, the first reactive chemical moiety may comprise an alkyne and a phosphine. In some embodiments, the first reactive chemical moiety may comprise an alkyne and an epoxide. In some embodiments, the first reactive chemical moiety may comprise an alkyne and an aldehyde. In some embodiments, the first reactive chemical moiety may comprise an amine and a carboxyl. In some embodiments, the first reactive chemical moiety may comprise an amine and a sulfhydryl. In some embodiments, the first reactive chemical moiety may comprise an amine and a carboxylic acid. In some embodiments, the first reactive chemical moiety may comprise an amine and a maleimide. In some embodiments, the first reactive chemical moiety may comprise an amine and an NHS-ester. In some embodiments, the first reactive chemical moiety may comprise an amine and a carbodiimide. In some embodiments, the first reactive chemical moiety may comprise an amine and an imidoester. In some embodiments, the first reactive chemical moiety may comprise an amine and a haloacetyl. In some embodiments, the first reactive chemical moiety may comprise an amine and a pyridyldisulfide. In some embodiments, the first reactive chemical moiety may comprise an amine and a hydrazide. In some embodiments, the first reactive chemical moiety may comprise an amine and an alkoxyamine. In some embodiments, the first reactive chemical moiety may comprise an amine and a diazirine. In some embodiments, the first reactive chemical moiety may comprise an amine and a phosphine. In some embodiments, the first reactive chemical moiety may comprise an amine and an epoxide. In some embodiments, the first reactive chemical moiety may comprise an amine and an aldehyde. In some embodiments, the first reactive chemical moiety may comprise a carboxyl and a sulfhydryl. In some embodiments, the first reactive chemical moiety may comprise a carboxyl and a carboxylic acid. In some embodiments, the first reactive chemical moiety may comprise a carboxyl and a maleimide. In some embodiments, the first reactive chemical moiety may comprise a carboxyl and an NHS-ester. In some embodiments, the first reactive chemical moiety may comprise a carboxyl and a carbodiimide. In some embodiments, the first reactive chemical moiety may comprise a carboxyl and an imidoester. In some embodiments, the first reactive chemical moiety may comprise a carboxyl and a haloacetyl. In some embodiments, the first reactive chemical moiety may comprise a carboxyl and a pyridyldisulfide. In some embodiments, the first reactive chemical moiety may comprise a carboxyl and a hydrazide. In some embodiments, the first reactive chemical moiety may comprise a carboxyl and an alkoxyamine. In some embodiments, the first reactive chemical moiety may comprise a carboxyl and a diazirine. In some embodiments, the first reactive chemical moiety may comprise a carboxyl and a phosphine. In some embodiments, the first reactive chemical moiety may comprise a carboxyl and an epoxide. In some embodiments, the first reactive chemical moiety may comprise a carboxyl and an aldehyde. In some embodiments, the first reactive chemical moiety may comprise a sulfhydryl and a carboxylic acid. In some embodiments, the first reactive chemical moiety may comprise a sulfhydryl and a maleimide. In some embodiments, the first reactive chemical moiety may comprise a sulfhydryl and an NHS-ester. In some embodiments, the first reactive chemical moiety may comprise a sulfhydryl and a carbodiimide. In some embodiments, the first reactive chemical moiety may comprise a sulfhydryl and an imidoester. In some embodiments, the first reactive chemical moiety may comprise a sulfhydryl and a haloacetyl. In some embodiments, the first reactive chemical moiety may comprise a sulfhydryl and a pyridyldisulfide. In some embodiments, the first reactive chemical moiety may comprise a sulfhydryl and a hydrazide. In some embodiments, the first reactive chemical moiety may comprise a sulfhydryl and an alkoxyamine. In some embodiments, the first reactive chemical moiety may comprise a sulfhydryl and a diazirine. In some embodiments, the first reactive chemical moiety may comprise a sulfhydryl and a phosphine. In some embodiments, the first reactive chemical moiety may comprise a sulfhydryl and an epoxide. In some embodiments, the first reactive chemical moiety may comprise a sulfhydryl and an aldehyde. In some embodiments, the first reactive chemical moiety may comprise a carboxylic acid and a maleimide. In some embodiments, the first reactive chemical moiety may comprise a carboxylic acid and an NHS-ester. In some embodiments, the first reactive chemical moiety may comprise a carboxylic acid and a carbodiimide. In some embodiments, the first reactive chemical moiety may comprise a carboxylic acid and an imidoester. In some embodiments, the first reactive chemical moiety may comprise a carboxylic acid and a haloacetyl. In some embodiments, the first reactive chemical moiety may comprise a carboxylic acid and a pyridyldisulfide. In some embodiments, the first reactive chemical moiety may comprise a carboxylic acid and a hydrazide. In some embodiments, the first reactive chemical moiety may comprise a carboxylic acid and an alkoxyamine. In some embodiments, the first reactive chemical moiety may comprise a carboxylic acid and a diazirine. In some embodiments, the first reactive chemical moiety may comprise a carboxylic acid and a phosphine. In some embodiments, the first reactive chemical moiety may comprise a carboxylic acid and an epoxide. In some embodiments, the first reactive chemical moiety may comprise a carboxylic acid and an aldehyde. In some embodiments, the first reactive chemical moiety may comprise a maleimide and an NHS-ester. In some embodiments, the first reactive chemical moiety may comprise a maleimide and a carbodiimide. In some embodiments, the first reactive chemical moiety may comprise a maleimide and an imidoester. In some embodiments, the first reactive chemical moiety may comprise a maleimide and a haloacetyl. In some embodiments, the first reactive chemical moiety may comprise a maleimide and a pyridyldisulfide. In some embodiments, the first reactive chemical moiety may comprise a maleimide and a hydrazide. In some embodiments, the first reactive chemical moiety may comprise a maleimide and an alkoxyamine. In some embodiments, the first reactive chemical moiety may comprise a maleimide and a diazirine. In some embodiments, the first reactive chemical moiety may comprise a maleimide and a phosphine. In some embodiments, the first reactive chemical moiety may comprise a maleimide and an epoxide. In some embodiments, the first reactive chemical moiety may comprise a maleimide and an aldehyde. In some embodiments, the first reactive chemical moiety may comprise an NHS-ester and a carbodiimide. In some embodiments, the first reactive chemical moiety may comprise an NHS-ester and an imidoester. In some embodiments, the first reactive chemical moiety may comprise an NHS-ester and a haloacetyl. In some embodiments, the first reactive chemical moiety may comprise an NHS-ester and a pyridyldisulfide. In some embodiments, the first reactive chemical moiety may comprise an NHS-ester and a hydrazide. In some embodiments, the first reactive chemical moiety may comprise an NHS-ester and an alkoxyamine. In some embodiments, the first reactive chemical moiety may comprise an NHS-ester and a diazirine. In some embodiments, the first reactive chemical moiety may comprise an NHS-ester and a phosphine. In some embodiments, the first reactive chemical moiety may comprise an NHS-ester and an epoxide. In some embodiments, the first reactive chemical moiety may comprise an NHS-ester and an aldehyde. In some embodiments, the first reactive chemical moiety may comprise a carbodiimide and an imidoester. In some embodiments, the first reactive chemical moiety may comprise a carbodiimide and a haloacetyl. In some embodiments, the first reactive chemical moiety may comprise a carbodiimide and a pyridyldisulfide. In some embodiments, the first reactive chemical moiety may comprise a carbodiimide and a hydrazide. In some embodiments, the first reactive chemical moiety may comprise a carbodiimide and an alkoxyamine. In some embodiments, the first reactive chemical moiety may comprise a carbodiimide and a diazirine. In some embodiments, the first reactive chemical moiety may comprise a carbodiimide and a phosphine. In some embodiments, the first reactive chemical moiety may comprise a carbodiimide and an epoxide. In some embodiments, the first reactive chemical moiety may comprise a carbodiimide and an aldehyde. In some embodiments, the first reactive chemical moiety may comprise an imidoester. In some embodiments, the first reactive chemical moiety may comprise an imidoester and a haloacetyl. In some embodiments, the first reactive chemical moiety may comprise an imidoester and a pyridyldisulfide. In some embodiments, the first reactive chemical moiety may comprise an imidoester and a hydrazide. In some embodiments, the first reactive chemical moiety may comprise an imidoester and an alkoxyamine. In some embodiments, the first reactive chemical moiety may comprise an imidoester and a diazirine. In some embodiments, the first reactive chemical moiety may comprise an imidoester and a phosphine. In some embodiments, the first reactive chemical moiety may comprise an imidoester and an epoxide. In some embodiments, the first reactive chemical moiety may comprise an imidoester and an aldehyde. In some embodiments, the first reactive chemical moiety may comprise a haloacetyl and a pyridyldisulfide. In some embodiments, the first reactive chemical moiety may comprise a haloacetyl and a hydrazide. In some embodiments, the first reactive chemical moiety may comprise a haloacetyl and an alkoxyamine. In some embodiments, the first reactive chemical moiety may comprise a haloacetyl and a diazirine. In some embodiments, the first reactive chemical moiety may comprise a haloacetyl and a phosphine. In some embodiments, the first reactive chemical moiety may comprise a haloacetyl and an epoxide. In some embodiments, the first reactive chemical moiety may comprise a haloacetyl and an aldehyde. In some embodiments, the first reactive chemical moiety may comprise a pyridyldisulfide and a hydrazide. In some embodiments, the first reactive chemical moiety may comprise a pyridyldisulfide and an alkoxyamine. In some embodiments, the first reactive chemical moiety may comprise a pyridyldisulfide and a diazirine. In some embodiments, the first reactive chemical moiety may comprise a pyridyldisulfide and a phosphine. In some embodiments, the first reactive chemical moiety may comprise a pyridyldisulfide and an epoxide. In some embodiments, the first reactive chemical moiety may comprise a pyridyldisulfide and an aldehyde. In some embodiments, the first reactive chemical moiety may comprise a hydrazide and an alkoxyamine. In some embodiments, the first reactive chemical moiety may comprise a hydrazide and a diazirine. In some embodiments, the first reactive chemical moiety may comprise a hydrazide and a phosphine. In some embodiments, the first reactive chemical moiety may comprise a hydrazide and an epoxide. In some embodiments, the first reactive chemical moiety may comprise a hydrazide and an aldehyde. In some embodiments, the first reactive chemical moiety may comprise an alkoxyamine and a diazirine. In some embodiments, the first reactive chemical moiety may comprise an alkoxyamine and a phosphine. In some embodiments, the first reactive chemical moiety may comprise an alkoxyamine and an epoxide. In some embodiments, the first reactive chemical moiety may comprise an alkoxyamine and an aldehyde. In some embodiments, the first reactive chemical moiety may comprise a diazirine and a phosphine. In some embodiments, the first reactive chemical moiety may comprise a diazirine and an epoxide. In some embodiments, the first reactive chemical moiety may comprise a diazirine and an aldehyde. In some embodiments, the first reactive chemical moiety may comprise a phosphine and an epoxide. In some embodiments, the first reactive chemical moiety may comprise a phosphine and an aldehyde. In some embodiments, the first reactive chemical moiety may comprise an epoxide and an aldehyde.

In some cases, a second chemical reactive moiety (e.g., the second chemical reactive moiety) may be incorporated into an amplicon. The first chemical reactive moiety and the second chemical reactive moiety may be present in an amplicon. The first chemical reactive moiety may be in proximity to the second chemical reactive moiety. The first chemical reactive moiety may react with the second chemical reactive moiety because of their proximity within the amplicon. In some cases, the first chemical reactive moiety may be in proximity to the second chemical reactive moiety in relation to their three-dimensional locations. For example, the first and second chemical reactive moieties may be incorporated into an amplicon during amplification. The first chemical reactive moiety may be spatially proximal to the second chemical reactive moiety within the volume of the amplicon. In some cases, the first reactive chemical moiety may be proximally close to the second chemical reactive moiety based on their location on the nucleic acid strand of the amplicon. The first chemical reactive moiety may be separated by one or more nucleotides on the nucleic acid of the amplicon form the second chemical reactive moiety. In some cases, the first chemical reactive moiety may be separated from the second chemical reactive moiety of the amplicon by at least about 1 nucleotide, at least about 2 nucleotides, at least about 5 nucleotides, at least about 10 nucleotides, at least about 20 nucleotides, at least about 50 nucleotides, at least about 100 nucleotides, at least about 500 nucleotides, at least about 1,000 nucleotides, at least about 5,000 nucleotides, at least about 10,000 nucleotides, or more nucleotides. In some cases, the first chemical reactive moiety may be separated from the second chemical reactive moiety of the amplicon by at most about 1 nucleotide, at most about 2 nucleotides, at most about 5 nucleotides, at most about 10 nucleotides, at most about 20 nucleotides, at most about 50 nucleotides, at most about 100 nucleotides, at most about 500 nucleotides, at most about 1,000 nucleotides, at most about 5,000 nucleotides, at most about 10,000 nucleotides, or fewer nucleotides.

In some cases, the second reactive chemical moiety may comprise an azide. In some cases, the second reactive chemical moiety may comprise an alkyne (e.g., a cyclooctyne). In some cases, the second reactive chemical moiety may comprise an amine. In some cases, the second reactive chemical moiety may comprise a carboxyl. In some cases, the second reactive chemical moiety may comprise a sulfhydryl. In some cases, the second reactive chemical moiety may comprise a carboxylic acid. In some cases, the second reactive chemical moiety may comprise a maleimide. In some cases, the second reactive chemical moiety may comprise an NHS-ester. In some cases, the second reactive chemical moiety may comprise a carbodiimide. In some cases, the second reactive chemical moiety may comprise an imidoester. In some cases, the second reactive chemical moiety may comprise a haloacetyl. In some cases, the second reactive chemical moiety may comprise a pyridyldisulfide. In some cases, the second reactive chemical moiety may comprise a hydrazide. In some cases, the second reactive chemical moiety may comprise an alkoxyamine. In some cases, the second reactive chemical moiety may comprise a diazirine. In some cases, the second reactive chemical moiety may comprise a phosphine. In some cases, the second reactive chemical moiety may comprise an epoxide. In some cases, the second reactive chemical moiety may comprise an aldehyde. In some embodiments, the second reactive chemical moiety may comprise an azide and an alkyne. In some embodiments, the second reactive chemical moiety may comprise an azide and an amine. In some embodiments, the second reactive chemical moiety may comprise an azide and a carboxyl. In some embodiments, the second reactive chemical moiety may comprise an azide and a sulfhydryl. In some embodiments, the second reactive chemical moiety may comprise an azide and a carboxylic acid. In some embodiments, the second reactive chemical moiety may comprise an azide and a maleimide. In some embodiments, the second reactive chemical moiety may comprise an azide and an NHS-ester. In some embodiments, the second reactive chemical moiety may comprise an azide and a carbodiimide. In some embodiments, the second reactive chemical moiety may comprise an azide and an imidoester. In some embodiments, the second reactive chemical moiety may comprise an azide and a haloacetyl. In some embodiments, the second reactive chemical moiety may comprise an azide and a pyridyldisulfide. In some embodiments, the second reactive chemical moiety may comprise an azide and a hydrazide. In some embodiments, the second reactive chemical moiety may comprise an azide and an alkoxyamine. In some embodiments, the second reactive chemical moiety may comprise an azide and a diazirine. In some embodiments, the second reactive chemical moiety may comprise an azide and a phosphine. In some embodiments, the second reactive chemical moiety may comprise an azide and an epoxide. In some embodiments, the second reactive chemical moiety may comprise an azide and an aldehyde. In some embodiments, the second reactive chemical moiety may comprise an alkyne and an amine. In some embodiments, the second reactive chemical moiety may comprise an alkyne and a carboxyl. In some embodiments, the second reactive chemical moiety may comprise an alkyne and a sulfhydryl. In some embodiments, the second reactive chemical moiety may comprise an alkyne and a carboxylic acid. In some embodiments, the second reactive chemical moiety may comprise an alkyne and a maleimide. In some embodiments, the second reactive chemical moiety may comprise an alkyne and an NHS-ester. In some embodiments, the second reactive chemical moiety may comprise an alkyne and a carbodiimide. In some embodiments, the second reactive chemical moiety may comprise an alkyne and an imidoester. In some embodiments, the second reactive chemical moiety may comprise an alkyne and a haloacetyl. In some embodiments, the second reactive chemical moiety may comprise an alkyne and a pyridyldisulfide. In some embodiments, the second reactive chemical moiety may comprise an alkyne and a hydrazide. In some embodiments, the second reactive chemical moiety may comprise an alkyne and an alkoxyamine. In some embodiments, the second reactive chemical moiety may comprise an alkyne and a diazirine. In some embodiments, the second reactive chemical moiety may comprise an alkyne and a phosphine. In some embodiments, the second reactive chemical moiety may comprise an alkyne and an epoxide. In some embodiments, the second reactive chemical moiety may comprise an alkyne and an aldehyde. In some embodiments, the second reactive chemical moiety may comprise an amine and a carboxyl. In some embodiments, the second reactive chemical moiety may comprise an amine and a sulfhydryl. In some embodiments, the second reactive chemical moiety may comprise an amine and a carboxylic acid. In some embodiments, the second reactive chemical moiety may comprise an amine and a maleimide. In some embodiments, the second reactive chemical moiety may comprise an amine and an NHS-ester. In some embodiments, the second reactive chemical moiety may comprise an amine and a carbodiimide. In some embodiments, the second reactive chemical moiety may comprise an amine and an imidoester. In some embodiments, the second reactive chemical moiety may comprise an amine and a haloacetyl. In some embodiments, the second reactive chemical moiety may comprise an amine and a pyridyldisulfide. In some embodiments, the second reactive chemical moiety may comprise an amine and a hydrazide. In some embodiments, the second reactive chemical moiety may comprise an amine and an alkoxyamine. In some embodiments, the second reactive chemical moiety may comprise an amine and a diazirine. In some embodiments, the second reactive chemical moiety may comprise an amine and a phosphine. In some embodiments, the second reactive chemical moiety may comprise an amine and an epoxide. In some embodiments, the second reactive chemical moiety may comprise an amine and an aldehyde. In some embodiments, the second reactive chemical moiety may comprise a carboxyl and a sulfhydryl. In some embodiments, the second reactive chemical moiety may comprise a carboxyl and a carboxylic acid. In some embodiments, the second reactive chemical moiety may comprise a carboxyl and a maleimide. In some embodiments, the second reactive chemical moiety may comprise a carboxyl and an NHS-ester. In some embodiments, the second reactive chemical moiety may comprise a carboxyl and a carbodiimide. In some embodiments, the second reactive chemical moiety may comprise a carboxyl and an imidoester. In some embodiments, the second reactive chemical moiety may comprise a carboxyl and a haloacetyl. In some embodiments, the second reactive chemical moiety may comprise a carboxyl and a pyridyldisulfide. In some embodiments, the second reactive chemical moiety may comprise a carboxyl and a hydrazide. In some embodiments, the second reactive chemical moiety may comprise a carboxyl and an alkoxyamine. In some embodiments, the second reactive chemical moiety may comprise a carboxyl and a diazirine. In some embodiments, the second reactive chemical moiety may comprise a carboxyl and a phosphine. In some embodiments, the second reactive chemical moiety may comprise a carboxyl and an epoxide. In some embodiments, the second reactive chemical moiety may comprise a carboxyl and an aldehyde. In some embodiments, the second reactive chemical moiety may comprise a sulfhydryl and a carboxylic acid. In some embodiments, the second reactive chemical moiety may comprise a sulfhydryl and a maleimide. In some embodiments, the second reactive chemical moiety may comprise a sulfhydryl and an NHS-ester. In some embodiments, the second reactive chemical moiety may comprise a sulfhydryl and a carbodiimide. In some embodiments, the second reactive chemical moiety may comprise a sulfhydryl and an imidoester. In some embodiments, the second reactive chemical moiety may comprise a sulfhydryl and a haloacetyl. In some embodiments, the second reactive chemical moiety may comprise a sulfhydryl and a pyridyldisulfide. In some embodiments, the second reactive chemical moiety may comprise a sulfhydryl and a hydrazide. In some embodiments, the second reactive chemical moiety may comprise a sulfhydryl and an alkoxyamine. In some embodiments, the second reactive chemical moiety may comprise a sulfhydryl and a diazirine. In some embodiments, the second reactive chemical moiety may comprise a sulfhydryl and a phosphine. In some embodiments, the second reactive chemical moiety may comprise a sulfhydryl and an epoxide. In some embodiments, the second reactive chemical moiety may comprise a sulfhydryl and an aldehyde. In some embodiments, the second reactive chemical moiety may comprise a carboxylic acid and a maleimide. In some embodiments, the second reactive chemical moiety may comprise a carboxylic acid and an NHS-ester. In some embodiments, the second reactive chemical moiety may comprise a carboxylic acid and a carbodiimide. In some embodiments, the second reactive chemical moiety may comprise a carboxylic acid and an imidoester. In some embodiments, the second reactive chemical moiety may comprise a carboxylic acid and a haloacetyl. In some embodiments, the second reactive chemical moiety may comprise a carboxylic acid and a pyridyldisulfide. In some embodiments, the second reactive chemical moiety may comprise a carboxylic acid and a hydrazide. In some embodiments, the second reactive chemical moiety may comprise a carboxylic acid and an alkoxyamine. In some embodiments, the second reactive chemical moiety may comprise a carboxylic acid and a diazirine. In some embodiments, the second reactive chemical moiety may comprise a carboxylic acid and a phosphine. In some embodiments, the second reactive chemical moiety may comprise a carboxylic acid and an epoxide. In some embodiments, the second reactive chemical moiety may comprise a carboxylic acid and an aldehyde. In some embodiments, the second reactive chemical moiety may comprise a maleimide and an NHS-ester. In some embodiments, the second reactive chemical moiety may comprise a maleimide and a carbodiimide. In some embodiments, the second reactive chemical moiety may comprise a maleimide and an imidoester. In some embodiments, the second reactive chemical moiety may comprise a maleimide and a haloacetyl. In some embodiments, the second reactive chemical moiety may comprise a maleimide and a pyridyldisulfide. In some embodiments, the second reactive chemical moiety may comprise a maleimide and a hydrazide. In some embodiments, the second reactive chemical moiety may comprise a maleimide and an alkoxyamine. In some embodiments, the second reactive chemical moiety may comprise a maleimide and a diazirine. In some embodiments, the second reactive chemical moiety may comprise a maleimide and a phosphine. In some embodiments, the second reactive chemical moiety may comprise a maleimide and an epoxide. In some embodiments, the second reactive chemical moiety may comprise a maleimide and an aldehyde. In some embodiments, the second reactive chemical moiety may comprise an NHS-ester and a carbodiimide. In some embodiments, the second reactive chemical moiety may comprise an NHS-ester and an imidoester. In some embodiments, the second reactive chemical moiety may comprise an NHS-ester and a haloacetyl. In some embodiments, the second reactive chemical moiety may comprise an NHS-ester and a pyridyldisulfide. In some embodiments, the second reactive chemical moiety may comprise an NHS-ester and a hydrazide. In some embodiments, the second reactive chemical moiety may comprise an NHS-ester and an alkoxyamine. In some embodiments, the second reactive chemical moiety may comprise an NHS-ester and a diazirine. In some embodiments, the second reactive chemical moiety may comprise an NHS-ester and a phosphine. In some embodiments, the second reactive chemical moiety may comprise an NHS-ester and an epoxide. In some embodiments, the second reactive chemical moiety may comprise an NHS-ester and an aldehyde. In some embodiments, the second reactive chemical moiety may comprise a carbodiimide and an imidoester. In some embodiments, the second reactive chemical moiety may comprise a carbodiimide and a haloacetyl. In some embodiments, the second reactive chemical moiety may comprise a carbodiimide and a pyridyldisulfide. In some embodiments, the second reactive chemical moiety may comprise a carbodiimide and a hydrazide. In some embodiments, the second reactive chemical moiety may comprise a carbodiimide and an alkoxyamine. In some embodiments, the second reactive chemical moiety may comprise a carbodiimide and a diazirine. In some embodiments, the second reactive chemical moiety may comprise a carbodiimide and a phosphine. In some embodiments, the second reactive chemical moiety may comprise a carbodiimide and an epoxide. In some embodiments, the second reactive chemical moiety may comprise a carbodiimide and an aldehyde. In some embodiments, the second reactive chemical moiety may comprise an imidoester. In some embodiments, the second reactive chemical moiety may comprise an imidoester and a haloacetyl. In some embodiments, the second reactive chemical moiety may comprise an imidoester and a pyridyldisulfide. In some embodiments, the second reactive chemical moiety may comprise an imidoester and a hydrazide. In some embodiments, the second reactive chemical moiety may comprise an imidoester and an alkoxyamine. In some embodiments, the second reactive chemical moiety may comprise an imidoester and a diazirine. In some embodiments, the second reactive chemical moiety may comprise an imidoester and a phosphine. In some embodiments, the second reactive chemical moiety may comprise an imidoester and an epoxide. In some embodiments, the second reactive chemical moiety may comprise an imidoester and an aldehyde. In some embodiments, the second reactive chemical moiety may comprise a haloacetyl and a pyridyldisulfide. In some embodiments, the second reactive chemical moiety may comprise a haloacetyl and a hydrazide. In some embodiments, the second reactive chemical moiety may comprise a haloacetyl and an alkoxyamine. In some embodiments, the second reactive chemical moiety may comprise a haloacetyl and a diazirine. In some embodiments, the second reactive chemical moiety may comprise a haloacetyl and a phosphine. In some embodiments, the second reactive chemical moiety may comprise a haloacetyl and an epoxide. In some embodiments, the second reactive chemical moiety may comprise a haloacetyl and an aldehyde. In some embodiments, the second reactive chemical moiety may comprise a pyridyldisulfide and a hydrazide. In some embodiments, the second reactive chemical moiety may comprise a pyridyldisulfide and an alkoxyamine. In some embodiments, the second reactive chemical moiety may comprise a pyridyldisulfide and a diazirine. In some embodiments, the second reactive chemical moiety may comprise a pyridyldisulfide and a phosphine. In some embodiments, the second reactive chemical moiety may comprise a pyridyldisulfide and an epoxide. In some embodiments, the second reactive chemical moiety may comprise a pyridyldisulfide and an aldehyde. In some embodiments, the second reactive chemical moiety may comprise a hydrazide and an alkoxyamine. In some embodiments, the second reactive chemical moiety may comprise a hydrazide and a diazirine. In some embodiments, the second reactive chemical moiety may comprise a hydrazide and a phosphine. In some embodiments, the second reactive chemical moiety may comprise a hydrazide and an epoxide. In some embodiments, the second reactive chemical moiety may comprise a hydrazide and an aldehyde. In some embodiments, the second reactive chemical moiety may comprise an alkoxyamine and a diazirine. In some embodiments, the second reactive chemical moiety may comprise an alkoxyamine and a phosphine. In some embodiments, the second reactive chemical moiety may comprise an alkoxyamine and an epoxide. In some embodiments, the second reactive chemical moiety may comprise an alkoxyamine and an aldehyde. In some embodiments, the second reactive chemical moiety may comprise a diazirine and a phosphine. In some embodiments, the second reactive chemical moiety may comprise a diazirine and an epoxide. In some embodiments, the second reactive chemical moiety may comprise a diazirine and an aldehyde. In some embodiments, the second reactive chemical moiety may comprise a phosphine and an epoxide. In some embodiments, the second reactive chemical moiety may comprise a phosphine and an aldehyde. In some embodiments, the second reactive chemical moiety may comprise an epoxide and an aldehyde.

In some embodiments, the amplification reaction comprises a rolling circle amplification reaction using the circular nucleic acid to form an amplicon. In some embodiments, the rolling circle amplification reaction comprises a polymerase. In some embodiments, the polymerase is a phi29 DNA polymerase. In some embodiments, the polymerase may comprise a DNA polymerase. In some embodiments, the rolling circle amplification reaction may be performed at a temperature of from about 30° C. to about 65° C., from about 30° C. to about 37° C., from about 60° C. to about 65° C., from about 20° C. to about 80° C., from about 30° C. to about 70° C., from about 40° C. to about 60° C., or from about 30° C. to about 50° C. In some embodiments, the rolling circle amplification reaction comprises buffer and salts. In some embodiments, the salt comprises Mg²⁺, or another divalent cation. In some embodiments, the amplicon may be from about 1 nucleotide to about 100,000 nucleotides, from about 10 nucleotides to about 90,000 nucleotides from about 50 nucleotides to about 80,000 nucleotides, from about 100 nucleotides to about 70,000 nucleotides, from about 1,000 nucleotides to about 60,000 nucleotides, from about 10,000 nucleotides to about 50,000 nucleotides, from about 20,000 nucleotides to about 40,000 nucleotides, greater than or equal to 10,000 nucleotides, greater than or equal to 20,000 nucleotides, greater than or equal to 30,000 nucleotides, greater than or equal to 40,000 nucleotides, greater than or equal to 50,000 nucleotides, greater than or equal to 60,000 nucleotides, greater than or equal to 70,000 nucleotides, greater than or equal to 80,000 nucleotides, greater than or equal to 90,000 nucleotides, greater than or equal to 100,000 nucleotides, or greater than or equal to 150,000 nucleotides, or more.

### Amplicon Compaction

The methods described herein may comprise compacting one or more amplicons. Compacting the one or more amplicons generated using any one of the methods described herein may have certain advantages. In some cases, compacting one or more amplicons may result in a stronger detection signal of the one or more amplicons. For example, the one or more amplicons may be compacted and the size of an amplicon of the one or more amplicons may be decreased. The decreased size of the amplicon may increase the intensity of signal associated with the amplicon. The stronger signal associated with the amplicon may enable detection of the amplicon with a lower exposure time. In some cases, the stronger signal associated with the amplicon may enable detection of a low-expressing analyte that may not be detectable without compaction. In some cases, the stronger signal may enable a faster experimental time. For example, compacting the amplicon may result in a stronger signal that can be detected with a lower exposure time. The lower exposure time may enable a faster experimental time due to faster imaging scans. In some cases, compacting one or more amplicons may result in a rounder amplicon shape. For example, an amplicon of the one or more amplicons may be compacted and may be round in shape as a result of the compacting. The round shape may enable detecting more amplicons in a unit area. In some cases, more amplicons can be detecting in a unit area after compacting due to a smaller, more round shape of the amplicons as a result of compacting. Compacting one or more amplicons may result in a lower FWHM. A lower FWHM may enable faster imaging of a sample. In some cases, a lower FWHM may enable detecting more amplicons in a unit area. For example, a cell of a tissue sample may be analyzed using methods as described herein. The cell may comprise a plurality of transcripts. Amplicons may be generated corresponding to the plurality of transcripts. The amplicons may be compacted to generate compacted amplicons. More compacted amplicons may be detectable and discernable from other amplicons compared to the non-compacted amplicons in the cell. More transcripts of the plurality of transcripts may be detectable when compacted.

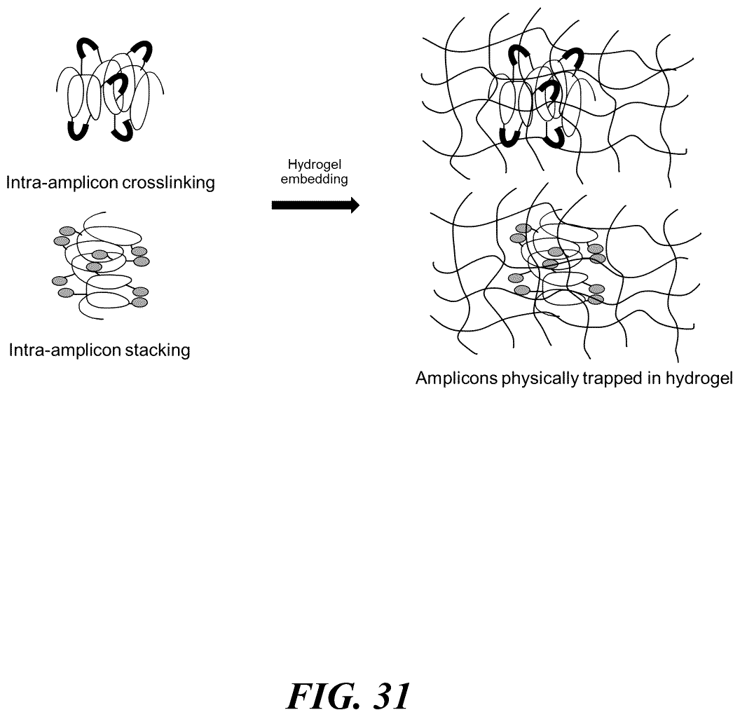

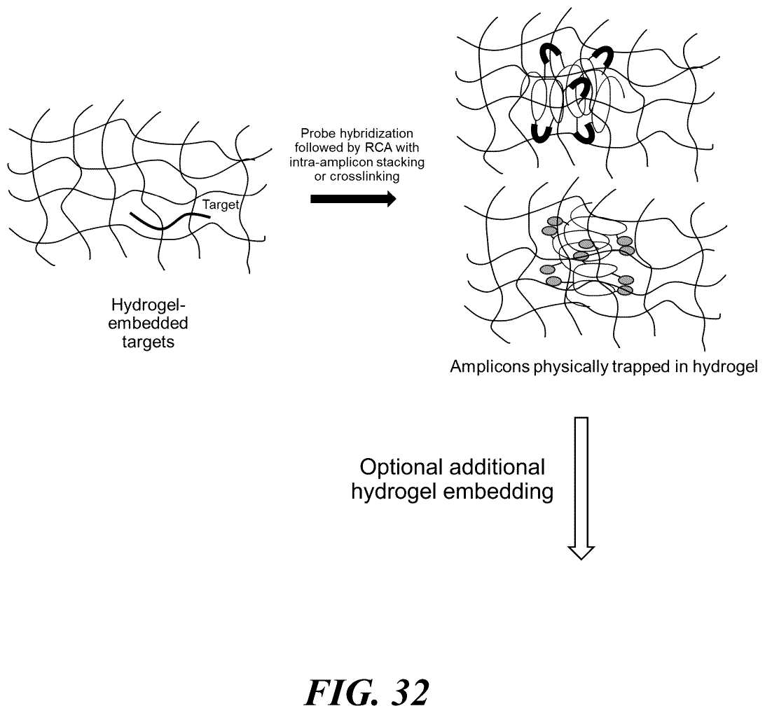

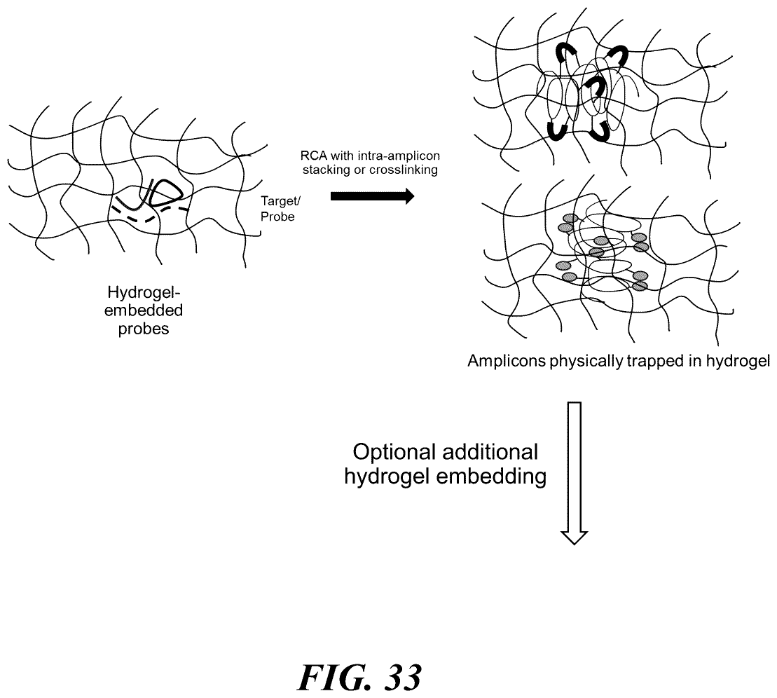

The methods described herein may comprise various methods for compacting one or more amplicons. In some cases, one or more amplicons may be compacted using intra-amplicon cross-linking. Intra-amplicon cross-linking may comprise formation of a chemical cross-link between one or more chemical reactive moieties of an amplicon of the one or more amplicons. In some cases, a compaction agent may be used to form the crosslink between the one or more chemical reactive moieties of the amplicon of the one or more amplicons. In some cases, intra-amplicon cross-linking may comprise nucleic acid hybridization. For example, a compaction agent comprising nucleic acid may be added to a sample comprising one or more amplicons. The compaction agent comprising nucleic acid may bind to the one or more amplicons, thereby compacting the one or more amplicons. In some cases, compacting an amplicon may comprise formation of non-covalent interactions within the amplicon. In some cases, a hydrophobic moiety may be incorporated into an amplicon during amplification. In some cases, the hydrophobic moiety may comprise dibenzocyclooctyne (DBCO). In some cases, the hydrophobic moiety may comprise one or more of linear alkyl chains, cholesterol, aromatic hydrocarbons, fluorinated alkyl chains, tocopherol, steroid derivatives, alkylated aromatics, aliphatic diacyl chains, or any combination thereof. In some cases, the hydrophobic moiety may comprise an octyl group, a dodecyl group, a stearyl group, a naphthalene, an anthracene, a pyrene, a phenanthrene, a perfluorooctyl, a perflourorbutyl, an estradiol, a testosterone derivative, octylphenyl, tert-butylbenzyl, naphthylmethyl, palmitoyl-oleoyl, disterayl, or any combination thereof. The hydrophobic moieties may interact with each other within the amplicon, thereby compacting the amplicon. In some cases, a combination of intra-amplicon crosslinking and hydrophobic group interactions may both contribute to amplicon compaction. In some cases, compacted amplicons may be embedded in a hydrogel. In some cases, analytes may be embedded in a hydrogel before generation of one or more amplicons. An example of embedding compacted amplicons in a hydrogel is shown in FIG. 31. Compacted amplicons comprising intra-amplicon crosslinking and intra-amplicon stacking (e.g., interaction between hydrophobic moieties of the one or more amplicons) may be embedded in a hydrogel. The embedded compacted amplicons may be surrounded by a matrix. The embedded compacted amplicons may be crosslinked to the matrix of the hydrogel. The embedded compacted amplicons may not be crosslinked to the matrix of the hydrogel. FIG. 32 shows an example of performing amplicon formation and compaction after analyte hydrogel embedding. In this example, an analyte (e.g., a target) is hydrogel embedded. The hydrogel-embedded analyte (e.g., target) may be contacted with reagents and materials for performing RCA to generate one or more amplicons. The one or more amplicons may be compacted using formation of intra-amplicon crosslinks and/or intra-amplicon non-covalent interactions (e.g., interactions between hydrophobic chemical groups of the one or more amplicons). The compacted amplicons may be cross-linked to the hydrogel. The compacted amplicons may be subjected to an additional hydrogel embedding process to form an additional layer of hydrogel. FIG. 33 shows an example of performing amplicon formation and compaction after binding moiety-analyte binding and hydrogel embedding. In this example, a binding moiety that binds to an analyte (e.g., a target) is hydrogel embedded. The hydrogel-embedded binding moiety may be contacted with reagents and materials for performing RCA to generate one or more amplicons. The materials to perform RCA may comprise binding moieties and probes that bind to the analyte (e.g., the target). The one or more amplicons may be compacted using formation of intra-amplicon crosslinks and/or intra-amplicon non-covalent interactions (e.g., interactions between hydrophobic chemical groups of the one or more amplicons). The compacted amplicons may not be cross-linked to the hydrogel. The compacted amplicons may be physically trapped in the hydrogel in the absence of cross-linking. The compacted amplicons may be subjected to an additional hydrogel embedding process to form an additional layer of hydrogel.

The methods described herein may comprise incorporating one or more hydrophobic groups into the one or more amplicons during amplification. For example, a modified nucleotide comprising one or more hydrophobic groups may be added to the sample during amplification to generate one or more amplicons. The one or more amplicons may comprise the one or more hydrophobic groups. The one or more modified nucleotides added during amplification may be provided in solution (e.g., in a buffer). The one or more modified nucleotides added during amplification comprise a concentration of at least about 100 nM, at least about 500 nM, at least about 1 μM, at least about 5 μM, at least about 10 μM, at least about 15 μM, at least about 20 μM, at least about 25 μM, at least about 30 μM, at least about 40 μM, at least about 50 μM, at least about 75 μM, at least about 100 μM, at least about 250 μM, at least about 500 μM, at least about 750 μM, at least about 1000 μM, or more. The one or more modified nucleotides added during amplification comprise a concentration of at most about 100 nM, at most about 500 nM, at most about 1 μM, at most about 5 μM, at most about 10 μM, at most about 15 μM, at most about 20 μM, at most about 25 μM, at most about 30 μM, at most about 40 μM, at most about 50 μM, at most about 75 μM, at most about 100 μM, at most about 250 μM, at most about 500 μM, at most about 750 μM, at most about 1000 μM, or less.

In some embodiments, one or more amplicons may comprise one or more reactive chemical moieties. In some cases, the amplicon may comprise a reactive chemical moiety (e.g., a first reactive chemical moiety). The reactive chemical moiety (e.g. the first reactive chemical moiety) may comprise an azide, an alkyne, an amine, a carboxyl, a sulfydrul, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method may comprise cross-linking the first reactive chemical moiety to another reactive chemical moiety (e.g., a second reactive chemical moiety). In some cases, the methods described herein may comprise cross-linking one copy, two copies, three copies, four copies, five copies, or more of the first reactive chemical moiety to one or more copies of another reactive chemical moiety (e.g., a second reactive chemical moiety). In some embodiments, the cross-linking of the first reactive chemical moiety to another reactive chemical moiety (e.g., a second reactive chemical moiety) may comprise use of a linker. In some embodiments, the linker may comprise a polyethylene glycol. In some embodiments, the linker may comprise a methylene group. In some embodiments, a diameter of the amplicon may be reduced as a result of cross-linking.

In some embodiments, one or more amplicons may comprise another reactive chemical moiety (e.g., a second reactive chemical moiety. In some embodiments, the second reactive chemical moiety may comprise an azide, an alkyne, an amine, a carboxyl, a sulfydrul, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof. In some embodiments, the method may comprise cross-linking the first reactive chemical moiety and the second reactive chemical moiety. In some embodiments, the cross-linking may comprise use of a linker. In some embodiments, the linker may comprise a polyethylene glycol. In some embodiments, the linker may comprise a methylene group.

In some embodiments, the detection probe may bind to the amplicon. The detection probe may comprise a nucleic acid (e.g., a ribonucleic acid, deoxyribonucleic acid, or a combination thereof). The nucleic acid of the detection probe may hybridize to the amplicon at one or more sites. For example, the amplicon may comprise multiple copies of a binding site for the detection probe and the detection probe may hybridize to the multiple copies of the binding site for the detection probe on the amplicon. In some embodiments, the detection probe may comprise about 1-20 nucleotides, about 2-19 nucleotides, about 3-18 nucleotides, about 4-17 nucleotides, about 5-16 nucleotides, about 6-15 nucleotides, about 7-14 nucleotides, about 8-13 nucleotides, about 9-12 nucleotides, about 10-11 nucleotides, at least about 1 nucleotide, at least about 2 nucleotides, at least about 3 nucleotides, at least about 4 nucleotides, at least about 5 nucleotides, at least about 6 nucleotides, at least about 7 nucleotides, at least about 8 nucleotides, at least about 9 nucleotides, at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 25 nucleotides, at least about 30 nucleotides, or more nucleotides. In some embodiments, the detection probe comprises a fluorescent dye. In some embodiments, the detection probe comprises an enzyme. In some embodiments, the detection probe comprises a linker.

Compacting one or more amplicons to generate one or more compacted amplicons may comprise decreasing a size of an amplicon of the one or more amplicons, changing a shape of an amplicon of the one or more amplicons, increasing a density of an amplicon of the one or more amplicons, or a combination thereof. In some cases, the one or more compacted amplicons may have a smaller diameter, cross-section, radius, circumference, boundary, volume, or a combination thereof as a result of the compacting. In some cases, an amplicon of the one or more amplicons may be polymorphous in shape. For example, the amplicon may have an elongated shape, where the elongated shape may be longer in one dimension relative to another dimension. Compacting the amplicon to generate a compacted amplicon may result in changing the shape of the amplicon such that the compacted amplicon is more spherical in shape relative to the amplicon prior to the compacting. In some cases, an amplicon of the one or more amplicons may have a density of nucleic acid. The density of the nucleic acid may be increased after compacting. The increased density of the compacted amplicon may be a result of the decreased size of the compacted amplicon.

In some cases, compacting an amplicon to generate a compacted amplicon may result in the compacted amplicon having a decreased diameter relative to the diameter of the amplicon. The decreased diameter of the compacted amplicon relative to the diameter of the amplicon may be at least about 5% less than the diameter of the amplicon before compaction, at least about 10% less than the diameter of the amplicon before compaction, at least about 15% less than the diameter of the amplicon before compaction, at least about 20% less than the diameter of the amplicon before compaction, at least about 25% less than the diameter of the amplicon before compaction, at least about 30% less than the diameter of the amplicon before compaction, at least about 35% less than the diameter of the amplicon before compaction, at least about 40% less than the diameter of the amplicon before compaction, at least about 45% less than the diameter of the amplicon before compaction, at least about 50% less than the diameter of the amplicon before compaction, at least about 60% less than the diameter of the amplicon before compaction, at least about 70% less than the diameter of the amplicon before compaction, at least about 80% less than the diameter of the amplicon before compaction, at least about 90% less than the diameter of the amplicon before compaction, at least about 95% less than the diameter of the amplicon before compaction, at most about 5% less than the diameter of the amplicon before compaction, at most about 10% less than the diameter of the amplicon before compaction, at most about 15% less than the diameter of the amplicon before compaction, at most about 20% less than the diameter of the amplicon before compaction, at most about 25% less than the diameter of the amplicon before compaction, at most about 30% less than the diameter of the amplicon before compaction, at most about 35% less than the diameter of the amplicon before compaction, at most about 40% less than the diameter of the amplicon before compaction, at most about 45% less than the diameter of the amplicon before compaction, at most about 50% less than the diameter of the amplicon before compaction, at most about 60% less than the diameter of the amplicon before compaction, at most about 70% less than the diameter of the amplicon before compaction, at most about 80% less than the diameter of the amplicon before compaction, at most about 90% less than the diameter of the amplicon before compaction, at most about 95% less than the diameter of the amplicon before compaction, about 5%-95% less than the diameter of the amplicon before compaction, about 10%-90% less than the diameter of the amplicon before compaction, about 15%-80% less than the diameter of the amplicon before compaction, about 20%-70% less than the diameter of the amplicon before compaction, about 25%-60% less than the diameter of the amplicon before compaction, about 30%-50% less than the diameter of the amplicon before compaction, or about 35%-45% less than the diameter of the amplicon before compaction. In some cases, compacting an amplicon to generate a compacted amplicon may result in the compacted amplicon having a decreased volume relative to the volume of the amplicon. The decreased volume of the compacted amplicon relative to the volume of the amplicon may be at least about 5% less than the volume of the amplicon before compaction, at least about 10% less than the volume of the amplicon before compaction, at least about 15% less than the volume of the amplicon before compaction, at least about 20% less than the volume of the amplicon before compaction, at least about 25% less than the volume of the amplicon before compaction, at least about 30% less than the volume of the amplicon before compaction, at least about 35% less than the volume of the amplicon before compaction, at least about 40% less than the volume of the amplicon before compaction, at least about 45% less than the volume of the amplicon before compaction, at least about 50% less than the volume of the amplicon before compaction, at least about 60% less than the volume of the amplicon before compaction, at least about 70% less than the volume of the amplicon before compaction, at least about 80% less than the volume of the amplicon before compaction, at least about 90% less than the volume of the amplicon before compaction, at least about 95% less than the volume of the amplicon before compaction, at most about 5% less than the volume of the amplicon before compaction, at most about 10% less than the volume of the amplicon before compaction, at most about 15% less than the volume of the amplicon before compaction, at most about 20% less than the volume of the amplicon before compaction, at most about 25% less than the volume of the amplicon before compaction, at most about 30% less than the volume of the amplicon before compaction, at most about 35% less than the volume of the amplicon before compaction, at most about 40% less than the volume of the amplicon before compaction, at most about 45% less than the volume of the amplicon before compaction, at most about 50% less than the volume of the amplicon before compaction, at most about 60% less than the volume of the amplicon before compaction, at most about 70% less than the volume of the amplicon before compaction, at most about 80% less than the volume of the amplicon before compaction, at most about 90% less than the volume of the amplicon before compaction, at most about 95% less than the volume of the amplicon before compaction, about 5%-95% less than the volume of the amplicon before compaction, about 10%-90% less than the volume of the amplicon before compaction, about 15%-80% less than the volume of the amplicon before compaction, about 20%-70% less than the volume of the amplicon before compaction, about 25%-60% less than the volume of the amplicon before compaction, or about 30%-50% less than the volume of the amplicon before compaction, about 35%-45% less than the volume of the amplicon before compaction.

Compacting one or more amplicons of the methods described herein may comprise a variety of methods. In some cases, the one or more amplicons may comprise reactive chemical moieties. The reactive chemical moieties of an amplicon may be cross-linked to each other to generate a cross-link (e.g., a conjugate). The cross-link may result in compacting the amplicon being compacted (e.g., generating a compacted amplicon). In some cases, reactive chemical moieties may be cross-linked directly (e.g., without a linker). In some cases, reactive chemical moieties may be cross-linked with a linker. In some cases, a compaction agent comprising a nucleic acid may be added to a sample comprising an amplicon. The compaction agent comprising the nucleic acid may bind to the amplicon at one or more locations, thereby compacting the amplicon. For example, the compaction agent comprising the nucleic acid may hybridize to a first location of the amplicon and a second location of the amplicon. The first location of the amplicon and the second location of the amplicon may be separated by a distance within the amplicon before being contacted with the compaction agent comprising nucleic acid. Upon the compaction agent comprising the nucleic acid hybridizing to the amplicon, the distance separating the first location of the amplicon, and the second location of the amplicon may be changed (e.g., decreased) thereby resulting in compacting the amplicon. In some cases, a combination of compacting methods may be used. For example, an amplicon may comprise reactive chemical moieties that form a cross-link and a compaction agent comprising a nucleic acid that binds to the amplicon may be added to the sample, thereby compacting the amplicon. In some cases, an amplicon may comprise multiple different reactive chemical moieties. The multiple different reactive chemical moieties may form cross-links, thereby generating a compacted amplicon.

The methods described herein may comprise compacting an amplicon of one or more amplicons using reactive chemical moieties. The reactive chemical moieties may be introduced into the one or more amplicons during amplification, as described above. The reactive chemical moieties may comprise one or more of the reactive chemical moieties described herein. In some cases, the amplicon may comprise multiple copies of a reactive chemical moiety (e.g., two or more copies). A compaction agent may be added to the sample comprising the amplicon. The compaction agent may comprise a linker and one or more reactive chemical moieties. In some cases, the compaction agent may comprise a linker and a first reactive chemical moiety on a first end of the linker and a second reactive chemical moiety on a second end of the linker. In some cases, the first reactive chemical moiety may be the same reactive chemical moiety as the second reactive chemical moiety (e.g., the same chemical group). In some cases, the first reactive chemical moiety may be different than the second reactive chemical moiety (e.g., the first reactive chemical moiety comprises a different chemical group than a chemical group of the second reactive chemical moiety.) The first reactive chemical moiety of the compaction agent may react with a first copy of the reactive chemical moiety of the amplicon. The second reactive chemical moiety of the compaction agent may react with a second copy of the reactive chemical moiety of the amplicon, thereby forming a cross-link. The cross-link may result in compacting the amplicon to generate a compacted amplicon. In some cases, multiple cross-links may be formed between reactive chemical moieties of the amplicon and reactive chemical moieties of compaction agents.

In some cases, an amplicon may comprise a first reactive chemical moiety and a second reactive chemical moiety. The first reactive chemical moiety may react with the second reactive chemical moiety to form a cross-link. The cross-link may thereby compact the amplicon. In some cases, a compaction agent may be added to the amplicon. The compaction agent may comprise a linker and a third reactive chemical moiety and a fourth reactive chemical moiety. The first reactive chemical moiety of the amplicon may react with the third reactive chemical moiety of the compaction agent to form a first cross-link. The second chemical moiety of the amplicon may react with the fourth reactive chemical moiety of the compaction agent to form a second cross-link. The first cross-link and/or the second cross-link may compact the amplicon. In some cases, the first reactive chemical moiety may be the same as the second reactive chemical moiety (e.g., the first reactive chemical moiety comprises the same chemical group). In some cases, the first reactive chemical moiety may be different from the second reactive chemical moiety (e.g., the first reactive chemical moiety comprises different chemical groups). In some cases, the first reactive chemical moiety may be the same as the fourth reactive chemical moiety (e.g., the first reactive chemical moiety comprises the same chemical group). In some cases, the first reactive chemical moiety may be different from the fourth reactive chemical moiety (e.g., the first reactive chemical moiety comprises different chemical groups). In some cases, the third reactive chemical moiety may be the same as the fourth reactive chemical moiety (e.g., the third reactive chemical moiety comprises the same chemical group). In some cases, the third reactive chemical moiety may be different from the fourth reactive chemical moiety (e.g., the third reactive chemical moiety comprises different chemical groups). In some cases, the second reactive chemical moiety may be the same as the third reactive chemical moiety (e.g., the second reactive chemical moiety comprises the same chemical group). In some cases, the second reactive chemical moiety may be different from the third reactive chemical moiety (e.g., the second reactive chemical moiety comprises different chemical groups).

In some cases, one or more cross-links may form between one or more reactive chemical groups and/or between one or more reactive chemical groups and one or more compaction agents. The one or more cross-links may comprise a covalent bond. For example, an azide of an amplicon may cross-link with an alkyne of a compaction agent to form a triazole ring. The triazole ring may comprise a covalent bond. In some cases, the one or more cross-links may comprise one or more non-covalent interactions. For example, a compaction agent may comprise a nucleic acid. The nucleic acid may comprise double-stranded DNA comprising a non-covalent interaction.

In some cases, one or more compaction agents may be added to one or more amplicons of the methods described herein. For example, in some cases, a nucleic acid may be added to the one or more amplicons and the nucleic acid may hybridize to one or more portions of the one or more amplicons.

The one or more amplicons may be compacted using nucleic acid hybridization. In some cases, a compaction agent comprising nucleic acid may be added to a sample comprising an amplicon. The amplicon may comprise chemical reactive moieties. The compaction agent comprising nucleic acid may hybridize to nucleic acid of the amplicon at one or more locations to generate a compacted amplicon. In some cases, a different compaction agent may be added to the sample. The different compaction agent may react with one or more reactive chemical moieties of the amplicon to generate one or more cross-links, thereby compacting the amplicon. The compaction agent comprising nucleic acid (e.g., a sequence) may comprise singled-stranded nucleic acid, double-stranded nucleic acid, or a combination thereof. In some cases, the compaction agent comprising nucleic acid (e.g., a sequence) may comprise a length of at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, at least about 100 nucleotides, at least about 120 nucleotides, at least about 150 nucleotides, at least about 175 nucleotides, at least about 200 nucleotides, at least about 250 nucleotides, at least about 300 nucleotides, 10 nucleotides, at most about 20 nucleotides, at most about 30 nucleotides, at most about 40 nucleotides, at most about 50 nucleotides, at most about 60 nucleotides, at most about 70 nucleotides, at most about 80 nucleotides, at most about 90 nucleotides, at most about 100 nucleotides, at most about 120 nucleotides, at most about 150 nucleotides, at most about 175 nucleotides, at most about 200 nucleotides, at most about 250 nucleotides, at most about 300 nucleotides, about 10-300 nucleotides, about 20-250 nucleotides, about 30-200 nucleotides, about 40-175 nucleotides, about 50-150 nucleotides, about 60-120 nucleotides, about 70-100 nucleotides, or about 80-90 nucleotides.

In some cases, a compaction agent comprising nucleic acid (e.g., a condensing oligonucleotide) may be added to a sample comprising an amplicon and removed from the sample. For example, a compaction agent comprising a nucleic acid may be added to a sample comprising an amplicon. The compaction agent comprising the nucleic acid may hybridize to nucleic acid of the amplicon at one or more locations of the amplicon. Chemical reactive moieties of the amplicon may be reacted to each other to form cross-links (e.g., azides and alkynes of the amplicon may be reacted to form triazole rings). The compaction agent comprising the nucleic acid may be removed from the amplicon. In some cases, removing a compaction agent comprising nucleic acid from an amplicon of a sample may comprise heating the sample, incubating the sample with a chaotropic agent (e.g., sodium hydroxide, DMSO, formamide, or a combination thereof), incubating the sample with a solvent (e.g., methanol, acetonitrile, ethanol, or a combination thereof), or a combination thereof.

The methods described herein may comprise compacting an amplicon by reacting one or more reactive chemical moieties of the amplicon to each other, reacting one or more reactive chemical moieties of the amplicon to compaction agents, or a combination thereof, under conditions sufficient to promote formation of a cross-link between reactive chemical moieties and/or reactive chemical moieties and compaction agents. In some cases, an amplicon may comprise reactive chemical moieties. A first reactive chemical moiety of the amplicon may react with a second reactive chemical moiety that is in proximity to the first reactive chemical moiety (e.g., a proximity mediated reaction). For example, an amplicon may comprise an azide and a dibenzocyclooctyne (DBCO). The azide of the amplicon may react with the DBCO of the amplicon to form a crosslink. The crosslink may compact the amplicon.

The conditions sufficient to promote formation of a cross-link between reactive chemical moieties and/or reactive chemical moieties and compaction agents may comprise incubating the sample with a reaction mixture. The incubating may comprise incubating the sample for a period of time. The period of time may be at least about 5 minutes, at least about 10 minutes, at least about 15 minutes, at least about 20 minutes, at least about 25 minutes, at least about 30 minutes, at least about 40 minutes, at least about 45 minutes, at least about 50 minutes, at least about 55 minutes, at least about 60 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 1 day, at least about 2 days, at least about 3 days at least about 4 days or longer. The length of time may be at most about 5 minutes, at most about 10 minutes, at most about 15 minutes, at most about 20 minutes, at most about 25 minutes, at most about 30 minutes, at most about 40 minutes, at most about 45 minutes, at most about 50 minutes, at most about 55 minutes, at most about 60 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 1 day, at most about 2 days, at most about 3 days at most about 4 days or less. The length of time may be about 5 minutes-24 hours, about 10 minutes-23 hours, about 15 minutes-22 hours, about 20 minutes-21 hours, about 25 minutes-20 hours, about 30 minutes-19 hours, about 40 minutes-18 hours, about 45 minutes-17 hours, about 50 minutes-16 hours, about 55 minutes-15 hours, about 60 minutes-14 hours, about 1 hour-13 hours, about 2 hours-12 hours, about 3 hours-11 hours, about 4 hours-10 hours, about 5 hours-9 hours, or about 6 hours-8 hours. The incubating may comprise incubating the sample at one or more temperatures. The one or more temperatures may be at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., or higher. The one or more temperatures may be at most about 4° C., at most about 5° C., at most about 6° C., at most about 7° C., at most about 8° C., at most about 9° C., at most about 10° C., at most about 11° C., at most about 12° C., at most about 13° C., at most about 14° C., at most about 15° C., at most about 16° C., at most about 17° C., at most about 18° C., at most about 19° C., at most about 20° C., at most about 21° C., at most about 22° C., at most about 23° C., at most about 24° C., at most about 25° C., at most about 26° C., at most about 27° C., at most about 28° C., at most about 29° C., at most about 30° C., at most about 31° C., at most about 32° C., at most about 33° C., at most about 34° C., at most about 35° C., at most about 36° C., at most about 37° C., at most about 38° C., at most about 39° C., at most about 40° C., at most about 41° C., at most about 42° C., at most about 43° C., at most about 44° C., at most about 45° C., at most about 46° C., at most about 47° C., at most about 48° C., at most about 49° C., at most about 50° C., at most about 51° C., at most about 52° C., at most about 53° C., at most about 54° C., at most about 55° C., at most about 56° C., at most about 57° C., at most about 58° C., at most about 59° C., at most about 60° C., at most about 61° C., at most about 62° C., at most about 63° C., at most about 64° C., at most about 65° C., at most about 66° C., at most about 67° C., at most about 68° C., at most about 69° C., at most about 70° C., at most about 71° C., at most about 72° C., at most about 73° C., at most about 74° C., at most about 75° C., at most about 76° C., at most about 77° C., at most about 78° C., at most about 79° C., at most about 80° C., at most about 81° C., at most about 82° C., at most about 83° C., at most about 84° C., at most about 85° C., at most about 86° C., at most about 87° C., at most about 88° C., at most about 89° C., at most about 90° C., at most about 91° C., at most about 92° C., at most about 93° C., at most about 94° C., at most about 95° C., or lower. The one or more temperatures may be about 4-95° C., about 5-94° C., about 6-93° C., about 7-92° C., about 8-91° C., about 9-90° C., about 10-89° C., about 11-88° C., about 12-87° C., about 13-86° C., about 14-85° C., about 15-84° C., about 16-83° C., about 17-82° C., about 18-81° C., about 19-80° C., about 20-79° C., about 21-78° C., about 22-77° C., about 23-76° C., about 24-75° C., about 25-74° C., about 26-73° C., about 27-72° C., about 28-71° C., about 29-70° C., about 30-69° C., about 31-68° C., about 32-67° C., about 33-66° C., about 34-65° C., about 35-64° C., about 36-63° C., about 37-62° C., about 38-61° C., about 39-60° C., about 40-59° C., about 41-58° C., about 42-57° C., about 43-56° C., about 44-55° C., about 45-54° C., about 46-53° C., about 47-52° C., about 48-51° C., or about 49-50° C.

The conditions sufficient to promote formation of a cross-link between reactive chemical moieties and/or reactive chemical moieties and compaction agents may comprise one or more buffers. The one or more buffers may comprise MES (4-Morpholineethanesulfonic acid), Bis-Tris (Bis(2-hydroxyethyl)amino-tris (hydroxymethyl) methane), ADA, ACES, PIPES, MOSO, Bis-Tris Propane, BES, MOPS, TES, HEPES, DIPSO, MOBS, TAPSO, Tris, HEPPSO, POPSO, TEA, EPPS, Tricine, Gly-Gly, Bicine, HEPBS, TAPS, AMPD, TABS, AMPSO, CHES, CAPSO, AMP, CAPS, CAPS, Phosphate buffered saline, or a combination thereof. The conditions sufficient to promote formation of a cross-link between reactive chemical moieties and/or reactive chemical moieties and compaction agents may comprise one or more salts. The one or more salts may comprise NaCl, CaCl2, MgCl2, or a combination thereof. The conditions sufficient to promote formation of a cross-link between reactive chemical moieties and/or reactive chemical moieties and compaction agents may comprise one or more detergents. The one or more detergents of the reaction mixture may comprise SDS, Triton X-100, CHAPS, NP-40, Tween-20, Digitonin, or a combination thereof. The conditions sufficient to promote formation of a cross-link between reactive chemical moieties and/or reactive chemical moieties and compaction agents may comprise one or more solvents. The one or more solvents may comprise methanol, ethanol, ethyl acetate, DMSO, acetonitrile, water, or a combination thereof. The conditions sufficient to promote formation of a cross-link between reactive chemical moieties and/or reactive chemical moieties and compaction agents may comprise DMSO, formamide, urea, thiourea, 2-propanol, guanidinium chloride, n-butanol, or a combination thereof. The conditions sufficient to promote formation of a cross-link between reactive chemical moieties and/or reactive chemical moieties and compaction agents may comprise a pH. The pH may be at least about 2, at least about 2.4, at least about 2.6, at least about 2.8, at least about 3, at least about 3.2, at least about 3.4, at least about 3.6, at least about 3.8, at least about 4, at least about 4.1, at least about 4.2, at least about 4.3, at least about 4.4, at least about 4.5, at least about 4.6, at least about 4.7, at least about 4.8, at least about 4.9, at least about 5, at least about 5.1, at least about 5.2, at least about 5.3, at least about 5.4, at least about 5.5, at least about 5.6, at least about 5.7, at least about 5.8, at least about 5.9, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.2, at least about 9.4, at least about 9.6, at least about 9.8, at least about 10, at least about 10.2, at least about 10.4, at least about 10.6, at least about 10.8, at least about 11, at least about 11.2, at least about 11.4, at least about 11.6, at least about 11.8, at least about 12, or more. The pH may be at most about 2, at most about 2.4, at most about 2.6, at most about 2.8, at most about 3, at most about 3.2, at most about 3.4, at most about 3.6, at most about 3.8, at most about 4, at most about 4.1, at most about 4.2, at most about 4.3, at most about 4.4, at most about 4.5, at most about 4.6, at most about 4.7, at most about 4.8, at most about 4.9, at most about 5, at most about 5.1, at most about 5.2, at most about 5.3, at most about 5.4, at most about 5.5, at most about 5.6, at most about 5.7, at most about 5.8, at most about 5.9, at most about 6, at most about 6.1, at most about 6.2, at most about 6.3, at most about 6.4, at most about 6.5, at most about 6.6, at most about 6.7, at most about 6.8, at most about 6.9, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.2, at most about 9.4, at most about 9.6, at most about 9.8, at most about 10, at most about 10.2, at most about 10.4, at most about 10.6, at most about 10.8, at most about 11, at most about 11.2, at most about 11.4, at most about 11.6, at most about 11.8, at most about 12, or lower.

The methods described herein may comprise contacting a sample comprising one or more amplicons with one or more compaction agents. In some cases, the one or more compaction agents may comprise multiple copies of the same compaction agent (e.g., the same molecule). In some cases, the one or more compaction agents may comprise different compaction agents (e.g., different molecules). In some cases, a compaction agent of the one or more compaction agents may comprise a nucleic acid. The nucleic acid may bind to (e.g., hybridize to) one or more locations of an amplicon comprising nucleic acid in a sample. The compaction agent comprising nucleic acid may promote compaction of the amplicon by altering the shape and/or density of the nucleic acid of the amplicon.

A compaction agent of the one or more compaction agents may comprise one or more linkers, one or more reactive chemical moieties, one or more polymers, or a combination thereof. The one or more linkers of the compaction agent may comprise one or more methylene groups, one or more ethylene groups, one or more polyethylene glycol groups, one or more ethers, or a combination thereof. The one or more reactive chemical moieties may comprise an NHS-ester, a maleimide, an alkyne, and azide, an amine, a thiol, a hydroxyl, an aldehyde, a carboxylic acid, or a combination thereof. The one or more reactive chemical moieties may comprise one or more of any reactive chemical moieties described herein. The one or more polymers may comprise a nucleic acid, a polypeptide, or a combination thereof.

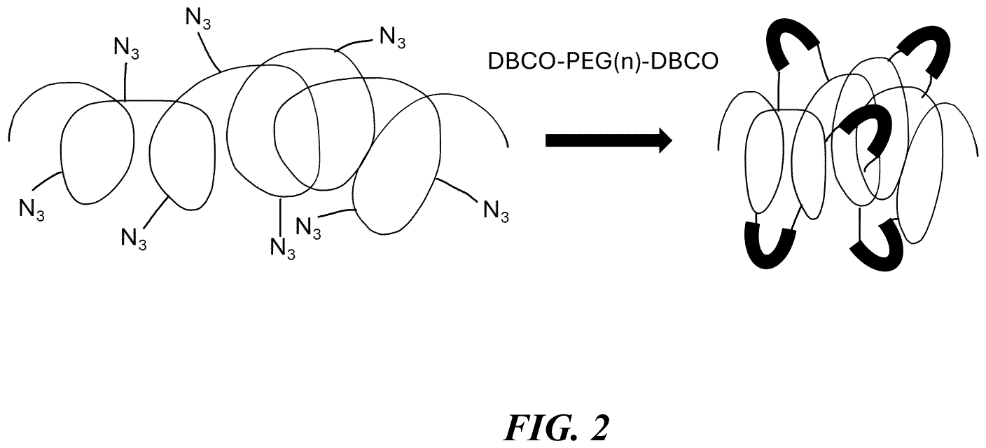

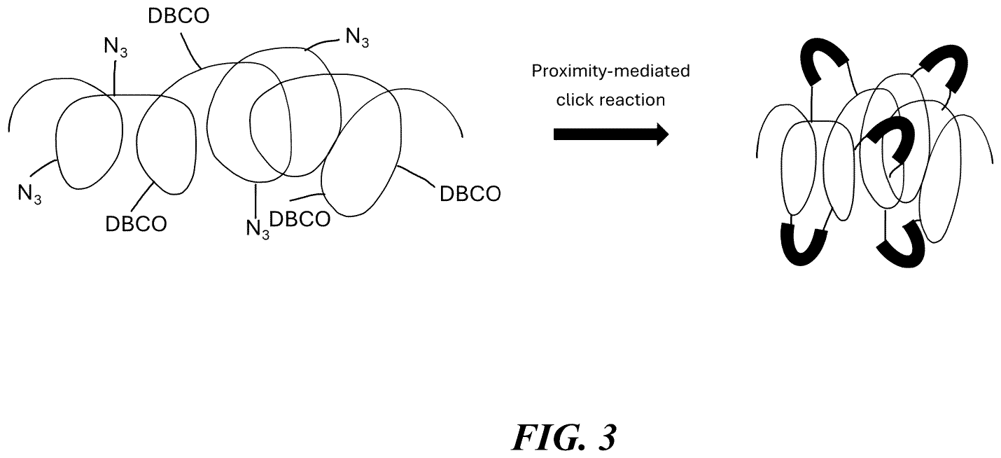

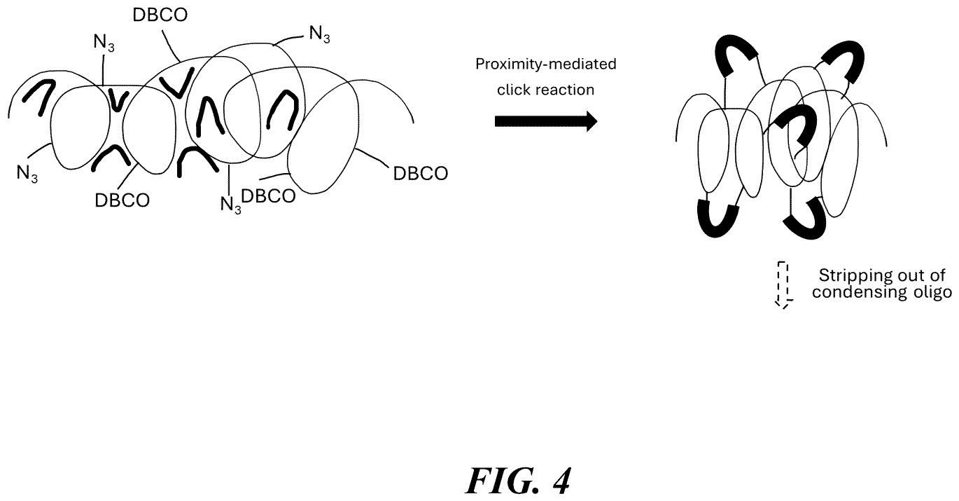

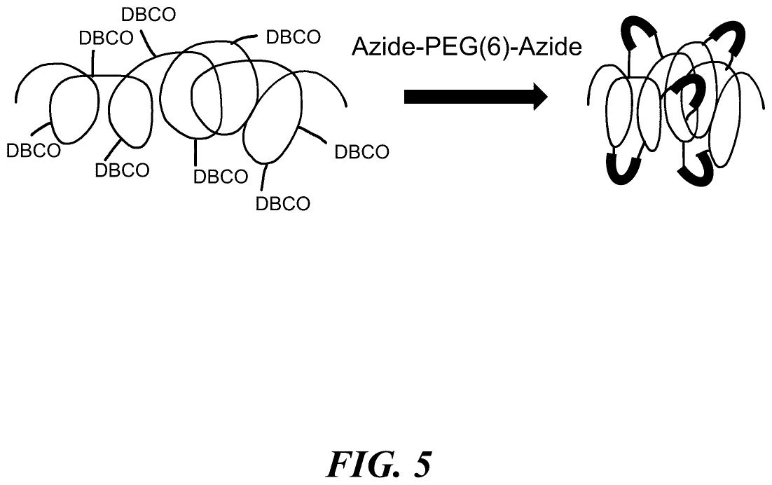

An example of use of a compaction agent is shown in FIG. 2. In this case, an amplicon comprises one or more azide groups. A compaction agent comprising dibenzocyclooctyne (DBCO)-polyethylene glycol (PEG)ₙ-DBCO may be added to the azide containing amplicon. The DBCO-PEGₙ-DBCO may react with the azides of the amplicon to generate a compacted amplicon. Another example of use of a compaction agent is shown in FIG. 4. In this case, an amplicon comprises one or more azide groups and one or more DBCO groups. The amplicon may also be complexed with a nucleic acid (e.g., a condensing oligonucleotide), which may hybridize to the amplicon at one or more locations. A click reaction may be performed based on proximity of the DBCO and azide groups of the amplicon to generate a compacted amplicon. In some cases, the condensing oligonucleotide may be removed after formation of the triazole ring of the azide and DBCO. In some cases, the condensing oligonucleotide may be removed before formation of the triazole ring of the azide and DBCO. In some cases, the condensing oligonucleotide may be removed at the same time as the formation of the triazole ring of the azide and DBCO. FIG. 3 shows an example where a proximity mediated click reaction takes place between azides and DBCO groups of an amplicon to generate a condensed amplicon. The compaction agent may bind to an amplicon, a barcode, a reverse complement of the barcode, or a combination thereof.

### Hydrogel

The methods described herein may comprise embedding a sample, in hydrogel. Embedding the sample in hydrogel may be useful for detecting and/or identifying one or more analytes. For example, embedding the sample in a hydrogel may maintain a three-dimensional relationship of the analytes in a sample. In some cases, embedding the sample in a hydrogel may maintain a three-dimensional relationship of amplicons in a sample. For example, one or more amplicons may be generated using any one of the methods described herein. The one or more amplicons may be embedded in a hydrogel thereby forming a matrix around the one or more amplicons. The matrix around the one or more amplicons may fix the three-dimensional relationship of the one or more amplicons. In some cases, the one or more amplicons may be cross-linked to the hydrogel. In some cases, the one or more amplicons may not be cross-linked to the hydrogel. In some cases, embedding the one or more amplicons in a hydrogel may compact the one or more amplicons.

The methods described herein may comprise embedding the sample in a hydrogel. In some cases, the methods may comprise obtaining a sample that is embedded. The hydrogel may be formed by polymerizing monomers in the presence of the sample. The hydrogel may comprise one or more polymers. The one or more polymers may comprise poly (vinyl alcohol) (PVA), poly (ethylene glycol) (PEG), poly (ethylene oxide) (PEO), poly (2-hydroxyethyl methacrylate) (PHEMA), poly (acrylic acid) (PAA), poly (acrylamide) (PAAm), or a combination thereof. Embedding the sample in a hydrogel may comprise adding any one of the monomers and/or polymers to the sample. A hydrogel may be formed during any operation of the methods described herein. For example, a hydrogel may be formed before or after cross-linking a sample, before or after contacting a sample with one or more binding moieties, before or after contacting a sample within one or probes, before or after performing an amplification reaction (e.g., a rolling circle amplification reaction), before or after performing a ligation reaction, before or after compacting one or more amplicons, before or after contacting a sample with one or more detection probes, or before or after imaging a sample. Embedding a sample in hydrogel may provide certain advantages. For example, embedding a sample in hydrogel may create an optically clear sample (e.g., a tissue sample). Having an optically clear sample may enable imaging a thicker sample (e.g., a tissue sample). In some cases, embedding a sample in hydrogel may retain components of the sample in a fixed spatial location.

### Amplicon Detection

The methods described herein may comprise detecting one or more amplicons (e.g., one or more compacted amplicons) Detecting one or more amplicons may comprise detecting a signal associated with one or more amplicons. The one or more amplicons (e.g., one or more compacted amplicon) may comprise one or more barcodes or derivative thereof (e.g., reverse complement thereof). The one or more barcodes or derivative thereof may be associated with one or more analytes. Detecting the one or more barcodes or derivatives thereof may enable identifying the one or more analytes associated with the one or more barcodes or derivatives thereof.

Detecting one or more amplicons may comprise imaging a sample comprising the one or more amplicons. Imaging the sample comprising the one or more amplicons may comprise use of an imaging system as described herein. The sample comprising the one or more amplicons may be placed on a stage of the imaging system. The imaging system may illuminate regions of the sample with a light beam. The light beam may interact with fluorescent molecules associated with one or more detection probes bound to the one or more amplicons. The fluorescent molecules may emit light as a result of interacting with the light beam emitted by the imaging system. In some cases, the light beam may comprise multiple wavelengths. In some cases, the multiple wavelengths may interact with multiple fluorescent molecules of the one or more detection probes bound to the one or more amplicons. The emitted light from the fluorescent molecules may be detected by the imaging system to generate imaging data. In some cases, the imaging system may acquire imaging data at multiple X-Y regions of the sample. In some cases, the imaging system may acquire volume imaging data at the X-Y regions of the sample (e.g., imaging data across multiple z planes at the X-Y regions of the sample). The imaging data may comprise images. The images may comprise pixels. The pixels of the images may comprise fluorescent intensities. In some cases, the images may comprise pixels comprising fluorescent intensities for one or more fluorescent channels (e.g., 488 nm, 550 nm, 647 nm, 740 nm, or any combination thereof). The Fluorescent intensities of the pixels of the images may be analyzed to detect the one or more amplicons (e.g., compacted amplicons). For example, an image comprising pixels comprising fluorescence intensities may be analyzed and regions of the image comprising a fluorescent intensity above a threshold may be identified. The identified regions of the image comprising a fluorescent intensity above a threshold may be determined to be amplicons. Image data may be analyzed for multiple fluorescent channels, multiple regions of the sample, multiple cycles of the method, or any combination thereof. In some cases, a location of the sample may be imaged one or more times across cycles of a method described herein. Imaging data from the one or more cycles of the method may be analyzed for the location. Based on the imaging data, an identify of an analyte may be identified (e.g., by correlating the imaging data to a codebook comprising information about barcodes corresponding to analytes).

Detecting the one or more barcodes or derivatives thereof may comprise contacting the sample with materials to perform an in situ sequencing based reaction. A variety of in situ sequencing based reactions may be performed, including but not limited to sequencing by synthesis, sequencing by hybridization, Multiplexed Error-Robust Fluorescence In Situ Hybridization (MERFISH), Sequencing by Oligonucleotide Ligation and Detection (SOLID), sequencing with error-reduction by dynamic annealing and ligation (SEDAL), or a combination thereof. Details of performing SEDAL sequencing can be found in PCT/US2019/025835, which is incorporated by reference herein in its entirety.

In some cases, detecting the one or more barcodes or reverse complements thereof may comprise using a plurality of probes. The plurality of probes may comprise a detection probe, an anchor probe, or a combination thereof. The anchor probe may comprise nucleic acid. The anchor probe may be used for detection of the barcode or derivative thereof (e.g., reverse complement thereof) by hybridizing to a portion of an amplicon (e.g., compacted amplicon) that is adjacent to a binding site on the amplicon of the detection probe to enable a ligation event. The ligation between the anchor probe and the detection probe may increase the melting temperature associated with the ligated product as compared to the melting temperature of the detection probe alone. The increased melting temperature of the ligated product may provide enhanced stability of the duplex formed between the detection probe and the amplicon (e.g., compacted amplicon), and thereby enable more specific detection of the detection probe bound to the amplicon. The anchor probes may bind to the amplicon formed during amplification. An anchor probe of the anchor probes may bind to all or a portion of the barcode or derivative thereof. A detection probe may bind to all or a portion of the barcode or derivative thereof. The anchor probe of the anchor probes may bind to a sequence adjacent to the barcode or derivative thereof. The detection probe may bind to a sequence adjacent to the barcode or derivative thereof.

The detection probe may comprise a nucleic acid. The nucleic acid may comprise DNA, RNA, or a combination thereof. The nucleic acid of the detection probe may be single-stranded, double-stranded, or a combination thereof. The nucleic acid of the detection probe may comprise LNA. The detection probe may comprise a label. The label may be a detectable label, a linker, or a combination thereof. The detectable label may comprise a fluorescent molecule.

Detecting the barcode or reverse complement of the barcode may involve reading a signal associated with the barcode or reverse complement of the barcode. A variety of methods can use used to read a signal associated with the reverse complement of the barcode including hybridization-based detection, sequencing based detection, or a combination thereof. For example, in situ base-by-base sequencing may be performed to detect one or more barcodes or reverse complement thereof. In situ base-by-base sequencing may comprise adding sequencing primers, nucleotides, one or more polymerases, or any combination thereof to a sample comprising one or more amplicons comprising one or more barcodes or reverse complements thereof. Nucleotides comprising fluorescent labels may be added to sequencing primers bound to the one or more amplicons. Signals associated with the fluorescent labels added to the sequencing primers bound to the one or more amplicons may be detected using an imaging system described herein. The signals may correspond to nucleotides of the one or more barcodes or reverse complements thereof of the one or more amplicons of the sample. One or more rounds of adding and/or removing fluorescent labels associated with nucleotides to and/or from the sequencing primers may be performed, thereby providing sequence information of the one or more barcodes or reverse complements thereof of the one or more amplicons of the sample. In another example, probes comprising a fluorescent label may be used to detect and/or identify one or more barcodes or reverse complements thereof of one or more amplicons of a sample. The probes comprising the fluorescent label (e.g., detection probes) may be added to the sample. The probes comprising the fluorescent label may bind to the one or more amplicons in a sequence dependent manner (e.g., the probes comprising the fluorescent label may hybridize to the one or more amplicons. The fluorescent label of the probes may be detected using an imaging system as described herein thereby generating a signal. The signal may be used to identify one or more analytes associated with the one or more amplicons of the sample. Reading a signal associated with the barcode or the reverse complement of the barcode may be performed in situ. One or more rounds of reading may be performed to collect one or more signals associated with the barcode or the reverse complement of the barcode. In some cases where one or more round of reading may be performed, the signal may be removed in between rounds.

In some cases, a detection probe may be added to the sample which binds to the barcode or reverse complement of the barcode. The detection probe may comprise a nucleic acid. The detection probe may comprise one or more labels. The label may comprise a one or more fluorescent molecules, one or more quantum dots, one or more proteins, one or more mass tags, one or more chromophores, or a combination thereof. In some cases, the one or more proteins may comprise an enzyme, e.g., a horseradish peroxidase. The enzyme may generate a signal indicative of the label. Examples of a fluorescent molecule include, but are not limited to, AlexaFluor Texas Red, rhodamine B, rhodamine 6G, rhodamine 10, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, Cy3, Cy5, and Cy7, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, TEX 615, TYE™ 665, TYE 705, SUN, ATTO™ 425, ATTO™ 488, ATTO™ 532, ATTO™ 550, ATTO™ 565, ATTO™ Rho101, ATTO™ 590, ATTO™ 633, ATTO™ 647, ATTO™ 700, Alexa Fluor® 488 (NHS Ester), Alexa Fluor® 532 (NHS Ester), Alexa Fluor® 546 (NHS Ester), Alexa Fluor® 594 (NHS Ester), Alexa Fluor® 647 (NHS Ester), Alexa Fluor® 660 (NHS Ester), Alexa Fluor® 750 (NHS Ester), IRDye® 700, IRDye® 800, Rhodamine Red™, 5-TAMRA™, Texas Red®-X, Lightcycler® 640, Dy 750, or a combination thereof. The one or more labels may be connected to the detection probe at one or more ends, within the detection probe, or a combination hereof. In some cases, where the detection probe comprises a nucleic acid, the one or more labels may be connected to the nucleic acid at a 5′ end, at a 3′ end, internal within the nucleic acid, or a combination thereof.

The label of the detection probe may be connected to a nucleic acid with a linker. The linker may comprise a variety of chemical groups including one or more ethylene groups, one or more methylene groups, one or more poly-ethylene glycol groups, or a combination thereof. The detection probe may bind to the barcode or reverse complement of the barcode reversibly. The detection probe may bind to the barcode or reverse complement of the barcode irreversibly. The detection probe may bind to the barcode or reverse complement of the barcode through nucleic acid hybridization.

In some cases, one or more detection probes may be added to the sample. Each of the one or more detection probes may bind to the barcode or the reverse complement of the barcode. In some cases, each of the more one or more detection probes may bind to one or more barcodes or one or more reverse complements of a barcode in the sample. Each of the barcodes or reverse complements of the barcodes may relate to a different analyte or set of analytes. In some cases, each of the one or more detection probes may comprise a different label. In some cases, at least two of the detection probes may comprise a different label. In some cases, all of the one or more detection probes may comprise a different label. In some cases, the one or more detection probes may comprise the same label.

### Imaging

The methods described herein may comprise imaging a sample using an imaging system as described herein. Imaging the sample may enable detection of one or more amplicons (e.g., one or more compacted amplicons). Imaging the sample may enable identification of one or more analytes of the sample by detecting one or more amplicons. For example, a sample may be contacted with binding moieties. One or more amplicons may be generated with the aid of the binding moieties. The one or more amplicons may be detected using one or more detection probes using any one of the imaging systems described herein.

Imaging the sample may comprise illuminating the sample with light. Illuminating the sample with light may enable detection of one or more labels associated with one or more amplicons and/or compacted amplicons as described herein. The light used to illuminate the sample may comprise one or more wavelengths. An imaging system as described herein may collect light emitted by the sample at one or more wavelengths. The one or more wavelengths of illumination or collection may include, but is not limited to, light with the following wavelengths: at least about 260 nm, at least about 265 nm, at least about 270 nm, at least about 280 nm, at least about 285 nm, at least about 290 nm, at least about 295 nm, at least about 300 nm, at least about 305 nm, at least about 310 nm, at least about 315 nm, at least about 320 nm, at least about 325 nm, at least about 330 nm, at least about 335 nm, at least about 340 nm, at least about 345 nm, at least about 350 nm, at least about 355 nm, at least about 360 nm, at least about 365 nm, at least about 370 nm, at least about 375 nm, at least about 380 nm, at least about 385 nm, at least about 390 nm, at least about 395 nm, at least about 400 nm, at least about 405 nm, at least about 410 nm, at least about 415 nm, at least about 420 nm, at least about 425 nm, at least about 430 nm, at least about 435 nm, at least about 440 nm, at least about 445 nm, at least about 450 nm, at least about 455 nm, at least about 460 nm, at least about 465 nm, at least about 470 nm, at least about 475 nm, at least about 480 nm, at least about 485 nm, at least about 490 nm, at least about 495 nm, at least about 500 nm, at least about 505 nm, at least about 510 nm, at least about 515 nm, at least about 520 nm, at least about 525 nm, at least about 530 nm, at least about 535 nm, at least about 540 nm, at least about 545 nm, at least about 550 nm, at least about 555 nm, at least about 560 nm, at least about 565 nm, at least about 570 nm, at least about 575 nm, at least about 580 nm, at least about 585 nm, at least about 590 nm, at least about 595 nm, at least about 600 nm, at least about 605 nm, at least about 610 nm, at least about 615 nm, at least about 620 nm, at least about 625 nm, at least about 630 nm, at least about 635 nm, at least about 640 nm, at least about 645 nm, at least about 650 nm, at least about 655 nm, at least about 660 nm, at least about 665 nm, at least about 670 nm, at least about 675 nm, at least about 680 nm, at least about 685 nm, at least about 690 nm, at least about 695 nm, at least about 700 nm, at least about 705 nm, at least about 710 nm, at least about 715 nm, at least about 720 nm, at least about 725 nm, at least about 730 nm, at least about 735 nm, at least about 740 nm, at least about 745 nm, at least about 750 nm, at least about 755 nm, at least about 760 nm, at least about 765 nm, at least about 770 nm, at least about 775 nm, at least about 780 nm, at least about 785 nm, at least about 790 nm, at least about 795 nm, at least about 800 nm, or more nm. The one or more wavelengths of illumination or collection may comprise light with the following wavelengths: at most about 260 nm, at most about 265 nm, at most about 270 nm, at most about 280 nm, at most about 285 nm, at most about 290 nm, at most about 295 nm, at most about 300 nm, at most about 305 nm, at most about 310 nm, at most about 315 nm, at most about 320 nm, at most about 325 nm, at most about 330 nm, at most about 335 nm, at most about 340 nm, at most about 345 nm, at most about 350 nm, at most about 355 nm, at most about 360 nm, at most about 365 nm, at most about 370 nm, at most about 375 nm, at most about 380 nm, at most about 385 nm, at most about 390 nm, at most about 395 nm, at most about 400 nm, at most about 405 nm, at most about 410 nm, at most about 415 nm, at most about 420 nm, at most about 425 nm, at most about 430 nm, at most about 435 nm, at most about 440 nm, at most about 445 nm, at most about 450 nm, at most about 455 nm, at most about 460 nm, at most about 465 nm, at most about 470 nm, at most about 475 nm, at most about 480 nm, at most about 485 nm, at most about 490 nm, at most about 495 nm, at most about 500 nm, at most about 505 nm, at most about 510 nm, at most about 515 nm, at most about 520 nm, at most about 525 nm, at most about 530 nm, at most about 535 nm, at most about 540 nm, at most about 545 nm, at most about 550 nm, at most about 555 nm, at most about 560 nm, at most about 565 nm, at most about 570 nm, at most about 575 nm, at most about 580 nm, at most about 585 nm, at most about 590 nm, at most about 595 nm, at most about 600 nm, at most about 605 nm, at most about 610 nm, at most about 615 nm, at most about 620 nm, at most about 625 nm, at most about 630 nm, at most about 635 nm, at most about 640 nm, at most about 645 nm, at most about 650 nm, at most about 655 nm, at most about 660 nm, at most about 665 nm, at most about 670 nm, at most about 675 nm, at most about 680 nm, at most about 685 nm, at most about 690 nm, at most about 695 nm, at most about 700 nm, at most about 705 nm, at most about 710 nm, at most about 715 nm, at most about 720 nm, at most about 725 nm, at most about 730 nm, at most about 735 nm, at most about 740 nm, at most about 745 nm, at most about 750 nm, at most about 755 nm, at most about 760 nm, at most about 765 nm, at most about 770 nm, at most about 775 nm, at most about 780 nm, at most about 785 nm, at most about 790 nm, at most about 795 nm, at most about 800 nm, or more. The one or more wavelengths of illumination or collection may comprise light with the following wavelengths about 260 to about 800 nm, about 265 to about 795 nm, about 270 to about 790 nm, about 280 to about 785 nm, about 285 to about 780 nm, about 290 to about 775 nm, about 295 to about 770 nm, about 300 to about 765 nm, about 305 to about 760 nm, about 310 to about 755 nm, about 315 to about 750 nm, about 320 to about 745 nm, about 325 to about 740 nm, about 330 to about 735 nm, about 335 to about 730 nm, about 340 to about 725 nm, about 345 to about 720 nm, about 350 to about 715 nm, about 355 to about 710 nm, about 360 to about 705 nm, about 365 to about 700 nm, about 370 to about 695 nm, about 375 to about 690 nm, about 380 to about 685 nm, about 385 to about 680 nm, about 390 to about 675 nm, about 395 to about 670 nm, about 400 to about 665 nm, about 405 to about 660 nm, about 410 to about 655 nm, about 415 to about 650 nm, about 420 to about 645 nm, about 425 to about 640 nm, about 430 to about 635 nm, about 435 to about 630 nm, about 440 to about 625 nm, about 445 to about 620 nm, about 450 to about 615 nm, about 455 to about 610 nm, about 460 to about 605 nm, about 465 to about 600 nm, about 470 to about 595 nm, about 475 to about 590 nm, about 480 to about 585 nm, about 485 to about 580 nm, about 490 to about 575 nm, about 495 to about 570 nm, about 500 to about 565 nm, about 505 to about 560 nm, about 510 to about 555 nm, about 515 to about 550 nm, about 520 to about 545 nm, about 525 to about 540 nm, or about 530 to about 535 nm.

### Cycles

Additional probes may be added to the sample during the detection operations. In some cases, one or more anchor probes may be added to the sample. The one or more anchor probes may bind to all or a portion of the barcode or the reverse complement of the barcode. The one or more anchor probes may bind to a region adjacent to the barcode or reverse complement of the barcode. The region adjacent to the barcode or reverse complement of the barcode may be part of a nucleic acid. The nucleic acid may be an amplicon or amplification product. In some cases, the one or more anchor probe may bind to both the barcode and a sequence adjacent to the barcode. In some cases, the one or more anchor probes may bind to both the reverse complement of the barcode and a sequence adjacent to the reverse complement. The one or more anchor probes may comprise a nucleic acid. The nucleic acid may comprise RNA, DNA, or a combination thereof. The nucleic acid may comprise one or more modifications. The one or more modifications may comprise a phosphorylation modification, an LNA, or a combination thereof. The phosphorylation modification may be a 5′ phosphorylation modification. In some cases, a detection probe and an anchor probe are hybridized to an amplicon generated from rolling circle amplification. The detection probe and the anchor probe may be ligated after hybridization to the amplicon.

The one or more anchor probes may bind to the barcode or reverse complement of the barcode, or sequence adjacent to the barcode or reverse complement of the barcode such that the one or more anchor probes is adjacent to one or more detection probes. The one or more anchor probes may be directly adjacent to the one or more detection probes, such that there are no intervening nucleotides. In some cases, the one or more anchor probes may be adjacent to the one or more detection probes such that there is one or more intervening nucleotides. A gap-filling reaction may be performed to fill in the intervening nucleotides between one or more anchor probes and one or more detection probes. The one or more anchor probes may be ligated to the one or more detection probes. The ligation may be performed using enzymatic ligation, chemical ligation, or a combination thereof. The enzymatic ligation may be performed by a ligase. The ligase may comprise one or more of the following: T4 DNA ligase, SplintR ligase, T3 DNA ligase, T7 DNA ligase, E. coli DNA ligase, Taq ligase, RtcB ligase, or a combination thereof.

Detecting a signal associated with one or more detection probes may be performed after ligation of one or more detection probes with one or more anchor probes. In some cases, the one or more detection probes may transiently bind to the sample. In some cases, the one or more detection probes may bind to the sample for a duration of time without appreciable dissociation. The one or more detection probes ligated to one or more anchor probes may bind to the sample more strongly than the one or more detection probes alone.

In some cases, after signal detection, one or more labels associated with the sample may be removed. The label may be removed from the sample with chemical or enzymatic means. In some cases, the label may be cleaved from the detection probe using an enzyme, a chemical cleavage reagent, or a combination thereof. The enzyme may comprise a restriction enzyme, a polymerase, a ligase, a transposon, on a combination thereof. The chemical cleavage reagent may comprise a reducing reagent, a reactive oxygen species, or a combination thereof. The label may be removed from the sample by removing, digesting, or disrupting the one or more detection probes from the sample. In some cases, the one or more detection probes may be ligated to one or more anchor probes. The one or more detection probes may be removed from the sample using temperature and incubating the sample at an elevated temperature for a duration of time. The one or more detection probes may be removed from the sample by incubating the sample with a chemical that disrupts nucleic acid hybridization, e.g., one or more chaotropic agents. In some cases, the one or more chaotropic agents may comprise glycine, arginine, histidine hydrochloride, sodium hydroxide, formamide, dimethyl sulfoxide (DMSO), guanidinium chloride, or a combination thereof. The one or more detection probes may be removed from the sample by incubating the sample with one or more chaotropic reagents and incubating the sample at an elevated temperature. The one or more detection probes may be removed from the sample using an enzyme, including but not limited to a DNAse, an RNAse, a restriction enzyme, or a combination thereof.

In some cases, after signal detection, one or more labels associate with the sample may be altered such that the label is no longer capable of emitting a signal. The label may be denatured, e.g., in cases where the label may comprise a polypeptide. Denaturation may be performed using heat, salt, a solvent (e.g., methanol, ethanol, or any combination thereof), an acid, a base, or a combination thereof. The label may be photobleached, e.g., in cases where the label may comprise a fluorescent moiety. Photobleaching may be performed by exposing the sample to light, including ambient light, to a reactive chemical species, a reducing reagent, a base, an acid, or a combination thereof.

One or more rounds of detection may be performed. Each round of detection may comprise contacting the sample with one or more detection probes, one or more anchor probes, or a combination thereof. For example, a sample may comprise an amplicon comprising a reverse complement of a barcode sequence comprising 8 nucleotides. A round of detection may comprise adding a plurality of anchor probes and a plurality of detection probes to the sample to enable binding of anchor probes and detection probes to the amplicon, where the sequence of the detection probe provides information related to the first two nucleotides of the 8-nucleotide reverse complement of the barcode sequence. After binding, the anchor probe and detection probe may be ligated, and the sample may be washed to remove all unbound detection probes. The sample may be imaged to detect the signal associated with the detection probe. After imaging, the detection probe ligated to the anchor probe may be removed by incubation of the sample with a chaotropic reagent. The combination of binding of the detection probe, ligating the detection probe, imaging the sample, and removing the detection probe comprise a round of detection in this case. In some cases, the one or more detection probes may be the same across one or more rounds. For example, detection probes with the same sequence may be added in a first and a second cycle. In some cases, the one or more detection probes may be different across one or more rounds. For example, detection probes with difference sequences may be added in a first round as compared to a second round. In some cases, the one or more anchor probes may be the same across one or more rounds. For example, anchor probes with the same sequence may be added in a first and second cycle. In some cases, the one or more anchor probes may be different across one or more rounds. For example, anchor probes with difference sequences may be added in a first round as compared to a second round. Each round of detection may comprise adding probes to the sample, imaging the sample using an imaging system to generate one or more images, optionally, and removing the label from the sample. The methods described herein may comprise performing at least about 1 cycle of detection, at least about 2 cycles of detection, at least about 3 cycles of detection, at least about 4 cycles of detection, at least about 5 cycles of detection, at least about 6 cycles of detection, at least about 7 cycles of detection, at least about 8 cycles of detection, at least about 9 cycles of detection, at least about 10 cycles of detection, at least about 12 cycles of detection, at least about 15 cycles of detection, at least about 20 cycles of detection, at least about 25 cycles of detection, at least about 30 cycles of detection, at least about 35 cycles of detection, at least about 40 cycles of detection, at least about 45 cycles of detection, at least about 50 cycles of detection, at least about 60 cycles of detection, at least about 70 cycles of detection, at least about 80 cycles of detection, at least about 90 cycles of detection, at least about 100 cycles of detection, or more cycles of detection. The methods described herein may comprise performing at most about 1 cycle of detection, at most about 2 cycles of detection, at most about 3 cycles of detection, at most about 4 cycles of detection, at most about 5 cycles of detection, at most about 6 cycles of detection, at most about 7 cycles of detection, at most about 8 cycles of detection, at most about 9 cycles of detection, at most about 10 cycles of detection, at most about 12 cycles of detection, at most about 15 cycles of detection, at most about 20 cycles of detection, at most about 25 cycles of detection, at most about 30 cycles of detection, at most about 35 cycles of detection, at most about 40 cycles of detection, at most about 45 cycles of detection, at most about 50 cycles of detection, at most about 60 cycles of detection, at most about 70 cycles of detection, at most about 80 cycles of detection, at most about 90 cycles of detection, at most about 100 cycles of detection, or fewer cycles of detection. The methods described herein may comprise performing about 1-100 cycles of detection, about 2-90 cycles of detection, about 3-80 cycles of detection, about 4-70 cycles of detection, about 5-60 cycles of detection, about 6-50 cycles of detection, about 7-45 cycles of detection, about 8-40 cycles of detection, about 9-35 cycles of detection, about 10-30 cycles of detection, about 12-25 cycles of detection, or about 15-20 cycles of detection.

### Imaging Systems

The methods described herein may comprise using an imaging system to image a sample comprising one or more amplicons (e.g., one or more compacted amplicons. Imaging the sample using an imaging system may generate one or more images. The one or more images may comprise a two-dimensional (2D) image, a three-dimensional (3D) image, or a combination thereof. Various imaging systems may be used to generate one or more images. The imaging system may comprise a microscope. In some embodiments, the microscope may be a confocal microscope. In some embodiments, the microscope may be a two-photon microscope. In some embodiments, the microscope may be a structured illumination microscope. In some embodiments, the microscope may be a light sheet microscope.

In an aspect, the present disclosure provides system for analyzing one or more samples as described herein. In some cases, the system may comprise a system for volumetric imaging of a sample. In some cases, the sample may comprise a two-dimensional sample or a three-dimensional sample. In some instances, the system may comprise a stage to hold the sample. Additionally, the system may comprise an imaging module, imager, imaging device, or imaging system that creates an image of one or more portions of the sample. The one or more portions of the sample may comprise one or more adjacent portions across a surface of the sample. In some instances, the one or more portions of the sample may comprise one or more adjacent portions of the sample along an axis normal to the surface of the sample. In some cases, the one or more portions of the sample may comprise one or more object planes within the sample. In some cases, the imaging module, imager, imaging device, or imaging system may comprise an objective lens that transmits photons from one or more object planes within the sample to a sensor of the imaging module, imager, imaging device, or imaging system. In some cases, the objective lens may collect, relay, or direct photons scattered from the sample to one or more optical system components when an illumination of the illumination source is provided to the sample. The object plane within the sample may comprise a plane within the sample from which scattered photons from the one or more portions of the sample are imaged onto one or more sensors of the imaging module, imager, imaging device, or imaging system.

In some cases, the imaging module, imager, imaging device, or imaging system may move the objective lens relative to the sample along an optical axis of the objective. In some cases, the system may move the objective lens relative to sample in a direction parallel or substantially parallel to the optical axis of the objective. In some cases, moving the objective lens relative to the sample in a direction substantially parallel to the optical axis of the objective may comprise moving the objective lens along an axis that is parallel within up to about 1%, up to about 2%, up to about 3%, up to about 4%, or up to about 5% parallel with the optical axis of the objective. In some cases, moving the objective lens relative to the sample in a direction substantially parallel to the optical axis of the objective may comprise moving the objective lens along an axis that is parallel within up to about 0.01°, up to about 0.05°, up to about 0.1°, up to about 0.5°, up to about 1.0°, or up to about 3.0° with the optical axis of the objective

In some cases, the objective lens is moved relative to the sample, but not along an optical axis of the objective. The objective lens can be moved substantially toward or substantially away from the sample. For example, moving the objective lens toward the sample at an angle relative to the optical axis can image a trapezoidal field of view of the sample.

In some cases, the system may continuously move the objective lens relative to the sample along an optical axis of the objective. The optical axis of the objective lens may comprise an axis that is co-linear or parallel with an axis through the center of the objective lens. In some instances, the system may continuously move the objective lens relative to the sample in a direction parallel or substantially parallel to the optical axis of the objective. In some cases, while the objective lens of the system is moving (e.g., continuously) relative to the sample along the optical axis of the objective, the imaging module, imager, imaging device, or imaging system may acquire a series of images. In some cases, the series of images may correspond to adjacent object planes within the sample.

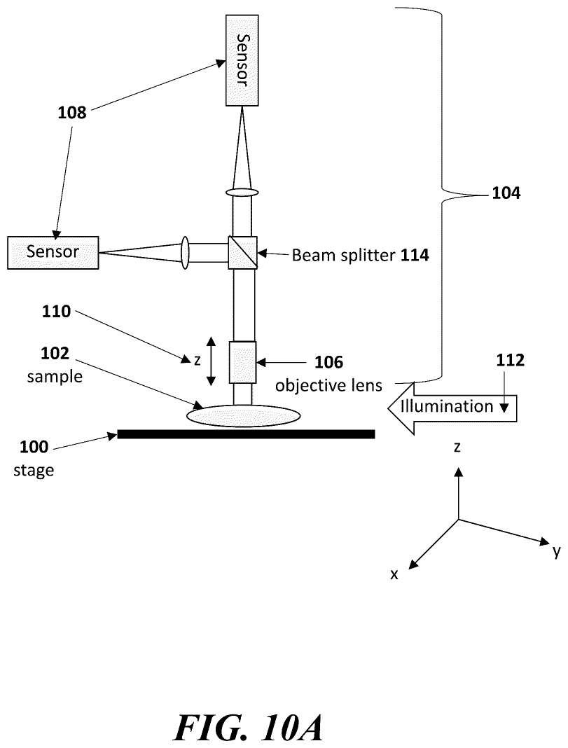

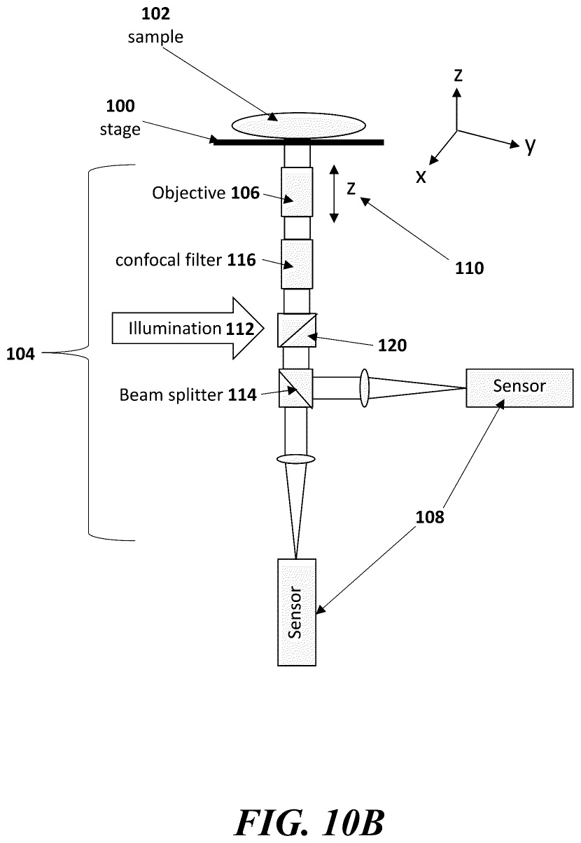

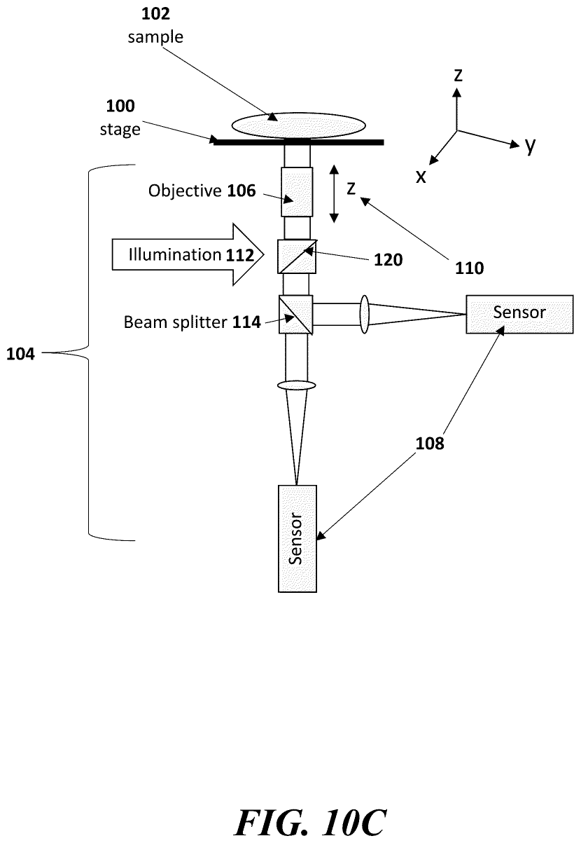

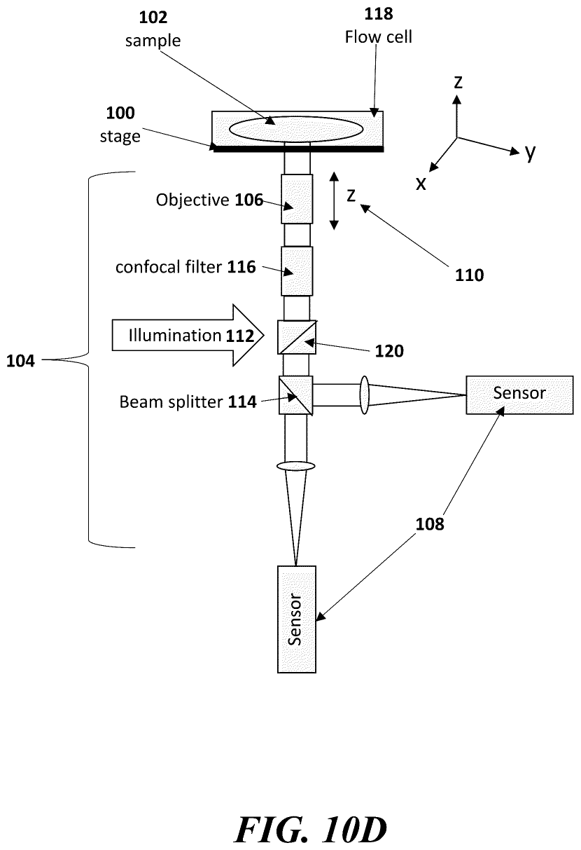

FIGS. 10A-10D illustrate a schematic drawing of an example microscopy system for use as described herein. In some cases, the microscopy system may comprise an upright microscopy system, as shown in FIG. 10A. In some instances, the microscopy system may comprise an inverted microscopy system as shown in FIGS. 10B-10D.

The system described herein can be used for volumetric imaging of a sample. With reference to FIG. 10A, the system can include a stage 100 configured to hold a three-dimensional or two-dimensional sample 102. In some cases, the sample 102 may be housed, provided, held, or contained, or any combination thereof, within a holder, as described herein.

The system can include an imaging module, imager, imaging device, or imaging system 104 configured to create an image. The imaging module, imager, imaging device, or imaging system can include an objective lens 106 configured to transmit photons from one or more object planes within the sample to one or more sensors 108. The system can be configured to continuously move the objective lens relative to the sample in a direction substantially parallel to an optical axis of the objective, e.g., a z-direction 110, described herein. In some cases, the system may move the objective lens 106 relative to the sample 102 with a z-axis scanner coupled to the objective lens. In some cases, the z-axis scanner may comprise a stage or an actuator (e.g., linear actuator). A control unit in electrical communication with the z-axis scanner and the system may provide a command to the z-axis scanner to move or translate the objective lens 106, as described herein. The system can be configured to simultaneously use the imaging module, imager, imaging device, or imaging system to acquire a series of images corresponding to adjacent object planes within the sample. In some cases, one or more sensors may simultaneously collect or acquire photons scattered from a plurality of object planes (e.g., a plurality of adjacent object planes) of the sample onto one or more sensors while the objective lens of the imaging system is moved in a direction towards a surface of one or more portions of the sample, or away from a surface of the one or more portions of the sample, or any combination thereof. The sensor can be a camera. The series of images can comprise a video.

The system may comprise one or more light sources configured to illuminate the one or more samples, or the one or more portions of the one or more samples. Continuing with FIG. 10A, the imaging module, imager, imaging device, or imaging system can include a system or device for illuminating 112 the sample (e.g., a laser). In some cases, the imaging system may comprise an illumination source 112 for illuminating the sample. In some cases, the illumination from an illumination source 112 may be provided orthogonal to an optical axis of the objective lens 106. The sample can be illuminated at one or more portions of the sample corresponding to the object plane. However, other portions of the sample can also be illuminated. The illuminated sample can produce one or more photons or a plurality of photons that are transmitted to the one or more sensors 108. For example, when the one or more portions of the sample are illuminated by the illumination source 112, the sample may scatter, absorb, or reflect, one or more photons of the illumination source. The objective lens 106 can transmit the scattered or reflected photons to a plurality of sensors. In some cases, the objective lens 106 may collect, direct, or relay the one or more photons scattered or reflected from the one or more portions of the sample onto one or more sensors.

In some cases, the disclosure provides an inverted confocal microscopy system for analyzing one or more samples, as shown in FIG. 10B. In some cases, the inverted confocal microscopy system may comprise an imaging module, imager, imaging device, or imaging system 104. The imaging module, imager, imaging device, or imaging system 104 may comprise a confocal filter 116. The confocal filter 116 may comprise a pinhole. In some cases, the confocal filter 116 may comprise a plurality of pin holes. In some cases, the confocal filter 116 may comprise one or more confocal spinning disks, as described herein. In some cases, the confocal filter 116 may couple light from an illumination source 112 through the one or more pin holes of the confocal filter 116 to an objective lens 106 that focuses the illumination source onto one or more portions of the sample 102. In some instances, the confocal filter 116 may couple, direct, or relay illumination 112 photons reflected or scattered from the sample 102 through one or more pin holes of the confocal filter 116 to one or more sensors 108. In some cases, the photons reflected or scattered from the sample 102 directed through the one or more pin holes of the confocal filter 116 may be optically coupled to, transmitted through, or a combination thereof, a filter 120 (e.g., a dichroic filter), beam splitter 114, or a combination thereof. In some cases, the confocal filter 116 may be optically coupled to the objective lens 106, the filter 120, or a combination thereof. In some cases, imaging module, imager, imaging device, or imaging system 104 may comprise a beam splitter 114, where the beam splitter may transmit or reflect the one or more photons scattered or reflected from the one or more portions of the sample based on at least a wavelength or wavelength band of the one or more photons. In some cases, the beam splitter 114 may transmit a first one or more photons of a first wavelength or a first wavelength band to a first sensor and transmit a second one or more photons of a second wavelength or a second wavelength band to a second sensor. In some cases, the imaging module, imager, imaging device, or imaging system 104 may comprise an illumination source 112 that illuminates one or more portions of the sample. In some cases, an output illumination of the illumination source 112 may be optically coupled to the sample 102 by a filter 120. The filter 120 may reflect an illumination output of the illumination source 112 towards the confocal filter 116 and objective lens 106 to focus the illumination onto the sample 102. In some instances, the filter 120 may be optically coupled to the illumination source 112, the confocal filter 116, or the beam splitter 114, or any combination thereof. In some cases, the filter 120 may transmit the one or more scattered or reflected photons from the sample to the beam splitter 114 when the one or more portions of the sample are illuminated by the illumination source 112. In some cases, the objective lens 106 may be translated or moved along a z-direction 110 towards a surface of the sample 102, away from a surface of the sample 102, or any combination thereof. In some cases, the objective lens 106 may be coupled to a z-axis scanner, described herein, that may translate or move the objective lens 106. In some cases, the sample 102 may be provided on a substrate (e.g., a stage 100), where the substrate may translate or move a position of a sample on a two-dimensional plane.

In some cases, the disclosure provides an inverted microscopy system for analyzing one or more samples, as shown in FIG. 10C. In some cases, the inverted confocal microscopy system may comprise an imaging module, imager, imaging device, or imaging system 104. In some cases, the imaging module, imager, imaging device, or imaging system 104 may comprise an illumination source 112 that illuminates one or more portions of the sample 102. In some cases, an output illumination of the illumination source 112 may be optically coupled to the sample 102 by a filter 120. The filter 120 may reflect an illumination output of the illumination source 112 towards the object lens 106 to focus the illumination onto the sample 102. In some instances, the filter 120 may be optically coupled to the illumination source 112, or the beam splitter 114, or any combination thereof. In some cases, the filter 120 may transmit the one or more scattered or reflected photons from the sample to the beam splitter 114 when the one or more portions of the sample 102 are illuminated by the illumination source 112. In some cases, the beam splitter 114 may transmit a first one or more photons of a first wavelength or a first wavelength band to a first sensor and transmit a second one or more photons of a second wavelength or a second wavelength band to a second sensor. In some cases, the objective lens 106 may be translated or moved along a z-direction 110 towards a surface of the sample 102, away from a surface of the sample 102, or any combination thereof. In some cases, the objective lens 106 may be coupled to a z-axis scanner, described herein, that may translate or move the objective lens 106. In some cases, the sample 102 may be provided on a substrate (e.g., a stage 100), where the substrate may translate or move a position of the sample 102 on a two-dimensional plane.

In some instances, the disclosure provides an inverted confocal microscopy system for analyzing one or more samples provided in a flow cell 118, as shown in FIG. 10D. In some cases, the inverted confocal microscopy system shown in FIG. 10D may comprise an imaging module, imager, imaging device, or imaging system 104. The imaging module, imager, imaging device, or imaging system 104 may comprise a confocal filter 116. The confocal filter 116 may comprise a pinhole. In some cases, the confocal filter 116 may comprise a plurality of pin holes. In some cases, the confocal filter 116 may comprise one or more confocal spinning disks, as described herein. In some cases, the confocal filter 116 may couple light from an illumination source 112 through the one or more pin holes of the confocal filter 116 to an objective lens 106 that focuses the illumination source 112 onto one or more portions of the sample 102. In some instances, the confocal filter 116 may couple, direct, or relay illumination 112 photons reflected or scattered from the sample 102 through one or more pin holes of the confocal filter 116 to one or more sensors 108 through the filter 120 and beam splitter 114. In some cases, the confocal filter 116 may be optically coupled to the objective lens 106, the filter 120, or a combination thereof. The imaging module, imager, imaging device, or imaging system 104 of the inverted microscopy system may comprise one or more sensors 108 that may image one or more photons scattered or reflected from one or more portions of the sample 102 when the one or more portions of the sample 102 are illuminated by the illumination source 112. In some cases, the imaging module, imager, imaging device, or imaging system 104 may comprise a beam splitter 114, where the beam splitter may transmit a first one or more photons of a first wavelength or a first wavelength band to a first sensor and transmit a second one or more photons of a second wavelength or a second wavelength band to a second sensor. In some cases, the imaging module, imager, imaging device, or imaging system 104 may comprise an illumination source 112 that illuminates one or more portions of the sample 102. In some cases, an output illumination of the illumination source 112 may be optically coupled to the sample 102 by a filter 120. The filter 120 may reflect an illumination output of the illumination source 112 towards the objective lens 106 to focus the illumination onto the sample 102. In some instances, the filter 120 may be optically coupled to the illumination source 112, the objective lens 106, or the beam splitter 114, or any combination thereof. In some cases, the filter 120 may transmit the one or more scattered or reflected photons from the sample 102 when the one or more portions of the sample are illuminated by the illumination source 112, to the beam splitter 114. In some cases, the sample 102 may be provided, contained, or housed in a flow cell 118.

The illumination from the illumination source 112 provided or directed to the sample (e.g., of the one or more samples) 102 may cause the one or more samples to reflect or scatter one or more photons. In some cases, collecting or detecting and processing the reflected or scattered one or more photons from the sample may generate one or more signals in one or more voxels of the sample. In some cases, the one or more voxels of the sample may correspond to a position (e.g., a three-dimensional position or coordinate) and corresponding volume within the sample where the one or more scattered or reflected photons are detected or collected. The illumination from the illumination source 112 provided or directed to one or more object planes (e.g., one or more adjacent object planes) of the sample may cause the sample to reflect or scatter one or more photons. In some cases, collecting or detecting, and processing the reflected or scattered one or more photons from the one or more object planes may generate one or more signals from the one or more object planes of the one or more samples. The one or more voxels may comprise a plurality of object planes of the sample. The illumination may comprise an illumination in a plurality of wavelength bands (e.g., in a plurality of colors). The plurality of wavelength bands may correspond to the excitation wavelengths for a plurality of fluorophores or other labels within the sample, described herein. In some cases, the plurality of wavelength bands of the illumination provided to the sample may cause the plurality of fluorophores or other labels of the sample to absorb the plurality of wavelength bands and emit or scatter one or more photons. The one or more photons emitted by the plurality of fluorophores or other labels of the sample may be detected or collected and processed as one or more signals of the one or more voxels of the sample. The one or more photons emitted by the plurality of fluorophores or other labels may be detected or collected and processed as one or more signals of the one or more object planes of the sample. The light source may be configured to provide light sheet illumination to the sample as described elsewhere herein. In some cases, the light source may comprise a lens (e.g., a cylindrical lens), whereby the output illumination of the light source transmitting through the lens may form a light sheet illumination. For example, the light source can comprise a laser and optical elements (e.g., spatial light modulator, cylindrical lens, etc.) to generate or provide a flat sheet of light to the sample.

The one or more light sources may be configured to continuously operate (e.g., may be configured to continuously illuminate the voxel that is being detected by the one or more sensors). For example, the one or more light sources may be configured to illuminate the one or more samples throughout a detecting process as described herein. For example, the one or more light sources may illuminate throughout a detecting process of the system. In some cases, the detecting process may comprise collecting, detecting, or integrating, or any combination thereof, one or more scattered or reflected photons from the sample in response to an illumination of the sample by an illumination source. In some cases, the detecting process may comprise integrating, reading, or a combination thereof operations, of the sensor to convert the detected one or more photons into a signal (e.g., an electrical signal).

The imaging module, imager, imaging device, or imaging system can be a light sheet microscope or a confocal microscope. For example, the system may be configured to confocally image the one or more samples. The confocal microscopy system may comprise a spinning disk confocal microscopy system. For example, the confocal microscopy system may use a spinning disk configured to reject one or more portions of the light moving through the system to achieve the confocal focus. In some cases, the spinning disk may enrich one or more photons that arise from the sample (e.g., are scattered or reflected) and transmitted to one or more sensors. For example, by utilizing the spinning disk, the one or more photons that are transmitted from the sample to the one or more sensors may be limited to only the photons that arise from the depth of focus based on a size of one or more pinholes of the spinning disk, the depth of focus of the objective lens, or a combination thereof. The geometric restriction to the scattered or reflected photons of the sample enabled by the spinning disks with one or more pin holes may increase the resolution of the signal collected or detected in the one or more voxels of the sample compared to a resolution of a confocal imaging system that did not use the spinning disk with one or more pin holes. In some cases, the confocal microscopy system may use one or more spinning disks to reject one or more portions of the light moving through the system to achieve the confocal focus. The confocal microscopy system may comprise a plurality of spinning disks each comprising a plurality of pinholes configured to reject out of angle light for one or more illumination or one or more detection channels. The plurality of spinning wheels can enable parallel acquisition in a plurality of wavelength bands. For example, each wheel of the plurality of spinning wheels can acquire a different wavelength band of the plurality of wavelength bands.

The imaging module, imager, imaging device, or imaging system can include a beam splitter 114 configured to split the beam of transmitted photons into a plurality of sensors (e.g., cameras). The imaging module, imager, imaging device, or imaging system can include sensors imaging in multiple passbands. In some cases, each of the plurality of sensors may integrate photons having a different wavelength. Each sensor can be oriented to produce parallel object planes.

The system may comprise one or more sensors as described elsewhere herein. The one or more sensors may be configured to receive the one or more signals from the voxel of the sample, described herein. The one or more sensors may comprise one or more global shutter sensors. The sensor can comprise an array of pixels. The array of pixels may be organized into multiple groups of pixels, where each group of pixels are read in series while the remaining groups of pixels are integrating photons. For example, all of the rows of pixels (e.g., bins) in the sensor can be read out at the same time. The one or more sensors may comprise one or more rolling shutter sensors. The sensor may comprise a charge coupled device (CCD) detector, a complementary metal oxide semiconductor (CMOS) sensor, or the like. The system may comprise a plurality of sensors.

The system can achieve a high overall efficiency. For example, efficiency can be determined by what percentage of time the most expensive portions of the system (e.g., the image acquisition module) is in use. Some methods may require performing one or more fluidic operations on a sample (e.g., labeling, stripping, aspirating, dispensing, or incubating, or any combination thereof). In some cases, the one or more fluidic operations may comprise aspirating fluid, providing fluid, or dispensing fluid, or any combination thereof, to one or more samples. In such cases, multiple samples can be processed in parallel on the system (e.g., loaded onto multiple areas of a stage). In this way, the series of images can be acquired from a first sample while a second sample is undergoing a fluidic operation or an incubation period. For example, as a first sample of a plurality of samples provided on the stage is imaged by the systems described herein, a second sample of the plurality of samples may undergo one or more fluidic operations simultaneously, in parallel or concurrently. In some cases, the order or sequence of the one or more fluidic operations and the imaging operations may be scheduled or ordered with an order based on at least an assay or reaction conducted on the one or more samples. By imaging and conducting the one or more fluidic operations simultaneously or in parallel, the imaging system may operate with a high throughput in a cost-effective manner with reduced downtime and waiting periods between the one or more fluidic operations conducted on one or more samples and subsequent imaging of the one or more samples.

### Movement of Objective and Continuous Integration

The system may comprise one or more z-axis scanners configured to continuously or substantially continuously move a focal volume of the imaging module, imager, imaging device, or imaging system through the sample. In some cases, the z-axis scanner may be releasably coupled to an objective lens. In some cases, the z-axis scanner may be mechanically coupled to the objective lens. Movement or translation of the objective lens can be achieved by moving the objective lens relative to the sample (e.g., in a direction substantially parallel to the optical axis of the objective). In some cases, a relative distance between the objective lens and the sample may be changing as the objective lens moves relative to the sample. The relative distance between the objective lens and the sample may increase or decreased between a first position on a surface of the sample and a second position on the objective lens during imaging or acquiring a series of images from the plurality of objective planes within the sample. The objective lens can be moved (while the sample is stationary) or the sample can be moved (while the optical lens is stationary), or a combination thereof. The objective can be moved toward or away from the sample e.g., a relative distance between the objective lens and the sample can be increasing or decreasing.

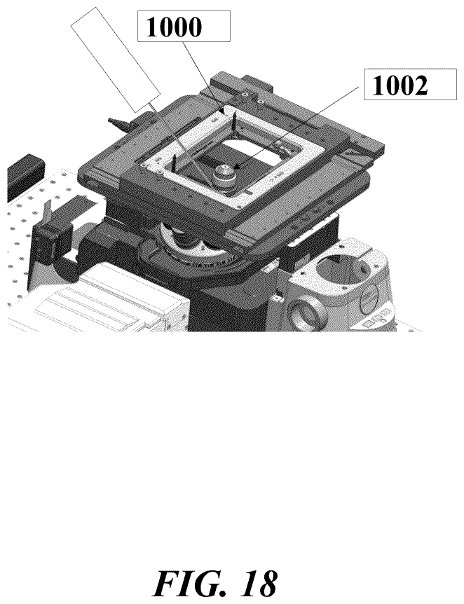

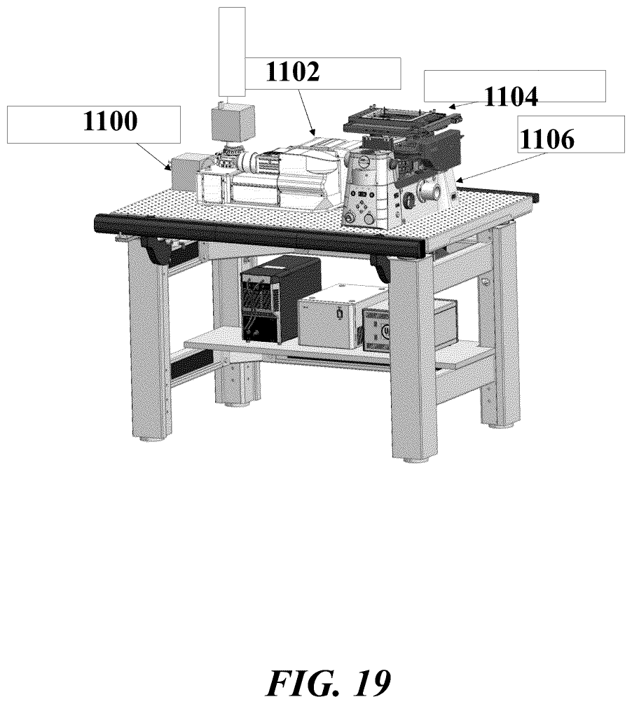

FIG. 18 shows an example schematic drawing of a path of movement of the objective lens for imaging of a sample, according to some embodiments of the present disclosure. Here, the objective lens 200 may be moved along a path (represented by block arrows) relative to the sample 202. The objective lens can be moved toward 204 the sample (while imaging) until a field of view 206 has been imaged to a chosen depth. Then, the method can further comprise (stopping imaging and) moving the objective lens relative to the sample in a direction substantially perpendicular 208 to the optical axis of the objective, such that the image module, imager, imaging device, or imaging system is capable of imaging a second field of view 210 of the sample. The second field of view can be imaged while moving the objective lens away from 212 the sample. The movement can continue in this way until one or more suitable portions of the sample are imaged. In some cases, imaging may be performed in a consistent direction relative to the sample (e.g., either toward or away from the sample).

There can be advantages of continuous movement while imaging (e.g., compared to imaging a focal plane while the sensor is stationary, moving the objective to an adjacent focal plane, capturing a second planar image while stationary, etc.). One advantage may be that time is not wasted waiting for the objective to accelerate, move, decelerate, and then settle its motion for each layer. Instead, the objective can move at a relatively constant velocity. A second advantage may be that the sensor may be used (e.g., photons are being integrated) at substantially all times (e.g., instead of just when the objective is being held steady at each focal plane).

The system may not settle (e.g., pause after movement to reduce artifacts in the images or signals due to movement) between imaging operations. The settling may comprise a time where a stage comprising the sample settles after movement of the stage. The one or more z-axis scanners may be configured to shift (e.g., move) one or more sample stages of the system (e.g., be configured to traverse the one or more sample stages along an optical axis of the one or more sensors), one or more optical elements in optical communication with the one or more sensors (e.g., one or more optical elements (e.g., lenses, filters, micromirror arrays, etc.) configured to optically shift the object plane through the one or more samples), or move the one or more sensors (e.g., shift the one or more sensors to shift the one or more object planes), or any combination thereof.

Use of continuous integration through a volume of a sample via z-axis scanning can improve system throughput and reduce sensor overhead (e.g., cost overhead, time overhead). In this way, large amounts of spatially resolved three-dimensional data can be acquired for a large sample (e.g., a plurality of cells). In some cases, the one or more sensors may be integrating continuously while the one or more z-axis scanners provide new voxels of the one or more samples to detect. In this way, the one or more sensors can be continuously on while not wasting energy or imaging time.

In some cases, a field of view may comprise a field of view of an imaging module, imager, imaging device, imaging system, or any combination thereof, described herein. In some cases, the field of view may comprise a field of view of the objective lens of the imaging module, imager, imaging device, imaging system, or any combination thereof. In some cases, a field of view may comprise a field of view of a sample (206, 207). In some cases, the series of images acquired using the imaging module, imager, imaging device, imaging system, or any combination thereof described herein can cover one or more fields of view of a sample. The one or more fields of view of the sample may comprise a field of view dimension of the field of view of the imaging module, imager, imaging device, imaging system, or any combination thereof. The imaging method can be repeated to provide a volumetric image at a plurality of adjacent fields of view of the sample. The plurality of image fields of view may be mathematically joined into a continuous imaged volume. In the case of rolling shutter, the joining may involve removing the skew angle induced in the scan to create cartesian axes. Then, image volumes can be corrected for field effects such as pin cushion or barrel distortion, allowing seamless stitching between adjacent volumes. The plurality of fields of view can be imaged spanning an imaged volume of the sample.

The objective can be moved substantially continuously while imaging. For example, the velocity of the objective lens moving relative to the sample can increase or decrease by less than about 5%, less than about 3%, less than about 1%, less than about 0.5%, less than about 0.3%, less than about 0.1%, less than about 0.05%, less than about 0.01%, less than about 0.005%, or less than about 0.001%, during a period of time when the objective lens is continuously moved relative to the sample.

The objective can be moved at any suitable constant velocity. For example, the objective lens may be moved relative to the sample at a velocity such that a second object plane is stacked on a first object plane.

Each of the series of images can have a depth of focus. Each of the series of images can be separated by approximately one depth of focus. In some cases, the series of images are separated by about 1 depth of focus to about 4 depths of focus. In some cases, the series of images are separated by about 1 depth of focus to about 2 depths of focus, about 1 depth of focus to about 3 depths of focus, about 1 depth of focus to about 4 depths of focus, about 2 depths of focus to about 3 depths of focus, about 2 depths of focus to about 4 depths of focus, or about 3 depths of focus to about 4 depths of focus. In some cases, the series of images are separated by about 1 depth of focus, about 2 depths of focus, about 3 depths of focus, or about 4 depths of focus. In some cases, the series of images are separated by at least about 1 depth of focus, about 2 depths of focus, or about 3 depths of focus. In some cases, the series of images are separated by at most about 2 depths of focus, about 3 depths of focus, or about 4 depths of focus.

In some cases, the velocity of the objective lens relative to the sample is coordinated with a frame rate of the sensor such that the series of images are separated by approximately one depth of focus. In some cases, the depth of focus may comprise the depth of focus of the objective lens. In some instances, the depth of focus may comprise the depth of focus of the imaging module, imager, imaging device, or imaging system. In some instances, the depth of focus may comprise the depth of focus of the objective lens in combination with one or more pin holes of the one or more spinning disks.

### Volumetric Images

Continuously moving the objective while simultaneously using the imaging module, imager, imaging device, or imaging system to acquire a series of images can produce a volumetric image of the sample at a plurality of object planes of the sample. FIG. 12 is a schematic of a volumetric (z-stack) image comprising multiple object planes in one field of view. The volumetric image can be a video. Here, the objective may be moved relative to the sample in the z-direction 300. Simultaneously, the sensor may be operating in rolling shutter mode 302, reading the pixels from left to right. FIG. 12 is a cross-sectional diagram, e.g., showing the width of several objective planes 304, 306, 308 for a single field of view 310. Each object plane may be angled 312 in relation to the stage 314.

In other words, the object plane may not be perfectly orthogonal to the optical axis (e.g., the tilt of the object plane in relation to the optical axis may be a small angle). This angle can be any suitable angle, such as less than about 1 milliradian. Additional angles are described below. As described herein the method can further comprise applying a mathematical transformation to the series of images to correct for the angle relative to the optical axis. In the case of rolling shutter, the mathematical transformation can include correcting for the skew angle and re-sampling in cartesian axes.

Continuing with FIG. 12, once the rolling shutter acquisition has proceeded across all pixels, the full field of view may have been imaged for the first object plane 304. The sensors can be integrating one or more pixels of the sensor during a period of time (e.g., except when the one or more pixels of the sensor are being read), such that the second object plane 306 can begin being read once the first object plane 304 is completely imaged. This can be continued for subsequent object planes 308, up to a chosen depth of imaging 316. This z-stack image may be acquired while the objective moves in the z-direction relative to the sample (e.g., 204 or 212 in FIG. 18).

Following imaging of a field of view, the objective can be moved to a position configured to image a second field of view. The second field of view may be adjacent to the first field of view (e.g., movement 208 in FIG. 18).

FIG. 13 is an example schematic drawing of z-stack images (volume videos) comprising three adjacent fields of view 400, according to some embodiments of the present disclosure. These volume videos may be taken using a rolling shutter sensor, resulting in angled object planes. In this example, the imaging may be performed when the objective is moving toward the sample 402. Therefore, the objective may return 404 to the original separation distance between imaging fields. The plurality of fields of view (imaged at a depth, to create volumes) may be acquired by a single sensor in a plurality of passes or by a plurality of sensors in a single pass.

### Imaging Methods

In an aspect, the present disclosure provides a method for analyzing one or more samples using a system disclosed herein. In another aspect, the present disclosure provides a method of volumetric imaging of a sample. In some cases, the method of volumetric imaging of a sample may comprise providing a sample disposed on a substrate (e.g., stage). In some cases, the sample may comprise a two-dimensional or a three-dimensional sample. In some cases, the method of volumetric imaging of a sample may comprise providing an imaging module, imager, imaging device, or imaging system that may acquire, create, or generate one or more images (e.g., a series of images) of a sample. In some cases, the image may comprise a series of images. In some cases, the imaging module, imager, imaging device, or imaging system may comprise a lens (e.g., an objective lens) that may transmit photons from one or more object planes or a plurality of object planes within the sample to one or more sensors or a plurality of sensors of the imaging module, imager, imaging device, or imaging system. In some cases, the method of volumetric imaging of a sample may comprise continuously or periodically moving the objective lens relative to the sample in a direction along the optical axis of the objective lens. In some cases, the method of volumetric imaging of the sample may comprise continuously or periodically moving the objective lens relative to the sample in a direction along an axis parallel to or substantially parallel to the optical axis of the objective. In some cases, the method of volumetric imaging of a sample may comprise, while simultaneously moving the objective lens, using the imaging module, imager, imaging device, or imaging system to acquire a series of images corresponding to one or more object planes with the sample. In some cases, the one or more object planes may comprise a plurality of object planes. In some cases, the one or more object planes may comprise a first object plane and a second object plane. In some cases, the one or more object planes or the plurality of object planes may be adjacent object planes within the sample. The first object plane and the second object plane may be adjacent object planes within the sample. In some cases, the method operation of providing a sample disposed on a substrate may be repeated one or more times to provide one or more volumetric images of one or more samples disposed on the substrate. In some instances, the method operation of continuously or periodically moving the objective lens relative to the sample in a direction along the optical axis of the objective may be repeated one or more times to provide a volumetric image at a plurality of adjacent fields of view of the sample. In some cases, using the imaging module, imager, imaging device, or imaging system while simultaneously moving the objective lens to acquire a series of images corresponding to one or more object planes with the sample, or any combination thereof, may be repeated one or more times to provide a volumetric image at a plurality of adjacent fields of view. In some cases, adjacent fields of view may comprise an adjacent one or more portions of the sample imaged within a field of view of the imaging module, imager, imaging device, or imaging system. In some instances, the field of view may comprise the field of view of the objective lens. In some cases, an adjacent field of view may comprise a one or more adjacent portions of a sample that comprise a width or length corresponding to the width or length of the field of view of the imaging module, imager, imaging device, or imaging system. In some cases, the objective lens may begin motion at a first point a distance away from the sample and move towards a second point a distance less than the distance of the first point from the sample. In some cases, the objective may move from the first point to the second point while acquiring the series of images corresponding to the one or more object planes of the sample. In some cases, the objective lens may acquire a series of images at a first portion of a sample moving from the first point to the second point. In some instances, the objective lens may move from the second point to the first point e.g., to reset to acquire another series of images of the sample or a different portion of the sample. In some cases, as the objective lens moves from the second point to the first point, the objective lens may simultaneously move or translate to an adjacent portion of the sample. In some cases, the objective lens may then move or translate from the first point to the second point while the imaging module, imager, imaging device, imaging system, acquires a series of images of the one or more object planes of the sample at the adjacent portion. In some cases, a skew angle, described herein, associated with one or more object planes of the sample may be generated when the series of images are acquired while the objective lens is continuously or periodically moving relative to the sample in a direction along the optical axis of the objective lens. In some cases, a mathematical transformation may be applied to the series of images to remove or correct the skew angle, described herein. In some cases, the series of images where the skew angle has been removed or corrected, may be a series of calibrated or corrected images.

The system may comprise one or more sensors, one or more z-axis scanners, or one or more sample holders, or any combination thereof.

In an aspect, provided herein is a method of volumetric imaging of a sample. The method can include providing a three-dimensional or a two-dimensional sample disposed on a stage (e.g., sample holder). The method can further include providing an imaging module, imager, imaging device, or imaging system. The imaging module, imager, imaging device, or imaging system can be configured to create an image. The imaging module, imager, imaging device, or imaging system can have an objective lens configured to transmit photons from one or more object planes within the sample to a sensor.

The method can further include continuously moving the objective lens relative to the sample in a direction substantially parallel to an optical axis of the objective, while simultaneously using the imaging module, imager, imaging device, or imaging system to acquire a series of images corresponding to adjacent object planes within the sample.

In some cases, a method of determining an identity of a biomolecule can comprise providing a system as described elsewhere herein. For example, the system may comprise one or more sensors and one or more z-axis scanners. The one or more sensors and the one or more z-axis scanners may be used to image a cell to generate a signal, and the signal may be used to determine an identity of the biomolecule as described elsewhere herein.

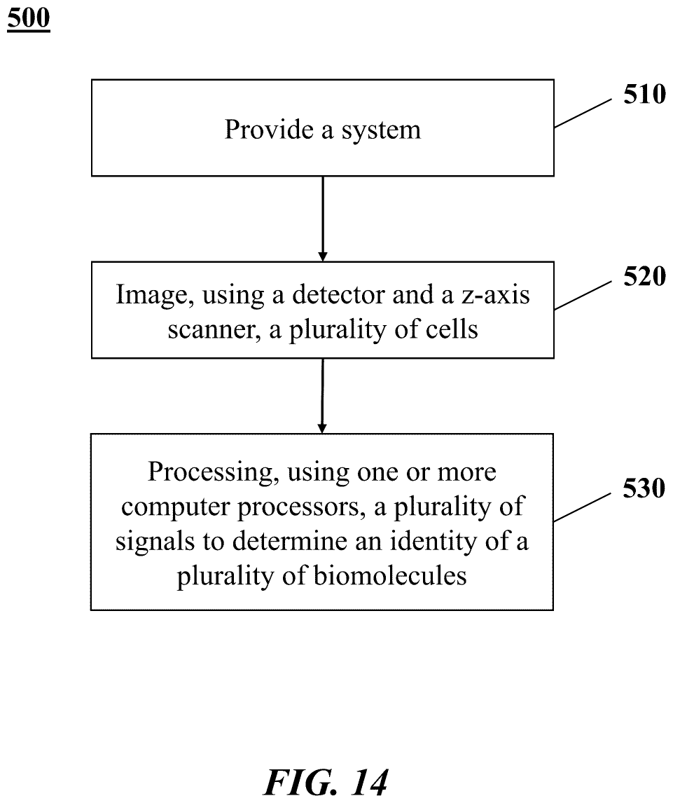

FIG. 14 is a flowchart of a method 500 of determining a property of a plurality of cells, according to some embodiments. In an operation 510, the method 500 may comprise providing a system. The system may comprise one or more sensors and one or more z-axis (e.g., optical axis) scanners.

In an operation 520, the method 500 may comprise imaging, using the one or more sensors and the one or more z-axis scanners, the plurality of cells to generate a plurality of signals. The plurality of signals may be a plurality of images of each cell of the plurality of cells. The plurality of signals may be a single image of each cell of the plurality of cells. The plurality of signals may be a plurality of signals from each cell of the plurality of cells. The plurality of signals may be a single signal from each cell of the plurality of cells.

In another operation 530, the method 500 may comprise using the plurality of signals to determine an identity of the plurality of biomolecules. The imaging or the determining may occur in at most about 72 hours, at most about 60 hours, at most about 48 hours, at most about 36 hours, at most about 35 hours, at most about 34 hours, at most about 33 hours, at most about 32 hours, at most about 31 hours, at most about 30 hours, at most about 29 hours, at most about 28 hours, at most about 27 hours, at most about 26 hours, at most about 25 hours, at most about 24 hours, at most about 23 hours, at most about 22 hours, at most about 21 hours, at most about 20 hours, at most about 19 hours, at most about 18 hours, at most about 17 hours, at most about 16 hours, at most about 15 hours, at most about 14 hours, at most about 13 hours, at most about 12 hours, at most about 11 hours, at most about 10 hours, at most about 9 hours, at most about 8 hours, at most about 7 hours, at most about 6 hours, at most about 5 hours, at most about 4 hours, at most about 3 hours, 2 hours, at most about 1 hours, at most about 0.5 hours, at most about 0.1 hours, or less. The imaging or the determining may occur in at least about 0.1 hours, at least about 0.5 hours, at least about 1 hours, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, or more.

The identity may be determined at least in part using the plurality of signals. For example, the imaging can provide data processed to determine the property. Examples of properties may include, but are not limited to, a structure of a cell of the plurality of cells, an identity or sequence of biomolecule (e.g., nucleic acid, protein, polypeptide, or fragment thereof) contained within a cell of the plurality of cells, or an identity or sequence of a plurality of nucleic acids of the plurality of cells (e.g., perform a panel on the plurality of cells), or any combination thereof. Each cell of the plurality of cells may have a same property determined. For example, each cell of the plurality of cells may have an identity of a nucleic acid determined.

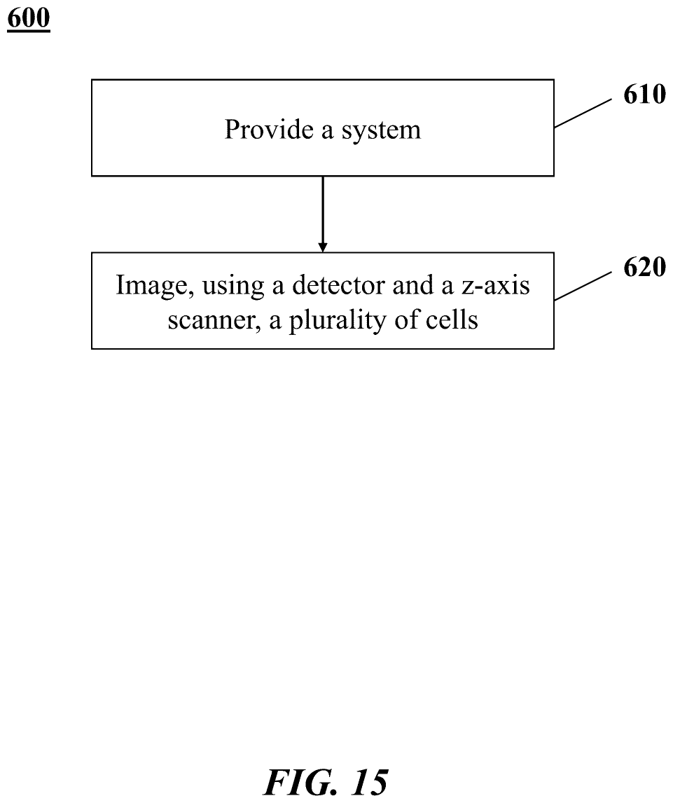

FIG. 15 is a flowchart of a method 600 of imaging a plurality of cells, according to some embodiments. In an operation 610, the method 600 may comprise providing a system. The system may comprise one or more sensors and one or more z-axis (e.g., optical axis) scanners as described elsewhere herein.

In another operation 620, the method 600 may comprise imaging, using the one or more sensors and the one or more z-axis scanners, the plurality of cells. The voxel may comprise the plurality of cells. For example, the imaged volume of the voxel can be of a sample chamber comprising the plurality of cells.

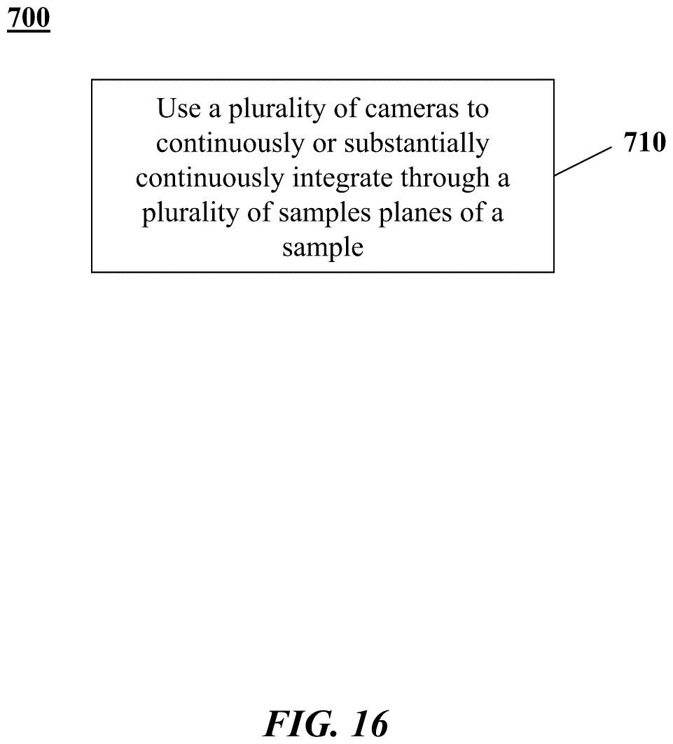

FIG. 16 is a flowchart of a method 700, according to some embodiments. In an operation 710, the method 700 may comprise using a plurality of sensors to continuously or substantially continuously integrate through a plurality of object planes of a sample (e.g., optical planes of the sample) to generate a volumetric measurement of the sample. The imaging of the sample can comprise identifying one or more barcode tags. For example, a sample with barcoded molecular tags can be labeled after a plurality of labeling operations to result in a barcode signal to be detected and decoded by the methods and systems of the present disclosure to determine the identity of the analyte associated with the barcoded molecular tags.

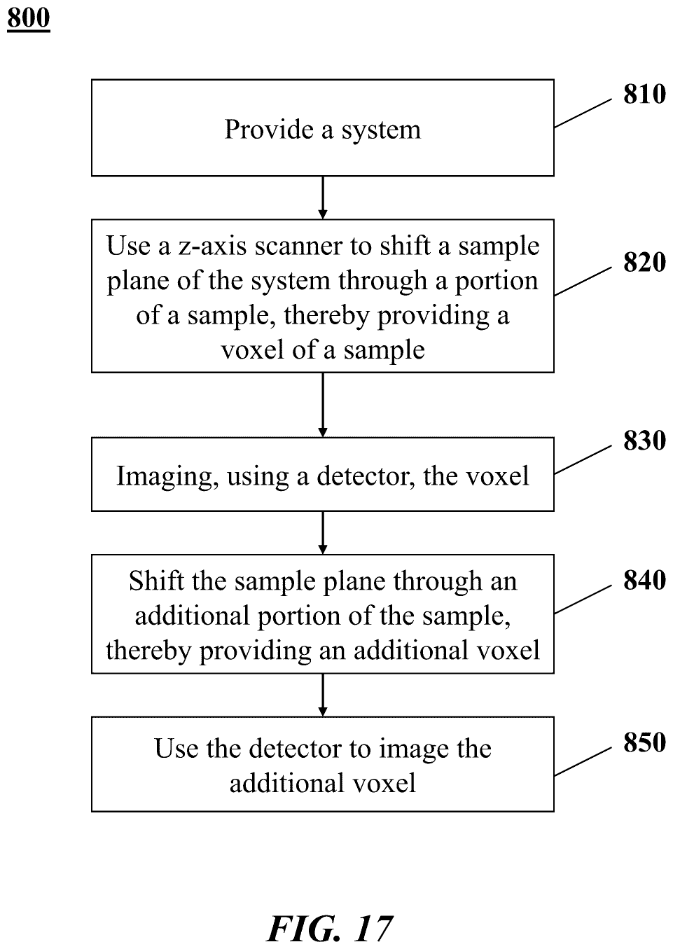

FIG. 17 is a flowchart of a method 800 of taking a volume video of a sample. In an operation 810, the method 800 may comprise providing a system. The system may comprise one or more sensors and one or more z-axis (e.g., optical axis) scanners as described elsewhere herein.

The one or more z-axis scanners may be configured to linearly move the object plane of the system through the sample. The one or more z-axis scanners may be configured to move the object plane non-linearly through the sample. The one or more z-axis scanners may comprise, for example, a motor, a stepper motor, a voice coil, a servo motor, a piezo electric actuator, a hydraulic actuator, or a pneumatic actuator, or any combination thereof. The one or more z-axis scanners may be configured to move one or more sample stages comprising one or more samples, or one or more optical elements configured to move the object plane, or a combination thereof.

In another operation 820, the method 800 may comprise using the one or more z-axis scanners to shift one or more object planes of the system through one or more portions of the one or more samples, thereby generating one or more voxels of the one or more samples. The one or more voxels may be one or more volumes of the one or more samples imaged by the one or more sensors. For example, the voxel may be a volume comprising one or more portions of the sample. In another example, the voxel may be a volume of a plurality of volumes of the sample. The sample may be as described elsewhere herein.

In another operation 830, the method 800 may comprise imaging, using the one or more sensors, the one or more voxels. The imaging may comprise continuous imaging. For example, the imaging of a sample may be continuous through a voxel of the sample. The imaging may comprise substantially continuous imaging. For example, the imaging may be continuously saved for a readout time of the sensor. The imaging may comprise no or substantially no settling time (e.g., less than about 50 milliseconds (ms). For example, the imaging can be performed without waiting for the system to settle (e.g., stop moving after stage or optical element movement of the system). In this example, the imaging can occur as the object plane is shifted through the sample and terminated during settling. The settling time may be due to, for example, waiting for the sample to settle after movement of the sample stage. In this example, the continuous movement of the z-axis scanner can remove settling time from the imaging time which can, in turn significantly improve the throughput of the system. For example, by removing settling time from the imaging cycle of a system, a significant portion of the imaging time can be removed, thereby increasing the throughput.

In another operation 840, the method 800 may comprise shifting the one or more object planes through an additional one or more portions of the one or more samples, thereby providing one or more additional voxels of the one or more samples. The one or more additional voxels and the one or more voxels may be adjoining or contiguous volumes of the sample. For example, one side of the voxel may also be a side of the additional voxel. The one or more additional voxels and the one or more voxels may not be adjacent or contiguous. For example, the additional voxel and the voxel may have space between them.

In another operation 850, the method 800 may comprise detecting, using the one or more sensors, the additional one or more voxels. The imaging of the one or more voxels and the imaging of the additional one or more voxels may occur substantially continuously.

### Data Extraction and Analysis

In one aspect, the present disclosure may provide a method of volumetric imaging of a sample (e.g., a biological tissue), the method comprising: (i) acquiring a series of rolling shutter video frames (e.g., fluorescence images) of the sample while under continuous motion in the z-direction; and (ii) assembling the video frames, thereby forming a skewed volumetric image of the sample.

In some instances, the video rate (e.g., frame rate) and velocity in the z-direction may be coordinated such that:

$$\frac{z - velocity ( μm {s}^{- 1} )}{frame rate ( Hz )} = desired voxel z ( μm ) .$$

In some cases, the sensor height of the camera and z-voxel size may be chosen such that, for a given video rate:

$${tan}^{- 1} ( \frac{voxel z ( μm )}{sensor height ( μm )} ) \leq 0.1 °$$

In some instances, (i) may comprise acquiring a series of video frames using a system (e.g., a microscope system) described herein. In some cases, (i) may comprise acquiring a series of video frames using a system comprising: a microscope device; a sensor comprising a rolling shutter functionality; a device or system for motion in the z-direction; and a control unit. In some case, the system may comprise a z-axis scanner to provide motion in the z-direction, as described herein.

In some instances, the sample of the method is a sample described herein.

In some cases, the collected images are JP2, JPG, TIFF, BMP, GIF, PNG, ND2, JFF, JTF, AVI, or ICS/IDS file types.

In some cases, the present disclosure provides a method for correcting rolling shutter volumetric data, the method comprising: (i) collecting data (e.g., fluorescence images) of a sample under rolling shutter video acquisition, while continuously moving in the z-direction; and (ii) resampling the data into a rectilinear space by removing a skew induced by rolling shutter video acquisition; thereby correcting rolling shutter volumetric data.

In some cases, the data may be collected using a system described herein (e.g., a microscope system described herein).

In some cases, the present disclosure provides a method for stitching adjacent rolling shutter volumetric data, the method comprising: (i) collecting data (e.g., fluorescence images) of a sample under rolling shutter video acquisition, while continuously moving in the z-direction; (ii) removing skews induced by rolling shutter video acquisition while in continuous motion for each field thereby creating a combined volume resampled in a rectilinear coordinate system; thereby stitching adjacent rolling shutter volumetric data.

In some instances, the data may be collected using a system described herein (e.g., a microscope system described herein).

The sensor can collect signals (e.g., that comprise an image). However, not all of the collected information may be useful in all instances. For example, the series of images can comprise signals acquired at fixed locations within the sample (e.g., nucleic acid sequencing loci). In some cases, the desired information is the nucleic acid sequence at a number of loci.

Thus, provided herein are methods that may associate the signals and locations with a reference database. The reference database can include structural information about the sample, reference nucleic acid sequences, or other information.

The signals (e.g., nucleic acid sequences at particular loci) can be extracted from the signals. In some instances, this is done quickly (e.g., within 20 seconds of acquiring the series of images). In some cases, signals may be extracted from the series of images without saving the series of images. The series of images can be kept in a temporary memory buffer while and until signals are extracted.

One or more images generated by imaging the sample during a detection round as described may be analyzed. The images may comprise information related to the sequence of a barcode or reverse complement of a barcode. In some cases, one or images from one round of detection may comprise information related to a portion of the sequence of one or more barcodes or one or more reverse complements of a barcode. In some cases, one or more images from another round of detection may comprise information related to another portion of the sequence or one or more barcodes or one or more reverse complements of a barcode. Analyzing images generated during one or more rounds of detection may result in identification of one or more barcodes or one or more reverse complement of one or more barcodes. Identification of one or more barcodes or one or more reverse complements of one or more barcodes may enable identification of one or more analytes, as described herein. A spatial map, image, display, summary, table, or any combination thereof may be generated based on the analysis described herein. Information related to both the identity of an analyte and the spatial location may be determined based on the analysis described herein. The spatial location may comprise information related to the location of an analyte in an x-direction, y-direction, z-direction, or a combination thereof.

The methods described herein comprise detecting one or more signals associated with an amplicon (e.g., a compacted amplicon). The one or more signals may be used to identify an analyte corresponding to a barcode or reverse complement thereof of the amplicon. In some cases, the one or more signals may be compared to a codebook. The codebook may comprise signal combinations of barcode information corresponding to analytes. For example, the codebook may comprise a barcode and signal information related to the barcode. The codebook may comprise information related to the barcode corresponding to the analyte. Information related to the one or more signals may be compared to the barcode information and/or analyte information within the codebook to identify the analyte.

### Parameters

The stage can include one or more sample holders, which may be configured to retain the one or more samples. The one or more sample holders may comprise, for example, a flow cell, a plurality of flow cells, a well, a plurality of wells, a slide, or a plurality of slides, or any combination thereof. In one example, a flow cell may be configured to flow a plurality of cells to an imaging region of the system (e.g., a region where sensors are configured to image the region). In another example, a well may be configured to retain the plurality of cells. In another example, a plurality of wells may be configured to retain the plurality of cells. In another example, the at least one slide may be configured to retain the plurality of cells. The sample may comprise one or more cells. The one or more cells may comprise one or more cells of a single cell type. For example, the sample may comprise a plurality of epithelial cells. A plurality of cells may comprise a plurality of different types of cells. For example, a sample may comprise a tissue comprising a plurality of cell types.

One or more light sources may be used to illuminate the one or more samples (e.g., a plurality of cells). The one or more light sources may each illuminate a sample of the one or more samples. The one or more light sources may be configured to collectively illuminate a sample of the one or more samples. For example, a plurality of cells may be illuminated by at least one light source. The one or more light sources may comprise at least about 1 light source to 10 light sources. The one or more light sources may comprise at least about 1 light source to 2 light sources, 1 light source to 3 light sources, 1 light source to 4 light sources, 1 light source to 5 light sources, 1 light source to 6 light sources, 1 light source to 7 light sources, 1 light source to 8 light sources, 1 light source to 9 light sources, 1 light source to 10 light sources, 2 light sources to 3 light sources, 2 light sources to 4 light sources, 2 light sources to 5 light sources, 2 light sources to 6 light sources, 2 light sources to 7 light sources, 2 light sources to 8 light sources, 2 light sources to 9 light sources, 2 light sources to 10 light sources, 3 light sources to 4 light sources, 3 light sources to 5 light sources, 3 light sources to 6 light sources, 3 light sources to 7 light sources, 3 light sources to 8 light sources, 3 light sources to 9 light sources, 3 light sources to 10 light sources, 4 light sources to 5 light sources, 4 light sources to 6 light sources, 4 light sources to 7 light sources, 4 light sources to 8 light sources, 4 light sources to 9 light sources, 4 light sources to 10 light sources, 5 light sources to 6 light sources, 5 light sources to 7 light sources, 5 light sources to 8 light sources, 5 light sources to 9 light sources, 5 light sources to 10 light sources, 6 light sources to 7 light sources, 6 light sources to 8 light sources, 6 light sources to 9 light sources, 6 light sources to 10 light sources, 7 light sources to 8 light sources, 7 light sources to 9 light sources, 7 light sources to 10 light sources, 8 light sources to 9 light sources, 8 light sources to 10 light sources, or 9 light sources to 10 light sources. The one or more light sources may comprise at least about 1 light source, 2 light sources, 3 light sources, 4 light sources, 5 light sources, 6 light sources, 7 light sources, 8 light sources, 9 light sources, 10 light sources, or more light sources. The one or more light sources may comprise at most about 10 light sources, 9 light sources, 8 light sources, 7 light sources, 6 light sources, 5 light sources, 4 light sources, 3 light sources, 2 light sources, or less light sources. The one or more light sources may comprise one or more laser light sources, incandescent light sources, light emitting diode (LED) light sources, halogen light sources, or arc lamp illumination light sources, or any combination thereof. The one or more light sources may provide one or more portions of a confocal illumination system (e.g., configured to provide confocal illumination to the one or more samples). The confocal illumination system may comprise one or more spinning disks. A plurality of spinning disks may be used to enable parallel illumination with a plurality of wavelength bands.

The one or more light sources may have a flux of at least about 100 milli-Watts (mW). The one or more light sources may have a flux of at most about 2 Watts. The one or more light sources may provide light sheet illumination (e.g., illumination perpendicular to the imaging axis of the system). The one or more light sources may be configured to provide illumination continuously or substantially continuously through a detection operation as described elsewhere herein. The one or more light sources may have an on/off fraction (e.g., a ratio of time that the one or more light sources are on and in use) of at least about 0.6, 0.65, 0.7, 0.75, 0.8, 0.85, 0.9, 0.95, 0.96, 0.97, 0.98, 0.99, 0.999, or more. The one or more light sources may have an on/off fraction of at most about 0.999, 0.99, 0.98, 0.97, 0.96, 0.95, 0.94, 0.93, 0.92, 0.91, 0.9, 0.85, 0.8, 0.75, 0.7, 0.65, 0.6, or less. The one or more light sources may have an on/off fraction from about 0.8 to about 0.9, from about 0.85 to about 0.95, or from about 0.9 to about 0.99. The one or more light sources may be operated at a same time or a substantially same time as one or more sensors. For example, the one or more light sources can illuminate the one or more samples while the one or more sensors are detecting light from the one or more samples. In some cases, the illuminating may occur prior to the detecting. In some cases, the detecting and the illuminating may occur simultaneously or substantially simultaneously (e.g., within a few nanoseconds of illumination).

In some cases, the one or more z-axis scanners may shift one or more object planes of the system through at least a portion of the one or more samples (e.g., a plurality of cells). The object plane may be a plane of the one or more samples detected by one or more sensors (e.g., a plane of the one or more samples that is sampled by one or more focal planes of the one or more sensors). For example, one or more object planes can be an optical plane of the one or more sensors. In another example, the object plane can be a skewed plane that is not orthogonal to the optical axis of the system. In this example, the object plane may be skewed due to use of one or more rolling shutter sensors. The shifting of the object plane may generate a voxel of one or more voxels associated with the one or more samples. In other cases, the object plane may be shifted through at least an additional portion of the one or more samples. The shifting through the at least the additional portion may generate an additional voxel of the one or more voxels associated with the one or more samples. The one or more z-axis scanners may shift the object plane through a shifting of the one or more sample holders, or a shifting of one or more optical elements disposed in the optical system (e.g., one or more lenses configured to adjust the object plane through focusing of light), or a combination thereof. The one or more z-axis scanners may comprise a motion element. Examples of motion elements include, but are not limited to, a motor, a stepper motor, a voice coil, a servo motor, a piezo electric actuator, a hydraulic actuator, a linear motor, or a pneumatic actuator, or any combination thereof. The one or more voxels may be associated with at least a portion of one or more cells. The one or more voxels may be associated with at least a portion of a plurality of cells. The one or more voxels may comprise one or more biomolecules (e.g., as a part of one or more cells). The one or more z-axis scanners may be configured to linearly move the object plane. The one or more z-axis scanner may be configured to move the object plane non-linearly (e.g., exponentially, logarithmically, etc.). The object plane may have a height (e.g., dimension in the optical axis of the system) of at most about 500 micrometers (μm), at most about 450 μm, at most about 400 μm, at most about 350 μm, at most about 300 μm, at most about 250 μm, at most about 200 μm, at most about 150 μm, at most about 100 μm, at most about 90 μm, at most about 80 μm, at most about 70 μm, at most about 60 μm, at most about 50 μm, at most about 40 μm, at most about 30 μm, at most about 20 μm, at most about 10 μm, at most about 9 μm, at most about 8 μm, at most about 7 μm, at most about 6 μm, at most about 5 μm, at most about 4 μm, at most about 3 μm, at most about 2 μm, at most about 1 μm, at most about 0.9 μm, at most about 0.8 μm, at most about 0.7 μm, at most about 0.6 μm, at most about 0.5 μm, at most about 0.4 μm, at most about 0.3 μm, at most about 0.2 μm, at most about 0.1 μm, or less. The object plane may have a height of at least about 0.1 μm, at least about 0.2 μm, at least about 0.3 μm, at least about 0.4 μm, at least about 0.5 μm, at least about 0.6 μm, at least about 0.7 μm, at least about 0.8 μm, at least about 0.9 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 150 μm, at least about 200 μm, at least about 250 μm, at least about 300 μm, at least about 350 μm, at least about 400 μm, at least about 450 μm, at least about 500 μm, or more. The object plane may have a height from about 1 micrometer to about 10 micrometers. In some cases, the one or more z-axis scanners may shift one or more object planes of the system through at least a portion of one or more cells. For example, the shifting of the object plane can generate one or more voxels comprising at least a portion of the one or more cells. In some cases, each voxel can comprise a single cell or one or more portions thereof. In some cases, each voxel can comprise a plurality of cells. In some cases, a voxel can comprise one or more portions of a plurality of cells.

The voxel or the object plane may have a resolution in the optical axis of the system, the plane orthogonal to the optical axis, or a combination thereof of at least about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, or more. The voxel or the object plane may have a resolution in the optical axis of the system, the plane orthogonal to the optical axis, or a combination thereof of at most about 10 μm μm, 9 μm μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, 0.09 μm, 0.08 μm, 0.07 μm, 0.06 μm, 0.05 μm, 0.04 μm, 0.03 μm, 0.02 μm, 0.01 μm, or less. The voxel or the object plane may have a resolution in the optical axis of the system of about 0.1 micrometer to about 10 micrometers. In some cases, the voxel size is approximately 0.1 μm in the x-direction, 0.1 μm in the y-direction, and 0.5 μm in the z-direction.

In some cases, one or more sensors may be used to detect one or more signals associated with the one or more samples. The one or more sensors may be used to detect one or more signals associated with a plurality of cells. The one or more sensors may be configured to detect one or more signals associated with the one or more voxels. For example, the one or more sensors can detect signals from within the one or more voxels. The one or more sensors may comprise, for example, one or more rolling shutter sensors, or one or more global shutter sensors, or a combination thereof. In an example, as a rolling shutter sensor is used, the rows of pixels of the rolling shutter sensor can be sequentially read as, for example, the object plane is moved through the sample. The one or more sensors may comprise one or one or more complementary metal oxide semiconductor (CMOS) sensors, or a combination thereof. The one or more sensors may be configured to detect a fluorescent signal, or a non-fluorescent signal, or a combination thereof. Examples of signals may include, but are not limited to, light intensity, wavelength, fluorescent lifetime, absorption, cross-section or absorption lifetime. The one or more sensors may be configured to be continuously (e.g., read without whole sensor dead time on the one or more sensors) or substantially continuously read (e.g., read with partial sensor dead time on the one or more sensors due to, for example, readout time). For example, for a sensor comprising a plurality of pixel bins, the bins may be configured to be read any time the bins are not integrating. The one or more sensors may independently have a duty cycle (e.g., a ratio of detecting time to readout time) of at least about 50%, at least about 60%, at least about 70%, at least about 80%, at least about 90%, at least about 95%, at least about 99%, at least about 99.5%, or at least about 99.9%. The one or more sensors may have a duty cycle from about 95% to about 99.9%. The detecting may comprise no or substantially no settling time (e.g., time spent not detecting to decrease artifacts generated by system movement). For example, the system may not settle before using the one or more sensors to detect the one or more signals. The settling may be settling to reduce or remove artifacts induced by system acceleration or deceleration.

The one or more samples may be imaged using the one or more sensors or the one or more z-axis scanners. In some cases, the imaging may generate one or more images associated with the one or more samples. In other cases, the imaging may generate one or more signals associated with the one or more samples. For example, a plurality of cells may be imaged and the data generated by the imaging can comprise the one or more signals. In this example, the one or more signals may comprise raw data from the imaging. The imaging may then generate a plurality of images or a plurality of signals. In some cases, the voxel or the additional voxel may be imaged. The imaging may be continuous or substantially continuous (e.g., with a duty cycle as described elsewhere herein). The one or more images may comprise one or more two-dimensional images, or one or more three-dimensional images, or a combination thereof. The one or more images may comprise, for example, optical microscopy images of the one or more samples.

The system may comprise or be operably coupled to one or more computer processors. The one or more computer processors may perform processing on the one or more signals or the one or more images. The processing may comprise post-processing (e.g., processing the one or more signals or one or more images after acquisition). In some cases, the one or more signals may be used to determine an identity of one or more biomolecules associated with the one or more samples. Examples of processing may include, but is not limited to, background correction (e.g., removal of background signal), baseline correction (e.g., removal of baseline signal), removal of optical aberration (e.g., removal of chromatic aberration, diffraction, etc.), calibration (e.g., application of one or more calibration curves to the one or more signals), removal of imaging artifacts (e.g., removal of dead pixels, dust artifacts, etc.), removal of optical skew (e.g., skew relative to the optical axis of the system), denoising (e.g., application of one or more noise filters), or normalization (e.g., normalization of the one or more signals to an internal or external signal), registration to reference coordinates, resampling, de-warping, or any combination thereof. The background or baseline (e.g., dark state measurements) may be taken prior to the one or more signals or the one or more images. The background or baseline may be taken after the one or more signals or the one or more images.

The one or more signals or the one or more images may be used to determine one or more labels associated with the one or more samples, one or more properties of the one or more samples, or one or more identities of one or more biomolecules of the one or more samples, or any combination thereof. The one or more signals may be processed to determine an identity of a label within the one or more samples. For example, a fluorescent label can be identified based on the wavelength of the center of the peak of the fluorescent label. Alternatively, a fluorescent label may be identified by the ratios of signals between two or more imaging passbands. The identity of one or more labels may be used to determine one or more identities or one or more properties of one or more biomolecules in the one or more samples. For example, using an identity of the label or sequence of labels can enable a lookup of the biomolecule the label is configured to associated with in a reference database. In this example, the presence of the label can identify the presence of the biomolecule it is associated with. For one or more samples comprising a plurality of labels, a plurality of signals can identify each label of the plurality of labels. Examples of properties include, but are not limited to, a mutation or wildtype status of one or more biomolecules, a presence or absence of one or more biomolecules, a sequence of one or more biomolecules, or a gene status for a gene panel. The gene panel may comprise at least about 8 genes, 24 genes, 100 genes, 250 genes, 500 genes, 600 genes, 700 genes, 800 genes, 900 genes, 1,000 genes, 2,000 genes, 4,000 genes, 6,000 genes, 10,000 genes, 15,000 genes, 20,000 genes, or more. The gene panel may comprise at most about 1,000 genes, 900 genes, 800 genes, 700 genes, 600 genes, 500 genes, 250 genes, 100 genes, 50 genes, 24 genes, 8 genes or less genes. The gene panel may be a panel configured to determine a status of a plurality of genes in the one or more samples.

The plurality of cells may comprise at least about 50,000 cells, 100,000 cells, 150,000 cells, 200,000 cells, 250,000 cells, 300,000 cells, 350,000 cells, 450,000 cells, 500,000 cells, 550,000 cells, 600,000 cells, 650,000 cells, 700,000 cells, 750,000 cells, 800,000 cells, 850,000 cells, 900,000 cells, 950,000 cells, 1,000,000 cells, 1,100,000 cells, 1,200,000 cells, 1,300,000 cells, 1,400,000 cells, 1,500,000 cells, 1,600,000 cells, 1,700,000 cells, 1,800,000 cells, 1,900,000 cells, 2,000,000 cells, or more. The plurality of cells may comprise at most about 2,000,000 cells, 1,900,000 cells, 1,800,000 cells, 1,700,000 cells, 1,600,000 cells, 1,500,000 cells, 1,400,000 cells, 1,300,000 cells, 1,200,000 cells, 1,100,000 cells, 1,000,000 cells, 950,000 cells, 900,000 cells, 850,000 cells, 800,000 cells, 750,000 cells, 700,000 cells, 650,000 cells, 600,000 cells, 550,000 cells, 500,000 cells, 450,000 cells, 400,000 cells, 350,000 cells, 300,000 cells, 250,000 cells, 200,000 cells, 150,000 cells, 100,000 cells, 50,000 cells, or less. The imaging may be performed on the one or more samples in a time period of at most about 72 hours, 60 hours, 48 hours, 36 hours, 35 hours, 34 hours, 33 hours, 32 hours, 31 hours, 30 hours, 29 hours, 28 hours, 27 hours, 26 hours, 25 hours, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hours, 0.5 hours, 0.1 hours, or less. The imaging may be performed on the one or more samples in a time period of at least about 0.1 hours, 0.5 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 48 hours, 60 hours, 72 hours, or more. The imaging may be performed on the one or more samples in a time period from about 2 hours to about 36 hours, from about 18 hours to about 30 hours, or from about 22 hours to about 26 hours.

In another aspect, the present disclosure provides a method using one or more sensors. The one or more sensors may be used to integrate (e.g., substantially continuously integrate) through one or more object planes of one or more samples. The integration may generate one or more volumetric measurements of the one or more samples. One or more z-axis scanners may shift the one or more object planes through the one or more samples. During the shifting of the one or more object planes, the one or more sensors may integrate to generate the one or more volumetric measurements of the one or more samples.

The volumetric measurement may be of one or more voxels as described elsewhere herein. For example, the volumetric measurement may be of a volume comprising one or more samples. The volumetric measurement may comprise one or more images as described elsewhere herein, or one or more signals as described elsewhere herein, or a combination thereof. The volumetric measurement may comprise the one or more object planes. For example, the one or more object planes within the sample may be combined to form the volumetric measurement.

The one or more object planes may be illuminated by one or more light sources as described elsewhere herein. The one or more sensors may be as described elsewhere herein. The one or more sensors may detect one or more signals or one or more images from the one or more samples planes. The one or more images or one or more signals may form the volumetric measurement. The one or more sensors may be configured to detect one or more passbands (e.g., wavelength ranges). For example, each sensor of the one or more sensors may be configured to image a different passband of the one or more passbands. The use of a plurality of passbands can parallelize the detecting of a plurality of, for example, labels, which can increase system throughput.

Using one or more rolling shutter sensors can impart skew to the one or more object planes. The one or more object planes may be at a skew relative to an imaging axis (e.g., optical axis of the one or more sensors) of the one or more sensors. For example, one or more object planes can be parallel to one another and not perpendicular to the optical axis of the system. The one or more object planes may be skewed from the optical axis of the system by at least about 0.01 milliradians, 0.05 milliradians, 0.1 milliradians, 0.2 milliradians, 0.3 milliradians, 0.4 milliradians, 0.5 milliradians, 0.6 milliradians, 0.7 milliradians, 0.8 milliradians, 0.9 milliradians, 1 milliradians, 2 milliradians, 3 milliradians, 4 milliradians, 5 milliradians, 6 milliradians, 7 milliradians, 8 milliradians, 9 milliradians, or more. The one or more object planes may be skewed from the optical axis of the system by at most about 9 milliradians, 8 milliradians, 7 milliradians, 6 milliradians, 5 milliradians, 4 milliradians, 3 milliradians, 2 milliradians, 1 milliradians, 0.9 milliradians, 0.8 milliradians, 0.7 milliradians, 0.6 milliradians, 0.5 milliradians, 0.4 milliradians, 0.3 milliradians, 0.2 milliradians, 0.1 milliradians, 0.05 milliradians, 0.01 milliradians, or less. The skew may be from about 0.1 milliradians to about 10 milliradians. The one or more object planes may all have a same skew from the optical axis. The one or more object planes may have a plurality of different skew values. The skew of the one or more object planes may be removed in one or more post processing operations on the one or more volumetric measurements. The skew may be removed to enable mapping of the one or more object planes into three-dimensions. The one or more volumetric measurements may be processed as described elsewhere herein. The one or more volumetric measurements may be used to generate one or more signals associated with the one or more samples, or one or more images of the one or more samples, or a combination thereof. For example, the volumetric measurement may comprise a measurement of one or more signals (e.g., labels) within the volume of the volumetric measurement. In this example, the one or more signals can be extracted or processed as described elsewhere herein to generate the one or more signals.

The plurality of sensors can acquire a plurality of passbands. For example, the plurality of sensors can be configured to each image a different passband (e.g., spectral wavelength range). In this example, the parallelization of the spectral acquisition can improve the speed and throughput of the system. The plurality of sensors may be one or more portions of a confocal microscopy system. For example, the plurality of sensors can be the detection apparatus of a confocal microscopy system. The plurality of sensors may be configured to image a same object plane. For example, the plurality of sensors can be configured to have a same focal depth. The plurality of sensors may be configured to image a plurality of different object planes. For example, each sensor of the plurality of sensors can be configured to image a different object plane.

The volumetric measurement of the sample may be generated by a continuous integration through the sample. For example, the volumetric measurement can be formed by use of continuous integration through the sample. In this example, the continuous integration through the sample can generate a measurement of the sample that is spread through the volume of the sample. The volumetric measurement of the sample may be a substantially continuous integration through the sample. For example, the volumetric measurement may be continuous except for a readout of a sensor. In this example, the volumetric measurement may be of a volume of the sample detected by the sensor except for the portion of the volume where the sensor readout occurs. The plurality of sensors may each independently have a frame rate of at least about 1 ms (milliseconds), at least about 5 ms, at least about 10 ms, at least about 15 ms, at least about 20 ms, at least about 25 ms, at least about 30 ms, at least about 35 ms, at least about 40 ms, at least about 45 ms, at least about 50 ms, at least about 55 ms, at least about 60 ms, at least about 65 ms, at least about 70 ms, at least about 75 ms, at least about 80 ms, at least about 85 ms, at least about 90 ms, at least about 95 ms, at least about 100 ms, at least about 150 ms, at least about 200 ms, at least about 250 ms, at least about 300 ms, at least about 350 ms, at least about 400 ms, at least about 450 ms, at least about 500 ms, at least about 550 ms, at least about 600 ms, at least about 650 ms, at least about 700 ms, at least about 750 ms, at least about 800 ms, at least about 850 ms, at least about 900 ms, at least about 950 ms, at least about 1 ms, at least about 000 ms, at least about or more. The plurality of sensors may each independently have a frame rate of at most about 1,000 ms, at most about 950 ms, at most about 900 ms, at most about 850 ms, at most about 800 ms, at most about 750 ms, at most about 700 ms, at most about 650 ms, at most about 600 ms, at most about 550 ms, at most about 500 ms, at most about 450 ms, at most about 400 ms, at most about 350 ms, at most about 300 ms, at most about 250 ms, at most about 200 ms, at most about 150 ms, at most about 100 ms, at most about 95 ms, at most about 90 ms, at most about 85 ms, at most about 80 ms, at most about 75 ms, at most about 70 ms, at most about 65 ms, at most about 60 ms, at most about 55 ms, at most about 50 ms, at most about 45 ms, at most about 40 ms, at most about 35 ms, at most about 30 ms, at most about 25 ms, at most about 20 ms, at most about 15 ms, at most about 10 ms, at most about 5 ms, at most about 1 ms, at most about or less ms. The frame rate may represent a refresh time of the sensor (e.g., a time between when imaging ends and the sensor is available again for imaging). For example, the frame rate may represent a fastest time between integration cycles of the sensor. In this example, a sensor with a frame rate of 100 milliseconds may have a 100-millisecond time between integrations. A volumetric measurement may be a measurement of a volume of one or more samples. The volumetric measurement may contain spatially encoded data from the one or more samples, which can provide information about not only a presence or absence of a biomolecule, but also the location of the biomolecule within the one or more samples.

The series of images can be acquired at a rate of at least about 100,000,000 voxels/second on each of two or more wavelength channels.

The series of images may comprise at least 50 images, at least 100 images, at least 500 images, at least 1000 images, at least 5000 images, or at least 10,000 images.

### ADDITIONAL EMBODIMENTS

Conventional 3D volume microscopy may require a process in which two-dimensional (x-y) images are taken at a series of discrete z-positions (focus). To prevent crosstalk between z-steps, one typically may use a method to reject out of (x-y) plane features such as confocal or light sheet microscopy. For thin samples, a few layers can be assembled into a volume by just using depth of focus as a rejection mechanism although this still captures out of plane light as background.

In general, between each image acquisition, the system may make a step that is of order the depth of focus (or the thickness of the lightsheet). For a high numerical aperture (NA) confocal system the z-steps can be as small as 100 nm while for a light sheet system the steps can be 10 μm or larger. After each move, the system may stabilize (settle) before the next image is acquired. After each image, the camera(s) may readout and prepare for the next acquisition. The move and readout times may sometimes be concurrent but there may be dead time in the acquisition cycle.

The disclosure provided herein may largely eliminate the dead times of camera read out and z-motion during an image stack acquisition by acquiring data while the system is in continuous z-motion. This may be accomplished by operating the camera in a continuous video mode (e.g., rolling shutter or global shutter video) while making a continuous and coordinated movement z-direction. To maximize throughput, the coordinated movement in the z-direction may be at a velocity such that a frame of video is acquired over a desired z sampling. For example, if sampling at 500 nm in z (e.g., z-voxel size of 500 nm) with a 20 Hz video rate, the coordinated z velocity may be 500 nm×20 Hz=10 μm per second. If imaging with multiple cameras (and passbands) simultaneously, the video frames can be synchronized between the cameras, but it is not required.

In some aspects, the systems and methods described herein may utilize one or more types of microscopy (e.g., a microscope device). In some cases, the systems and methods described herein may utilize a type of microscopy selected from brightfield microscopy, dark field microscopy, phase contrast microscopy, differential interference contrast microscopy, fluorescence microscopy, confocal fluorescence microscopy, or lightsheet fluorescence microscopy.

Various methods exist for imaging samples (e.g., tissue, e.g., biological tissue) on microscopic length scales. These methods may include, inter alia, brightfield microscopy, dark field microscopy, phase contrast microscopy, differential interference contrast microscopy, fluorescence microscopy, confocal fluorescence microscopy, or lightsheet fluorescence microscopy. In traditional brightfield or epifluorescence microscopy, the entire sample may be irradiated with light and viewed through an eye piece or as a digital image. The viewed image may comprise signal from many focal planes of the sample and therefore may not be spatially resolved. While these techniques have many benefits, including low cost and ease of operation, it may not be suitable for applications that require knowledge of the origin of the signal.

Confocal microscopy may be a microscopy technique which, in its original conception, focuses light through an aperture (e.g., a pinhole) to illuminate a small region of a sample. Emitted light from a sample may be blocked by an aperture placed in front of a sensor. The blocking of out-of-focus light by the aperture may force only light from a small region of the sample to be recorded. This process can be continued by scanning the sample in the XY plane or Z direction to form a 2D and/or 3D image of the sample.

While original confocal systems may employe the use of dual apertures to spatially resolve a sample, modern techniques often rely on a laser source. Laser scanning confocal microscopy (LSCM) is a technique wherein a laser light spot, directed by a series of mirrors, may scan the sample point by point. The light detected by the sensor as the laser is then rastered across the sample may then be assembled into an overall image of the specimen.

Background information on laser scanning confocal microscopy can be found, inter alia, in Molecular Biotechnology 16 (2000): 127-149 or Biotechniques 39.6 (2005): S2-S5.

Spinning disk confocal microscopy (e.g., with a Nipkow disk) may use a series of moving pinholes to scan an area of a sample in parallel. In general, this may reduce the amount of time required to scan.

Background information on spinning disk confocal microscopy can be found, inter alia, in Current protocols in cytometry 92.1 (2020): e68 or Journal of biomolecular techniques: JBT 26.2 (2015): 54.

Rolling shutter may generally refer to a video mode of some digital cameras whereby each row of the image sensor is read out and reset in quick succession. Rolling shutter is a common video mode on consumer and commercial cameras, particularly inexpensive cameras. When the whole sensor has been read, e.g., top to bottom, the process may begin again at the top. For a 20 Hz frame rate on a camera having 1000 rows, the line read rate may be 20 Hz×1000=20 kHz, for example. This video mode may have the feature that each row integrates a slightly shifted time interval. In the example just given, each row may integrate for 50 milliseconds (ms), but the first row of the image may integrate an interval that may be almost 50 ms earlier than the last row. This can create startling effects when taking video with high-speed x-y motion in the frame. In this application where motion is relatively slow and, in the z-axis, the primary artifacts may be z-blurring and tilt of the sample x-y plane relative the z-axis normal. If the row direction is “x” then the effective “y” axis may be tilted by one z sample distance divided by the sensor height (number of rows×pixel size). If the pixel scale at the image is 100 nm and the z-step is 500 nm, then the tilt may be 500 nm/(1000 rows×100 nm)=0.005 radians or 0.29°. In some cases, this angle is about 0.1° or less. An additional artifact may be to slightly blur the z-dimension of the volumetric image by a fraction of the z-voxel size. This can be reduced by adjusting the z-voxel size.

The present disclosure provides methods and systems for producing high-throughput volumetric images of a sample (e.g., tissue, e.g., biological tissue).

In one aspect, the present disclosure provides a method of volumetric imaging of a sample (e.g., a tissue, e.g., biological tissue), the method may comprise: acquiring a series of video frames (e.g., fluorescence images) of the sample while under continuous motion in the z-direction; and, assembling the video frames, thereby forming a volumetric image of the sample. In some cases, e.g., with rolling shutter video, the volumetric image produced is skewed. In some cases, the video rate (e.g., frame rate) and velocity in the z-direction are coordinated such that

$$\frac{z - velocity ( μm {s}^{- 1} )}{frame rate ( Hz )} = desired voxel z ( μm ) .$$

In some instances, the sensor height of the camera and z-voxel size are chosen such that

$${tan}^{- 1} ( \frac{voxel z ( μm )}{sensor height ( μm )} ) \leq 0.1 °$$

for a given video rate. In some cases, the continuous video integration will induce a z blur of approximately ½ the z voxel size, analogous to transverse blur in time delay integration (TDI) scanning systems. To reduce this blurring one can pick the z-voxel size to 100%, 80%, 50%, 25% or 10% of the system depth of focus.

In some cases, the rolling shutter videos frames have a volumetric skew angle (e.g., angle of sample x-y plane relative to the z-direction) of about 0.1° or less (e.g., about 0.09°, 0.08°, 0.07°, 0.06°, 0.05°, 0.04°, 0.03°, 0.02°, 0.01° or less). In some instances, the skew angle is less than about 0.2°.

In some instances, the z-voxel size is greater than 100 nm (e.g., greater than about 200 nm, 400 nm, 1,000 nm, 2,000 nm, or 4,000 nm).

In some cases, the method further comprises the use of image processing software. In some instances, the method further comprises an image correction operation (e.g., correcting for a skew angle). In some cases, the skewed volumetric image is digitally transformed to rectilinear using a linear transform, affine transform, or other non-linear transform.

In another aspect, the disclosure provides a method for correcting rolling shutter volumetric data, the method may comprise acquiring data (e.g., fluorescence images) of a sample under rolling shutter video acquisition, while continuously moving in the z-direction; and, resampling the data into a rectilinear space by removing a skew induced by rolling shutter video acquisition; thereby correcting rolling shutter volumetric data.

In another aspect, the disclosure provides a method for stitching adjacent rolling shutter volumetric data, the method may comprise: acquiring data (e.g., fluorescence images) of a sample under rolling shutter video acquisition, while continuously moving in the z-direction; and, removing a skew induced by rolling shutter video acquisition while in continuous motion for each field thereby creating a combined volume resampled in a rectilinear coordinate system.

In another aspect, the present disclosure provides systems for volumetric imaging of a sample (e.g., tissue, e.g., biological tissue), where the systems may comprise: a visualization device; a device or system for motion in the z-direction; and a control unit. In some cases, the motion in the z-direction may be provided by a z-axis scanner. In some cases, the sample is a biological tissue. In some instances, the sample is greater than 50 μm in the z-direction (e.g., at least 50 μm, 60 μm, 70 μm, 80 μm, 90 μm, 100 μm, 120 μm, 140 μm, 160 μm, 180 μm, 200 μm, 250 μm). In some instances, the sample further comprises a detection tag.

In another aspect, the present disclosure provides systems for volumetric imaging of a sample (e.g., tissue, e.g., biological tissue), where the system may comprise: a microscope device; a sensor; a device or system for motion in the z-direction; and a control unit. In some cases, the motion in the z-direction may be provided by a z-axis scanner. In some cases, the microscope device is a confocal microscope device or a lightsheet microscope device (e.g., a microscope device as described herein). In some instances, the objective lens is moved continuously in the z-direction. In some instances, the stage is capable of continuous movement in the z-direction. In some cases, the stage is capable of movement in x, y, or z directions. In some cases, the stage is capable of movement in the z-direction. In some cases, the stage is capable of movement in the z direction at a constant velocity.

In some cases, the sensor comprises a complementary metal-oxide-semiconductor (CMOS) sensor, scientific complementary metal-oxide-semiconductor (sCMOS), or charge-coupled device (CCD). In some cases, the sensor comprises a rolling shutter functionality (e.g., a rolling shutter functionality as described herein) or other continuous integration modality (e.g., global shutter video). In some cases, the global shutter video may rely on a secondary readout array allowing for substantially continuous integration, e.g., greater than 90%, 95% 98% 99%, 99.5%, 99.9% integration duty cycle.

In another aspect, the present disclosure provides methods for volumetric imaging of a sample (e.g., tissue, biological tissue, or cleared and stabilized biological tissue), the method may comprise: providing a system for volumetric imaging of a sample (e.g., a system described herein); providing a sample comprising a detection tag; acquiring a continuous series of fluorescence images of the sample while under motion in the z-direction; and, assembling the images, thereby forming a volumetric image of the sample.

Lightsheet fluorescence microscopy may be characterized by the use of a “lightsheet,” a laser beam with a width of approximately a few hundred nanometers to a few micrometers, to irradiate a sample in a plane perpendicular to the direction of the imaging or observation direction.

Background information on spinning disk confocal microscopy can be found, inter alia, in Journal of Histochemistry & Cytochemistry 59.2 (2011): 129-138 or Nature Methods 14.4 (2017): 360-373.

In one aspect, the present invention provides systems for volumetric imaging of a sample (e.g., tissue, e.g., biological tissue), where the systems may comprise: (i) a microscope device; (ii) a sensor comprising a rolling shutter functionality; (iii) a stage capable of motion in the z-direction; and (iv) a control unit; optionally, wherein the tissue comprises one or more detection tags.

The system described herein may comprise a visualization device suitable for imaging a sample (e.g., a sample described herein). In some instances, the visualization device is a microscope device.

In some cases, the microscope device is a confocal microscope device or a lightsheet microscope device. In some cases, the microscope device is a confocal microscope device. In some instances, the microscope device is a light sheet microscope device.

In some instances, the microscope device is a laser scanning confocal microscope device, a spinning disk confocal microscope device, or a line-confocal microscope device. In some cases, the microscope device is a laser scanning confocal microscope device. In some instances, the microscope device is a spinning disk confocal microscope device. In some cases, the microscope device is r a line-confocal microscope device.

In some cases, the systems herein comprise (e.g., are combined with or integrated into) an existing microscope system (e.g., a confocal microscope system, e.g., a light sheet microscope system). In some cases, the systems herein comprise (e.g., are combined with or integrated into) an existing microscope system selected from: Nikon AX R MP, Nikon AX/AX R, Nikon AX NIR, Nikon CSU-X1, Nikon CSU-W1, Nikon CSUW1 SoRa, Crest X-Light V2, Crest X-Light V3, Crest X-Light V3/DeepSIM, ThorLabs Veneto, ThorLabs Cerna, Zeiss Axio Observer Z1, Zeiss Axiovert 40, Leica Stellaris 5, Leica Stellaris 8, Leica TCS SP8, Olympus BX61, Olympus FV3000, Olympus FVMPE-RS, Oxford Dragonfly 200, or Oxford Dragonfly 600.

In some instances, the microscope device comprises a light source, illumination optics, or imaging optics. In some instances, the microscope device comprises a light source. In some cases, the microscope device comprises illumination optics. In some cases, the microscope device comprises imaging optics.

In some cases, the light source illuminates a region on the sample after interaction with one or more of the objective lens and illumination optics. The microscope objective may then capture and magnify emitted light from the sample, which may be captured by a sensor (e.g., a sensor as described herein), optionally after interaction with imaging optics.

In some cases, the confocal microscope device comprises an objective, a light source, and additional optical elements.

In some instances, the microscope device comprises a feature or optical element disclosed in U.S. Pat. Nos. 5,535,052; 5,612,818; 6,094,300; 7,335,898; each of which is incorporated herein by reference in their entirety.

In some instances, the microscope device comprises a feature or optical element disclosed in U.S. Pat. No. 5,067,805, which is incorporated herein by reference in its entirety.

In some cases, the visualization device (e.g., microscope device) described herein comprises an objective lens. The objective lens may be generally considered the most important optical component of the microscope and may be responsible for the information content (e.g., resolution) of the resulting image.

In some cases, the objective lens is a high-performance objective suitable for use with a laser or non-coherent light source (e.g., a light source described herein).

In some cases, the objective lens is highly corrected for geometric and chromatic aberrations over the desired field of view.

In some cases, the objective lens transmits light from at least the near ultraviolet to the near infrared range.

In some instances, the objective lens has a suitable numerical aperture. The numerical aperture of an objective may be dimensionless unit that quantifies the objective's ability to gather light and may directly relate to the ability of the objective to resolve details in the sample (e.g., a sample described herein).

In some instances, the objective lens may have a numerical aperture of less than or equal to about 0.10, less than or equal to about 0.15, less than or equal to about 0.20, less than or equal to about 0.25, less than or equal to about 0.30, less than or equal to about 0.35, less than or equal to about 0.40, less than or equal to about 0.45, less than or equal to about 0.50, less than or equal to about 0.55, less than or equal to about 0.60, less than or equal to about 0.65, less than or equal to about 0.70, less than or equal to about 0.75, less than or equal to about 0.80, less than or equal to about 0.85, less than or equal to about 0.90, less than or equal to about 0.95, less than or equal to about 1, less than or equal to about 1.05, less than or equal to about 1.10, less than or equal to about 1.15, less than or equal to about 1.20, less than or equal to about 1.25, less than or equal to about 1.30, less than or equal to about 1.35, less than or equal to about 1.40, less than or equal to about 1.45, less than or equal to about or 1.50.

In some cases, the objective lens has a numerical aperture of more than or equal to about 0.60, more than or equal to about 0.65, more than or equal to about 0.70, more than or equal to about 0.75, more than or equal to about 0.80, more than or equal to about 0.85, more than or equal to about 0.90, more than or equal to about 0.95, more than or equal to about 1, more than or equal to about 1.05, more than or equal to about 1.10, more than or equal to about 1.15, more than or equal to about 1.20, more than or equal to about 1.25, more than or equal to about 1.30, more than or equal to about 1.35, more than or equal to about 1.40, more than or equal to about 1.45, more than or equal to about or 1.50.

In some instances, the objective lens is an achromat, plan achromat, fluor, or apochromat objective. The objective lens may have any magnification suitable for the sample (e.g., a sample described herein). In some cases, the objective lens has a magnification selected from: 4×, 10×, 20×, 40×, 60× or 100×.

In some instances, the objective lens is a water-immersion lens, glycerol immersion lens, oil immersion lens, or silicone immersion lens.

In some cases, the objective lens has a working distance (e.g., the distance between the top surface of the cover glass and the front lens element when a specimen plane in contact with the cover glass is in focus) suitable for confocal microscopy.

In some instances, the objective lens has a working distance of about 1 mm. In some instances, the objective lens has a working distance of less than 1 mm.

In some cases, the objective lens has a working distance of less than or equal to about 100 μm, less than or equal to about 150 μm, less than or equal to about 200 μm, less than or equal to about 250 μm, less than or equal to about 300 μm, less than or equal to about 350 μm, less than or equal to about 400 μm, less than or equal to about 450 μm, less than or equal to about 500 μm, less than or equal to about 550 μm, less than or equal to about 600 μm, less than or equal to about 650 μm, less than or equal to about 700 μm, less than or equal to about 750 μm, less than or equal to about 800 μm, less than or equal to about 850 μm, less than or equal to about 900 μm, less than or equal to about or 950 μm. In some cases, less than or equal to about the objective lens has a working distance of less than 100 μm, less than or equal to about 150 μm, less than or equal to about 200 μm, less than or equal to about 250 μm, less than or equal to about 300 μm, less than or equal to about 350 μm, less than or equal to about 400 μm, less than or equal to about 450 μm, less than or equal to about 500 μm, less than or equal to about 550 μm, less than or equal to about 600 μm, less than or equal to about 650 μm, less than or equal to about 700 μm, less than or equal to about 750 μm, less than or equal to about 800 μm, less than or equal to about 850 μm, less than or equal to about 900 μm, less than or equal to about or 950 μm. In some instances, less than or equal to about the objective lens has a working distance of greater than 100 μm, less than or equal to about 150 μm, less than or equal to about 200 μm, less than or equal to about 250 μm, less than or equal to about 300 μm, less than or equal to about 350 μm, less than or equal to about 400 μm, less than or equal to about 450 μm, less than or equal to about 500 μm, less than or equal to about 550 μm, less than or equal to about 600 μm, less than or equal to about 650 μm, less than or equal to about 700 μm, less than or equal to about 750 μm, less than or equal to about 800 μm, less than or equal to about 850 μm, less than or equal to about 900 μm, less than or equal to about 950 μm, or less. In some cases, the objective lens has a working distance of greater than or equal to about 100 μm, greater than or equal to about 150 μm, greater than or equal to about 200 μm, greater than or equal to about 250 μm, greater than or equal to about 300 μm, greater than or equal to about 350 μm, greater than or equal to about 400 μm, greater than or equal to about 450 μm, greater than or equal to about 500 μm, greater than or equal to about 550 μm, greater than or equal to about 600 μm, greater than or equal to about 650 μm, greater than or equal to about 700 μm, greater than or equal to about 750 μm, greater than or equal to about 800 μm, greater than or equal to about 850 μm, greater than or equal to about 900 μm, greater than or equal to about or 950 μm. In some cases, greater than or equal to about the objective lens has a working distance of greater than 100 μm, greater than or equal to about 150 μm, greater than or equal to about 200 μm, greater than or equal to about 250 μm, greater than or equal to about 300 μm, greater than or equal to about 350 μm, greater than or equal to about 400 μm, greater than or equal to about 450 μm, greater than or equal to about 500 μm, greater than or equal to about 550 μm, greater than or equal to about 600 μm, greater than or equal to about 650 μm, greater than or equal to about 700 μm, greater than or equal to about 750 μm, greater than or equal to about 800 μm, greater than or equal to about 850 μm, greater than or equal to about 900 μm, greater than or equal to about or 950 μm. In some instances, greater than or equal to about the objective lens has a working distance of greater than 100 μm, greater than or equal to about 150 μm, greater than or equal to about 200 μm, greater than or equal to about 250 μm, greater than or equal to about 300 μm, greater than or equal to about 350 μm, greater than or equal to about 400 μm, greater than or equal to about 450 μm, greater than or equal to about 500 μm, greater than or equal to about 550 μm, greater than or equal to about 600 μm, greater than or equal to about 650 μm, greater than or equal to about 700 μm, greater than or equal to about 750 μm, greater than or equal to about 800 μm, greater than or equal to about 850 μm, greater than or equal to about 900 μm, greater than or equal to about 950 μm, or more.

In some cases, the objective lens has a working distance of about 950 μm. In some instances, the objective lens has a working distance of about 800 μm. In some cases, the objective lens has a working distance of about 600 μm. In some cases, the objective lens has a working distance of about 300 μm. In some instances, the objective lens has a working distance of about 290 μm. In some instances, the objective lens has a working distance of about 170 μm. In some cases, the objective lens has a working distance of about 150 μm. In some cases, the objective lens has a working distance of about 130 μm. In some instances, the objective has a resolution of less than 2.00 μm at a relevant wavelength of light. In some cases, the objective has a resolution of about 0.20 μm, 0.30 μm, 0.40 μm, 0.50 μm, 0.60 μm, 0.70 μm, 0.80 μm, 0.90 μm, 1.00 μm, 1.10 μm, 1.20 μm, 1.30 μm, or 1.40 μm at a relevant wavelength of light. In some instances, the objective has a resolution of less than 0.20 μm, 0.30 μm, 0.40 μm, 0.50 μm, 0.60 μm, 0.70 μm, 0.80 μm, 0.90 μm, 1.00 μm, 1.10 μm, 1.20 μm, 1.30 μm, or 1.40 μm at a relevant wavelength of light. In some cases, the objective has a resolution of greater than 0.20 μm, 0.30 μm, 0.40 μm, 0.50 μm, 0.60 μm, 0.70 μm, 0.80 μm, 0.90 μm, 1.00 μm, 1.10 μm, 1.20 μm, 1.30 μm, or 1.40 μm at a relevant wavelength of light.

In some instances, the objective lens is selected from: CF175 Apochromat LWD 20XC W, CFI Apochromat Lambda S 40XCWI, CFI75 LWD 16XW, CFI Apochromat NIR 60XW, CF175 Apochromat 25XCW 1300, CFI Apochromat LWD Lambda S 20XCWI, CFI Apochromat NIR 40XW, CFI Plan Apochromat 10XC Glyc, CF190 20XC Glyc, CFI Plan Fluor 20XCMI, CFI Plan Apochromat Lambda D 10X, or CFI Plan Apochromat Lambda S 25XC Sil, CFI Plan Apochromat Lambda S 40XC Sil.

In some cases, the visualization device (e.g., microscope device) described herein comprises a light source. The light source may be any source capable of delivering light of the relevant excitation wavelength to the sample.

In some cases, the light source is a laser source or a non-coherent source.

In some instances, the light source comprises a laser source. In some instances, the light source comprises a plurality of laser sources. In some cases, the light source comprises lasers covering the optical spectrum from the near IR to the near UV.

In some cases, the light source is an argon-ion laser.

In some cases, the light is delivered as a collimated beam with a specified beam diameter.

In some instances, the light source is a non-coherent source. In some cases, the light source is an arc discharge lamp. In some instances, the light source is a broadband source.

In some instances, the light source is selected from: a xenon lamp, a mercury lamp, a light-emitting diode (LED), or a halogen lamp.

In some cases, the light source is an LED with a peak wavelength selected from: 365 nm, 400 nm, 450 nm, and 520 nm.

In some cases, the light source is an LED with a peak wavelength selected from: 395 nm, 430 nm, 470 nm, 555 nm, 585 nm 605 nm, 612 nm, 633 nm, 660 nm, and 880 nm.

In some instances, the light source is filtered (e.g., with a bandpass filter). In some cases, the light source is unfiltered.

In some instances, the visualization device (e.g., microscope device) described herein comprises an additional optical element.

In some instances, the microscope device comprises an optical filter. In some instances, the optical filter is an excitation filter, an emission filter, or a dichroic filter.

In some cases, the optical filter is a neutral-density filter, a polarizing filter, a UV-filter, or a near-IR filter.

In some cases, the optical filter efficiently transmits fluorescent light, while blocking the wavelength of the light source. In some instances, the optical filter ensures the illumination is near monochromatic and at the correct wavelength.

In some cases, the microscope device comprises an optical filter specific to the detection tag (e.g., fluorescent label) of the sample.

In some instances, the microscope device comprises an emission filter. In some instances, the emission filter efficiently transmits the fluorescent light while attenuating light at other wavelengths. In some instances, the optical filter blocks ambient light. In some cases, emission filter ensures none of the excitation light source reaches the detector.

In some cases, the microscope device comprises a single dichroic mirror which performs the function of both the excitation filter and the emission filter. In some cases, the microscope device comprises several dichroics, excitation sources and cameras configured for concurrent operation.

In some instances, the microscope device comprises a linear variable filter. In some instances, the microscope device comprises a filter cube. In some instances, the microscope device comprises a plurality of optical filters. In some cases, the microscope device does not comprise a filter.

In some cases, the microscope device comprises a photomultiplier tube (PMT).

The systems of the present invention comprise one or more sensors capable of detecting a signal (e.g., light) from a sample.

In some instances, the sensor comprises a charge-coupled device (CCD), an electron multiplying charge coupled device (EECCD), a complementary metal-oxide-semiconductor (CMOS) sensor or a scientific complementary metal-oxide-semiconductor (sCMOS). In some cases, the sensor comprises a charge-coupled device (CCD). In some cases, the sensor comprises an electron multiplying charge coupled device (EECCD). In some instances, the sensor comprises a complementary metal-oxide-semiconductor (CMOS). In some instances, the sensor comprises a scientific complementary metal-oxide-semiconductor (sCMOS).

In some instances, the sensor comprises a rolling shutter functionality. That is, the sensor may be capable of rolling shutter video. Rolling shutter detectors may start the exposure of each pixel row at a slightly different time. If, for example, the sample is moving rapidly (e.g., rapidly compared to the frame rate of the sensor), then image distortion can occur.

In some cases, fluorescence emission from a sample (e.g., a sample described herein) is focused on a CMOS or sCMOS detector comprising a rolling shutter functionality.

In some cases, the timing between the sample illumination and the rolling shutter exposure is synchronized (e.g., synchronized by methods known in the art).

In some cases, the rolling shutter exposes pixels in a row-by-row fashion during a frame exposure.

In some instances, the rolling shutter is linear (e.g., exposes pixels in one direction).

In some cases, the sensor comprises a global shutter functionality. In the global shutter method of detection, all pixels may be exposed to light simultaneously. In some cases, the sensor does not comprise a global shutter functionality.

In some cases, the sensor has a diagonal length of about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm 28 mm, about 29 mm, or about 30 mm.

In some cases, the sensor has a diagonal length less than about 4 mm, less than about 5 mm, less than about 6 mm, less than about 7 mm, less than about 8 mm, less than about 9 mm, less than about 10 mm, less than about 11 mm, less than about 12 mm, less than about 13 mm, less than about 14 mm, less than about 15 mm, less than about 16 mm, less than about 17 mm, less than about 18 mm, less than about 19 mm, less than about 20 mm, less than about 21 mm, less than about 22 mm, less than about 23 mm, less than about 24 mm, less than about 25 mm, less than about 26 mm, less than about 27 mm 28 mm, less than about 29 mm, less than about 30 mm, less than about 50 mm, or less.

In some instances, the sensor has a diagonal length greater than about 4 mm, greater than about 5 mm, greater than about 6 mm, greater than about 7 mm, greater than about 8 mm, greater than about 9 mm, greater than about 10 mm, greater than about 11 mm, greater than about 12 mm, greater than about 13 mm, greater than about 14 mm, greater than about 15 mm, greater than about 16 mm, greater than about 17 mm, greater than about 18 mm, greater than about 19 mm, greater than about 20 mm, greater than about 21 mm, greater than about 22 mm, greater than about 23 mm, greater than about 24 mm, greater than about 25 mm, greater than about 26 mm, greater than about 27 mm 28 mm, greater than about 29 mm, greater than about or 30 mm, greater than about 50 mm, or greater.

In some cases, the sensor comprises pixels of about 1 μm², about 2 μm², about 3 μm², about 4 μm², about 5 μm², about 6 μm², about 7 μm², about 8 μm², about 9 μm², about 10 μm², about 11 μm², about 12 μm², about 13 μm², about 14 μm², about 15 μm², about 16 μm², about 17 μm², about 18 μm², about 19 μm², about 20 μm², about 21 μm², about 22 μm², about 23 μm², about 24 μm², about 25 μm², about 26 μm², about 27 μm², about 28 μm², about 29 μm², about 30 μm², about 31 μm², about 32 μm², about 33 μm², about 34 μm², about 35 μm², about 36 μm², about 37 μm², about 38 μm², about 39 μm², or about 40 μm².

In some instances, the sensor has a pixel size of less than 2 μm², less than about 3 μm², less than about 4 μm², less than about 5 μm², less than about 6 μm², less than about 7 μm², less than about 8 μm², less than about 9 μm², less than about 10 μm², less than about 11 μm², less than about 12 μm², less than about 13 μm², less than about 14 μm², less than about 15 μm², less than about 16 μm², less than about 17 μm², less than about 18 μm², less than about 19 μm², less than about 20 μm², less than about 21 μm², less than about 22 μm², less than about 23 μm², less than about 24 μm², less than about 25 μm², less than about 26 μm², less than about 27 μm², less than about 28 μm², less than about 29 μm², less than about 30 μm², less than about 31 μm², less than about 32 μm², less than about 33 μm², less than about 34 μm², less than about 35 μm², less than about 36 μm², less than about 37 μm², less than about 38 μm², less than about 39 μm², less than about 40 μm², or less.

In some cases, the sensor has a pixel size of greater than 2 μm², greater than about 3 μm², greater than about 4 μm², greater than about 5 μm², greater than about 6 μm², greater than about 7 μm², greater than about 8 μm², greater than about 9 μm², greater than about 10 μm², greater than about 11 μm², greater than about 12 μm², greater than about 13 μm², greater than about 14 μm², greater than about 15 μm², greater than about 16 μm², greater than about 17 μm², greater than about 18 μm², greater than about 19 μm², greater than about 20 μm², greater than about 21 μm², greater than about 22 μm², greater than about 23 μm², greater than about 24 μm², greater than about 25 μm², greater than about 26 μm², greater than about 27 μm², greater than about 28 μm², greater than about 29 μm², greater than about 30 μm², greater than about 31 μm², greater than about 32 μm², greater than about 33 μm², greater than about 34 μm², greater than about 35 μm², greater than about 36 μm², greater than about 37 μm², greater than about 38 μm², greater than about 39 μm², greater than about 40 μm², greater than about 41 μm², greater than about 42 μm², greater than about 43 μm², greater than about 44 μm², greater than about 45 μm², greater than about 46 μm², greater than about 47 μm², greater than about 48 μm², greater than about 49 μm², greater than about 50 μm², or more

In some cases, the sensor has a pixel size of about 14 μm². In some instances, the sensor has a pixel size of about 25 μm². In some cases, the sensor has a pixel size of about 30 μm². In some instances, the sensor has a pixel size of about 35 μm². In some instances, the sensor has a pixel size of about 40 μm². In some instances, the sensor has a pixel size of about 42 μm². In some cases, the sensor has a pixel size of about 56 μm².

In some instances, the sensor has a pixel size of less than 25 μm². In some cases, the sensor has a pixel size of less than 30 μm². In some instances, the sensor has a pixel size of less than 35 μm². In some instances, the sensor has a pixel size of less than 40 μm². In some cases, the sensor has a pixel size of less than 42 μm². In some instances, the sensor has a pixel size of less than 45 μm².

In some instances, the sensor has a pixel size of greater than 25 μm². In some cases, the sensor has a pixel size of greater than 30 μm². In some instances, the sensor has a pixel size of greater than 35 μm². In some cases, the sensor has a pixel size of greater than 40 μm². In some instances, the sensor has a pixel size of greater than 42 μm². In some instances, the sensor has a pixel size of greater than 45 μm².

In some cases, the sensor height on the sample is about 200 μm, 400 μm, 1,000 μm, 2,000 μm, or 4,000 μm. In some cases, the sensor height on the sample is less than about 200 μm, 400 μm, 1,000 μm, 2,000 μm, or 4,000 μm. In some instances, the sensor height on the sample is greater than about 200 μm, 400 μm, 1,000 μm, 2,000 μm, or 4,000 μm.

In some cases, the sensor has a surface area of about 20 mm², about 22 mm², about 24 mm², about 26 mm², about 28 mm², about 30 mm², about 32 mm², about 34 mm², about 36 mm², about 38 mm², about 40 mm², about 42 mm², about 44 mm², about 46 mm², about 48 mm², about 50 mm², about 52 mm², about 54 mm², about 56 mm², about 58 mm², about 60 mm², about 62 mm², about 64 mm², about 66 mm², about 68 mm², about 70 mm², about 72 mm², about 74 mm², about 76 mm², about 78 mm², about 80 mm², about 82 mm², about 84 mm², about 86 mm², about 88 mm², about 90 mm², about 92 mm², about 94 mm², about 96 mm², about 98 mm², about 100 mm², about 105 mm², about 110 mm², about 115 mm², about 120 mm², about 125 mm², about 130 mm², about 135 mm², about 140 mm², about 145 mm², about 150 mm², about 155 mm², about 160 mm², about 165 mm², about 170 mm², about 175 mm², about 180 mm², about 185 mm², about 190 mm², about 195 mm², about 200 mm², about 500 mm², about 1000 mm², about 1500 mm², about 2000 mm², or about 2500 mm².

In some instances, the sensor has a surface area of less than about 20 mm², less than about 22 mm², less than about 24 mm², less than about 26 mm², less than about 28 mm², less than about 30 mm², less than about 32 mm², less than about 34 mm², less than about 36 mm², less than about 38 mm², less than about 40 mm², less than about 42 mm², less than about 44 mm², less than about 46 mm², less than about 48 mm², less than about 50 mm², less than about 52 mm², less than about 54 mm², less than about 56 mm², less than about 58 mm², less than about 60 mm², less than about 62 mm², less than about 64 mm², less than about 66 mm², less than about 68 mm², less than about 70 mm², less than about 72 mm², less than about 74 mm², less than about 76 mm², less than about 78 mm², less than about 80 mm², less than about 82 mm², less than about 84 mm², less than about 86 mm², less than about 88 mm², less than about 90 mm², less than about 92 mm², less than about 94 mm², less than about 96 mm², less than about 98 mm², less than about 100 mm², less than about 105 mm², less than about 110 mm², less than about 115 mm², less than about 120 mm², less than about 125 mm², less than about 130 mm², less than about 135 mm², less than about 140 mm², less than about 145 mm², less than about 150 mm², less than about 155 mm², less than about 160 mm², less than about 165 mm², less than about 170 mm², less than about 175 mm², less than about 180 mm², less than about 185 mm², less than about 190 mm², less than about 195 mm², less than about 200 mm², less than about 500 mm², less than about 1000 mm², less than about 1500 mm², less than about 2000 mm², less than about 2500 mm², or less.

In some instances, the sensor has a surface area of greater than about 20 mm², greater than about 22 mm², greater than about 24 mm², greater than about 26 mm², greater than about 28 mm², greater than about 30 mm², greater than about 32 mm², greater than about 34 mm², greater than about 36 mm², greater than about 38 mm², greater than about 40 mm², greater than about 42 mm², greater than about 44 mm², greater than about 46 mm², greater than about 48 mm², greater than about 50 mm², greater than about 52 mm², greater than about 54 mm², greater than about 56 mm², greater than about 58 mm², greater than about 60 mm², greater than about 62 mm², greater than about 64 mm², greater than about 66 mm², greater than about 68 mm², greater than about 70 mm², greater than about 72 mm², greater than about 74 mm², greater than about 76 mm², greater than about 78 mm², greater than about 80 mm², greater than about 82 mm², greater than about 84 mm², greater than about 86 mm², greater than about 88 mm², greater than about 90 mm², greater than about 92 mm², greater than about 94 mm², greater than about 96 mm², greater than about 98 mm², greater than about 100 mm², greater than about 105 mm², greater than about 110 mm², greater than about 115 mm², greater than about 120 mm², greater than about 125 mm², greater than about 130 mm², greater than about 135 mm², greater than about 140 mm², greater than about 145 mm², greater than about 150 mm², greater than about 155 mm², greater than about 160 mm², greater than about 165 mm², greater than about 170 mm², greater than about 175 mm², greater than about 180 mm², greater than about 185 mm², greater than about 190 mm², greater than about 195 mm², greater than about 200 mm², or more.

In some cases, the sensor has a surface area of about 46 mm². In some instances, the sensor has a surface area of about 52 mm². In some cases, the sensor has a surface area of about 56 mm². In some instances, the sensor has a surface area of about 170 mm². In some cases, the sensor has a surface area of about 175 mm².

In some cases, the sensor has a maximum frame rate (e.g., frame rate over the entire sensor) of about 5 Hz, about 10 Hz, about 20 Hz, about 25 Hz, about 30 Hz, about 35 Hz, about 40 Hz, about 45 Hz, about 50 Hz, about 55 Hz, about 60 Hz, about 65 Hz, about 70 Hz, about 75 Hz, about 80 Hz, about 85 Hz, about 90 Hz, about 95 Hz, about 100 Hz, about 110 Hz, about 120 Hz, about 130 Hz, about 140 Hz, about 150 Hz, about 160 Hz, about 170 Hz, about 180 Hz, about 119 Hz, about 200 Hz, about 210 Hz, about 220 Hz, about 230 Hz, about 240 Hz, about 250 Hz, about 260 Hz, about 270 Hz, about 280 Hz, about 290 Hz, about 300 Hz, about 350 Hz, or about 500 Hz at an exposure time of 1 ms.

In some instances, the sensor has a maximum frame rate (e.g., frame rate over the entire sensor) of less than about 5 Hz, less than about 10 Hz, less than about 20 Hz, less than about 25 Hz, less than about 30 Hz, less than about 35 Hz, less than about 40 Hz, less than about 45 Hz, less than about 50 Hz, less than about 55 Hz, less than about 60 Hz, less than about 65 Hz, less than about 70 Hz, less than about 75 Hz, less than about 80 Hz, less than about 85 Hz, less than about 90 Hz, less than about 95 Hz, less than about 100 Hz, less than about 110 Hz, less than about 120 Hz, less than about 130 Hz, less than about 140 Hz, less than about 150 Hz, less than about 160 Hz, less than about 170 Hz, less than about 180 Hz, less than about 119 Hz, less than about 200 Hz, less than about 210 Hz, less than about 220 Hz, less than about 230 Hz, less than about 240 Hz, less than about 250 Hz, less than about 260 Hz, less than about 270 Hz, less than about 280 Hz, less than about 290 Hz, less than about 300 Hz, less than about 350 Hz, 500 Hz, or more at an exposure time of 1 ms.

In some instances, the sensor has a maximum frame rate (e.g., frame rate over the entire detector) of greater than about 5 Hz, greater than about 10 Hz, greater than about 20 Hz, greater than about 25 Hz, greater than about 30 Hz, greater than about 35 Hz, greater than about 40 Hz, greater than about 45 Hz, greater than about 50 Hz, greater than about 55 Hz, greater than about 60 Hz, greater than about 65 Hz, greater than about 70 Hz, greater than about 75 Hz, greater than about 80 Hz, greater than about 85 Hz, greater than about 90 Hz, greater than about 95 Hz, greater than about 100 Hz, greater than about 110 Hz, greater than about 120 Hz, greater than about 130 Hz, greater than about 140 Hz, greater than about 150 Hz, greater than about 160 Hz, greater than about 170 Hz, greater than about 180 Hz, greater than about 119 Hz, greater than about 200 Hz, greater than about 210 Hz, greater than about 220 Hz, greater than about 230 Hz, greater than about 240 Hz, greater than about 250 Hz, greater than about 260 Hz, greater than about 270 Hz, greater than about 280 Hz, greater than about 290 Hz, greater than about 300 Hz, greater than about 350 Hz, 500 Hz, or more at an exposure time of 1 ms.

In some instances, the sensor has a maximum frame rate (e.g., frame rate over the entire sensor) of about 30 Hz at an exposure time of approximately 33 ms. In some cases, the sensor has a maximum frame rate (e.g., frame rate over the entire sensor) of about 50 Hz at an exposure time of about 20 ms.

In some cases, the sensor has an exposure time of about 0.1 ms, 0.2 ms, 0.3, ms, 0.4 ms, 0.5 ms, 0.6 ms, 0.7 ms, 0.8 ms, 0.9 ms, 1.0 ms, 1.1 ms, 1.2 ms, 1.3 ms, 1.4 ms, 1.5 ms, 1.6 ms, 1.7 ms, 1.8 ms, 1.9 ms, 2 ms, 3 ms, 4 ms, 5 ms, 6 ms, 7 ms, 8 ms, 9 ms, 10 ms, 12 ms, 14 ms, 16 ms, 18 ms, 20 ms, 25 ms, 30 ms, 35 ms, 40 ms, 45 ms, 50 ms, 55 ms, 60 ms, 65 ms, 70 ms, 75 ms, 80 ms, 85 ms, 90 ms, 95 ms, 100 ms, 110 ms, 120 ms, 130 ms, 140 ms, 150 ms, 160 ms, 170 ms, 180 ms, 190 ms, 200 ms, 220 ms, 240 ms, 260 ms, 280 ms, 300 ms, 320 ms, 340 ms, 360 ms, 380 ms, 400 ms, 420 ms, 440 ms, 460 ms, 480 ms, or 500 ms.

In some cases, the sensor has an exposure time of less than about 0.1 ms, 0.2 ms, 0.3, ms, 0.4 ms, 0.5 ms, 0.6 ms, 0.7 ms, 0.8 ms, 0.9 ms, 1.0 ms, 1.1 ms, 1.2 ms, 1.3 ms, 1.4 ms, 1.5 ms, 1.6 ms, 1.7 ms, 1.8 ms, 1.9 ms, 2 ms, 3 ms, 4 ms, 5 ms, 6 ms, 7 ms, 8 ms, 9 ms, 10 ms, 12 ms, 14 ms, 16 ms, 18 ms, 20 ms, 25 ms, 30 ms, 35 ms, 40 ms, 45 ms, 50 ms, 55 ms, 60 ms, 65 ms, 70 ms, 75 ms, 80 ms, 85 ms, 90 ms, 95 ms, 100 ms, 110 ms, 120 ms, 130 ms, 140 ms, 150 ms, 160 ms, 170 ms, 180 ms, 190 ms, 200 ms, 220 ms, 240 ms, 260 ms, 280 ms, 300 ms, 320 ms, 340 ms, 360 ms, 380 ms, 400 ms, 420 ms, 440 ms, 460 ms, 480 ms, or 500 ms.

In some instances, the sensor has an exposure time of greater than about 0.1 ms, 0.2 ms, 0.3, ms, 0.4 ms, 0.5 ms, 0.6 ms, 0.7 ms, 0.8 ms, 0.9 ms, 1.0 ms, 1.1 ms, 1.2 ms, 1.3 ms, 1.4 ms, 1.5 ms, 1.6 ms, 1.7 ms, 1.8 ms, 1.9 ms, 2 ms, 3 ms, 4 ms, 5 ms, 6 ms, 7 ms, 8 ms, 9 ms, 10 ms, 12 ms, 14 ms, 16 ms, 18 ms, 20 ms, 25 ms, 30 ms, 35 ms, 40 ms, 45 ms, 50 ms, 55 ms, 60 ms, 65 ms, 70 ms, 75 ms, 80 ms, 85 ms, 90 ms, 95 ms, 100 ms, 110 ms, 120 ms, 130 ms, 140 ms, 150 ms, 160 ms, 170 ms, 180 ms, 190 ms, 200 ms, 220 ms, 240 ms, 260 ms, 280 ms, 300 ms, 320 ms, 340 ms, 360 ms, 380 ms, 400 ms, 420 ms, 440 ms, 460 ms, 480 ms, or 500 ms.

In some cases, the sensor is a Zyla 4.2PLUS, Zyla Sona-6, Zyla Sona-12, Andor Neo, pco.edge 26 CLHS, pco.edge 10 bi CLHS, pco.edge 10 bi LT CLHS, pco.edge 5.5 CLHS, pco.edge 4.2 CLHS, Thorlabs CC215MU, or Hamamatsu ORCA-Flash4.0 V3.

In some instances, the sensor is a Sony IMX571BLR-J.

In some instances, the sensor is a Teledyne Kinetix sensor.

In some instances, systems of the present disclosure comprise a stage device that provides or is capable of continuous motion in the z-direction. In some cases, the system may comprise a stage to provide continuous motion in the z-direction. In some instances, the stage is capable of continuous motion in the z-direction. In some instances, the objective lens is capable of continuous motion in the z-direction. The continuous motion of the stage or objective may allow for constant imaging acquisition, eliminating the dead time typically required in volumetric imaging.

In some cases, the stage or objective is capable of continuous motion in the z-direction at a velocity of about 0.5 μm s⁻¹, 1 μm s⁻¹, 2 μm s⁻¹, 3 μm s⁻¹, 4 μm s⁻¹, 5 μm s⁻¹, 6 μm s⁻¹, 7 μm s⁻¹, 8 μm s⁻¹, 9 μm s⁻¹, 10 μm s⁻¹, 11 μm s⁻¹, 12 μm s⁻¹, 13 μm s⁻¹, 14 μm s⁻¹, 15 μm s⁻¹, 16 μm s⁻¹, 17 μm s⁻¹, 18 μm s⁻¹, 19 μm s⁻¹, 20 μm s⁻¹, 25 μm s⁻¹, 30 μm s⁻¹, 35 μm s⁻¹, 40 μm s⁻¹, 45 μm s⁻¹, 50 μm s⁻¹, 55 μm s⁻¹, 60 μm s⁻¹, 65 μm s⁻¹, 70 μm s⁻¹, 75 μm s⁻¹, 80 μm s⁻¹, 90 μm s⁻¹, 95 μm s⁻¹, 100 μm s⁻¹, 110 μm s⁻¹, 120 μm s⁻¹, 130 μm s⁻¹, 140 μm s⁻¹, 150 μm s⁻¹, 160 μm s⁻¹, 170 μm s⁻¹, 180 μm s⁻¹, 190 μm s⁻¹, or 200 μm s⁻¹.

In some cases, the stage or objective is capable of continuous motion in the z-direction at a velocity of less than 0.5 μm s⁻¹, 1 μm s⁻¹, 2 μm s⁻¹, 3 μm s⁻¹, 4 μm s⁻¹, 5 μm s⁻¹, 6 μm s⁻¹, 7 μm s 1, 8 μm s⁻¹, 9 μm s 1, 10 μm s⁻¹, 11 μm s⁻¹, 12 μm s⁻¹, 13 μm s⁻¹, 14 μm s⁻¹, 15 μm s⁻¹, 16 μm s⁻¹, 17 μm s⁻¹, 18 μm s 1, 19 μm s⁻¹, 20 μm s⁻¹, 25 μm s⁻¹, 30 μm s⁻¹, 35 μm s⁻¹, 40 μm s⁻¹, 45 μm s⁻¹, 50 μm s 1, 55 μm s⁻¹, 60 μm s⁻¹, 65 μm s⁻¹, 70 μm s⁻¹, 75 μm s⁻¹, 80 μm s⁻¹, 90 μm s⁻¹, 95 μm s⁻¹, 100 μm s⁻¹, 110 μm s⁻¹, 120 μm s⁻¹, 130 μm s⁻¹, 140 μm s⁻¹, 150 μm s⁻¹, 160 μm s⁻¹, 170 μm s⁻¹, 180 μm s⁻¹, 190 μm s⁻¹, or 200 μm s⁻¹.

In some cases, the stage or objective is capable of continuous motion in the z-direction at a velocity of greater than 0.5 μm s⁻¹, 1 μm s⁻¹, 2 μm s⁻¹, 3 μm s⁻¹, 4 μm s⁻¹, 5 μm s⁻¹, 6 μm s⁻¹, 7 μm s⁻¹, 8 μm s⁻¹, 9 μm s⁻¹, 10 μm s⁻¹, 11 μm s⁻¹, 12 μm s⁻¹, 13 μm s⁻¹, 14 μm s⁻¹, 15 μm s⁻¹, 16 μm s 1, 17 μm s 1, 18 μm s⁻¹, 19 μm s⁻¹, 20 μm s 1, 25 μm s⁻¹, 30 μm s⁻¹, 35 μm s⁻¹, 40 μm s⁻¹, 45 μm s⁻¹, 50 μm s⁻¹, 55 μm s⁻¹, 60 μm s⁻¹, 65 μm s⁻¹, 70 μm s⁻¹, 75 μm s⁻¹, 80 μm s⁻¹, 90 μm s⁻¹, 95 μm s⁻¹, 100 μm s⁻¹, 110 μm s⁻¹, 120 μm s⁻¹, 130 μm s⁻¹, 140 μm s⁻¹, 150 μm s⁻¹, 160 μm s⁻¹, 170 μm s 1, 180 μm s⁻¹, 190 μm s⁻¹, or 200 μm s⁻¹.

In some cases, the stage or objective is capable of continuous motion in the z-direction at a velocity of about 10 μm s⁻¹. In some cases, the stage is capable of continuous motion in the z-direction at a velocity of about 100 μm s⁻¹.

In some instances, the stage or objective is capable of continuous motion in the z-direction at a velocity of greater than 10 μm s⁻¹. In some instances, the stage is capable of continuous motion in the z-direction at a velocity of greater than 100 μm s⁻¹.

In some instances, the stage or objective has a z-range (e.g., total movable distance in the z-direction) of about 100 μm, 200 μm, 300 μm, 400 μm, 500 μm, 600 μm, 700 μm, 800 μm, 900 μm, 1000 μm, 1200 μm, 1400 μm, 1600 μm, 1800 μm, 2000 μm, 2500 μm, or 3000 μm.

In some cases, the stage or objective has a z-range (e.g., total movable distance in the z-direction) of less than about 100 μm, 200 μm, 300 μm, 400 μm, 500 μm, 600 μm, 700 μm, 800 μm, 900 μm, 1000 μm, 1200 μm, 1400 μm, 1600 μm, 1800 μm, 2000 μm, 2500 μm, or 3000 μm.

In some instances, the objective stage has a z-range (e.g., total movable distance in the z-direction) of greater than about 100 μm, 200 μm, 300 μm, 400 μm, 500 μm, 600 μm, 700 μm, 800 μm, 900 μm, 1000 μm, 1200 μm, 1400 μm, 1600 μm, 1800 μm, 2000 μm, 2500 μm, or 3000 μm.

In some instances, the stage or objective is further capable of motion in a plurality of directions. In some cases, the stage is further capable of motion in the x-direction or y direction. In some instances, the stage is further capable of motion in the x-direction. In some cases, the stage is further capable of motion in the y-direction.

In some cases, the stage is selected from: Dover Motion DOF-5 vertical Z-stage, Dover Motion ZE Vertical Stage, Pimars P-561, Pimars P-562, or Pimars P-563.

In some instances, the stage is an ASI Stage XY stage with piezo insert (e.g., PZ-2000 FT).

In some instances, the stage or objective further comprises an element or component that reduces noise. In some cases, the stage, substrate, or objective lens, or any combination thereof, may comprise one or more noise reducing feature, e.g., a dampener, gimbal, or ball bearing, or any combination thereof, to reduce noise from the movement of the stage, substrate, or objective lens, or any combination thereof.

The systems of the present disclosure may comprise a control unit comprising hardware and software relevant to the volumetric imaging of a sample (e.g., a sample described herein).

In some cases, the control unit comprises a computer comprising a microprocessor.

In some cases, the control unit comprises an image processing software (e.g., ImageJ or LAS X).

### Kits

Provided herein are compositions related to identifying and/or detecting one or more analytes (e.g., detecting an analyte in situ). The one or more analytes may comprise any one of the analytes described herein. For example, in some cases, the one or more analytes may comprise nucleic acid, polypeptides, or a combination thereof. In some embodiments, the compositions may be included in a kit with instructions for identifying and/or detecting the one or more analytes based on any of the methods described herein.

The kits described herein may comprise one or more samples, as described herein. The one or more samples may comprise a variety of formats. In some cases, the one or more samples may comprise a tissue sample (e.g., a tissue slice). The tissue slice may comprise a microarray. In some cases, the one or more samples may be provided on a substrate (e.g., a slide, a well plate, or any combination thereof.).

The kits described herein may comprise one or more substrates, as described herein. For example, a kit may comprise a slide, a coverslip, a well-plate, a flow cell, or any combination thereof.

The kits described herein may comprise one or more binding moieties. The one or more binding moieties may comprise one or a combination of any one of the binding moieties described herein. In some cases, the one or more binding moieties may be stored in a container (e.g., a tube, a vial, a box, an ampule, or any combination thereof. The one or more binding moieties may be stored at various temperatures including at least about −80° C., at least about −70° C., at least about −60° C., at least about −50° C., at least about −40° C., at least about −30° C., at least about −20° C., at least about −10° C., at least about 0° C., at least about 10° C., at least about 20° C., or at least about 30° C. In some embodiments, the one or more binding moieties may be stored at most about −80° C., at most about −70° C., at most about −60° C., at most about −50° C., at most about −40° C., at most about −30° C., at most about −20° C., at most about −10° C., at most about 0° C., at most about 10° C., at most about 20° C., or at most about 30° C. The one or more binding moieties may be stored in solution, lyophilized, frozen, flash-frozen, cryopreserved, or a combination thereof. In some cases, the ligase may be stored in combination with a glycerol solution.

The kits described herein may comprise one or more ligases. The one or more ligases may comprise one or a combination of any one of the ligases described herein. In some cases, the one or more ligases may be stored in a container (e.g., a tube, a vial, a box, an ampule, or any combination thereof. The one or more ligases may be stored at various temperatures including at least about −80° C., at least about −70° C., at least about −60° C., at least about −50° C., at least about-40° C., at least about −30° C., at least about −20° C., at least about −10° C., at least about 0° C., at least about 10° C., at least about 20° C., or at least about 30° C. In some embodiments, the one or more ligases may be stored at most about −80° C., at most about −70° C., at most about −60° C., at most about −50° C., at most about −40° C., at most about −30° C., at most about −20° C., at most about −10° C., at most about 0° C., at most about 10° C., at most about 20° C., or at most about 30° C. The one or more ligases may be stored in solution, lyophilized, frozen, flash-frozen, cryopreserved, or a combination thereof. In some cases, the one or more ligases may be stored in combination with a glycerol solution.

The kits described herein may comprise one or more polymerases. The one or more polymerases may comprise one or a combination of any one of the polymerases described herein. In some cases, the one or more polymerases may be stored in a container (e.g., a tube, a vial, a box, an ampule, or any combination thereof. The one or more polymerases may be stored at various temperatures including at least about −80° C., at least about −70° C., at least about −60° C., at least about −50° C., at least about −40° C., at least about −30° C., at least about −20° C., at least about −10° C., at least about 0° C., at least about 10° C., at least about 20° C., or at least about 30° C. In some embodiments, the one or more polymerases may be stored at most about −80° C., at most about-70° C., at most about −60° C., at most about −50° C., at most about −40° C., at most about −30° C., at most about −20° C., at most about −10° C., at most about 0° C., at most about 10° C., at most about 20° C., or at most about 30° C. The one or more polymerases may be stored in solution, lyophilized, frozen, flash-frozen, cryopreserved, or a combination thereof. In some cases, the one or more polymerases may be stored in combination with a glycerol solution.

The kits described herein may comprise one or more probes. The one or more probes may comprise one or a combination of any one of the probes described herein. In some cases, the one or more probes may be stored in a container (e.g., a tube, a vial, a box, an ampule, or any combination thereof. The one or more probes may be stored at various temperatures including at least about −80° C., at least about −70° C., at least about −60° C., at least about −50° C., at least about-40° C., at least about −30° C., at least about −20° C., at least about −10° C., at least about 0° C., at least about 10° C., at least about 20° C., or at least about 30° C. In some embodiments, the one or more probes may be stored at most about −80° C., at most about −70° C., at most about −60° C., at most about −50° C., at most about −40° C., at most about −30° C., at most about −20° C., at most about −10° C., at most about 0° C., at most about 10° C., at most about 20° C., or at most about 30° C. The one or more probes may be stored in solution, lyophilized, frozen, flash-frozen, cryopreserved, or a combination thereof. In some cases, the one or more probes may be stored in combination with a glycerol solution.

The kits described herein may comprise one or more detection probes. The one or more detection probes may comprise one or a combination of any one of the detection probes described herein. In some cases, the one or more detection probes may be stored in a container (e.g., a tube, a vial, a box, an ampule, or any combination thereof. The one or more detection probes may be stored at various temperatures including at least about −80° C., at least about −70° C., at least about-60° C., at least about −50° C., at least about −40° C., at least about −30° C., at least about −20° C., at least about −10° C., at least about 0° C., at least about 10° C., at least about 20° C., or at least about 30° C. In some embodiments, the one or more detection probes may be stored at most about −80° C., at most about −70° C., at most about −60° C., at most about −50° C., at most about −40° C., at most about −30° C., at most about −20° C., at most about −10° C., at most about 0° C., at most about 10° C., at most about 20° C., or at most about 30° C. The one or more detection probes may be stored in solution, lyophilized, frozen, flash-frozen, cryopreserved, or a combination thereof. In some cases, the one or more detection probes may be stored in combination with a glycerol solution.

The kits described herein may comprise one or more compaction agents. The one or more compaction agents may comprise one or a combination of any one of the compaction agents described herein. In some cases, the one or more compaction agents may be stored in a container (e.g., a tube, a vial, a box, an ampule, or any combination thereof. The one or more compaction agents may be stored at various temperatures including at least about −80° C., at least about −70° C., at least about −60° C., at least about −50° C., at least about −40° C., at least about −30° C., at least about −20° C., at least about −10° C., at least about 0° C., at least about 10° C., at least about 20° C., or at least about 30° C. In some embodiments, the one or more compaction agents may be stored at most about −80° C., at most about −70° C., at most about −60° C., at most about −50° C., at most about −40° C., at most about −30° C., at most about −20° C., at most about −10° C., at most about 0° C., at most about 10° C., at most about 20° C., or at most about 30° C. The one or more compaction agents may be stored in solution, lyophilized, frozen, flash-frozen, cryopreserved, or a combination thereof. In some cases, the one or more compaction agents may be stored in combination with a glycerol solution.

In some embodiments, the kit may include a buffer. The buffer may comprise a variety of components. In some embodiments, the buffer may comprise one or more of any one of the chemicals disclosed herein. The buffer may comprise a salt. In some cases, the salt may comprise NaCl, MgCl2, or CaCl2, or a combination there. In some cases, the buffer may comprise a solvent. The solvent may be polar. In some embodiments, the buffer may comprise a chaotropic reagent, as described herein. In some embodiments, the kit may include a reagent. The reagent may comprise a variety of components. In some embodiments, the reagent may comprise one or more of any one of the chemicals disclosed herein. The reagent may comprise a salt. In some cases, the salt may comprise NaCl, MgCl2, or CaCl2, or a combination there. In some cases, the reagent may comprise a solvent. The solvent may be polar. In some embodiments, the reagent may comprise a chaotropic reagent, as described herein. The buffer and/or reagent may be contained within a vial, a tube, a container, a box, a bottle, or a combination thereof.

### Computer Systems

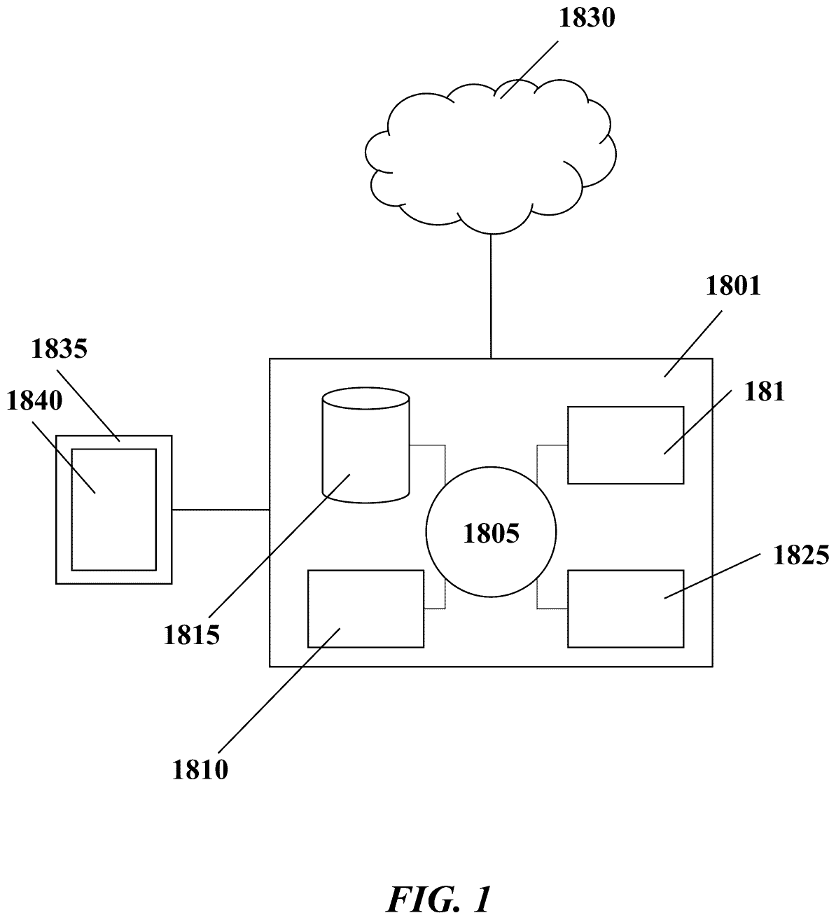

The present disclosure provides computer systems that are programmed to implement methods of the disclosure. FIG. 1 shows a computer system 1801 that is programmed or otherwise configured to control detection of analyte proximity or analysis thereof. The computer system 1801 can regulate various aspects of analyte proximity detection and analysis thereof, such as, for example, temperature control, humidity control, fluid control, camera settings, image analysis, etc. The computer system 1801 can be an electronic device of a user or a computer system that is remotely located with respect to the electronic device. The electronic device can be a mobile electronic device.

The computer system 1801 includes a central processing unit (CPU, also “processor” and “computer processor” herein) 1805, which can be a single core or multi core processor, or a plurality of processors for parallel processing. The computer system 1801 also includes memory or memory location 1810 (e.g., random-access memory, read-only memory, flash memory), electronic storage unit 1815 (e.g., hard disk), communication interface 181 (e.g., network adapter) for communicating with one or more other systems, and peripheral devices 1825, such as cache, other memory, data storage and/or electronic display adapters. The memory 1810, storage unit 1815, interface 181 and peripheral devices 1825 are in communication with the CPU 1805 through a communication bus (solid lines), such as a motherboard. The storage unit 1815 can be a data storage unit (or data repository) for storing data. The computer system 1801 can be operatively coupled to a computer network (“network”) 1830 with the aid of the communication interface 181. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the computer system 1801, can implement a peer-to-peer network, which may enable devices coupled to the computer system 1801 to behave as a client or a server.

The CPU 1805 can execute a sequence of machine-readable instructions, which can be embodied in a program or software. The instructions may be stored in a memory location, such as the memory 1810. The instructions can be directed to the CPU 1805, which can subsequently program or otherwise configure the CPU 1805 to implement methods of the present disclosure. Examples of operations performed by the CPU 1805 can include fetch, decode, execute, and writeback.

The CPU 1805 can be part of a circuit, such as an integrated circuit. One or more other components of the system 1801 can be included in the circuit. In some cases, the circuit is an application specific integrated circuit (ASIC).

The storage unit 1815 can store files, such as drivers, libraries, and saved programs. The storage unit 1815 can store user data, e.g., user preferences and user programs. The computer system 1801 in some cases can include one or more additional data storage units that are external to the computer system 1801, such as located on a remote server that is in communication with the computer system 1801 through an intranet or the Internet.

The computer system 1801 can communicate with one or more remote computer systems through the network 1830. For instance, the computer system 1801 can communicate with a remote computer system of a user (e.g., an automated greenhouse). Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the computer system 1801 via the network 1830.

Methods as described herein can be implemented by way of machine (e.g., computer processor) executable code stored on an electronic storage location of the computer system 1801, such as, for example, on the memory 1810 or electronic storage unit 1815. The machine executable or machine-readable code can be provided in the form of software. During use, the code can be executed by the processor 1805. In some cases, the code can be retrieved from the storage unit 1815 and stored on the memory 1810 for ready access by the processor 1805. In some situations, the electronic storage unit 1815 can be precluded, and machine-executable instructions are stored on memory 1810.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1801, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1801 can include or be in communication with an electronic display 1835 that comprises a user interface (UI) 1840 for providing, for example, operating parameters and conditions, options to control the temperature, humidity level or flow rate, volume dispensed, process progress, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1805. The algorithm can, for example, calculate values, measure variances, analyze image data, analyze tabulated data, measure minimum values, measure maximum values, analyze mass spectrometry data, or calculate flow rates.

### EXEMPLARY EMBODIMENTS

The following embodiments recite nonlimiting permutations of a combination of features disclosed herein. Other permutations of a combinations of features are also contemplated. In particular, each of these numbered embodiments is contemplated as depending from or related to every previous or subsequent numbered embodiments, independent of their order listed.

Embodiment 1: A method of volumetric imaging of a sample, the method comprising: (a) providing a three-dimensional sample disposed on a stage; (b) providing an imaging module, imager, imaging device, or imaging system configured to create an image, the imaging module, imager, imaging device, or imaging system comprising an objective lens configured to transmit photons from one or more object planes within the sample to one or more sensors; and (c) moving the objective lens relative to the sample while simultaneously using the imaging module, imager, imaging device, or imaging system, to acquire a series of images corresponding to a plurality of object planes within the sample.

Embodiment 2: A system for volumetric imaging of a sample, the system comprising: a stage configured to hold a three-dimensional sample; and an imaging module, imager, imaging device, or imaging system configured to create an image, the imaging module, imager, imaging device, or imaging system, comprising an objective lens configured to transmit photons from one or more object planes within the sample to one or more sensors; wherein the imaging module, imager, imaging device, or imaging system, is configured to move the objective lens relative to the sample in a direction substantially toward or away from the sample while simultaneously using the imaging module, imager, imaging device, or imaging system to acquire a series of images corresponding to the one or more object planes within the sample.

Embodiment 3: The method or system of any of the preceding embodiments, wherein the objective lens is moved toward the sample.

Embodiment 4: The method or system of any of the preceding embodiments, wherein the objective lens is moved away from the sample.

Embodiment 5: The method or system of any of the preceding embodiments, wherein the objective lens is moved in a direction substantially parallel to an optical axis of the objective.

Embodiment 6: The method or system of any of the preceding embodiments, wherein the objective lens is moved substantially continuously during a time period at which the imaging module simultaneously acquires the series of images.

Embodiment 7: The method or system of any of the preceding embodiments, wherein the series of images correspond to a plurality of adjacent object planes within the sample.

Embodiment 8: The method or system of any of the preceding embodiments, wherein the series of images comprise a video.

Embodiment 9: The method or system of any of the preceding embodiments, wherein the sensor is a complementary metal-oxide-semiconductor (CMOS) sensor.

Embodiment 10: The method or system of any of the preceding embodiments, wherein the sensor is a rolling shutter sensor.

Embodiment 11: The method or system of any of the preceding embodiments, wherein the sensor is a global shutter sensor.

Embodiment 12: The method or system of any of the preceding embodiments, wherein the sensor comprises an array of pixels.

Embodiment 13: The method or system of any of the preceding embodiments, wherein the array of pixels is organized into multiple groups of pixels, whereby each group of pixels are read in series while the remaining groups of pixels are integrating photons.

Embodiment 14: The method or system of any of the preceding embodiments, wherein the sensor integrates photons with a duty cycle of greater than about 90%.

Embodiment 15: The method or system of any of the preceding embodiments, wherein the imaging module, imager, imaging device, or imaging system is a confocal microscope.

Embodiment 16: The method or system of any of the preceding embodiments, wherein the imaging module, imager, imaging device, or imaging system is a light sheet microscope.

Embodiment 17: The method or system of any of the preceding embodiments, wherein the imaging module, imager, imaging device, or imaging system comprises sensors imaging in multiple passbands.

Embodiment 18: The method or system of any of the preceding embodiments, wherein the objective lens transmits photons to a plurality of sensors.

Embodiment 19: The method or system of any of the preceding embodiments, wherein each sensor of the plurality of sensors integrates photons having a different wavelength.

Embodiment 20: The method or system of any of the preceding embodiments, wherein each sensor of the plurality of sensors is oriented to produce parallel object planes.

Embodiment 21: The method or system of any of the preceding embodiments, wherein the object plane is angled relative to the stage.

Embodiment 22: The method or system of any of the preceding embodiments, wherein the object plane is not orthogonal to the optical axis.

Embodiment 23: The method or system of any of the preceding embodiments, wherein an angle of the object plane relative to the optical axis is less than about 1 milliradian.

Embodiment 24: The method of any of the preceding embodiments, further comprising applying a mathematical transformation to the series of images to correct for an angle relative to the optical axis.

Embodiment 25: The method or system of any of the preceding embodiments, wherein the objective lens is moved, and the sample is stationary.

Embodiment 26: The method or system of any of the preceding embodiments, wherein the sample is moved, and the optical lens is stationary.

Embodiment 27: The method or system of any of the preceding embodiments, wherein a relative distance between the objective lens and the sample is increasing.

Embodiment 28: The method or system of any of the preceding embodiments, wherein a relative distance between the objective lens and the sample is decreasing.

Embodiment 29: The method or system of any of the preceding embodiments, wherein the objective lens is moved relative to the sample until a field of view has been imaged to a chosen depth.

Embodiment 30: The method or system of any of the preceding embodiments, wherein the series of images cover a field of view.

Embodiment 31: The method of any of the preceding embodiments, further comprising repeating (a)-(d) to provide a volumetric image at a plurality of adjacent fields of view.

Embodiment 32: the method or system of any of the preceding embodiments, wherein the volumetric image at the plurality of adjacent fields of view are mathematically joined into a continuous imaged volume.

Embodiment 33: The method or system of any of the preceding embodiments, wherein the plurality of adjacent fields of view are imaged spanning an imaged volume of the sample.

Embodiment 34: The method of any of the preceding embodiments, further comprising moving the objective lens relative to the sample in a direction substantially perpendicular to the optical axis of the objective, such that the imaging module, imager, imaging device, or imaging system is capable of imaging a second field of view of the sample.

Embodiment 35: The method or system of any of the preceding embodiments, wherein a velocity of the objective lens relative to the sample varies by less than about 5% during a period of time when the objective lens is continuously moved relative to the sample.

Embodiment 36: The method or system of any of the preceding embodiments, wherein the objective lens is moved relative to the sample at a velocity such that a second object plane of the adjacent object planes is stacked on a first object plane of the adjacent object planes.

Embodiment 37: The method or system of any of the preceding embodiments, wherein each of the series of images has a depth of focus.

Embodiment 38: The method or system of any of the preceding embodiments, wherein each of the series of images is separated by approximately one depth of focus.

Embodiment 39: The method or system of any of the preceding embodiments, wherein a velocity of the objective lens relative to the sample is coordinated with a frame rate of the sensor such that the series of images are separated by approximately one depth of focus.

Embodiment 40: The method or system of any of the preceding embodiments, wherein the sample is illuminated.

Embodiment 41: The method or system of any of the preceding embodiments, wherein the sample is illuminated with a laser.

Embodiment 42: The method or system of any of the preceding embodiments, wherein the sample is illuminated at one or more portions of the sample corresponding to the object plane.

Embodiment 43: The method or system of any of the preceding embodiments, wherein the imaging module, imager, imaging device, or imaging system comprises a spinning disk.

Embodiment 44: The method or system of any of the preceding embodiments, wherein the spinning disk is configured to enrich the transmitted photons to photons that arise from the object plane within the sample.

Embodiment 45: The method or system of any of the preceding embodiments, wherein the sample is a tissue sample.

Embodiment 46: The method or system of any of the preceding embodiments, wherein the sample is a cleared and hydrogel stabilized tissue sample.

Embodiment 47: The method or system of any of the preceding embodiments, wherein the sample comprises fluorescently labeled loci.

Embodiment 48: The method or system of any of the preceding embodiments, wherein the fluorescently labeled loci are associated with locations of a biomolecule.

Embodiment 49: The method or system of any of the preceding embodiments, wherein the volumetric image of the sample indicates locations at which an RNA is expressed, or a protein is produced.

Embodiment 50: The method or system of any of the preceding embodiments, wherein the volumetric image comprises locations at which a fluorescently labeled nucleotide has been incorporated into a growing nucleic acid strand.

Embodiment 51: The method or system of any of the preceding embodiments, wherein the volumetric image comprises locations at which a fluorescently labeled oligonucleotide has been ligated onto a nucleic acid template.

Embodiment 52: The method or system of any of the preceding embodiments, wherein the growing nucleic acid strand is at least partially complimentary to a sequence of interest.

Embodiment 53: The method of any of the preceding embodiments, wherein (b)-(d) are repeated for incorporation of each of type of nucleotide (A, C, T, or G, or any combination thereof), thereby determining a nucleotide sequence of the sequence of interest.

Embodiment 54: The method or system of any of the preceding embodiments, wherein the tissue sample comprises at least 500,000 cells.

Embodiment 55: The method or system of any of the preceding embodiments, wherein at least about 500 sequences of interest are sequenced.

Embodiment 56: The method or system of any of the preceding embodiments, wherein the series of images are acquired with a duty cycle of at least about 90%.

Embodiment 57: The method or system of any of the preceding embodiments, wherein the series of images are acquired at a rate of at least about 100,000,000 voxels/second on each of two or more wavelength channels.

Embodiment 58: The method or system of any of the preceding embodiments, wherein the series of images comprise at least 50 images.

Embodiment 59: The method of any of the preceding embodiments, further comprising performing fluidic operations on the sample.

Embodiment 60: The method of any of the preceding embodiments, wherein the fluidic operations comprise labeling, stripping, aspirating, dispensing, incubating, or any combination thereof.

Embodiment 61: The method or system of any of the preceding embodiments, wherein multiple samples are loaded onto multiple areas of the stage.

Embodiment 62: The method or system of any of the preceding embodiments, wherein the series of images are acquired from a first sample while a second sample is undergoing a fluidic operation or an incubation period.

Embodiment 63: The method or system of any of the preceding embodiments, wherein the series of images comprise signals acquired at fixed locations within the sample.

Embodiment 64: The method of any of the preceding embodiments, further comprising associating the signals and locations with a reference database.

Embodiment 65: The method of any of the preceding embodiments further comprising extracting signals from the series of images within 20 seconds of acquiring the series of images.

Embodiment 66: The method or system of any of the preceding embodiments, wherein the signals are extracted from the series of images without saving the series of images.

The following examples are included for illustrative purposes only and are not intended to limit the scope of the invention.

### EXAMPLES

The following examples are included for illustrative purposes only and are not intended to limit the scope of the invention.

### Example 1: Chemical-Mediated Intra-Amplicon Crosslinking for Amplicon Condensing with 250 mRNA Targets in Tissues

In this example, amplicons were crosslinked to themselves through incorporated reactive moieties and bivalent crosslinker to reduce amplicon size.

A 20 μm thick fresh frozen mouse brain tissue section was sliced onto a coverslip. The sample was fixed with formaldehyde and incubated with a set of padlock and primer probe sequences. The padlock probe sequences of the set of padlock probe sequences included a target binding sequence that recognized one of 250 transcripts, and a barcode sequence that was uniquely associated with one of the mRNA targets. An additional oligonucleotide sequence was added corresponding to a padlock probe sequence that included a target-binding sequence adjacent to the analyte binding sequence on the padlock probe and a padlock probe binding sequence that bound to 5′ and 3′ end of the corresponding padlock probe sequence. Upon binding, the padlock probe sequences, and additional oligonucleotides sequences formed complexes on the mRNA target sequences. A ligase was added to the sample to form a circular oligonucleotide and phi29 polymerase was added to initiate a rolling circle amplification to form an amplification product at a location on an mRNA bound by the padlock probe sequences. A modified dUTP, 5-DBCO-PEG4-dUTP or Azide-PEG4-dUTP (for intra-amplicon crosslinking) with an optional 5-Acrydite-aminoallyl-dUTP (for covalent hydrogel crosslinking, which may not be required for maintaining the amplicon size within certain time due to physical confinement of the amplicon in hydrogel) were spiked-in to the dNTP during the rolling circle amplification to incorporate the reactive moiety into the RCA product. Unreacted modified-dUTP was washed out, a crosslinker solution was added to the tissue sample to crosslink the incorporated DBCO or azide moiety with each other (a scheme is shown in FIG. 2). The tissue sample was further embedded in acrylamide hydrogel and cleared using Protease K. Detection of the amplicons was performed by adding multiple rounds of detection probes to the sample. A representative image of the detected amplicons is shown in FIG. 3. Multiple rounds of decoding were performed and demonstrated that covalent crosslinking to the hydrogel was not essential for the stability of amplicons in hydrogel.

### Example 2: Proximity-Mediated Intra-Amplicon Crosslinking for Amplicon Condensing with 250 mRNA Targets in Tissues

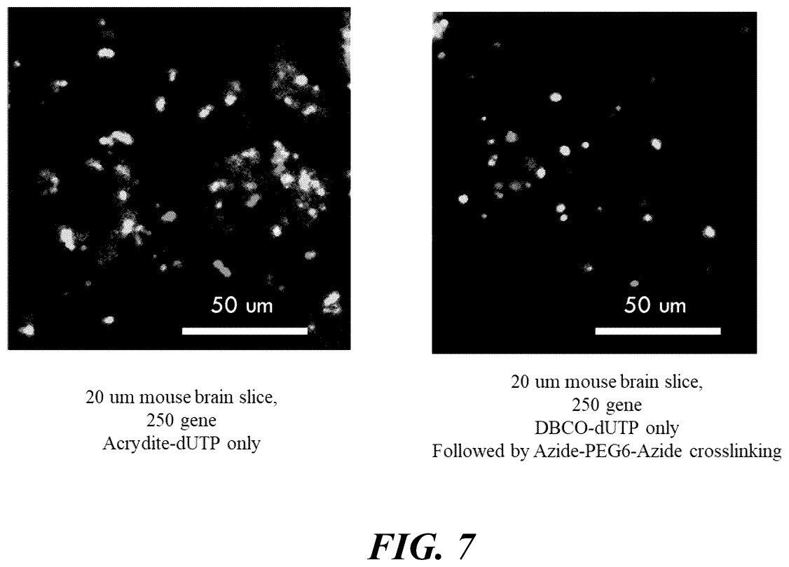

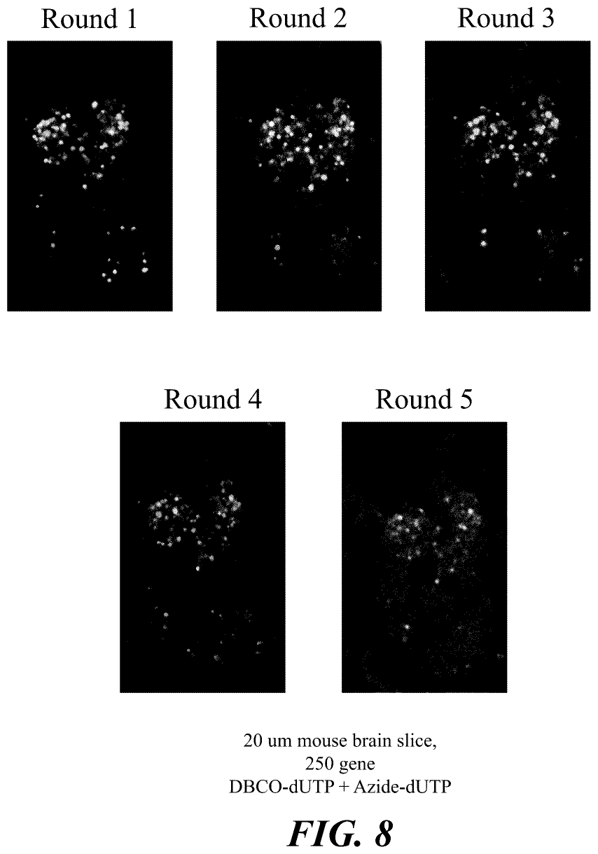

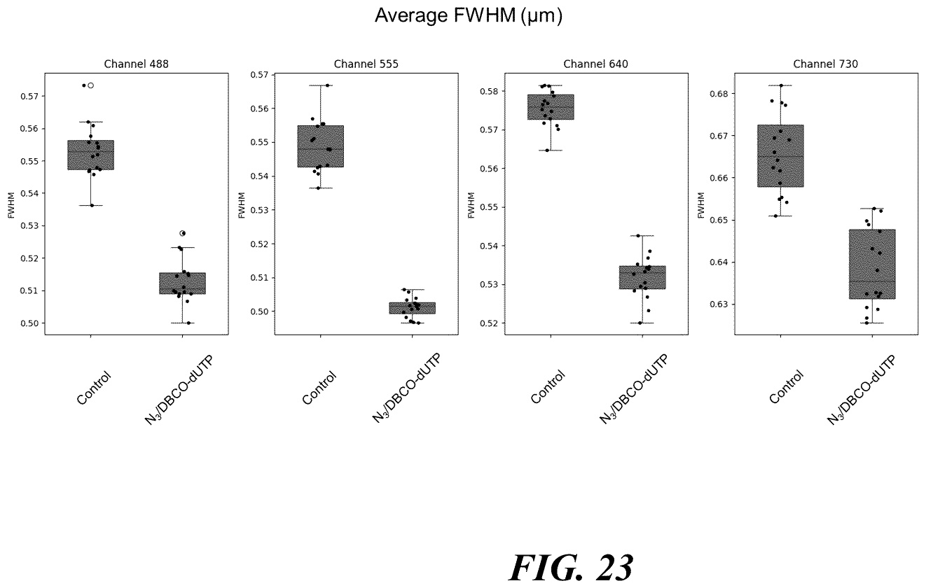

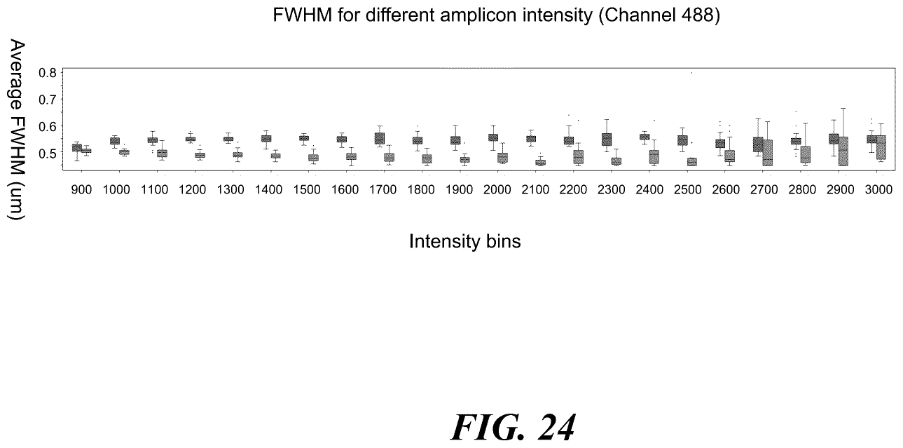

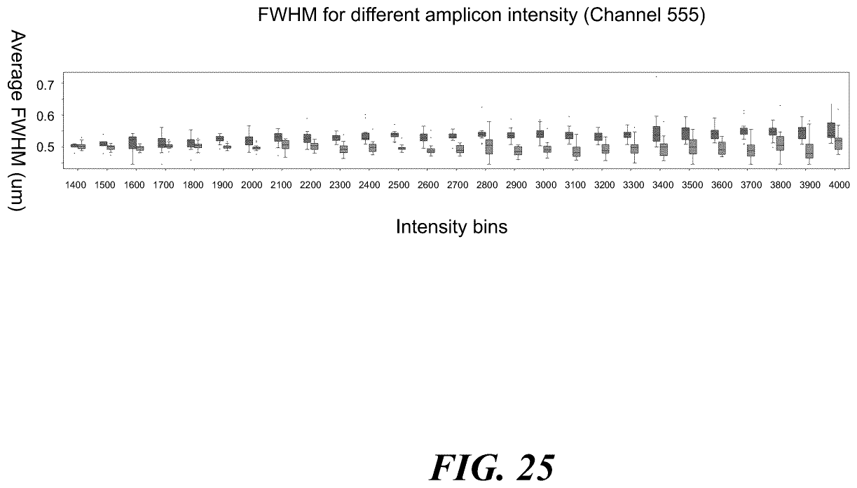

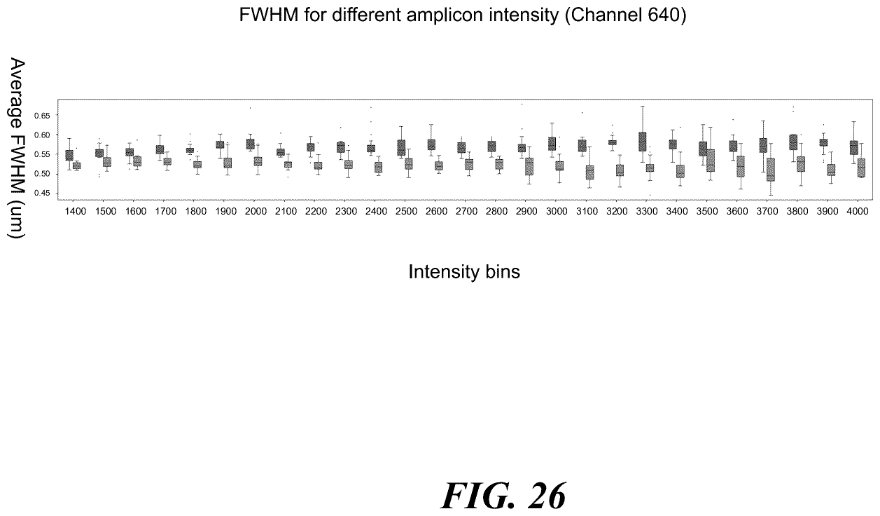

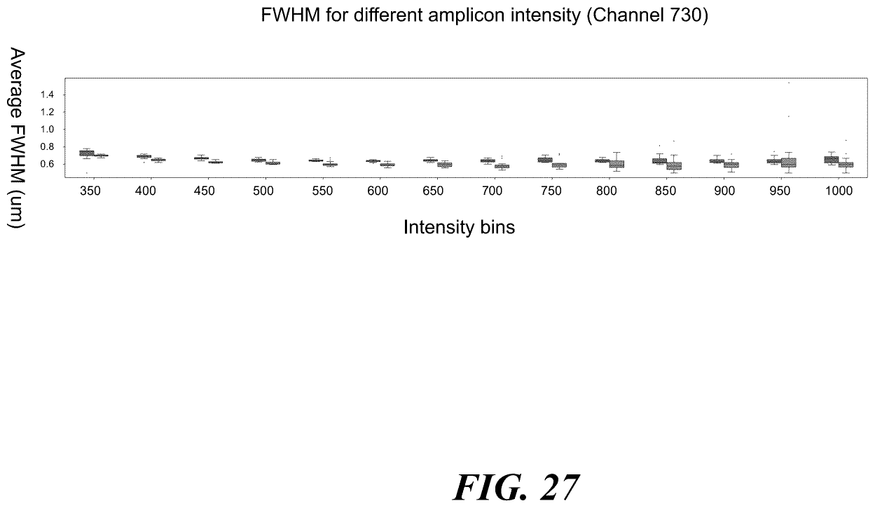

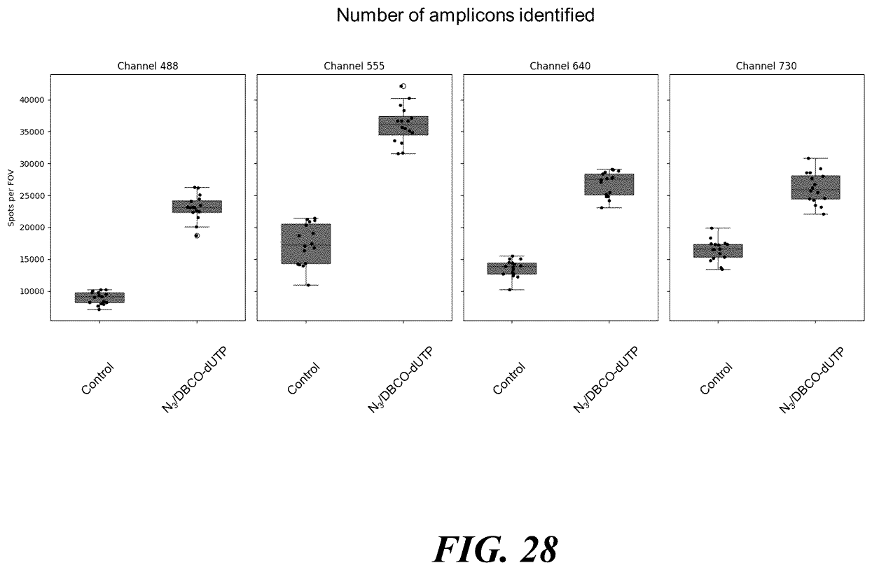

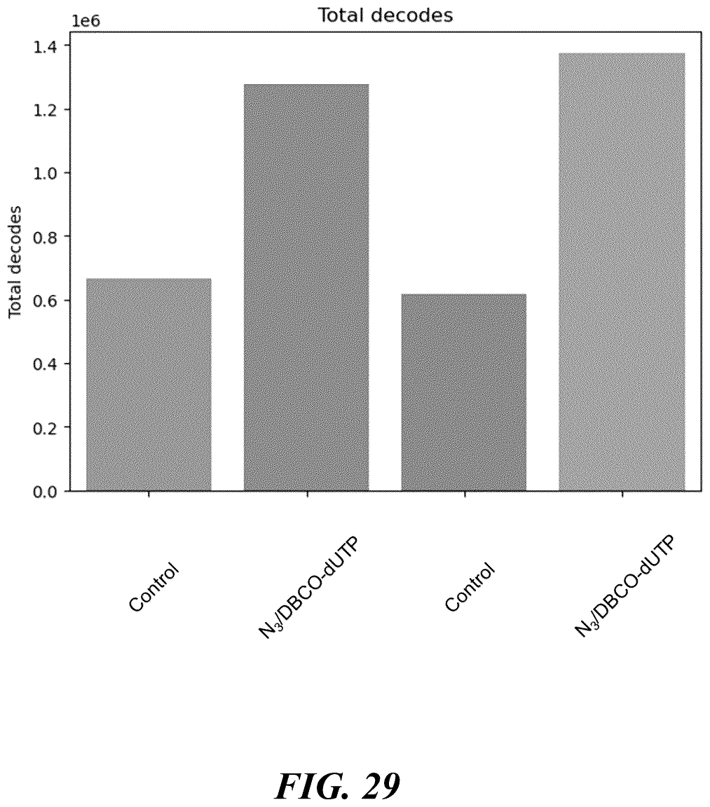

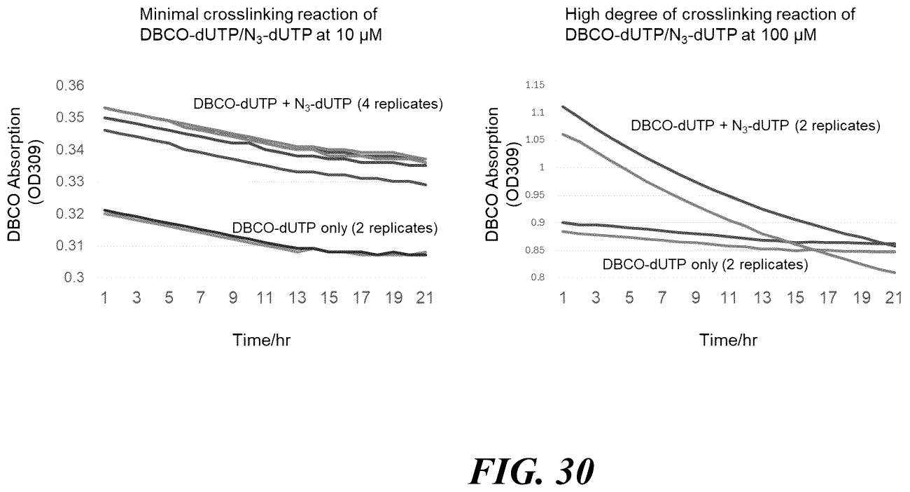

In this example, amplicons were crosslinked to themselves through two incorporated reactive moieties promoted by proximity without additional crosslinker to reduce amplicon size. A 20 μm thick fresh frozen mouse brain tissue section was sliced onto a coverslip. The sample was fixed with formaldehyde and incubated with a set of padlock and primer probe sequences. One of the padlock probe sequences of the set of padlock probe sequences included a target binding sequence that recognized one of 250 transcripts, and a barcode sequence that was uniquely associated with one of the mRNA targets. An additional oligonucleotide sequence was added corresponding to a padlock probe sequence that included a target-binding sequence adjacent to the analyte binding sequence on the padlock probe and a padlock probe binding sequence that bound to 5′ and 3′ end of the corresponding padlock probe sequence. Upon binding, the padlock probe sequences, and additional oligonucleotides sequences formed complexes on the mRNA target sequences. A ligase was added to the sample to form a circular oligonucleotide and phi29 polymerase was added to initiate a rolling circle amplification to form an amplification product at a location on an mRNA bound by the padlock probe sequences. Two modified dUTPs, 5-DBCO-PEG4-dUTP and Azide-PEG4-aminoallyl-dUTP (for intra-amplicon crosslinking) with an optional 5-Acrydite-aminoallyl-dUTP (for covalent hydrogel crosslinking) were spiked-in to the dNTP during the rolling circle amplification to incorporate the reactive moiety into the RCA product. Unreacted modified-dUTP was washed out, the tissue sample was incubated at room temperature for 3 hours to allow the incorporated DBCO and azide groups to crosslink with each other within a single amplicon (a scheme was shown in FIG. 3). Additional samples were processed similarity but without the modified dUTPs during amplification (controls samples). The controls samples were not expected to produce compacted amplicons because of the omission of the modified dUTPs with either the DBCO or azide modifications. Additionally, solutions comprising either just DBCO-dUTP or a combination of DBCO-dUTP and azide (N₃) dUTP were prepared at two different dUTP concentration: 10 μM and 100 μM. These solutions did not comprise a tissue sample nor amplicons. An absorbance reading at an optical density of 309 nm was analyzed over time for the different experimental conditions. Decreased optical density at 309 nm suggests a formation of a triazole ring between the DBCO-dUTP and azide-dUTP. FIG. 30 shows data from this experiment. The solutions comprising 10 μM dUTP concentration showed less of a decrease of absorbance at 309 nm over time, which suggest minimal crosslinking between DBCO-dUTP and azide-dUTP. The solution comprising 100 μM dUTP concentration showed a much greater decrease of absorbance at 309 nm over time, which suggest a higher degree of crosslinking between DBCO-dUTP and azide-dUTP. This data may suggest that intra-amplicon cross-linking (e.g., compacting) between a DBCO chemical reactive moiety and an azide chemical reactive moiety within an amplicon may occur on a time scale more similar to the 100 μM solution based dUTP condition compared to the lower concentration condition. Detection of the amplicons was performed by adding multiple rounds of detection probes to the sample. A representative image of the detected amplicons is shown in the image FIG. 8. Multiple rounds of decoding were performed and demonstrated that amplicons remained stable in the hydrogel across multiple rounds of decoding. Another representative image of the detected amplicons is shown in FIG. 7 to show increased amplicon intensities compared to with acrydite-dUTP only. Image data from these rounds of decoding was analyzed to assess an average FWHM of signals associated with amplicons. Spots of fluorescent intensities were measured based on size and intensity for data acquired across four fluorescent channels: 488 nm, 555 nm, 640 nm, and 730 nm. FWHM values were determined for fluorescent signals for each fluorescent channel for the experimental conditions with compacting (with inclusion of dUTPs with DBCO and azide) and without compacting (control samples). The average FWHM values across multiple analyzed amplicons is shown in FIG. 23. As shown, the average FWHM was decreased for the compacted amplicons compared to the control amplicons. This data was separated into intensity bins to evaluate the FWHM of compacted amplicons and not compacted amplicons (control amplicons) having similar intensities. The binned data for amplicons detected in the 488 nm fluorescent channel is shown in FIG. 24. The plotted bars in FIG. 24 represent the average FWHM of all amplicons in one field of view for the control sample (left bar) and the compacted sample (right bar) for each intensity bin. The values on x axis of FIG. 24 represent a maximum for the intensity bin at each value and the bin size is 100 arbitrary units. The binned data for amplicons detected in the 555 nm fluorescent channel is shown in FIG. 25. The plotted bars in FIG. 25 represent the average FWHM of all amplicons in one field of view for the control sample (left bar) and the compacted sample (right bar) for each intensity bin. The values on x axis of FIG. 25 represent a maximum for the intensity bin at each value and the bin size is 100 arbitrary units. The binned data for amplicons detected in the 640 nm fluorescent channel is shown in FIG. 26. The plotted bars in FIG. 26 represent the average FWHM of all amplicons in one field of view for the control sample (left bar) and the compacted sample (right bar) for each intensity bin. The values on x axis of FIG. 26 represent a maximum for the intensity bin at each value and the bin size is 100 arbitrary units. The binned data for amplicons detected in the 730 nm fluorescent channel is shown in FIG. 27. The plotted bars in FIG. 27 represent the average FWHM of all amplicons in one field of view for the control sample (left bar) and the compacted sample (right bar) for each intensity bin. The values on x axis of FIG. 27 represent a maximum for the intensity bin at each value and the bin size is 50 arbitrary units. The data in FIGS. 24-27 shows that compacted amplicons with similar intensities to non-compacted amplicons have lower average FWHM values. Image data for the compacted and non-compacted samples were analyzed to identify a total number of amplicons across each fluorescent channel by identifying the number of separable fluorescent spots above a threshold value within a field of view. The field of view (FOV) was the same size for the image analysis of compacted data and non-compacted data. FIG. 28 shows the spot counts per FOV for each florescent channel for the compacted samples (N₃/DBCO-dUTP) and non-compacted samples (control). Higher number of spots (amplicons) were detected in the FOVs associated with the compacted amplicons compared with the non-compacted amplicons. Image data across multiple cycles of detection to reveal information about barcodes associated with the amplicons of the compacted samples and the non-compacted samples was analyzed to identify (e.g., decode) analytes associated with the amplicons. The total number of amplicons that were identified as being associated with an analyte based on identifying a correct barcode sequence was calculated for the compacted and the non-compacted samples. The total count of identified amplicons (e.g., decoded amplicons) is shown in FIG. 29 for two sample replicates of each condition. The first two bars of FIG. 29 show the number of identified amplicons for the first sample replicate of the control sample and the compacted sample (N₃/DBCO-dUTP). The third and fourth bars of FIG. 29 show the number of identified amplicons for the second sample replicate of the control sample and the compacted sample (N₃/DBCO-dUTP), respectively.

### Example 3: Non-Covalent Chemical Interaction-Mediated Amplicon Condensing with 459 mRNA Targets in Tissues

In this example, amplicons were condensed through incorporated hydrophobic moieties to reduce amplicon size followed by hydrogel confinement.

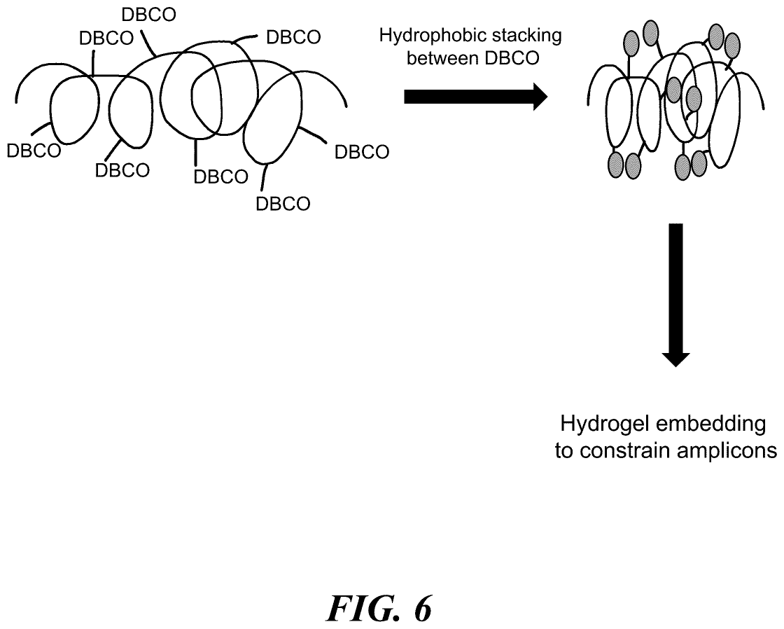

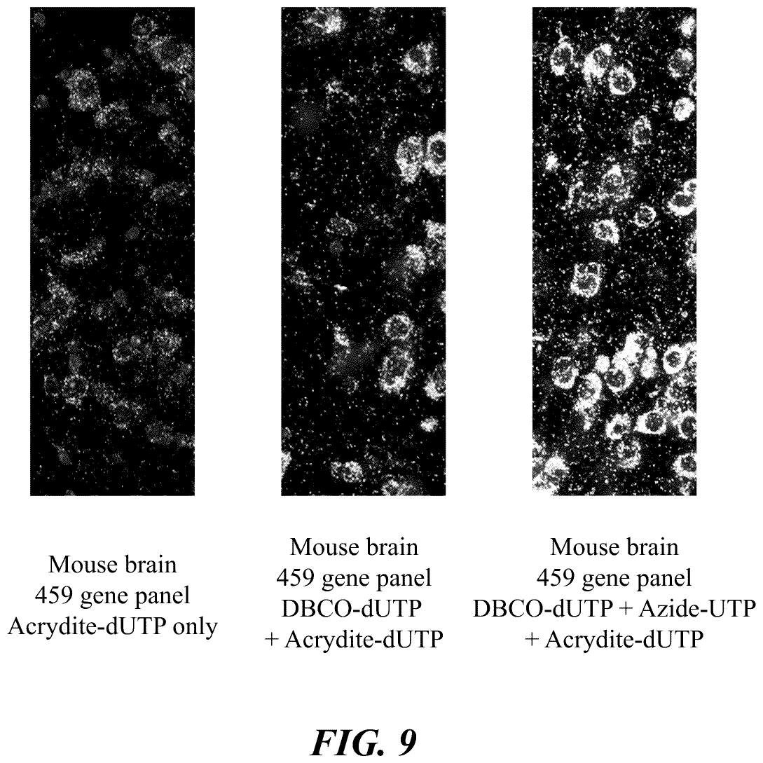

A 20 μm thick fresh frozen mouse brain tissue section was sliced onto a coverslip. The sample was fixed with formaldehyde and incubated with a set of padlock and primer probe sequences. The padlock probe sequences of the set of padlock probe sequences included a target binding sequence that recognized one of 459 transcripts, and a barcode sequence that was uniquely associated with one of the mRNA targets. An additional oligonucleotide sequence was added corresponding to a padlock probe sequence that included a target-binding sequence adjacent to the analyte binding sequence on the padlock probe and a padlock probe binding sequence that binds to 5′ and 3′ end of the corresponding padlock probe sequence. Upon binding, the padlock probe sequences, and additional oligonucleotide sequences formed complexes on the mRNA target sequences. A ligase was added to the sample to form a circular oligonucleotide and phi29 polymerase was added to initiate a rolling circle amplification to form an amplification product at a location on an mRNA bound by the padlock probe sequences. A modified dUTP, 5-DBCO-PEG4-dUTP or TCO-PEG4-dUTP (for intra-amplicon condensing) with and optionally 5-Acrydite-aminoallyl-dUTP (for covalent hydrogel crosslinking, which may not be required for maintaining the amplicon size within certain time due to physical confinement of the amplicon in hydrogel) were spiked-in to the dNTP during the rolling circle amplification to incorporate the reactive moiety into the RCA product (a scheme was shown in FIG. 6). Unreacted modified-dUTP was washed out. The tissue sample was further embedded in acrylamide hydrogel and cleared using Protease K. Detection of the amplicons was performed by adding multiple rounds of detection probes to the sample. A representative image of the detected amplicons is shown in the image FIG. 9 to show increased signal intensities compared to with acrydite-dUTP only.

### Example 4—Imaging System

FIG. 18 is a schematic of the sample stage and objective lens, according to some embodiments of the present disclosure. Included herein is a piezo Z insert 1000 and a 60× WI objective 1002.

FIG. 18 is a schematic of confocal microscopy system, according to some embodiments of the present disclosure. The system can include a Sony IMX571BLR-J camera or kinetix camera 1100, an x-light V3 confocal microscope 1102, an ASI stage with piezo Z insert 1104, and a Nikon Ti2e 1106.

### Example 5—Volume Video Exposure Parameters

A system of the present disclosure configured to image a 500-nanometer z portion of a sample with a 20 hertz video can have a focal shift along the z axis of the acquisition at a rate of 10 micrometers per second. This speed of shift can result in the predetermined 500 nanometer z size of the voxel, as a single frame of the video taken of the sample would achieve the 500-nanometer value. Similarly, the above parameters can be used to determine the scanning speed used to achieve a different given z axis resolution.

Using a rolling shutter sensor with this system can provide facile and cost-effective detection for the volume video system by integrating each row of the sensor individually as the acquisition is taken. Such a system can induce artifacts via a tilt in the x-y (e.g., orthogonal to the optical axis) plane. In this example, a 1000 row sensor with 100 nanometer pixels can have a 5 milliradian skew induced by use of a rolling shutter sensor. Such skew can be removed using post processing algorithms, which can produce high quality data out of a simple and cost-effective instrument.

### Example 6—Volume Video Acquisition

A system of the present disclosure can be provided having a sensor, a sample stage holding a sample comprising a plurality of cells, and a z-axis scanner operably coupled to a lens in an optical path between the sample and the sensor. The plurality of cells can be illuminated while the z-axis scanner moves a focal plane of the sensor through the plurality of cells to generate a plurality of images or signals of the voxel sampled by the focal plane. The shifting, illuminating, and detecting can be repeated for an additional voxel, thereby sampling additional cells of the plurality of cells. In this way, the sample can be quickly imaged with high resolution. The images or signals can be processed to identify a label associated with a nucleic acid in the sample, which can then be used to determine an identity or property of the nucleic acid or the sample by comparing the label to a reference database and associating the property of the reference database indicated by the label with the sample.

In another example, the sensor can be continuously operated to integrate through a voxel generated by the shifting of the focal plane of the sensor by the z-axis scanner through the sample. The continuous integration can generate a volumetric measurement of the entire voxel of the sample, thereby providing a three-dimensional measurement of, for example, one or more signals or one or more images of the sample.

### Example 7—Single Camera Confocal Video Scan

In this example, an intact tissue section was imaged by confocal microscopy with continuous motion in the z-direction. Samples were e imaged in the x-y plane while continuously moving in the z-direction. In some cases, the sample may be moved in the x-y plane and the scan is repeated, leading to a complete volumetric image of a sample, or of a sample array.

Images were acquired using a confocal microscope system (e.g., as shown in FIG. 18 and FIG. 18) with a Sony IMX571BLR-J 2×2 binned CMOS sensor operating with a rolling shutter video mode. The sensor in this binned mode had an effective array of 3122×2084 active pixels with a 7.5 μm pixel size. Using a 60× water immersion objective, this yielded a 390.25 μm×260.5 μm array size on the sample with a 125 nm pixel size. For a 500 nm z voxel size, this gave 0.0019 radians or 0.11° skew between consecutive image planes as the sample was imaged under continuous z-motion.

The experiment was repeated using different combinations of video rate and z-velocity as shown in Table 1. Each of these combinations led to successful volumetric image acquisition and reconstruction after appropriate image correction operations (e.g., correcting for a tilt angle).

TABLE 1
Summary of video rates and z-velocity conditions

| Video Rate (Hz) | Z-velocity (μm/s) |
|---|---|
| 1 | 0.5 |
| 5 | 2.5 |
| 10 | 5 |
| 20 | 10 |
| 40 | 20 |
| 80 | 40 |
| 100 | 50 |
| 150 | 75 |
| 200 | 100 |

The experiment was repeated at other combinations of z-voxel size and sensor height to test different volumetric skew angles. A summary of tested conditions is shown in Table 2. The best images were achieved for skew angles less than about 0.1° skew.

TABLE 2
Volumetric skew angles for representative z-voxel size and sensor height

| Volumetric skew angle | | z-voxel size (nm) | | | | | |
|---|---|---|---|---|---|---|---|
| in volume video (deg) | | 100 | 200 | 400 | 1,000 | 2,000 | 4,000 |
| Sensor height | 100 | 0.0573 | 0.1146 | 0.2292 | 0.5729 | 1.1458 | 2.2906 |
| on sample (μm) | 200 | 0.0286 | 0.0573 | 0.1146 | 0.2865 | 0.5729 | 1.1458 |
| | 400 | 0.0143 | 0.0286 | 0.0573 | 0.1432 | 0.2865 | 0.5729 |
| | 1,000 | 0.0057 | 0.0115 | 0.0229 | 0.0573 | 0.1146 | 0.2292 |
| | 2,000 | 0.0029 | 0.0057 | 0.0115 | 0.0286 | 0.0573 | 0.1146 |
| | 4,000 | 0.0014 | 0.0029 | 0.0057 | 0.0143 | 0.0286 | 0.0573 |

### Example 8—Single Camera Light Sheet Video Scan

In this example, instead of rejecting out of focal plane light with a confocal filter, scattered or fluorescent emission was limited to a plane by light sheet microscopy (e.g., light sheet microscopy as described herein). In this system, illumination and detection objectives were physically decoupled, allowing for visualization of a selective plane of a sample (e.g., as shown in FIG. 10A). This system can be scanned in the z-direction as described in Example 1 above to create 3D image stack but with substantially increased z-voxel size (10 μm to 1 mm).

### Example 9—Analyzing a Tissue Sample Using Amplicon Compacting and a Video Imaging

In this example, a tissue sample will be analyzed.

A 100 μm formalin-fixed paraffin embedded (FFPE) tissue slice will be placed onto the surface of a well plate. The FFPE tissue slice will be processed using an antigen retrieval process. The FFPE tissue slice will be fixed using formaldehyde. The FFPE tissue slice will be treated with methanol. A plurality of binding moieties will be added to the FFPE tissue slice. The plurality of binding moieties will comprise nucleic acids that hybridize to a plurality of RNA targets within the FFPE tissue slice. The nucleic acids of the plurality of binding moieties will comprise sequences that do not hybridize to RNA targets of the plurality of RNA targets within the FFPE tissue slice. The nucleic acids of the plurality of binding moieties will comprise barcodes. The barcodes will correlate to analytes that are bound by the binding moieties. A plurality of probes will be added to the FFPE tissue slice. The plurality of probes will comprise nucleic acids and hybridize to RNA targets of the plurality of RNA targets within the FFPE tissue slice. Probes of the plurality of probes will hybridize to binding moieties of the plurality of binding moieties. Binding of the probes of the plurality of probes to the RNA targets of the plurality of RNA targets and the binding moieties of the plurality of binding moieties will bring ends of the binding moieties of the plurality of binding moieties in close proximity to each other. A ligase will be added to the FFPE tissue slice. The ligase will ligate ends of the binding moieties to each other to generate circular nucleic acids. A polymerase will be added to the FFPE tissue slice. Nucleotides, including modified nucleotides comprising azides and DBCO will be added to the FFPE tissue slice. The polymerase will amplify the circular nucleic acids using the probes of the plurality of probes as primers for amplification. The modified nucleotides will be incorporated into amplicons that are formed as a product of amplification. The modified nucleotides of the amplicons will form intra-amplicon cross-links, thereby compacting the amplicons to generate compacted amplicons. Detection probes will be added to the FFPE tissue slice. The detection probes will bind to the compacted amplicons in a sequence dependent manner based on detection of the barcodes or reverse complement thereof in the compacted amplicons. Volume image data of the FFPE tissue slice will be generated using an imaging system. The imaging system will image multiple regions of the FFPE tissue slice. At each region of multiple regions of the FFPE tissue slice, the imaging system will continuously image through z-planes of the FFPE tissue slice to generate the volume image data. Multiple rounds of detection will be performed. During each round of detection, detection probes will be added to the FFPE tissue slice, volume image data will be acquired, and the detection probes will be removed from the FFPE tissue slice. The volume image data from each round of detection will be analyzed. Amplicons will be identified based on spots in the volume image data. Signals associated with the volume image data at locations within the sample across multiple rounds of detection and across multiple fluorescent channels acquired per round will be analyzed and compared to barcode information. Barcode will be assigned to spots within the volume image data. The barcodes will correlate to analytes of the FFPE sample. Based on the barcode and analyte correlations, a spatial map comprising a location of analytes within the FFPE sample will be generated.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein can be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for detecting analytes the method comprising:
(a) providing a matrix comprising a plurality of cells, and wherein the plurality of cells comprises a plurality of analytes;
(b) contacting the matrix with a plurality of binding moieties, wherein a binding moiety of the plurality of binding moieties recognizes and binds to an analyte of the plurality of analytes;
(c) subsequent to (b), contacting the matrix with a plurality of detection probes to form a plurality of complexes between binding moieties of the plurality of binding moieties and detection probes of the plurality of detection probes or derivatives of the binding moieties of the plurality of binding moieties and detection probes of the plurality of detection probes; and
(d) detecting the plurality of complexes to thereby identify the plurality of analytes, wherein identifying the plurality of analytes comprises identifying more than 120 analytes on average per cell.

2. The method of claim 1, wherein said matrix is a tissue sample.

3. The method of claim 2, wherein said tissue sample is a formalin-fixed paraffin embedded tissue sample.

4. The method of claim 3, wherein said formalin-fixed paraffin embedded tissue sample is 5-250 micrometers (μm) thick.

5. The method of claim 1, wherein said matrix is embedded in a hydrogel.

6. The method of claim 1, wherein a binding moiety of said plurality of binding moieties comprises a nucleic acid.

7. The method of claim 6, wherein said nucleic acid comprises a ribonucleic acid.

8. The method of claim 1, wherein a binding moiety of said plurality of binding moieties comprises a nucleic acid barcode.

9. The method of claim 1, after (b), further performing an amplification reaction.

10. The method of claim 9, wherein said amplification reaction comprises a rolling circle amplification reaction to form a plurality of amplicons.

11. The method of claim 10, wherein an amplicon of said plurality of amplicons comprises a first reactive chemical moiety and a second reactive chemical moiety.

12. The method of claim 11, wherein said first reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof.

13. The method of claim 11, wherein said second reactive chemical moiety comprises an azide, an alkyne, an amine, a carboxyl, a sulfhydryl, a carboxylic acid, a maleimide, an NHS-ester, a carbodiimide, an imidoester, a haloacetyl, a pyridyldisulfide, a hydrazide, an alkoxyamine, a diazirine, a phosphine, an epoxide, an aldehyde, or a combination thereof.

14. The method of claim 11, further comprising cross-linking said first reactive chemical moiety and said second reactive chemical moiety.

15. The method of claim 14, wherein said cross-linking comprises use of a linker.

16. The method of claim 15, wherein said linker comprises a polyethylene glycol.

17. The method of claim 15, wherein said linker comprises a methylene group.

18. The method of claim 14, wherein a diameter of said amplicon is reduced after cross-linking.

19. The method of claim 18, wherein said diameter of said amplicon is reduced by at least 20% after cross-linking.

20. The method of claim 1, wherein (d) comprises imaging said matrix using an imaging system.

21. The method of claim 19, wherein said imaging system is a light sheet microscope.

22. The method of claim 19, wherein said imaging system is a confocal microscope.
